(19) United States
(12) Patent Application Publication
(10) Pub. No.: US 2025/0066286 A1
(43) Pub. Date: Feb. 27, 2025

(54) COMPOSITIONS OF (Z)-ENDOXIFEN AND METHODS OF ENRICHMENT THEREOF

(71) Applicant: Atossa Therapeutics, Inc., Seattle, WA (US)

(72) Inventors: Steven C. Quay, Seattle, WA (US); Yao-Lin SUN, Kaohsiung City (TW); Wei-Jen LO, Tainan City (TW); Kun-Chih HUNG, Tainan City (TW); Chia-Chen HSU, Tainan City (TW)

(21) Appl. No.: 18/727,495

(22) PCT Filed: Jan. 11, 2023
(86) PCT No.: PCT/US2023/010567

§ 371 Date: Jul. 9, 2024

Related U.S. Application Data
(60) Provisional application No. 63/298,908, filed on Jan. 12, 2022.
(60) Provisional application No. 63/437,045, filed on Jan. 4, 2023.

(51) Int. Cl. C07C 217/20

(52) U.S. Cl. CPC C07C 217/20

(57) ABSTRACT

Described herein are methods of making (Z)-endoxifen, a salt thereof, crystalline forms of endoxifen, and compositions comprising them. Also described herein are crystalline forms of (Z)-endoxifen. A method of making (Z)-endoxifen may include one or more enrichment steps to enrich the amount of (Z)-endoxifen present in a composition. Enrichment may include one or more steps of crystallization, recrystallization, or fractional recrystallization to reduce the level of one or more impurities in the composition. These methods may be industrially scalable. Also described herein are compositions enriched for (Z)-endoxifen produced by the methods described herein.

### CROSS-REFERENCE

The present application claims the benefit of U.S. Provisional Application No. 63/298,908, entitled “METHODS OF ENRICHING (Z)-ENDOXIFEN,” filed on Jan. 12, 2022, and U.S. Provisional Application No. 63/437,045, entitled “METHODS OF ENRICHING (Z)-ENDOXIFEN,” filed on Jan. 4, 2023, which applications are herein incorporated by reference in their entireties for all purposes.

### BACKGROUND

Breast cancer is by far the most common form of cancer in women, and it is the second leading cause of cancer death in humans. Selective estrogen receptor modulators, such as Tamoxifen, are used for the treatment of women with endocrine responsive breast cancer, e.g., hormone dependent or hormone-sensitive breast cancer. Endoxifen is a potent selective estrogen receptor modulator (SERM). However, the (Z)-isomer ((Z)-endoxifen) has more potent anti-estrogen activity than the related (E)-isomer ((E)-endoxifen). Hence, there remains a need for methods to purify (Z)-endoxifen and to enrich compositions for (Z)-endoxifen.

### SUMMARY

The present disclosure provides industrially scalable synthetic methods for producing highly pure (Z)-endoxifen, as well as pharmaceutical compositions the synthesized (Z)-endoxifen. The methods provided herein are particularly useful for reducing and/or removing mesityl oxide as an impurity. Also provided herein are crystalline forms of (Z)-endoxifen.

In various aspects, the present disclosure provides a method of purifying a compound of Formula (IV),

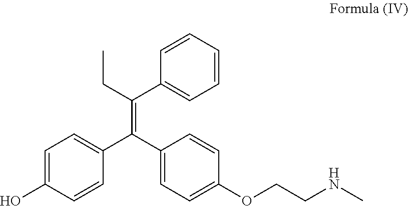 [SMILES: CC/C(=C(\c1ccc(O)cc1)c1ccc(OCCNC)cc1)c1ccccc1]

the method comprising: providing a compound of Formula (III) that comprises the compound of Formula (IV);

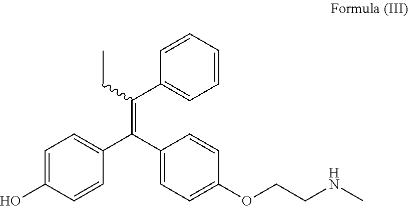 [SMILES: CCC(=C(c1ccc(O)cc1)c1ccc(OCCNC)cc1)c1ccccc1]

performing an ethyl acetate fractional crystallization of the compound of Formula (III) to produce a filtrate comprising the compound of Formula (IV); performing an acetone recrystallization using the filtrate; and performing a tetrahydrofuran recrystallization to purify the compound of Formula (IV) with a purity of at least 94% (w/w).

In some aspects, the providing the compound of Formula (III) comprises: providing a compound of Formula (II);

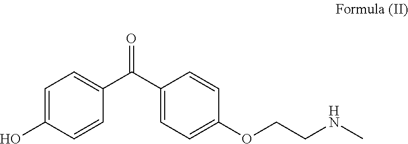 [SMILES: CNCCOc1ccc(C(=O)c2ccc(O)cc2)cc1]

and performing a McMurry reaction on the compound of Formula (II) and propiophenone to produce the compound of Formula (III).

In some aspects, the McMurry reaction comprises contacting the compound of Formula (II) and the propiophenone with zinc and titanium (IV) chloride. In some aspects, the McMurry reaction further comprises maintaining a temperature of not less than 60° C. for not less than 8 hours. In some aspects, the zinc, the titanium (IV) chloride, or both are dissolved or suspended in tetrahydrofuran. In some aspects, the zinc and the titanium (IV) chloride are mixed at a temperature of not less than 60° C. for not less than 2 hours prior to contacting the compound of Formula (II).

In some aspects, the compound of Formula (II) is in a suspension comprising the propiophenone. In some aspects, the suspension is suspended in an organic solvent. In some aspects, the organic solvent is selected from the group consisting of 2-methyltetrahydrofuran, dichloromethane, dichloroethane, chloroform, carbon tetrachloride, chlorobenzene, diethyl ether, 1,4-dioxane, tert-butyl methyl ether, tetrahydrofuran, N,N-dimethylformamide, N-methylpyrrolidone, diglyme, nitromethane, 1,2-dimethoxyethane, pyridine, acetone, acetonitrile, benzene, o-xylene, m-xylene, p-xylene, xylenes, hexanes, cyclohexane, heptane, octane, nonane, decane, and combinations thereof. In some aspects, the organic solvent is tetrahydrofuran. In some aspects, the suspension is mixed at a temperature of not less than 60° C. prior to contacting the zinc and the titanium (IV) chloride.

In some aspects, the McMurry reaction further comprises extracting with an organic solvent a product produced upon the contacting the compound of Formula (II) with the zinc and the titanium (IV) chloride. In some aspects, the organic solvent is selected from the group consisting of 2-methyltetrahydrofuran, dichloromethane, dichloroethane, chloroform, carbon tetrachloride, chlorobenzene, diethyl ether, 1,4-dioxane, tert-butyl methyl ether, tetrahydrofuran, N,N-dimethylformamide, N-methylpyrrolidone, diglyme, nitromethane, 1,2-dimethoxyethane, pyridine, acetone, acetonitrile, benzene, o-xylene, m-xylene, p-xylene, xylenes, hexanes, cyclohexane, heptane, octane, nonane, decane, and combinations thereof. In some aspects, the organic solvent is 2-methyltetrahydrofuran. In some aspects, the product produced upon the contacting the compound of Formula (II) with the zinc and the titanium (IV) chloride is further extracted with solution comprising a carbonate salt. In some aspects, the carbonate salt is selected from the group consisting of potassium carbonate, sodium carbonate, lithium carbonate, magnesium carbonate, and calcium carbonate. In some aspects, the carbonate salt is potassium carbonate.

In some aspects, the product produced upon the contacting the compound of Formula (II) with the zinc and the titanium (IV) chloride is further extracted with a solution comprising an Arrhenius base. In some aspects, the Arrhenius base is selected from the group consisting of sodium hydroxide, potassium hydroxide, calcium hydroxide, and lithium hydroxide. In some aspects, the Arrhenius base is sodium hydroxide.

In some aspects, the product produced upon the contacting the compound of Formula (II) with the zinc and the titanium (IV) chloride is further extracted with a solution comprising a neutral salt. In some aspects, the neutral salt is selected from the group consisting of sodium chloride, potassium chloride, potassium sulfate, sodium sulfate, potassium nitrate, sodium nitrate, potassium chlorate, sodium chlorate, potassium perchlorate, and sodium perchlorate. In some aspects, the neutral salt is sodium chloride.

In some aspects, the providing the compound of Formula (III) further comprises concentrating a product produced by the McMurry reaction in acetone, acetonitrile, or both. In some aspects, the providing the compound of Formula (III) further comprises washing a product produced by the McMurry reaction in acetone, acetonitrile, or both.

In some aspects, the providing the compound of Formula (II) comprises: providing a compound of Formula (I);

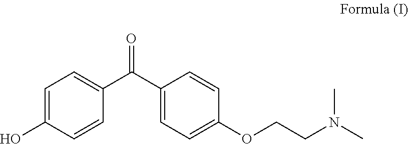 [SMILES: CN(C)CCOc1ccc(C(=O)c2ccc(O)cc2)cc1]

and performing a demethylation reaction on the compound of Formula (I) to produce the compound of Formula (II).

In some aspects, the demethylation reaction comprises contacting the compound of Formula (I) with N-ethyldiisopropylamine. In some aspects, the compound of Formula (I), the N-ethyldiisopropylamine, or both are dissolved in an organic solvent. In some aspects, the organic solvent is selected from the group consisting of 2-methyltetrahydrofuran, dichloromethane, dichloroethane, chloroform, carbon tetrachloride, chlorobenzene, diethyl ether, 1,4-dioxane, tert-butyl methyl ether, tetrahydrofuran, N,N-dimethylformamide, N-methylpyrrolidone, diglyme, nitromethane, 1,2-dimethoxyethane, pyridine, acetone, acetonitrile, benzene, o-xylene, m-xylene, p-xylene, xylenes, hexanes, cyclohexane, heptane, octane, nonane, decane, and combinations thereof. In some aspects, the organic solvent is tetrahydrofuran.

In some aspects, the demethylation reaction comprises contacting the compound of Formula (I) with 1-chloroethyl chloroformate. In some aspects, the compound of Formula (I) and the 1-chloroethyl chloroformate are heated to reflux. In some aspects, the compound of Formula (I) and the 1-chloroethyl chloroformate are mixed at not less than 60° C. for not less than 12 hours.

In some aspects, the demethylation reaction comprises contacting the compound of Formula (I) with a solution comprising an Arrhenius acid. In some aspects, the Arrhenius acid is selected from the group consisting of hydrochloric acid, hydrobromic acid, hydroiodic acid, hydrosulfuric acid, acetic acid, nitric acid, nitrous acid, sulfuric acid, sulfurous acid, chloric acid, perchloric acid, chlorous acid, phosphoric acid, phosphorous acid, and carbonic acid. In some aspects, the Arrhenius acid is hydrochloric acid.

In some aspects, the method further comprises increasing a pH of the demethylation reaction to at least about 13 or at least about 13.4. In some aspects, increasing the pH comprises adding solution comprising an Arrhenius base to the demethylation reaction. In some aspects, the Arrhenius base is selected from the group consisting of sodium hydroxide, potassium hydroxide, calcium hydroxide, and lithium hydroxide. In some aspects, the Arrhenius base is sodium hydroxide.

In some aspects, the method further comprises extracting the compound of Formula (II) with ethyl acetate. In some aspects, the ethyl acetate fractional recrystallization comprises contacting the compound of Formula (III) with ethyl acetate and an Arrhenius acid to extract the compound of Formula (IV). In some aspects, the Arrhenius acid is selected from the group consisting of hydrochloric acid, hydrobromic acid, hydroiodic acid, hydrosulfuric acid, acetic acid, nitric acid, nitrous acid, sulfuric acid, sulfurous acid, chloric acid, perchloric acid, chlorous acid, phosphoric acid, phosphorous acid, and carbonic acid. In some aspects, the Arrhenius acid is hydrochloric acid. In some aspects, the compound of Formula (III), the ethyl acetate, and the Arrhenius acid are mixed at from 50° C. to 70° C., or from 55° C. to 65° C. for not less than 6 hours. In some aspects, the ethyl acetate fractional recrystallization further comprises increasing the pH of a solution comprising the compound of Formula (III) to not less than 12 by adding an Arrhenius base. In some aspects, the Arrhenius base is selected from the group consisting of sodium hydroxide, potassium hydroxide, calcium hydroxide, and lithium hydroxide. In some aspects, the Arrhenius base is sodium hydroxide.

In some aspects, the ethyl acetate fractional recrystallization further comprises adding ethyl acetate to extract the compound of Formula (IV). In some aspects, the ethyl acetate fractional recrystallization further comprises washing the compound of Formula (IV) with a solution comprising a neutral salt. In some aspects, the neutral salt is selected from the group consisting of sodium chloride, potassium chloride, potassium sulfate, sodium sulfate, potassium nitrate, sodium nitrate, potassium chlorate, sodium chlorate, potassium perchlorate, and sodium perchlorate. In some aspects, the neutral salt is sodium chloride. In some aspects, the ethyl acetate fractional recrystallization further comprises filtering the compound of Formula (IV).

In some aspects, the acetone crystallization comprises contacting the compound of Formula (IV) with acetone. In some aspects, the acetone crystallization further comprises contacting the compound of Formula (IV) and the acetone with 2-propanol.

In some aspects, the tetrahydrofuran crystallization comprises contacting the compound of Formula (IV) with tetrahydrofuran. In some aspects, the tetrahydrofuran crystallization further comprises contacting the compound of Formula (IV) and the tetrahydrofuran with 2-propanol.

In some aspects, the ethyl acetate fractional recrystallization, the acetone crystallization, the tetrahydrofuran crystallization, or a combination thereof reduces a level of (E)-endoxifen in the compound of Formula (III) or the compound of Formula (IV). In some aspects, the ethyl acetate fractional recrystallization, the acetone crystallization, the tetrahydrofuran crystallization, or the combination thereof reduces the level of (E)-endoxifen in the compound of Formula (III) or the compound of Formula (IV) by at least about 99.9%, 99%, 97%, 75%, 60%, 50%, 45%, 40%, 30%, 20%, 10%, or 5%. In some aspects, the level of (E)-endoxifen is measured by HPLC. In some aspects, the ethyl acetate fractional recrystallization, the acetone crystallization, the tetrahydrofuran crystallization, or a combination thereof reduces a level of mesityl oxide in the compound of Formula (III) or the compound of Formula (IV). In some aspects, the ethyl acetate fractional recrystallization, the acetone crystallization, the tetrahydrofuran crystallization, or the combination thereof reduces the level of mesityl oxide in the compound of Formula (III) or the compound of Formula (IV) by at least about 99.9%, 99%, 97%, 75%, 60%, 50%, 40%, 30%, 20%, 15%, 10%, 5%, or 1%. In some aspects, the level of mesityl oxide is measured by HPLC.

In some aspects, the ethyl acetate fractional recrystallization, the acetone crystallization, the tetrahydrofuran crystallization, or a combination thereof reduces a level of 4-(1-(4-methoxyphenyl)-2-phenylbut-1-en-1-yl)phenol, a level of 4-(1-(4-isopropoxyphenyl)-2-phenylbut-1-en-1-yl)phenol, or a level of 1-(4-(2-hydroxyethoxy)phenyl)-1,2-bis(4-hydroxyphenyl)-2-(4-(2-(methylamino)ethoxy)phenyl)ethane-1,2-diol in the compound of Formula (III) or the compound of Formula (IV). In some aspects, the ethyl acetate fractional recrystallization, the acetone crystallization, the tetrahydrofuran crystallization, or the combination thereof reduces the level 4-(1-(4-methoxyphenyl)-2-phenylbut-1-en-1-yl)phenol by at least about 99.9%, 99%, 97%, 75%, 60%, 50%, 40%, 30%, 20%, 15%, 10%, 5%, or 1%. In some aspects, the ethyl acetate fractional recrystallization, the acetone crystallization, the tetrahydrofuran crystallization, or the combination thereof reduces the level 4-(1-(4-isopropoxyphenyl)-2-phenylbut-1-en-1-yl)phenol by at least about 99.9%, 99%, 97%, 75%, 60%, 50%, 40%, 30%, 20%, 15%, 10%, 5%, or 1%. In some aspects, the ethyl acetate fractional recrystallization, the acetone crystallization, the tetrahydrofuran crystallization, or the combination thereof reduces the level 1-(4-(2-hydroxyethoxy)phenyl)-1,2-bis(4-hydroxyphenyl)-2-(4-(2-(methylamino)ethoxy)phenyl)ethane-1,2-diol by at least about 99.9%, 99%, 97%, 75%, 60%, 50%, 40%, 30%, 20%, 15%, 10%, 5%, or 1%.

In some aspects, the level of 1-(4-(2-hydroxyethoxy)phenyl)-1,2-bis(4-hydroxyphenyl)-2-(4-(2-(methylamino)ethoxy)phenyl)ethane-1,2-diol comprises a level of (1S,2R)-1-(4-(2-hydroxyethoxy)phenyl)-1,2-bis(4-hydroxyphenyl)-2-(4-(2-(methylamino)ethoxy)phenyl)ethane-1,2-diol; a level of (1R,2S)-1-(4-(2-hydroxyethoxy)phenyl)-1,2-bis(4-hydroxyphenyl)-2-(4-(2-(methylamino)ethoxy)phenyl)ethane-1,2-diol; a level of (1S,2S)-1-(4-(2-hydroxyethoxy)phenyl)-1,2-bis(4-hydroxyphenyl)-2-(4-(2-(methylamino)ethoxy)phenyl)ethane-1,2-diol; a level of (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof. In some aspects, the level of 4-(1-(4-methoxyphenyl)-2-phenylbut-1-en-1-yl)phenol, the level of 4-(1-(4-isopropoxyphenyl)-2-phenylbut-1-en-1-yl)phenol, or the level of 1-(4-(2-hydroxyethoxy)phenyl)-1,2-bis(4-hydroxyphenyl)-2-(4-(2-(methylamino)ethoxy)phenyl)ethane-1,2-diol is measured by HPLC.

In some aspects, the compound of Formula (IV) is purified with a purity of at least 96% (w/w).

In various aspects, the present disclosure provides a composition, comprising: at least 96% w/w of a compound of Formula (IV),

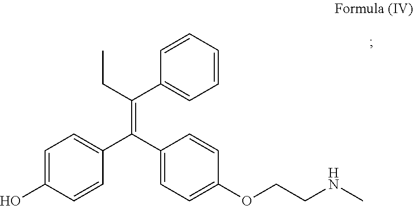 [SMILES: CC/C(=C(\c1ccc(O)cc1)c1ccc(OCCNC)cc1)c1ccccc1]

not more than 4% w/w of impurities, wherein the impurities comprise not more than 25 ppm mesityl oxide, with respect to a total amount of the composition.

In some aspects, the impurities comprise not more than 3% (w/w) (E)-endoxifen, with respect to the total amount of the composition.

In various aspects, the present disclosure provides a composition comprising a compound of Formula (IV), produced by a method as described herein.

In some aspects, the composition comprises not more than 3%, 2.5%, 2%, 1.5%, or 1% (E)-endoxifen by weight. In some aspects, the composition comprises not less than 97%, 97.5%, 98%, 98.5%, or 99% (Z)-endoxifen by weight. In some aspects, the composition comprises not more than 5 ppm, 10 ppm, 15 ppm, 20 ppm, or 25 ppm mesityl oxide. In some aspects, the composition comprises not more than 0.1%, 0.15%, 0.2%, 0.25%, 0.3%, 0.4%, or 0.5% 4-(1-(4-methoxyphenyl)-2-phenylbut-1-en-1-yl)phenol by weight. In some aspects, the composition comprises not more than 0.1%, 0.15%, 0.2%, 0.25%, 0.3%, 0.4%, or 0.5% 4-(1-(4-isopropoxyphenyl)-2-phenylbut-1-en-1-yl)phenol by weight. In some aspects, the composition comprises not more than 0.1%, 0.15%, 0.2%, 0.25%, 0.3%, 0.4%, 0.5%, 0.6%, 0.7%, 0.8%, 0.9%, or 1% 1-(4-(2-hydroxyethoxy)phenyl)-1,2-bis(4-hydroxyphenyl)-2-(4-(2-(methylamino)ethoxy)phenyl)ethane-1,2-diol by weight. In some aspects, the composition comprises not more than 0.1%, 0.15%, 0.2%, 0.25%, 0.3%, 0.4%, or 0.5% (1S,2R)-1-(4-(2-hydroxyethoxy)phenyl)-1,2-bis(4-hydroxyphenyl)-2-(4-(2-(methylamino)ethoxy)phenyl)ethane-1,2-diol by weight. In some aspects, the composition comprises not more than 0.1%, 0.15%, 0.2%, 0.25%, 0.3%, 0.4%, or 0.5% (1R,2S)-1-(4-(2-hydroxyethoxy)phenyl)-1,2-bis(4-hydroxyphenyl)-2-(4-(2-(methylamino)ethoxy)phenyl)ethane-1,2-diol by weight. In some aspects, the composition comprises not more than 0.1%, 0.15%, 0.2%, 0.25%, 0.3%, 0.4%, or 0.5% (1S,2S)-1-(4-(2-hydroxyethoxy)phenyl)-1,2-bis(4-hydroxyphenyl)-2-(4-(2-(methylamino)ethoxy)phenyl)ethane-1,2-diol by weight In some aspects the composition comprises not more than 0.1%, 0.15%, 0.2%, 0.25%, 0.3%, 0.4%, or 0.5% (1R,2R)-1-(4-(2-hydroxyethoxy)phenyl)-1,2-bis(4-hydroxyphenyl)-2-(4-(2-(methylamino)ethoxy)phenyl)ethane-1,2-diol by weight.

In some aspects, the composition comprises not more than 5000 ppm ethanol. In some aspects, the composition comprises not more than 3000 ppm methanol. In some aspects, the composition comprises not more than 5000 ppm acetone. In some aspects, the composition comprises not more than 5000 ppm 2-propanol. In some aspects, the composition comprises not more than 410 ppm acetonitrile. In some aspects, the composition comprises not more than 5000 ppm ethyl acetate. In some aspects, the composition comprises not more than 720 ppm tetrahydrofuran. In some aspects, the composition comprises not more than 520 ppm 2-methyltetrahydrofuran. In some aspects, the composition comprises not more than 5000 ppm n-heptane. In some aspects, the composition comprises not more than 130 ppm zinc. In some aspects, the composition comprises not more than 2 ppm benzene.

In various aspects, the present disclosure provides a composition comprising a crystalline form of a compound of Formula (III):

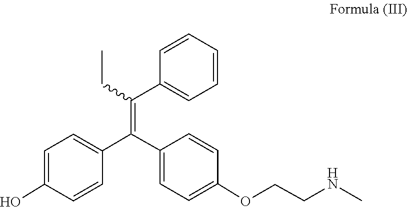 [SMILES: CCC(=C(c1ccc(O)cc1)c1ccc(OCCNC)cc1)c1ccccc1]

wherein the crystalline form of the compound of Formula (III) comprises: Form IV as characterized by an x-ray powder diffraction pattern comprising major peaks at 4.7±0.3° 2θ, 23.3±0.3° 2θ, and 13.6±0.3° 2θ; Form V as characterized by x-ray powder diffraction pattern comprising major peaks at 12.5±0.3° 2θ, 19.6±0.3° 2θ, and 8.9±0.3° 2θ; Form VI as characterized by x-ray powder diffraction pattern comprising major peaks at 9.9±0.3° 2θ, 13.4±0.3° 2θ, and 13.7±0.3° 2θ; Form VII as characterized by x-ray powder diffraction pattern comprising major peaks at 20.0±0.3° 2θ, 22.6±0.3° 2θ, and 10.6±0.3° 2θ; Form VIII as characterized by x-ray powder diffraction pattern comprising major peaks at 4.8±0.3° 2θ, 18.9±0.3° 2θ, and 9.5±0.3° 2θ; Form IX as characterized by x-ray powder diffraction pattern comprising major peaks at 19.0±0.3° 2θ, 12.9±0.3° 2θ, and 15.9±0.3° 2θ; Form X as characterized by x-ray powder diffraction pattern comprising major peaks at 7.2±0.3° 2θ, 14.3±0.3°2θ, 18.7±0.3°2θ, 21.5±0.3°2θ, and 22.7±0.3°2θ; Form XI as characterized by x-ray powder diffraction pattern comprising major peaks at 14.0±0.3°2θ, 17.7±0.3°2θ, 11.9 0.3°2θ, 18 4±0 3°2θ, 23 9±0.3°2θ, 17.3±0 3°2θ, 21.8±0.3°2θ, 20.8±0.3°2θ, and 23.0±3°2θ. Form XII as characterized by x-ray powder diffraction pattern comprising major peaks at 12.5±0.3°2θ, 15.6±0.3°2θ, and 19.0±0.3°2θ; Form XIV as characterized by x-ray powder diffraction pattern comprising major peaks at 11.6±0.3° 2θ, 21.3±0.3°2θ, and 19.3±0.3°2θ; Form XV as characterized by x-ray powder diffraction pattern comprising major peaks at 9.8±0.3°2θ, 4.7±0.3°2θ, and 14.0±0.3°2θ; Form XIX as characterized by x-ray powder diffraction pattern comprising major peaks at 4.7±0.3°2θ, 23.6±0.3°2θ, and 18.9±0.3°2θ; or a combination thereof.

In some aspects, at least about 80%, at least about 90%, at least about 95%, at least about 98%, or at least about 99% by weight of the compound of Formula (III) in the composition is the (Z)-isomer. In some aspects, at least about 50%, at least about 60%, at least about 70%, at least about 80%, at least about 90%, at least about 95%, at least about 98%, or at least about 99% of the compound of Formula (III) is Form IV, Form V, Form VI, Form VII, Form VIII, Form IX, Form X, Form XI, Form XII, Form XIII, Form XIV, Form XV, Form XIX, or a combination thereof.

In some aspects, the crystalline form of the compound of Formula (III) is stable at ambient temperature for at least 1 day, at least 3 days, is stable at ambient temperature for at least 7 days, is stable at ambient temperature for at least 14 days, is stable at ambient temperature for at least 21 days, is stable at ambient temperature for at least 30 days, is stable at ambient temperature for at least 60 days, or is stable at ambient temperature for at least 90 days.

In some aspects, the crystalline form comprises Form IV, or Form VII. In some aspects, the crystalline form comprises Form IV, Form VI, Form VII, Form VIII, Form IX, Form X, or Form XIV. In some aspects, the crystalline form comprises Form IV, Form V, Form VI, Form VII, Form VIII, Form IX, Form XII, Form XIV, Form XV, or Form XIX.

In some aspects, the crystalline form of the compound of Formula (III) is Form IV. In some aspects, the crystalline form of the compound of Formula (III) is characterized by an x-ray powder diffraction pattern comprising at least one peak, at least two peaks, at least three peaks, or at least four peaks selected from 23.8±0.3°2θ, 14.2±0.3°2θ, 22.5±0.3°2θ, and 15.7±0.3°2θ.

In some aspects, the x-ray powder diffraction pattern further comprises at least one peak or at least two peaks selected from 7.1±0.3°2θ and 20.2±0.3°2θ. In some aspects, the x-ray powder diffraction pattern further comprises at least one peak, comprises a peak at 9.5±0.3°2θ. In some aspects, the crystalline form of the compound of Formula (III) is characterized by an x-ray diffraction pattern substantially as set forth in FIG. 13.

In some aspects, the crystalline form of the compound of Formula (III) comprises between about 1% and about 30% solvent by weight as determined by TGA analysis. In some aspects, the crystalline form loses the solvent upon heating to a temperature between 70° C. and 130° C. as determined by TGA analysis. In some aspects, the solvent comprises 2-propanol, heptane, or a combination thereof.

In some aspects, the crystalline form of the compound of Formula (III) is Form V. In some aspects, the crystalline form of the compound of Formula (III) is characterized by an x-ray powder diffraction pattern comprising at least one peak, at least two peaks, at least three peaks, or at least four peaks selected from 21.7±0.3° 2θ, 20.8±0.3° 2θ, 19.8±0.3° 2θ, and 16.0±0.3° 2θ.

In some aspects, the x-ray powder diffraction pattern further comprises at least one peak, at least two peaks, or at least three peaks selected from 22.0±0.3° 2θ, 13.5±0.3° 2θ, or 14.4±0.3° 2θ. In some aspects, the crystalline form of the compound of Formula (III) is characterized by an x-ray diffraction pattern substantially as set forth in FIG. 27.

In some aspects, the crystalline form of the compound of Formula (III) is Form VI. In some aspects, the crystalline form of the compound of Formula (III) is characterized by an x-ray powder diffraction pattern comprising at least one peak, at least two peaks, at least three peaks, or at least four peaks selected from 17.6±0.3° 2θ, 18.6±0.3° 2θ, 17.3±0.3° 2θ, and 21.8±0.3° 2θ.

In some aspects, the x-ray powder diffraction pattern further comprises at least one peak, at least two peaks, or at least three peaks selected from 10.2±0.3° 2θ, 19.5±0.3° 2θ, and 14.2±0.3° 2θ. In some aspects, the crystalline form of the compound of Formula (III) is characterized by an x-ray diffraction pattern substantially as set forth in FIG. 29.

In some aspects, the crystalline form of the compound of Formula (III) is Form VII. In some aspects, the crystalline form of the compound of Formula (III) is characterized by an x-ray powder diffraction pattern comprising at least one peak, at least two peaks, at least three peaks, or at least four peaks selected from 11.4±0.3° 2θ, 16.4±0.3° 2θ, 9.6±0.3° 2θ, and 13.3±0.3° 2θ.

In some aspects, the x-ray powder diffraction pattern further comprises at least one peak, at least two peaks, or at least three peaks selected from 18.2±0.3° 2θ, 13.1±0.3° 2θ, and 27.0±0.3° 2θ. In some aspects, the crystalline form of the compound of Formula (III) is characterized by an x-ray diffraction pattern substantially as set forth in FIG. 31.

In some aspects, the crystalline form of the compound of Formula (III) is Form VIII. In some aspects, the crystalline form of the compound of Formula (III) is characterized by an x-ray powder diffraction pattern comprising at least one peak, at least two peaks, at least three peaks, or at least four peaks selected from 23.7±0.3° 2θ, 21.9±0.3° 2θ, 21.2±0.3° 2θ, and 12.9±0.3° 2θ.

In some aspects, the x-ray powder diffraction pattern further comprises at least one peak, at least two peaks, or at least three peaks selected from 25.0±0.3° 2θ, 21.5±0.3° 2θ, and 16.4±0.3° 2θ. In some aspects, the crystalline form of the compound of Formula (III) is characterized by an x-ray diffraction pattern substantially as set forth in FIG. 37.

In some aspects, the crystalline form of the compound of Formula (III) is Form IX. In some aspects, the crystalline form of the compound of Formula (III) is characterized by an x-ray powder diffraction pattern comprising at least one peak, at least two peaks, at least three peaks, or at least four peaks selected from 21.7±0.3° 2θ, 20.8±0.3° 2θ, 21.1±0.3° 2θ, and 8.9±0.3° 2θ.

In some aspects, the x-ray powder diffraction pattern further comprises at least one peak, at least two peaks, or at least three peaks selected from 16.4±0.3° 2θ, 4.2±0.3° 2θ, and 12.7±0.3° 2θ. In some aspects, the crystalline form of the compound of Formula (III) is characterized by an x-ray diffraction pattern substantially as set forth in FIG. 40.

In some aspects, the crystalline form of the compound of Formula (III) is Form X. In some aspects, the crystalline form of the compound of Formula (III) is characterized by an x-ray powder diffraction pattern comprising at least one peak or at least two peaks selected from 17.1±0.3° 2θ, and 22.7±0.3° 2θ. In some aspects, the x-ray powder diffraction pattern further comprises at least one peak, at least two peaks, or at least three peaks selected from 21.8±0.3°2θ, 27.3±0.3°2θ, and 29.4±0.3° 2θ. In some aspects, the crystalline form of the compound of Formula (III) is characterized by an x-ray diffraction pattern substantially as set forth in FIG. 52.

In some aspects, the crystalline form of the compound of Formula (III) is Form XI. In some aspects, the crystalline form of the compound of Formula (III) is characterized by an x-ray powder diffraction pattern comprising at least one peak or at least two peaks selected from 22.2±0.3° 20 and 16.6±0.3° 2θ. In some aspects, the crystalline form of the compound of Formula (III) is characterized by an x-ray diffraction pattern substantially as set forth in FIG. 54.

In some aspects, the crystalline form of the compound of Formula (III) is Form XII. In some aspects, the crystalline form of the compound of Formula (III) is characterized by an x-ray powder diffraction pattern comprising at least one peak, at least two peaks, at least three peaks, or at least four peaks selected from 21.9±0.3°2θ, 20.2±0.3°2θ, 16.0±0.3°2θ, and 21.6±0.3°2θ.

In some aspects, the x-ray powder diffraction pattern further comprises at least one peak, at least two peaks, or at least three peaks selected from 22.4±0.3°2θ, 16.8±0.3°2θ, and 12.8±0.3°2θ. In some aspects, the crystalline form of the compound of Formula (III) is characterized by an x-ray diffraction pattern substantially as set forth in FIG. 56.

In some aspects, the crystalline form of the compound of Formula (III) is Form XIV. In some aspects, the crystalline form of the compound of Formula (III) is characterized by an x-ray powder diffraction pattern comprising at least one peak, at least two peaks, at least three peaks, or at least four peaks selected from 17.5±0.3° 2θ, 15.4±0.3° 2θ, 21.6±0.3° 2θ, and 5.8±0.3° 2θ.

In some aspects, the x-ray powder diffraction pattern further comprises at least one peak, at least two peaks, or at least three peaks selected from 16.3±0.3° 2θ, 21.9±0.3° 2θ, and 23.9±0.3° 2θ. In some aspects, the crystalline form of the compound of Formula (III) is characterized by an x-ray diffraction pattern substantially as set forth in FIG. 71.

In some aspects, the crystalline form of the compound of Formula (III) is Form XV. In some aspects, the crystalline form of the compound of Formula (III) is characterized by an x-ray powder diffraction pattern comprising at least one peak, at least two peaks, at least three peaks, or at least four peaks selected from 20.2±0.3° 2θ, 7.1±0.3° 2θ, 23.4±0.3° 2θ, and 22.4±0.3° 2θ.

In some aspects, the x-ray powder diffraction pattern further comprises at least one peak, at least two peaks, or at least three peaks selected from 21.7±0.3° 2θ, 22.7±0.3° 2θ, and 18.8±0.3° 2θ. In some aspects, the crystalline form of the compound of Formula (III) is characterized by an x-ray diffraction pattern substantially as set forth in FIG. 6.

In some aspects, the crystalline form of the compound of Formula (III) is Form XIX. In some aspects, the crystalline form of the compound of Formula (III) is characterized by an x-ray powder diffraction pattern comprising at least one peak, at least two peaks, at least three peaks, or at least four peaks selected from 9.4±0.3° 2θ, 23.3±0.3° 2θ, 22.3±0.3° 2θ, and 20.1±0.3° 2θ.

In some aspects, the crystalline form of the x-ray powder diffraction pattern further comprises at least one peak, at least two peaks, or at least three peaks selected from 19.6±0.3° 2θ, 7.1±0.3° 2θ, and 15.7±0.3° 2θ. In some aspects, the crystalline form of the compound of Formula (III) is characterized by an x-ray diffraction pattern substantially as set forth in FIG. 25.

In various aspects, the present disclosure provides a composition comprising a crystalline form of a compound of Formula (III):

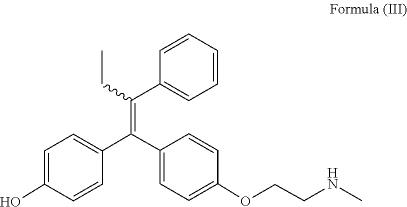 [SMILES: CCC(=C(c1ccc(O)cc1)c1ccc(OCCNC)cc1)c1ccccc1]

wherein the crystalline form of the compound of Formula (III) is Form XVII as characterized by an x-ray diffraction pattern substantially as set forth in FIG. 21.

In various aspects, the present disclosure provides a composition comprising a crystalline form of a compound of Formula (III):

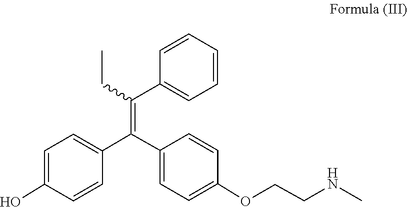 [SMILES: CCC(=C(c1ccc(O)cc1)c1ccc(OCCNC)cc1)c1ccccc1]

wherein the crystalline form of the compound of Formula (III) is Form XVIII as characterized by an x-ray diffraction pattern substantially as set forth in FIG. 23.

In various aspects, the present disclosure provides a method of generating a crystalline form of a compound of Formula (III):

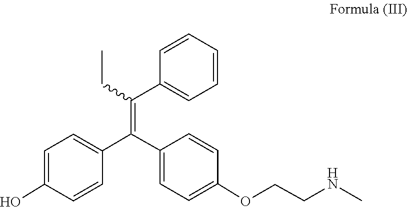 [SMILES: CCC(=C(c1ccc(O)cc1)c1ccc(OCCNC)cc1)c1ccccc1]

the method comprising: incubating a solid form of the compound of Formula (III) in a solvent system in which at least a portion of the solid form of the compound of Formula (III) remains solid; converting the portion of the solid form of the compound of Formula (III) to the crystalline form; and collecting the crystalline form.

In some aspects, the crystalline form of the compound of Formula (III) is Form I. In some aspects, the incubating is for between about 3 and about 120 hours. In some aspects, the solid form of the compound of Formula (III) comprises at least about 90% isomeric purity prior to the incubating. In some aspects, the crystalline form of the compound of Formula (III) comprises at least about 90% isomeric purity.

In some aspects, the solid form of the compound of Formula (III) comprises at least one of Forms IV-XIX. In some aspects, the solid form of the compound of Formula (III) is at least 25%, at least 50%, at least 75%, at least 90%, at least 95%, or at least 99% amorphous. In some aspects, between about 0.1% and about 75%, between about 0.1% and about 10%, between about 0.1% and about 25%, between about 1% and about 25%, between about 5% and about 50%, between about 20% and about 50%, or between about 25% and about 75% of the solid form of the compound of Formula (III) dissolves in the solvent system.

In some aspects, during the incubating the solvent system comprises a temperature between about 5° C. and about 110° C., between about 5° C. and about 90° C., between about 5° C. and about 25° C., between about 10° C. and about 60° C., between about 25° C. and about 50° C., between about 15° C. and about 50° C., between about 15° C. and about 70° C., between about 25° C. and about 40° C., or between about 35° C. and about 60° C. In some aspects, the incubating is performed for between about 0.1 and about 700 hours, between about 1 and about 360 hours, between about 3 and about 360 hours, between about 3 and about 36 hours, between about 6 and about 72 hours, between about 12 and about 150 hours, between about 24 and about 150 hours, between about 48 and about 240 hours, or between about 100 and about 700 hours.

In some aspects, the solvent system comprises between about 5 and about 10000 mg/ml, between about 5 and about 1000 mg/ml, between about 5 and about 300 mg/ml, between about 5 and about 50 mg/ml, between about 20 and about 300 mg/ml, between about 50 and about 300 mg/ml, between about 100 and about 1000 mg/ml, or between about 300 and about 10000 mg/ml of the solid form of the compound of Formula (III) during the incubating. In some aspects, the solvent system comprises 2-propanol, acetonitrile, acetone, butyl acetate, butyl methyl ether, dimethylformamide, ethanol, ethyl acetate, water, heptane, methanol, methyl isobutyl ketone, tetrahydrofuran, toluene, or a combination thereof.

In some aspects, the solvent system comprises a single solvent. In some aspects, the solvent comprises a plurality of solvents. In some aspects, the solvent system comprises two solvents in a ratio of between about 99:1 and about 1:1, between about 19:1 and about 1:1, between about 19:1 and 5:1, between about 8:1 and about 1:1, between about 4:1 and about 1:1, or between about 2:1 and about 1:1. In some aspects, the solvent system comprises less than about 25%, less than about 10%, less than about 5%, less than about 2%, less than about 1%, or less than about 0.25% water.

In various aspects, the present disclosure provides a method for producing a crystalline form of a compound of Formula (III), the method comprising: (a) dissolving the compound of Formula (III) in 2-propanol, and evaporating at least a portion of the 2-propanol; (b) dissolving the compound of Formula (III) in a solvent system comprising 2-propanol and tetrahydrofuran at a first temperature, and cooling the solvent system to a second temperature; (c) dissolving the compound of Formula (III) in a solvent system comprising heptane and 2-propanol at a first temperature, and cooling the solvent system to a second temperature; (d) dissolving the compound of Formula (III) in a solvent system comprising 2-propanol and ethyl acetate at a first temperature, and cooling the solvent system to a second temperature; (e) dissolving the compound of Formula (III) in tetrahydrofuran, and combining the solvent system with water; (f) dissolving the compound of Formula (III) in a solvent system comprising 2-propanol and tetrahydrofuran, and evaporating at least a portion of the solvent system; (g) dissolving the compound of Formula (III) in acetone, and evaporating at least a portion of the acetone; (h) dissolving the compound of Formula (III) in a solvent system comprising acetone and tetrahydrofuran, and evaporating at least a portion of the solvent system; or (i) a combination thereof, thereby producing the crystalline form of the compound of Formula (III).

In some aspects, the method comprises any one of (a)-(f) or (h). In some aspects, the crystalline form of the compound of Formula (III) is Form IV.

In some aspects, the cooling is at a rate of between about 0.02° C./minute and about 10° C./minute. In some aspects, the first temperature is between about 20° C. and about 100° C., and the second temperature is between about −20° C. and about 25° C.

In various aspects, the present disclosure provides a method for producing a crystalline form of a compound of Formula (III), the method comprising: dissolving the compound of Formula (III) in acetonitrile at a first temperature, and cooling the acetonitrile to a second temperature, thereby producing the crystalline form of the compound of Formula (III).

In some aspects, the crystalline form of the compound of Formula (III) is Form V. In some aspects, the cooling is at a rate of between about 0.02° C./minute and about 10° C./minute.

In various aspects, the present disclosure provides a method for producing a crystalline form of a compound of Formula (III), the method comprising: storing Form V at a temperature of between about 20° C. and about 50° C., thereby producing the crystalline form of the compound of Formula (III).

In some aspects, the crystalline form of the compound of Formula (III) is Form VI.

In various aspects, the present disclosure provides a method for producing a crystalline form of a compound of Formula (III), the method comprising: dissolving the compound of Formula (III) in ethyl acetate, and adding the ethyl acetate to heptane; dissolving the compound of Formula (III) in toluene, and evaporating at least a portion of the toluene; dissolving the compound of Formula (III) in toluene, and adding the toluene to heptane; dissolving the compound of Formula (III) in a solvent system comprising heptane and toluene, and heating the solvent system to between about 20° C. and about 50° C.; or a combination thereof, thereby producing the crystalline form of the compound of Formula (III).

In some aspects, the crystalline form of the compound of Formula (III) is Form VII.

In various aspects, the present disclosure provides a method for producing a crystalline form of a compound of Formula (III), the method comprising: dissolving the compound of Formula (III) in toluene at a first temperature, and cooling the solvent system to a second temperature; dissolving the compound of Formula (III) in toluene, adding butyl methyl ether to the toluene to form a solvent system, and evaporating at least a portion of the solvent system; or a combination thereof, thereby producing the crystalline form of the compound of Formula (III).

In some aspects, the crystalline form of the compound of Formula (III) is Form VIII. In some aspects, the cooling is at a rate of between about 0.02° C./minute and about 10° C./minute.

In various aspects, the present disclosure provides a method for producing a crystalline form of a compound of Formula (III), the method comprising: dissolving the compound of Formula (III) in a solvent system comprising ethyl acetate, adding heptane to the solvent system, and incubating the solvent system at a temperature of between about 20° C. and about −20° C.; dissolving the compound of Formula (III) in a solvent system comprising butyl acetate, adding heptane to the solvent system; dissolving the compound of Formula (III) in ethyl acetate, adding the ethyl acetate to butyl methyl ether to form a solvent system, and evaporating at least a portion of the solvent system; dissolving the compound of Formula (III) in ethyl acetate, adding butyl methyl ether to the ethyl acetate to form a solvent system, and evaporating at least a portion of the solvent system; dissolving the compound of Formula (III) in acetone, combining the acetone with butyl methyl ether to form a solvent system, and evaporating at least a portion of the solvent system; or a combination thereof; thereby producing the crystalline form of the compound of Formula (III).

In some aspects, the crystalline form of the compound of Formula (III) is Form IX.

In various aspects, the present disclosure provides a method for producing a crystalline form of a compound of Formula (III), the method comprising: dissolving the compound of Formula (III) in 4-methyl-2-pentanone at a first temperature, and cooling the 4-methyl-2-pentanone to a second temperature, thereby producing the crystalline form of the compound of Formula (III).

In some aspects, the crystalline form of the compound of Formula (III) is Form X. In some aspects, the cooling is at a rate of between about 0.02° C./minute and about 10° C./minute.

In various aspects, the present disclosure provides a method for producing a crystalline form of a compound of Formula (III), the method comprising: dissolving less than about 100 micrograms of the compound of Formula (III) in tetrahydrofuran, adding the tetrahydrofuran to acetone to form a solvent system, and evaporating at least a portion of the solvent system, thereby producing the crystalline form of the compound of Formula (III).

In some aspects, the crystalline form of the compound of Formula (III) is Form XII.

In various aspects, the present disclosure provides a method for producing a crystalline form of a compound of Formula (III), the method comprising: dissolving greater than about 100 micrograms of the compound of Formula (III) in tetrahydrofuran, adding the tetrahydrofuran to acetone to form a solvent system, and evaporating at least a portion of the solvent system, thereby producing the crystalline form of the compound of Formula (III).

In some aspects, the crystalline form of the compound of Formula (III) is Form XIV.

In various aspects, the present disclosure provides a method for producing a crystalline form of a compound of Formula (III), the method comprising: dissolving the compound of Formula (III) in ethyl acetate, and adding the ethyl acetate to heptane, thereby producing the crystalline form of the compound of Formula (III).

In some aspects, the crystalline form of the compound of Formula (III) is Form XV.

In various aspects, the present disclosure provides a method for producing a crystalline form of a compound of Formula (III), the method comprising: dissolving the compound of Formula (III) in acetone at a first temperature, combining the acetone with heptane to form a solvent system, and cooling the solvent system to a second temperature; dissolving the compound of Formula (III) in tetrahydrofuran at a first temperature, combining the tetrahydrofuran with heptane to form a solvent system, and cooling the solvent system to a second temperature; or a combination thereof; thereby producing the crystalline form of the compound of Formula (III).

In some aspects, the crystalline form of the compound of Formula (III) is Form XIX. In some aspects, the cooling is at a rate of between about 0.02° C./minute and about 10° C./minute.

In various aspects, the present disclosure provides a method for producing a crystalline form of a compound of Formula (III), the method comprising: dissolving the compound of Formula (III) in ethyl acetate at a first temperature, combining the ethyl acetate with a heptane to form a solvent system, and cooling the solvent system to a second temperature, thereby producing the crystalline form of the compound of Formula (III).

In some aspects, the crystalline form of the compound of Formula (III) is Form XVI. In some aspects, the cooling is at a rate of between about 0.02° C./minute and about 10° C./minute.

In various aspects, the present disclosure provides a method for producing a crystalline form of a compound of Formula (III), the method comprising: dissolving the compound of Formula (III) in ethanol, and evaporating at least a portion of the ethanol, thereby producing the crystalline form of the compound of Formula (III).

In some aspects, the crystalline form of the compound of Formula (III) is Form XVII.

In various aspects, the present disclosure provides a method for producing a crystalline form of a compound of Formula (III), the method comprising: dissolving the compound of Formula (III) in tetrahydrofuran, and evaporating at least a portion of the tetrahydrofuran, thereby producing the crystalline form of the compound of Formula (III).

In some aspects, the crystalline form of the compound of Formula (III) is Form XVIII.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides several X-ray powder diffraction (XRPD) spectra of polymorphs consistent with the present disclosure including Form I, Form IV, and a slurry at high temperature experiment (SLHT-1D-2PR/THF(100 mg/ml)).

FIG. 2. provides several X-ray powder diffraction (XRPD) spectra of polymorphs consistent with the present disclosure including Form I and several slurries at high temperature experiments (SLHT-1D-H₂O/ACN(100 mg/ml) and SLHT-1D-H₂O/ACN(40 mg/ml)).

FIG. 3 provides several X-ray powder diffraction (XRPD) spectra of polymorphs consistent with the present disclosure including Form I and several slurries at high temperature experiments (SLHT-1D-H₂O/THF(40 mg/ml), SLHT-1D-ACT/ACN(40 mg/ml), SLHT-1D-ACT/ETA(40 mg/ml), and SLHT-1D-H₂O/2PR(40 mg/ml)).

FIG. 4 provides several X-ray powder diffraction (XRPD) spectra of polymorphs consistent with the present disclosure including Form I and a slurry at high temperature experiment (SLHT-1D-HEP/ETA(40 mg/ml)).

FIG. 5. provides several X-ray powder diffraction (XRPD) spectra of polymorphs consistent with the present disclosure including Form VII, Form I, and a slurry at high temperature experiment (SLHT-D-HEP/TOL(40 mg/ml)).

FIG. 6 provides an X-ray powder diffraction (XRPD) spectrum of a polymorph of Form XV, consistent with the present disclosure.

FIG. 7 provides an X-ray powder diffraction (XRPD) spectrum of a Kapton film, consistent with this present disclosure.

FIG. 8 provides an X-ray powder diffraction (XRPD) spectra of Form XI and E-endoxifen.

FIG. 9 provides an X-ray powder diffraction (XRPD) spectrum of a polymorph of Form I, consistent with the present disclosure.

FIG. 10 provides several XRPD spectra of Form I collected at several time points during an incubation including 7 days (Form 1-7D-storage) and 42 days (Form I-42D-storage).

FIG. 11 provides a differential scanning calorimetry (DSC) profile of a polymorph consistent with the present disclosure.

FIG. 12 provides a thermogravimetric analysis (TGA) profile of a polymorph consistent with the present disclosure.

FIG. 13 provides an X-ray powder diffraction (XRPD) spectrum of a polymorph of Form IV, consistent with the present disclosure.

FIG. 14 provides several XRPD spectra of a polymorph consistent with the present disclosure collected at several time points during an incubation.

FIG. 15 provides several XRPD spectra of a polymorph consistent with the present disclosure collected at several time points during an incubation.

FIG. 16 provides several XRPD spectra of a polymorph consistent with the present disclosure collected at several time points during an incubation.

FIG. 17 provides a differential scanning calorimetry (DSC) profile of a polymorph consistent with the present disclosure.

FIG. 18A provides a thermogravimetric analysis (TGA) profile of a polymorph consistent with the present disclosure.

FIG. 18B provides a 2-dimensional gas evolution analysis of a polymorph consistent with the present disclosure.

FIG. 18C provides a 3-dimensional gas evolution analysis of a polymorph consistent with the present disclosure.

FIG. 18D provides a kinetic gas evolution profile of a polymorph consistent with the present disclosure.

FIG. 19 provides the Fourier-transform infrared (FT-IR) spectra of 2-propanol, heptane, and a sample of gas evolved from a polymorph consistent with the present disclosure.

FIG. 20 provides several XRPD spectra of polymorphs consistent with the present disclosure.

FIG. 21 provides an XRPD spectrum of a polymorph consistent with the present disclosure.

FIG. 22 provides several XRPD spectra of a polymorph consistent with the present disclosure collected at several time points during an incubation.

FIG. 23 provides an XRPD spectrum of a polymorph consistent with the present disclosure.

FIG. 24 provides several XRPD spectra of a polymorph consistent with the present disclosure collected at several time points during an incubation.

FIG. 25 provides an XRPD spectrum of a polymorph of Form XIX, consistent with the present disclosure.

FIG. 26 provides several XRPD spectra of a polymorph consistent with the present disclosure collected at several time points during an incubation.

FIG. 27 provides an XRPD spectrum of a polymorph of Form V, consistent with the present disclosure.

FIG. 28 provides several XRPD spectra of a polymorph consistent with the present disclosure collected at several time points during an incubation.

FIG. 29 provides an XRPD spectrum of a polymorph of Form VI, consistent with the present disclosure.

FIG. 30 provides several XRPD spectra of a polymorph consistent with the present disclosure collected at several time points during an incubation.

FIG. 31 provides an XRPD spectrum of a polymorph of Form VII, consistent with the present disclosure.

FIG. 32 provides several XRPD spectra of a polymorph consistent with the present disclosure collected at several time points during an incubation.

FIG. 33 provides a differential scanning calorimetry (DSC) profile of a polymorph consistent with the present disclosure.

FIG. 34 provides a thermogravimetric analysis (TGA) profile of a polymorph consistent with the present disclosure.

FIG. 35 provides several XRPD spectra of a polymorph consistent with the present disclosure.

FIG. 36 provides several XRPD spectra of a polymorph consistent with the present disclosure.

FIG. 37 provides an XRPD spectrum of a polymorph of Form VIII, consistent with the present disclosure.

FIG. 38 provides several XRPD spectra of a polymorph consistent with the present disclosure collected at several time points during an incubation.

FIG. 39 provides several XRPD spectra of a polymorph consistent with the present disclosure collected at several time points during an incubation.

FIG. 40 provides an XRPD spectrum of a polymorph of Form IX, consistent with the present disclosure.

FIG. 41 provides several XRPD spectra of a polymorph consistent with the present disclosure collected at several time points during an incubation.

FIG. 42 provides several XRPD spectra of a polymorph consistent with the present disclosure collected at several time points during an incubation.

FIG. 43 provides several XRPD spectra of a polymorph consistent with the present disclosure collected at several time points during an incubation.

FIG. 44 provides several XRPD spectra of a polymorph consistent with the present disclosure collected at several time points during an incubation.

FIG. 45 provides several XRPD spectra of a polymorph consistent with the present disclosure collected at several time points during an incubation.

FIG. 46 provides several XRPD spectra of a polymorph consistent with the present disclosure.

FIG. 47 provides several XRPD spectra of a polymorph consistent with the present disclosure.

FIG. 48 provides a thermogravimetric analysis (TGA) profile of a polymorph consistent with the present disclosure.

FIG. 49 provides several XRPD spectra of a polymorph consistent with the present disclosure.

FIG. 50 provides several XRPD spectra of a polymorph consistent with the present disclosure.

FIG. 51 provides several XRPD spectra of a polymorph consistent with the present disclosure.

FIG. 52 provides an XRPD spectrum of a polymorph of Form X, consistent with the present disclosure.

FIG. 53 provides several XRPD spectra of a polymorph consistent with the present disclosure collected at several time points during an incubation.

FIG. 54 provides an XRPD spectrum of a polymorph of Form XI, consistent with the present disclosure.

FIG. 55 provides several XRPD spectra of a polymorph consistent with the present disclosure collected at several time points during an incubation.

FIG. 56 provides an XRPD spectrum of a polymorph of Form XII, consistent with the present disclosure.

FIG. 57 provides several XRPD spectra of a polymorph consistent with the present disclosure collected at several time points during an incubation.

FIG. 58 provides an XRPD spectrum of a mixture of polymorphic consistent with the present disclosure.

FIG. 59 provides several XRPD spectra of a polymorph consistent with the present disclosure.

FIG. 60 provides several XRPD spectra of a polymorph consistent with the present disclosure.

FIG. 61 provides several XRPD spectra of a polymorph consistent with the present disclosure.

FIG. 62 provides an XRPD spectrum of a mixture of polymorphic consistent with the present disclosure.

FIG. 63 provides a magnification of FIG. 58.

FIG. 64 provides several XRPD spectra of a polymorph consistent with the present disclosure collected at several time points during an incubation.

FIG. 65 provides several XRPD spectra of a polymorph consistent with the present disclosure collected at several time points during an incubation.

FIG. 66 provides several XRPD spectra of a polymorph consistent with the present disclosure collected at several time points during an incubation.

FIG. 67 provides a differential scanning calorimetry (DSC) profile of a polymorph consistent with the present disclosure.

FIG. 68 provides several differential scanning calorimetry (DSC) profiles of a polymorph consistent with the present disclosure.

FIG. 69 provides a thermogravimetric analysis (TGA) profile of a polymorph consistent with the present disclosure.

FIG. 70 provides a magnification of FIG. 69.

FIG. 71 provides an XRPD spectrum of a polymorph of Form XIV, consistent with the present disclosure.

FIG. 72 provides several XRPD spectra of a polymorph consistent with the present disclosure.

FIG. 73 provides several XRPD spectra of a polymorph consistent with the present disclosure.

FIG. 74 provides several XRPD spectra of a polymorph consistent with the present disclosure.

FIG. 75 provides several XRPD spectra of a polymorph consistent with the present disclosure.

FIG. 76 provides several XRPD spectra of a polymorph consistent with the present disclosure.

FIG. 77 provides several XRPD spectra of a polymorph consistent with the present disclosure.

FIG. 78 provides several XRPD spectra of a polymorph consistent with the present disclosure.

FIG. 79 provides several XRPD spectra of a polymorph consistent with the present disclosure.

FIG. 80 provides several XRPD spectra of a polymorph consistent with the present disclosure.

FIG. 81 provides several XRPD spectra of a polymorph consistent with the present disclosure.

FIG. 82 provides several XRPD spectra of a polymorph consistent with the present disclosure.

FIG. 83 provides several XRPD spectra of a polymorph consistent with the present disclosure.

FIG. 84 provides several XRPD spectra of a polymorph consistent with the present disclosure.

FIG. 85 provides several XRPD spectra of a polymorph consistent with the present disclosure.

FIG. 86 provides several XRPD spectra of a polymorph consistent with the present disclosure.

FIG. 87 provides several XRPD spectra of a polymorph consistent with the present disclosure.

FIG. 88 provides several XRPD spectra of a polymorph consistent with the present disclosure.

FIG. 89 provides several XRPD spectra of a polymorph consistent with the present disclosure.

FIG. 90 provides several XRPD spectra of a polymorph consistent with the present disclosure.

FIG. 91 provides several XRPD spectra of a polymorph consistent with the present disclosure.

FIG. 92 provides several XRPD spectra of a polymorph consistent with the present disclosure.

### DETAILED DESCRIPTION

Described herein are highly pure compositions comprising (Z)-endoxifen and methods of synthesizing (Z)-endoxifen with a high level of purity, e.g., isomeric purity. In certain aspects, the present disclosure provides a composition comprising a crystalline form of a compound of Formula (III), and methods of synthesizing a compound of Formula (III) with a high level of purity:

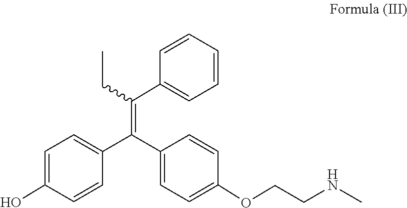 [SMILES: CCC(=C(c1ccc(O)cc1)c1ccc(OCCNC)cc1)c1ccccc1]

A compound of Formula (III) may comprise a mixture of (E)-endoxifen and (Z)-endoxifen.

In certain aspects, the present disclosure provides a composition comprising a crystalline form of a compound of Formula (IV), and methods of synthesizing a compound of Formula (IV) with a high level of purity:

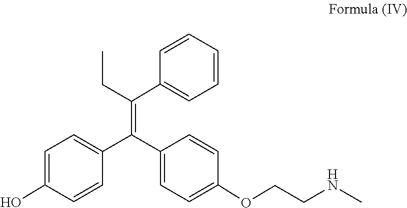 [SMILES: CC/C(=C(\c1ccc(O)cc1)c1ccc(OCCNC)cc1)c1ccccc1]

A compound of Formula (IV) may comprise (Z)-endoxifen.

In certain aspects, the present disclosure provides crystalline forms of (Z)-endoxifen free base, pharmaceutically acceptable salts of isomerically pure (Z)-endoxifen, and crystalline forms of mixtures of (E)-endoxifen and (Z)-endoxifen, as well as pharmaceutical compositions of endoxifen comprising the crystalline forms described herein. In some aspects, the crystalline forms of (Z)-endoxifen free base or mixture of (E)-endoxifen and (Z)-endoxifen are purified to reduce impurities.

In one aspect, the present disclosure provides methods of making (Z)-endoxifen free base, mixtures of (E)-endoxifen and (Z)-endoxifen (E/Z-mix), and pharmaceutically acceptable salts thereof. In an aspect, the methods are synthetic methods of making stable (Z)-endoxifen free base, E/Z-mix, and pharmaceutically acceptable salts thereof. In some embodiments, the methods may be industrially scalable. For example, a method described herein may be scaled to produce up to 5 kg of the (Z)-endoxifen free base, E/Z-mix, or pharmaceutically acceptable salts thereof. In another aspect, the present disclosure provides methods of making compositions comprising stable (Z)-endoxifen free base, polymorphs thereof, E/Z-mix, and pharmaceutically acceptable salts thereof. In some embodiments, the methods provided herein may be used to synthesize (Z)-endoxifen free base, polymorphs thereof, mixtures of (E)-endoxifen and (Z)-endoxifen (E/Z-mix), and pharmaceutically acceptable salts thereof with a high level of purity (e.g., ≥94%, ≥95%, ≥96%, ≥97%, ≥98%, or ≥99%).

### Synthesis of (Z)-Endoxifen

Several methods may be used for the synthetic preparation of endoxifen a. For example, methods of synthetic preparations of endoxifen and their prodrugs and salts may be prepared as described in U.S. Pat. No. 9,333,190 (Ahmad, Jina Pharmaceuticals); WO 2008/070463 (Ahmad, Jina Pharmaceuticals), U.S. Pub. No. 2010/0112041 (Ahmad, Jina Pharmaceuticals), WO 2012/050263 (Ahmad, Jina Pharmaceuticals), WO 2014/141292 (Desai, Intas Pharmaceuticals), WO 2017/070651 (USA/Alchem Lab. Corp.); WO 2009/120999A2 (Kushner), U.S. Pat. No. 8,063,249 (Kushner, Olema Pharmaceuticals), U.S. Pat. Nos. 7,531,578 and 8,119,695 (Forman and Yu), or WO 2012/050263 (Song, CJ Cheiljedang Corp).

Additional methods that may be used for the synthetic preparation of endoxifen include those described in Gauthier et al., J. Org. Chem, 61, 3890-3893 (1996), Fauq et al., Bioorg Med Chem Lett. 2010 May 15; 20 (10):3036-3038); Steams et al., J. Natl. Cancer Inst. Vol 95, No. 23, 2003; Johnson et al., Breast Cancer Research and Treatment. 85:151-159, 2004; and Ogawa, et al. Chem. Pharm. Bull. 39, 911-916, 1991. However, there remains an unmet need for large scale industrial scalable manufacturing. Furthermore, there is a need for methods, including industrially scalable methods, of synthesizing and purifying (Z)-endoxifen with high purity. The methods provided herein, such as a method according to SCHEME 1, may be used to prepare endoxifen in fewer steps than other synthetic methods. As shown in SCHEME 1, the methods described herein may be used to synthesize and purify (Z)-endoxifen in three steps. A three-step process may have increased (Z)-endoxifen yield compared to a method with more steps (e.g., a four-step or five-step process), since loss may occur at each step of the reaction.

The present disclosure addresses the demand by providing methods for synthesizing highly pure (e.g., isomerically pure) and stable (Z)-endoxifen. Specifically, the presently disclosed methods comprise a plurality of crystallization steps for the enrichment of (Z)-endoxifen from an isomeric mixture of (E)/(Z)-endoxifen. Such crystallization steps can comprise an EtOAc-based crystallization, an IPA/acetone-based crystallization, and a THF-IPA-based crystallization. Thus, in some embodiments, stable (Z)-endoxifen may be prepared accordance to SCHEME 1 as further described below and in EXAMPLE 1-EXAMPLE 3.

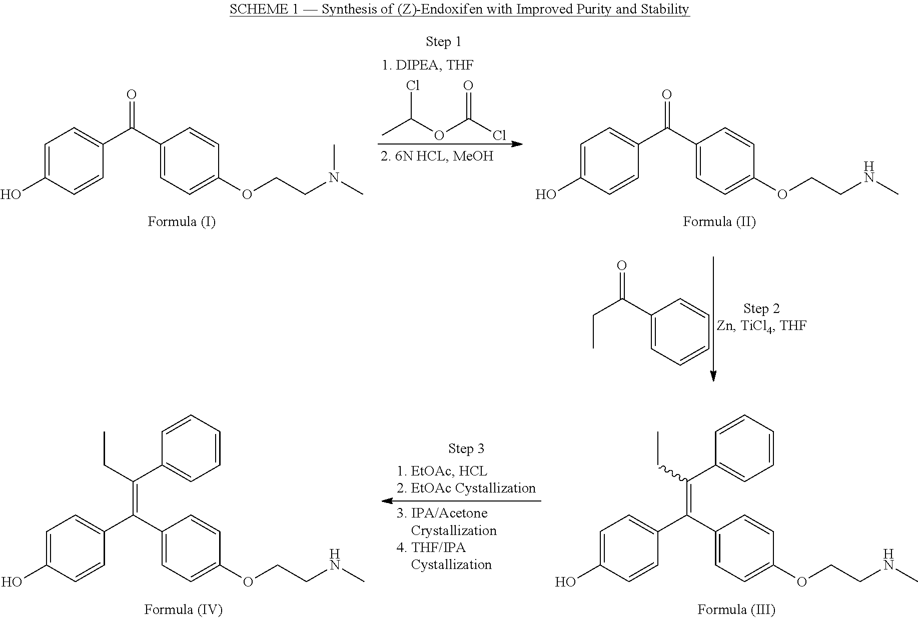

### Synthetic Step 1—Demethylation

In one aspect, the present disclosure relates to an industrially scalable process of making substantially purified (Z)-endoxifen free base, E/Z-mix, or pharmaceutically acceptable salts thereof, such process comprising demethylating the compound of Formula (I), [4-[2-(dimethylamino)ethoxy]phenyl](4-hydroxyphenyl)methanone), (e.g., available from AstaTech Pharmaceuticals, Inc., China) to form the compound of Formula (II), e.g., as described in EXAMPLE 1 herein. Thus, in some embodiments, the industrial process comprises preparing the compound of Formula (II) by demethylating [4-[2-(dimethylamino)ethoxy]phenyl](4-hydroxyphenyl)methanone) (i.e., the compound of Formula (I)) with a demethylating agent and a proton acceptor in an inert organic solvent to form the compound of Formula (II). The industrially scalable process for making the compound of Formula (II) can comprise demethylating the compound of Formula (I) in amounts ranging from 1 mg to 1000 kg per reaction. In some embodiments, from about 0.1 kg to about 10 kg of the compound of Formula (I) is demethylated using a process described herein.

In some embodiments, the compound of Formula (I) can be demethylated by reacting the compound of Formula (I) with a demethylating agent. The demethylating agent can be any agent suitable for the purpose. Examples of suitable demethylating agents include N-iodosuccinamide (NIS), ethyl chloroformates (such as 1-chloroethyl chloroformate, dichloroethyl chloroformate, trichloroethyl chloroformate, α-Chloroethylchloroformate (ACE-Cl)), vinyl chloroformate (VO—Cl), cyanogen bromide (BrCN: von Braun's reaction), diethyl azodicarboxylate, pyridinium chloride, and the like.

In some embodiments, the demethylating agent is a chloroformate selected from the group consisting of 1-chloroethyl chloroformate, dichloroethyl chloroformate, trichloroethyl chloroformate, α-Chloroethylchloroformate (ACE-Cl), and vinyl chloroformate (VO—Cl). In other embodiments, the demethylating agent is an ethyl chloroformate selected from the group consisting of ethyl chloroformates, such as 1-chloroethyl chloroformate, dichloroethyl chloroformate, trichloroethyl chloroformate, and α-Chloroethylchloroformate (ACE-Cl). In at least one embodiment, the demethylating agent is 1-chloroethyl chloroformate.

In some embodiments, a demethylating agent is added to the reaction mixture at a wt/wt ratio of the compound of Formula (I) to demethylating agent ranging from 1:0.5 to 1:10. In other embodiments, the demethylating agent is present at ratios (wt/wt) of the compound of Formula (I) to demethylating agent ranging from 1:2: to 1:5. In at least one embodiment, the demethylating agent is present at a ratio (wt/wt) of the compound of Formula (I) to demethylating agent of 1:2. In at least one embodiment, the demethylating agent is present at a ratio (wt/wt) of the compound of Formula (I) to demethylating agent of 1:3.3. In other embodiments, the demethylating agent 1-chloroethyl chloroformate is present at a ratio (wt/wt) of the compound of Formula (I) to demethylating agent ranging from 1:0.5 to 1:10.

The demethylation reaction can be carried out in an inert organic solvent suitable for the demethylation reaction in the presence of a proton acceptor. Such inert organic solvents include dichloromethane, dichloroethane, chloroform, carbon tetrachloride, chlorobenzene, diethyl ether, 1,4-dioxane, tert-butyl methyl ether (TBME), tetrahydrofuran (THF), N,N-dimethylformamide (DMF), N-methylpyrrolidone (NMP), diglyme, nitromethane, 1,2-dimethoxyethane (DME), pyridine, acetone, acetonitrile, benzene, o-xylene, m-xylene, p-xylene, xylenes, hexanes, cyclohexane, heptane, octane, nonane, and decane, or a combination thereof. In an aspect, the industrially scalable process comprises one or more inert organic solvents for demethylation at a (wt/wt) ratio of the compound of Formula (I) to inert organic solvent ranging from 1:1 to 1:50. In some embodiments, the demethylation solvent is THE at a ratio of the compound of Formula (I) to THE (wt/wt) ranging from 1:1 to 1:50. In other embodiments, the THF is present in ratios (wt/wt) of the compound of Formula (I) to THF ranging from 1:1 to 1:20.

Proton acceptors suitable for the purpose of the present disclosure include, but are not limited to carbonates, such as sodium carbonate and potassium carbonate, and bicarbonates, such as sodium bicarbonate and potassium bicarbonate, proton sponge, and ethyldiisopropylamine, (N,N-diisopropylethylamine, “DIPEA”). In an aspect, the industrially scalable process comprises a proton acceptor added to the reaction mixture at a wt/wt ratio of the compound of Formula (I) to proton acceptor ranging from 1:0.5 to 1:10. In some embodiments, the proton acceptor is present in a ratio (wt/wt) of the compound of Formula (I) to proton acceptor of 1:1:8.

One of skill in the art will be readily able to determine other solvents and proton acceptors known in the art suitable for demethylation reaction of the present disclosure.

Demethylating agents, solvents for demethylation reaction, and the compound of Formula (I) may be added in any order. Each reagent may be added to a suitable reactor in a single bolus or in multiple boluses and stirred.

In some embodiments, the compound of Formula (I) is charged to a suitable reactor to which THF, an inert organic solvent, and proton acceptor DIPEA are added for demethylation reaction, and heated to 55° C. to 65° C., or to 60° C., followed by addition of one or more demethylating agents. This addition may be performed under inert gas such as nitrogen. THF is added at a wt/wt ratio of 1:1 to 1:20, and proton acceptor DIPEA is added at a wt/wt ratio of 1:0.5 to 1:10 to the demethylation reaction mixture, wherein wt/wt is with respect to the compound of Formula (I). In some embodiments, the reaction mixture is heated to not less than (NLT) 60° C. followed by slow addition of one or more demethylating agents. The reaction may be carried out under inert conditions, such as under nitrogen or argon.

The reaction mixture comprising the compound of Formula (I), one or more demethylating agents (for example, 1-chloroethyl chloroformate) in one or more inert organic solvents can be heated at a temperature ranging from 20° C. to 200° C., such as from 40° C. to 80° C., from 50° C. to 230° C., from 50° C. to 120° C., and from 150° C. to 200° C. In some embodiments, the reaction mixture is heated under reflux. In other embodiments, the compound of Formula (I) is reacted with a demethylating agent and a proton acceptor for not less than (NLT) 5 hours, NLT 8 hours, NLT 12 hours, NLT 24 hours, NLT 36 hours, NLT 48 hour or NLT 72 hours. In at least one embodiment, the reaction is heated under reflux for NLT 12 hours. In some embodiments, the reaction mixture is held not more than (NLT) 12 hours under reflux conditions while stirring.

The mixture can be subjected to one or more rounds of distillation under reduced pressure. Distillation may be carried out at NMT 100° C., at NMT 95° C., at NMT 90° C., at NMT 85° C., at NMT 80° C., or at NMT 70° C. with solvents suitable for distillation such as ethyl acetate, lower alcohols (non-limiting examples include methanol, ethanol, n-propanol, and isopropanol), benzene, acetone, acetonitrile, toluene, dichloromethane, 1,2-dichloroethane, and chloroform. One of skill in the art will be able to readily determine additional suitable solvents useful for the purpose.

In at least one embodiment, the solvent used for distillation is methanol. As a non-limiting example, methanol may be used for solvent swap for 2 to 5 rounds of distillation under reduced pressure.

Solvents, such as methanol, may be used for distillation at a (wt/wt) ratio of the compound of Formula (I) to solvent ranging from 1:1 to 1:10 per round of distillation.

Then, the mixture can be reacted with a solvent/acid mixture by addition of an acid (wt/wt ratio of the compound of Formula (I) to acid ranging from 1:1 to 1:10) to the solvent while stirring and heating under reflux. The acid can be any suitable acid. HCl is an example of acid suitable for the purpose of the present disclosure. Non-limiting examples of suitable solvent/acid mixtures include methanol/HCl, ethanol/HCl, propanol/HCl, isopropanol/HCl, methanol/sulfuric acid, methanol/phosphoric acid, ethanol/sulfuric acid, ethanol/phosphoric acid, propanol/sulfuric acid, propanol/phosphoric acid, isopropanol/sulfuric acid, isopropanol/phosphoric acid, methanol/acetic acid, ethanol/acetic acid, propanol/acetic acid, isopropanol/acetic acid, methanol/formic acid, ethanol/formic acid, propanol/formic acid, and isopropanol/formic acid. In at least one embodiment, the solvent/acid mixture is methanol/6N HCl. As a non-limiting example, methanol in methanol/HCl mixture may be added at a wt/wt ratio of the compound of Formula (I) to methanol in solvent/acid mixture at 1:3.2 while HCl in the methanol/HCl mixture may be at a wt/wt ratio of the compound of Formula (I) to HCl in the solvent/acid mixture at 1:4. In at least one embodiment, the methanol/6N HCl mixture is added at a wt/wt ratio of the compound of Formula (I) to methanol/6N HCl ranging from 1:1 to 1:10, such as 1:1 to 1:5.

Distillation may be carried out for NLT 5 hours, NLT 8 hours, NLT 10 hours, NLT 12 hours, or NLT 14 hours under reduced pressure. Distillation may be carried out at a temperature of NMT 70° C., NMT 75° C., NMT 80° C., NMT 85° C., NMT 90° C. or NMT 90° C. Following distillation, the temperature of the mixture may be cooled to ambient temperature (e.g., from 15° C. to 25° C.).

The pH of the reaction mixture can be increased with an alkaline agent such as sodium hydroxide (NaOH), ammonium hydroxide, aminomethylpropanol and the like. In some embodiments, the pH of the reaction mixture is increased to at least pH 11, at least pH 12, at least pH 13, or at least pH 14. An organic solvent such as ethyl acetate (EtOAc) can be added to obtain (4-hydroxyphenyl)(4-(2-(methylamino)ethoxy)phenyl)methanone, the compound of Formula (II). Addition of the organic solvent (e.g., ethyl acetate) may produce a phase separation comprising an aqueous layer and an organic layer. The aqueous layer may be cooled to a temperature of from −5° C. to 5° C. and stirred for NLT 2 hours. The aqueous layer may be washed with water, the organic solvent, or a combination thereof.

The neutralizing agent may be at a wt/wt ratio of the compound of Formula (I) to the neutralizing agent ranging from 1:1 to 1:10. In some embodiments, the neutralizing agent is 8N sodium hydroxide at a wt/wt ratio ranging from 1:1 to 1:10. In other embodiments, the neutralizing agent is 8N sodium hydroxide at a wt/wt ratio ranging from 1:2 to 1:8.

In another aspect, the industrially scalable process for making (Z)-endoxifen free base, E/Z-mix or pharmaceutically acceptable salts thereof comprises one or more steps of washing a filtered product (e.g., the compound of Formula (II)) with purified water (e.g., 1:1 to 1:5 wt/wt) and organic solvents such as ethyl acetate (EtOAc) (using a ratio of 1:0.5 to 1:10 wt/wt) wherein the wt/wt is with respect to the compound of Formula (I). The wet cake can be dried under reduced pressure/vacuum. The temperature for the drying step can range from 25° C. to 60° C. In some embodiments, the drying is carried out at a temperature of NMT 50° C. or at a temperature of NMT 60° C.

The demethylating reactions described herein may have a chemical yield of NLT about 50%, NLT about 60%, NLT about 65%, NLT about 70%, NLT about 75%, NLT about 80%, NLT about 85%, or NLT about 90%, based on the starting amount used for the compound of Formula (I).

### Synthetic Step 2—McMurry Reaction

In another aspect, the present disclosure relates to an industrially scalable process of making (Z)-endoxifen free base, E/Z-mix, and pharmaceutically acceptable salts thereof, the process comprising subjecting the compound of Formula (II) to a McMurry reaction to afford an E/Z-mix of endoxifen (i.e., a mixture of (E)-endoxifen and (Z)-endoxifen free bases) described as Formula (III), e.g., as described in EXAMPLE 2 herein.

The McMurry reaction may be used to prepare tamoxifen, for example as described in European Patent Application No. 168175. The present disclosure relates to an industrially scalable process wherein the compound of Formula (II), (4-hydroxyphenyl)(4-(2-(methylamino)-ethoxy)phenyl) methanone, can be coupled to propiophenone mediated by a McMurry reaction using titanium salts such as chloride salts of titanium (for example, titanium trichloride and/or titanium tetrachloride (titanium (IV) chloride, TiCl₄)) and reducing agents in inert organic solvents to form an E/Z mix of Formula (III).

Salts of titanium that are useful for the present disclosure include titanium halides (such as titanium trichloride (TiCl₃), Titanium tetrachloride (TiCl₄), titanium iodides, titanium bromides, and titanium fluorides), titanium (IV) trichloride isopropoxide, and titanium isopropoxide. In some embodiments, the titanium salt is TiCl₄. Titanium salts, such as TiCl₄, are added at a wt/wt ratio of the compound of Formula (II) to titanium salt ranging from 1:0.1 to 1:12.

Reducing agents include zinc, zirconium, vanadium, niobium, molybdenum, tungsten, aluminum, magnesium, potassium, zinc-copper couple, alkali, and alkali earth metals, butyllithium, and lithium aluminum hydride. In at least one embodiment, the reducing agent is zinc. The McMurry synthesis can be conveniently carried out using a reducing agent such as zinc at a wt/wt ratio of the compound of Formula (II) to reducing agent ranging from 1:0.1 to 1:10. In some embodiments, the ratio of reducing agent is in excess compared to titanium salts.

The McMurry synthesis can be carried out in one or more inert organic solvents. Inert organic solvents useful for the McMurry reaction include dichloromethane, dichloroethane, chloroform, carbon tetrachloride, chlorobenzene, diethyl ether, 1,4-dioxane, tert-butyl methyl ether (TBME), tetrahydrofuran (THF), N,N-dimethylformamide (DMF), N-methylpyrrolidone (NMP), diglyme, nitromethane, 1,2-dimethoxyethane (DME), pyridine, acetone, acetonitrile, benzene, o-xylene, m-xylene, p-xylene, xylenes, hexanes, cyclohexane, heptane, octane, nonane, and decane, or a combination thereof. In some embodiments, the inert organic solvent is at wt/wt ratio of the compound of Formula (II) to solvent ranging from 1:1 to 1:50. In other embodiments, the inert organic solvent is at wt/wt ratios of the compound of Formula (II) to solvent ranging from 1:1 to 1:20. In some embodiments, the inert organic solvent used for the McMurry reaction is THF. THF, in some embodiments, is at wt/wt ratio of the compound of Formula (II) to THF ranging from 1:1 to 1:20.

It can be advantageous to combine one or more titanium salt(s) and one or more reducing agent(s) in the inert organic solvents to create a pre-mix. Titanium salts can be added to the reducing agent and inert organic solvents at such a rate so as to keep the internal temperature at NMT 75° C., such as NMT 65° C., NMT 55° C., NMT 45° C., NMT 40° C., NMT 35° C., NMT 30° C., NMT 25° C., NMT 20° C., or NMT 15° C. Accordingly, titanium salt(s) and reducing agent(s) in the inert organic solvents are combined to create a pre-mix. In some embodiments, Zn, TiCl₄ and THE are combined to create a Zn/TiCl₄/THF mixture. In at least one embodiment, TiCl₄ is slowly added to Zn and THE and mixed keeping the internal temperature at NMT 20° C.

Preparation of titanium salt and a reducing agent in an inert organic solvent may further comprise heating titanium salt and reducing agent in an inert organic solvent to a temperature ranging from 20° C. to 100° C., such as from 40° C. to 80° C., from 50° C. to 100° C., from 50° C. to 80° C., or from 50° C. to 70° C. In some embodiments, titanium salt and reducing agent present in an inert organic solvent are heated at NLT 60° C. In some embodiments, preparation of titanium salt and a reducing agent in an inert organic solvent further comprises heating titanium salt and reducing agent in an inert organic solvent. Titanium salt and reducing agent in inert organic solvent are heated for NLT 30 min, such as NLT 1 hour, NLT 2 hours, NLT 4 hours, NLT 6 hours and NLT 8 hours, under inert conditions such as under nitrogen (N₂) or argon (Ar).

It may also be advantageous to pre-mix the compound of Formula (II) with inert organic solvent such as THE and propiophenone and then reacting the compound of Formula (II) with pre-mixed reducing agent/titanium salt/solvent mixture such as a Zn/TiCl₄/THF mixture to furnish a mixture of (E)-endoxifen and (Z)-endoxifen, compounds of Formula (III).

Propiophenone can be added at a wt/wt ratio of the compound of Formula (II) to propiophenone ranging from 1:0.01 to 1:5. Inert organic solvent can be at a wt/wt ratio of the compound of Formula (II) to solvent ranging from 1:1 to 1:20. The compound of Formula (II) of step 1 is reacted with titanium salt and a reducing agent in an organic solvent refluxate a temperature of NLT 60° C. or NLT 70° C. for NLT 0.5 hours, NLT 1 hour, NLT 2 hours, NLT 4 hours, NLT 6 hours, NLT 8 hours, NLT 12 hours, NLT 24 hours, or NLT 48 hours. In at least one embodiment, the compound of Formula (II) is slowly mixed with THF and propiophenone for NLT 1 hour. The mixture may be reacted with a pre-mixture of Zn, TiCl₄, THF and heated to NLT 60° C. or NLT 70° C. for NLT 8 hours. In another embodiment, the compound of Formula (II) is mixed with THF and propiophenone and reacted with the Zn/THF/TiCL₄ mixture as described above and heated for NLT 8 hours.

The mixture of (E)-endoxifen and (Z)-endoxifen in the reaction mixture can be then subjected to extractive purification, distillation and crystallization to afford a purified mixture of (E)-endoxifen and (Z)-endoxifen. The mixture of (E)-endoxifen and (Z)-endoxifen in the reaction mixture can be subject to extractive purification by extracting with inert organic solvents, such as THE and 2-methyltetrahydrofuran (MeTHF), or by addition of salts, such as potassium carbonate, ammonium chloride, sodium chloride, sodium hydroxide, to the reaction mixture and extraction with an inert organic solvent, such as THE and MeTHF. The reaction mixture may then be allowed to cool to a temperature from about 0° C. to about 35° C. or NMT 30° C. An acid (e.g., HCl) may be added to the mixture, and the mixture may be stirred for NLT 30 minutes. In some embodiments, 1N of HCl is added. The mixture may then be filtered and phase separated.

In some embodiments, the reaction mixture is extracted one or more times with potassium carbonate (K₂CO₃), such as 40% K₂CO₃ (1:1 to 1:10 wt/wt) and MeTHF (1:1 to 1:10 wt/wt). In some embodiments, the reaction mixture is extracted one or more times with ammonium chloride, such as 25% ammonium chloride (1:1 to 1:30 wt/wt); silica (Celite®) bed (1:0.01 to 1:5 wt/wt); and/or solvent, such as THF (1:1 to 1:10 wt/wt). In some embodiments, the E/Z mixture may be further extracted with NaOH, such as 1N NaOH (1:1 to 1:20 wt/wt). In at least one embodiment, NaCl (1:0.1 to 1:0.5 wt/wt) may be added to 1N NaOH for the extraction step.

In some embodiments, the reaction mixture may be further extracted one or more times with THE or MeTHF. In at least one embodiment, the reaction mixture is extracted 3 or more times with MeTHF. Without being bound to any theory, MeTHF was found to be surprisingly suitable for the step of extraction of a mixture of (E)-endoxifen and (Z)-endoxifen, affording higher yields of purified mixtures of (Z)-endoxifen and (E)-endoxifen. In at least one embodiment, the mixture may be still further extracted with 20% sodium chloride (1:1 to 1:10 wt/wt).

The reaction mixture can be subjected to 2 to 5 rounds of solvent swap and distillation with a suitable solvent, such as acetone, acetonitrile, or a combination thereof (1:1 to 1:10 wt/wt with respect to the compound of Formula (III)). Distillation may be carried out under reduced pressure/vacuum at temperatures ranging from 30° C. to 90° C. In some embodiments, the distillation may be performed at a temperature of NMT 30° C., NMT 35° C., NMT 40° C., NMT 45° C., NMT 50° C., NMT 55° C., NMT 60° C., NMT 65° C., NMT 70° C., NMT 75° C., NMT 80° C. or NMT 90° C., and filtered. The filtered product may be washed with solvents such as pre-cooled acetonitrile and then crystallized with crystallization systems such as EtOAc/n-heptane (1:2 v/v) or IPA/n-heptane (1:2.7 v/v) at a wt/wt ratio of a compound of Formula (III) to EtOAc/n-heptane or IPA/n-heptane ranging from 1:1 to 1:20 and dried, for example, at NMT 60° C., to afford a crystalline solid mixture of (E)-endoxifen and (Z)-endoxifen free bases, compounds of Formula (III).

In yet another aspect, the present disclosure relates to an industrially scalable method of manufacturing or reequilibriating an E/Z-mix having an E/Z ratio of approximately 1:1 (45:55 to 55:45). A suitable reactor may be charged with the compounds of Formula (III) dissolved in an inert organic solvent, such as ethyl acetate, prepared in a McMurry reaction as described above.

The compounds of Formula (III) can have an E/Z ratio of 99:1 to 60:40. The mixture can be concentrated at temperatures ranging from 40° C. to 85° C. In some embodiments, the mixture is concentrated at a temperature of NMT 75° C. until the volume reaches about 5% of the initial volume. The mixture can be heated to reflux and then cooled to temperatures ranging from 40° C. to 60° C. In some embodiments, the temperature of the mixture is cooled to 50±5° C. n-heptane, at a ratio of the compounds of Formula (III) to n-heptane ranging from 1:1 to 1:20, may be added slowly to the mixture and the mixture may be then cooled to 0±5° C. The mixture may be stirred at 0±5° C. for NLT 0.5 hours, NLT 1 hour, NLT 2 hours, NLT 4 hours, NLT 8 hours, NLT 12 hours or NLT 24 hours. The mixture may be filtered and washed with ethyl acetate/n-Heptane (1:2 v/v) at a wt/wt ratio of the compounds of Formula (III) to ethyl acetate/n-heptane ranging from 1:1 to 1:10. The wet cake may be dried under reduced pressure to afford E/Z-endoxifen mixture having an E/Z-ratio of approximately 1:1. Drying may be carried out at temperatures ranging from 30° C. to 70° C. In at some embodiments, the wet cake is dried under reduced pressure or vacuum at NMT 60° C. to afford E/Z-endoxifen mixture having an E/Z-ratio of approximately 1:1 (e.g., from about 45:55 to about 55:45).

The McMurry reactions described herein may have a yield of NLT about 15%, NLT about 20%, NLT about 25%, NLT about 27%, NLT about 30%, NLT about 32%, NLT about 35%, or NLT about 40%, based on the amount used of the compound of Formula (II).

In still another aspect, the compounds of Formula (III) may be further purified or enriched or re-equilibrated to obtain substantially pure (Z)-endoxifen as described below.

### Synthetic Step 3—Enrichment Purification of (Z)-Endoxifen Free Base

In still another aspect, the present disclosure relates to industrially scalable methods of manufacturing—by enrichment and purification—(Z)-endoxifen free base. Industrially scalable enrichment and purification of (Z)-endoxifen may be carried out as described herein using the method of Step 3 of SCHEME 1, and as further described in EXAMPLE 3. The starting mixture of (E)-endoxifen and (Z)-endoxifen used for fractional crystallization can have any E/Z ratio, for example, E/Z ratio ranging from 99:1 to 1:10. In some embodiments, the E/Z-ratio of the starting (E)/(Z)-endoxifen mixtures ranges from about 30:70 to about 70:30. In some embodiments, the E/Z-ratio of the starting (E)/(Z)-endoxifen mixtures ranges from 99:1 to 1:99. In some embodiments, the E/Z-ratio of the starting (E)/(Z)-endoxifen mixtures is 51:1, 1:1.8 or 1:5.6.

The E/Z-mix can be the compounds of Formula (III) obtained as described above. A mixture of (E)-endoxifen and (Z)-endoxifen (E/Z-mix) can be subjected to fractional crystallization to obtain a first crystalline solid and a first mother liquor enriched with (Z)-endoxifen free base. Fractional crystallization can be carried using a first solvent which is capable of triturating endoxifen and its derivatives such that (Z)-endoxifen tends to remain in filtrate. Suitable first solvents are those that differentially solubilize the endoxifen isomers, and include, without limitation, ethyl acetate, isopropanol, isopropanol/PPW, acetonitrile, acetonitrile/PPW, and dichloromethane. In some embodiments, the first recrystallization solvent used in the methods described herein is ethyl acetate. The first solvent, for example, ethyl acetate, is added at a wt/wt ratio of the compounds of Formula (III) to first solvent ranging from 1:1 to 1:20. In some embodiments, the compounds of Formula (III) are dissolved in a solution comprising the first solvent and an acid, for example HCl. The resulting mixture may be heated to a temperature ranging from about 55° C. to about 65° C. and stirred for not less than about 6 hours.

It has been a surprising discovery that acidification of the mixture enhances the conversion of (Z)-endoxifen to (E)-endoxifen. Accordingly, in some embodiments, the compounds of formula (III) are pretreated with an acid and then adjusted with a base.

As a non-limiting example, a suitable reactor is charged with the compounds of Formula (III) to which a first solvent (e.g., ethyl acetate) is added and cooled to −5° C.-10° C. Next, an acid such as HCl or TFA may be added slowly while keeping the temperature at NMT 10° C. In some embodiments, the acid is added to the E/Z-endoxifen mixture at a wt/wt ratio of the compounds of Formula (III) to acid ranging from 1:1 to 1:5. The reaction mixture may then be heated at temperatures ranging from 50° C. to 70° C. while stirring. The reaction may be carried out for NLT 4 hours, such as NLT 6 hours, NLT 12 hours, NLT 24 hours, or NLT 48 hours. The reaction mixture may be cooled to −5° C.-10° C., and the pH may be adjusted to about pH 12.

In some embodiments, the neutralizing agent is added to the reaction mixture at a wt/wt ratio of the compounds of Formula (III) to neutralizing agent ranging from 1:1 to 1:10. Suitable neutralizing agents include sodium hydroxide, potassium hydroxide, ammonium hydroxide, aminomethylpropanol, and the like. In some embodiments, the neutralizing agent is 8N sodium hydroxide. The pH of the reaction mixture is preferably alkaline. In some embodiments, the pH is ≥10, such as ≥11 or ≥12. During the pH adjustment, the temperature of the mixture may be kept at a temperature of not less than about 20° C.

### EtOAc-Based Crystallization

Subsequently, in some embodiments, (Z)-endoxifen is extracted into the organic layer and collected, and the aqueous phase is washed one or more times with the first solvent such as ethyl acetate (added at a wt/wt ratio of the compounds of Formula (III) to first solvent ranging from 1:1 to 1:10). The organic layers are combined and washed one or more times with brine (20% NaCl; added at a wt/wt ratio of the compounds of Formula (III) to NaCl ranging from 1:1 to 1:10). The organic layer may be filtered through activated carbon/(Celite®) bed (for example, added at a wt/wt ratio of the compounds of Formula (III) to silica ranging from 1:0.01 to 1:0.5), and washed one or more times with first solvent, e.g., ethyl acetate (added at a wt/wt ratio of the compounds of Formula (III) to first solvent ranging from 1:1 to 1:10) and the filtrate concentrated to about 80%, 70%, or 60% of the washed filtrate's volume. First solvent, e.g., ethyl acetate, is added to the concentrated solution to increase its volume by about 20-50%, followed by another round of concentration under reduced pressure until about 80%, 70%, or 60% of initial volume. The resulting concentrated solution is cooled to NMT about 5° C., heated to about 40-60° C. and cooled again to NMT about 5° C. The cooled mixture is filtered and washed with first solvent (e.g., ethyl acetate) to furnish (i) a wet cake which can be dried at NMT 90° C. to recover the compound of Formula (III), and (ii) a filtrate. The filtrate fractions, which can be defined as a first mother liquor, can be combined and concentrated.

The first mother liquor can be enriched in (Z)-endoxifen as described herein, e.g., in EXAMPLE 3, e.g., using one, two, three, or more rounds of crystallization. In some embodiments, the first mother liquor is enriched by at least 50% as compared to the E/Z-ratio of the starting mixture of E-endoxifen and Z-endoxifen used in synthetic step 3. In other embodiments, the first mother liquor is enriched in (Z)-endoxifen by at least 70% as compared to the E/Z-ratio of the mixture of E-endoxifen and Z-endoxifen.

### IPA/Acetone-based Recrystallization

In some embodiments, the first mother liquor may be subjected to a second crystallization step using a second recrystallization solvent system (e.g., comprising a first and a second solvent) by concentrating the first mother liquor (e.g., comprising ethyl acetate), adding a first solvent, and/or swapping out the first solvent with a second solvent one or more times. Recrystallization can be carried out using the second solvent for swapping. The second solvent is added at a wt/wt ratio of the compounds of initially used Formula (III) to second solvent ranging from 1:1 to 1:10 to create a slurry. Suitable second solvents include IPA, IPA/PPW, acetone, acetone/MTBE, ethanol, EtOAc, EtOAc/n-heptane. In some embodiments, the first solvent can be acetone. In some embodiments, the second solvent is IPA. Thus, in some cases, both acetone (first solvent) and IPA (second solvent) can be used consecutively in this step. Surprisingly, IPA has been found to triturate (Z)-endoxifen into solid fraction at levels higher than EtOAc. Accordingly, in some embodiments, when the mother liquor solvent is EtOAc, the first solvent (e.g., acetone) can be used in a first swapping step, and the second solvent for swapping is IPA or IPA/PPW, or a combination of acetone and IPA (e.g., using first acetone followed by IPA). This may be useful in directing (Z)-endoxifen first into the filtrate (e.g., mother liquor(s)) from EtOAc and/or acetone, and then into the solid fraction from IPA or IPA/PPW (e.g., cooled IPA). In at least one embodiment, the second solvent comprises IPA/PPW. In yet another embodiment, the second solvent comprises acetone/MTBE. In another embodiment, the first solvent used in this step is acetone and the second solvent is IPA or IPA/PPW, as described in EXAMPLE 3.

Hence, in some embodiments, the concentrated first mother liquor can be diluted with acetone (e.g., about 7, 8, or 9 weight equivalents (“wt.”) relative to amount of initial Formula (III) used) at a temperature of not less than about 30° C. and concentrated. Another round of the same amount of acetone can be added, followed by concentration under reduced pressure to about 20-30% of initial volume. IPA (e.g., about 1-1.5 wt.) is then added to the concentrate at a temperature of NLT 40° C., following by concentration under reduced pressure until the solution reaches about the volume it had prior to IPA addition. The resulting mixture can be cooled to about 0±5° C. and stirred at that temperature for NLT 6 hours. The mixture is then filtered to and washed with pre-cooled IPA (about 1.6 wt.) to furnish a crude second crystalline solid. The second crystalline solid may be crude (Z)-endoxifen, (Z)-4-(1-(4-(2-(methylamino)ethoxy)phenyl)-2-phenylbut-1-enyl)phenol, the compound of Formula (IV) (Molecular weight 373.49; Molecular formula: C₂₅H₂₇NO₂; Melting point 139° C.-143° C.). The second crystalline solid may be ≥70% (Z)-endoxifen, such as ≥75% (Z)-endoxifen, ≥80% (Z)-endoxifen or ≥90% (Z)-endoxifen. In some embodiments, the (crude) second crystalline solid may be ≥90% (Z)-endoxifen. In some embodiments, the product may be dried at a temperature of NMT 70° C., such as NMT 75° C., NMT 80° C., or NMT 85° C. to produce a crude compound of Formula (IV). In some embodiments, the crude compound of Formula IV may comprise a polymorph of (Z)-endoxifen. The polymorph may be characterized, for example, by an X-ray powder diffraction pattern.

### THF/IPA-Based Crystallization

In some embodiments, the dried crude enriched (Z)-endoxifen product may be subjected to a third round of crystallization to reduce or remove remaining impurities. The dried crude product may be treated with a third recrystallization solvent system, which can comprise a first solvent and a second solvent. The dried crude product can be resuspended in the first solvent.

Suitable first solvents include, without limitation, acetone, THF, ethyl acetate, isopropanol, isopropanol/PPW, acetonitrile, acetonitrile/PPW, and dichloromethane. In some embodiments, the first solvent is THF. Recrystallization is carried out using a second solvent for swapping. The second solvent can be added at a wt/wt ratio of the compounds of starting material Formula (III) to second solvent ranging from 1:1 to 1:10 to create a slurry. Suitable second solvents include IPA, IPA/PPW, acetone, acetone/MTBE, ethanol, EtOAc, EtOAc/n-heptane. In some embodiments, the second solvent is IPA. In some embodiments, when the first solvent is THF, the second solvent is IPA or IPA/PPW, as described in EXAMPLE 3.

In some embodiments, the slurry may be filtered and washed with pre-cooled solvent, for example IPA, and the resulting wet cake may be dried at a temperature of NMT about 80° C. The dried enriched product may have a yield of NLT about 8%, NLT about 10%, NLT about 12%, NLT about 14%, NLT about 16%, NLT about 18%, NLT about 20%, NLT about 22%, or NLT about 25%. In some embodiments, the enriched product may be a crystalline solid comprising (Z)-endoxifen. The crystalline solid may be ≥90% (Z)-endoxifen, such as ≥91% (Z)-endoxifen, ≥92% (Z)-endoxifen, ≥93% (Z)-endoxifen, ≥94% (Z)-endoxifen, ≥95% (Z)-endoxifen, ≥96% (Z)-endoxifen, ≥97% (Z)-endoxifen, ≥98% (Z)-endoxifen, or ≥99% (Z)-endoxifen. In some embodiments, the third crystalline solid is ≥90% (Z)-endoxifen. In some embodiments, the third crystalline solid is ≥95% (Z)-endoxifen. In some embodiments, the crystalline solid is ≥90% (Z)-endoxifen. In some embodiments, the crystalline solid is ≥95% (Z)-endoxifen. In some embodiments, the crystalline solid is ≥97% (Z)-endoxifen.

The second crystalline solid may optionally be subjected to further recrystallization to obtain a third crystalline solid, (Z)-endoxifen. The third crystalline solid may be ≥90% (Z)-endoxifen, such as ≥91% (Z)-endoxifen, ≥92% (Z)-endoxifen, ≥93% (Z)-endoxifen, ≥94% (Z)-endoxifen, ≥95% (Z)-endoxifen, ≥96% (Z)-endoxifen, ≥97% (Z)-endoxifen, ≥98% (Z)-endoxifen, or ≥99% (Z)-endoxifen. In some embodiments, the third crystalline solid is ≥90% (Z)-endoxifen. In some embodiments, the purified crystalline solid is ≥95% (Z)-endoxifen. In some embodiments, the third crystalline solid is ≥97% (Z)-endoxifen. This optional recrystallization is carried out using a third solvent. The third solvent is selected from the group consisting of ethanol, methanol, ethyl acetate, IPA, IPA/PPW, THF, acetone, acetone/MTBE and EtOAc/n-heptane.

Additional recrystallization steps, for example a fourth, fifth, or sixth recrystallization step may be carried out to further enrich the crystalline product of (Z)-endoxifen. Further crystallization steps may be carried out in a solvent selected from the group consisting of ethanol, methanol, ethyl acetate, IPA, IPA/PPW, THF, acetone, acetone/MTBE and EtOAc/n-heptane.

In an aspect, the present disclosure relates to pre-heating first solvent, second solvent, and third solvent prior to use. In some embodiments, one or more of the first solvent, second solvent, and third solvent can each be independently preheated to a temperature ranging from 40° C. to 80° C. Fractional crystallization and recrystallization steps may also include steps of distilling at 60° C. to 80° C. and/or cooling the resulting solution to a temperature ranging from 0° C. to 35° C.

It is to be understood that in some embodiments, first, second, and third crystalline solids as well as the second mother liquor obtained as described herein may be further subjected to fractional crystallization and recrystallization as described herein one or more time(s) to obtain purified (Z)-endoxifen. It is also to be understood that first, second, and/or third crystalline solids obtained may be optionally reprocessed using column chromatography techniques to obtain more (Z)-endoxifen.

In certain embodiments, the industrially scalable methods described herein independently comprise additional steps or procedures (e.g., to remove reaction by-products, or to workup, isolate or purify reaction products) as detailed in examples herein. In some embodiments, the (Z)-endoxifen free base has <2%, <1%, and <0.5% impurity. In other embodiments, the compounds of Formula (III) have <2%, <1%, and <0.5% impurity.

A person of skill in the art will recognize several parameters of the foregoing process that may be varied in order to obtain a desirable outcome. These parameters include for example, the methods and means of purification of reaction components and solvents; the order of addition of said reaction components and solvents to the reaction mixture, duration of reaction of said reaction components and solvents; and temperature and rate of stirring, mixing or agitation of the reaction components and solvents during the reactions.

### Removal of Impurities

In some embodiments, one or more impurities may be present in the compounds formed in the methods described herein. In some instances, an impurity may be present at or below an allowable concentration limit. In some instances, an impurity may be present above an allowable concentration limit. Impurities, including those present at a concentration above an allowable limit, may be removed using the purification methods described herein, for example by fractional crystallization, recrystallization, or combinations thereof. In some embodiments, an amount of an impurity may be reduced using the purification methods described herein, e.g., one, two, three or more cycles of (re)-crystallization using one or more solvent systems as described herein. In some cases, an impurity may be present at a concentration above an allowable limit prior to purification and may be present at or below an allowable concentration, present below a limit of detection, or absent following purification. In some embodiments, a purified sample of a compound of Formula (IV) herein can contain at least 90%, 91%, 92%, 93%, 94%, 95%, 96%, 97%, 98%, 99%, 99.5%, or 99.9% (Z)-endoxifen, by weight, following purification.

Purification may be performed following any of the synthesis steps described herein. In some embodiments, purification may be performed following one or more steps of SCHEME 1. For example, purification may be performed following a demethylation reaction (e.g., the demethylation reaction described in EXAMPLE 1) or a McMurry reaction (e.g., the McMurry reaction described in EXAMPLE 2). In some embodiments, purification may be performed on a commercially obtained sample, for example a commercially sourced (E)/(Z)-endoxifen mixture of Formula (III). Purification may comprise one or more steps of crystallization, recrystallization, or combinations thereof. In various embodiments, purification of a compound of Formula (IV) may comprise the steps of an EtOAc crystallization, an IPA/acetone recrystallization and a THF/IPA recrystallization (e.g., as described in EXAMPLE 3). Conducting these crystallization steps has been shown to effectively remove impurities from crude Formula (IV) product, particularly impurities such as mesityl oxide, to furnish isomerically pure Formula (IV), (Z)-endoxifen, with purities of >95%, >96%, >97%, >98%, or >99% by weight.

Impurities that may be reduced or removed using the purification methods described herein include, without limitation: (E)-endoxifen; mesityl oxide; 4-(1-(4-methoxyphenyl)-2-phenylbut-1-en-1-yl)phenol; 4-(1-(4-isopropoxyphenyl)-2-phenylbut-1-en-1-yl)phenol; (1S,2R)-1-(4-(2-hydroxyethoxy)phenyl)-1,2-bis(4-hydroxyphenyl)-2-(4-(2-(methylamino)ethoxy)-phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; benzene; methanol; ethanol; acetone; 2-propanol; acetonitrile; ethyl acetate; THF; 2-methyltetrahydrofuran; n-heptane; zinc; titanium; or combinations thereof. For example, the purification methods described herein may reduce a level of mesityl oxide, (E)-endoxifen, or both in a composition comprising (Z)-endoxifen. In some cases, the level of mesityl oxide in the composition comprising (Z)-endoxifen may be reduced using the purification methods described herein.

An impurity may be present at or below an allowable concentration, present below a limit of detection, or absent in a compound described herein (e.g., a compound of Formula (I), a compound of Formula (II), a compound of Formula (III), and/or a compound of Formula (IV)) following purification. An allowable concentration of methanol in a compound may be not more than (NMT) about 3000 parts per million (ppm). An allowable concentration of ethanol in a compound may be NMT about 5000 ppm. An allowable concentration of acetone in a compound may be NMT about 5000 ppm. An allowable concentration of 2-propanol (IPA) in a compound may be NMT about 5000 ppm. An allowable concentration of acetonitrile in a compound may be NMT about 410 ppm. An allowable concentration of ethyl acetate in a compound may be NMT about 5000 ppm. An allowable concentration of THF in a compound may be NMT about 720 ppm. An allowable concentration of 2-methyltetrahydrofuran (MeTHF) in a compound may be NMT about 520 ppm. An allowable concentration of n-heptane in a compound may be NMT about 5000 ppm. An allowable concentration of zinc in a compound may be NMT about 130 ppm. An allowable concentration of benzene in a compound may be NMT about 2 ppm. An allowable concentration of mesityl oxide in a compound may be NMT about 25 ppm. In some embodiments, a concentration of an impurity may be measured using an appropriate analytical method, such as gas chromatography. In some embodiments, a purified batch of Formula (IV) produced using the methods described herein comprises NMT 3000 ppm methanol, NMT 5000 ppm ethanol, NMT 5000 ppm acetone, NMT 5000 ppm IPA, NMT 410 ppm acetonitrile, NMT 5000 ppm ethyl acetate, NMT 720 ppm THF, NMT 520 ppm MeTHF, NMT 5000 ppm n-heptane, NMT 130 ppm zinc, NMT 2 ppm benzene, and NMT 25 ppm mesityl oxide.

### Removal of (E)-Endoxifen

A level of (E)-endoxifen may be reduced in a compound of Formula (III) or a compound of Formula (IV) using one or more purification methods described herein. In some embodiments, (E)-endoxifen may be present in a compound of Formula (III) obtained commercially, a compound of Formula (III) synthesized as illustrated in SCHEME 1, or a compound of Formula (IV) synthesized as illustrated in SCHEME 1. (E)-endoxifen may form during a McMurry reaction (e.g., step 2 of SCHEME 1). Reduction of a level of (E)-endoxifen in a compound (e.g., a compound of Formula (III) or a compound of Formula (IV)) may comprise ethyl acetate fractional crystallization (e.g., as described in EXAMPLE 3). Reduction of a level of (E)-endoxifen in a compound may comprise acetone recrystallization (e.g., as described in EXAMPLE 3), e.g., using an acetone/IPA solvent system. Reduction of a level of (E)-endoxifen in a compound may comprise THE recrystallization, (e.g., as described in EXAMPLE 3), e.g., using a THF/IPA solvent system. Prior to purification, (E)-endoxifen may be present in a compound (e.g., a compound of Formula (III)) at a level of at least about 0.5%, 1%, 2%, 3%, 4%, 5%, 7%, 10%, 20%, 30%, 40%, 50%, 60%, 70%, 80%, or 90% of the compound by weight. Following purification, (E)-endoxifen may be present in a compound (e.g., a compound of Formula (IV)) at a level of no more than about 0.1%, 0.2%, 0.3%, 0.4%, 0.5%1, %, 1.5%, 2%, 2.5%, 3%, 3.5% 4%, 4.5% 5%, 10%, 15%, 20%, 25% of the compound by weight. In such embodiments, (E)-endoxifen may be present in a compound of Formula (IV) at a level of no more than about 1%, 2%, or 3% by weight. [0244](E)-endoxifen impurities in a compound, such as a compound of Formula (IV), may be identified by an analytical method, such as HPLC or LC-MS. In some embodiments, (E)-endoxifen may have a relative retention time (RRT) of about 0.95, relative to (Z)-endoxifen, as measured by HPLC and LC-MS.

### Removal of Mesityl Oxide

A level of mesityl oxide may be reduced in a compound described herein (e.g., a compound of Formula (III) or a compound of Formula (IV)) using a purification method described herein, e.g., a crystallization step. For example, mesityl oxide may be reduced using THF/IPA recrystallization. In some embodiments, mesityl oxide may be introduced from raw materials used in the reaction. In some embodiments, mesityl oxide may form during one or more steps of a reaction of SCHEME 1. For example, mesityl oxide may form via an aldol condensation of acetone, as illustrated in SCHEME 2:

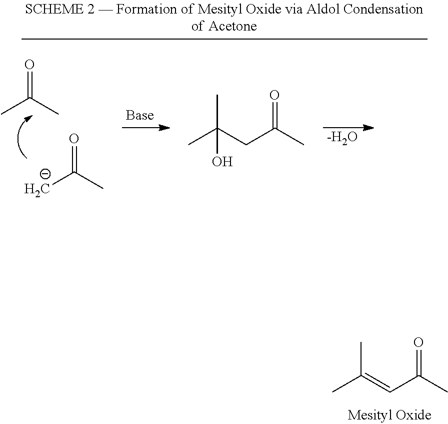

Prior to purification, mesityl oxide may be present in a compound described herein at a concentration above a permissible concentration. In some embodiments, mesityl oxide may be present in a compound formed during one or more steps of a reaction of SCHEME 1. For example, mesityl oxide may be present in a compound of Formula (IV) at a concentration above a permissible concentration prior to purification. A permissible concentration of mesityl oxide in a compound of Formula (IV) may be not more than about 25 ppm. In some embodiments, a permissible concentration of mesityl oxide in a compound may be not more than about 5 ppm, about 10 ppm, about 15 ppm, about 20 ppm, or about 25 ppm.

A level of mesityl oxide present in a compound, such as a compound synthesized as illustrated in SCHEME 1, may be reduced or removed using one or more purification methods described herein. Reduction of a level of mesityl oxide in a compound may comprise THE recrystallization, (e.g., as described in EXAMPLE 3), e.g., using a THF/IPA solvent system. Prior to purification (e.g., prior to THE recrystallization), mesityl oxide may be present in a compound (e.g., a compound of Formula (IV)) at a level of greater than about 25 ppm, about 30 ppm, about 35 ppm, about 40 ppm, about 45 ppm, about 50 ppm, about 60 ppm, about 70 ppm, about 80 ppm, about 90 ppm, about 100 ppm, about 150 ppm, about 200 ppm, about 250 ppm, about 300 ppm, about 350 ppm, about 400 ppm, about 450 ppm, or about 500 ppm. Following purification, mesityl oxide may be present in a compound (e.g., a compound of Formula (IV)) at a level of no more than about 5 ppm, about 10 ppm, about 15 ppm, about 20 ppm, or about 25 ppm, or the level of mesityl oxide may be below the detection limit of one or more analytical methods used, e.g., HPLC/HLPC-MS, GC/GC-MS, headspace-GC, etc.

Mesityl oxide impurities in a compound, such as a compound of Formula (IV), may be identified by an analytical method, such as headspace gas chromatography (GC). In some embodiments, mesityl oxide may have a retention time (RT) of about 14.1 minutes, as measured by headspace-GC.

Reduction of 4-(1-(4-methoxyphenyl)-2-phenylbut-1-en-1-yl)phenol

A level of 4-(1-(4-methoxyphenyl)-2-phenylbut-1-en-1-yl)phenol may be reduced in a compound described herein (e.g., a compound of Formula (III) or a compound of Formula (IV)) using one or more purification methods described herein. In some embodiments, 4-(1-(4-methoxyphenyl)-2-phenylbut-1-en-1-yl)phenol may form during one or more steps of a reaction of SCHEME 1. For example, 4-(1-(4-methoxyphenyl)-2-phenylbut-1-en-1-yl)phenol may form via substitution of a compound of Formula (II) in the presence of methanol followed by condensation with propiophenone in the presence of Zn/TiCl₄, as illustrated in SCHEME 3:

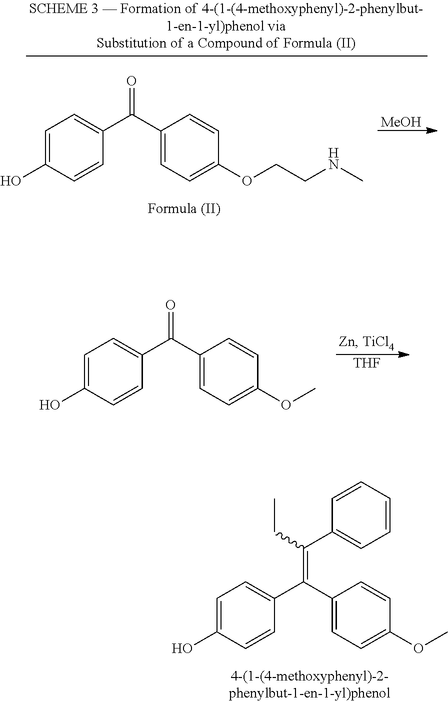 [SMILES: "MeOH" ; CNCCOc1ccc(C(=O)c2ccc(O)cc2)cc1 ; COc1ccc(C(=O)c2ccc(O)cc2)cc1 ; CCC(=C(c1ccc(O)cc1)c1ccc(OC)cc1)c1ccccc1 ; "THF"]

Prior to purification, 4-(1-(4-methoxyphenyl)-2-phenylbut-1-en-1-yl)phenol may be present in a compound described herein at a concentration above a permissible concentration. In some embodiments, 4-(1-(4-methoxyphenyl)-2-phenylbut-1-en-1-yl)phenol may be present in a compound formed during one or more steps of a reaction of SCHEME 1. For example, 4-(1-(4-methoxyphenyl)-2-phenylbut-1-en-1-yl)phenol may be present in a compound of Formula (I), a compound of Formula (II), a compound of Formula (III), or a compound of Formula (IV) at a concentration above a permissible concentration prior to purification. A permissible concentration of 4-(1-(4-methoxyphenyl)-2-phenylbut-1-en-1-yl)phenol in a compound may be not more than about 1%. In some embodiments, a permissible concentration of 4-(1-(4-methoxy-phenyl)-2-phenylbut-1-en-1-yl)phenol in a compound may be not more than about 0.1%, about 0.15%, about 0.2%, about 0.25%, about 0.3%, about 0.4%, about 0.5%, about 0.6%, about 0.7%, about 0.8%, about 0.9%, about 1%, about 1.5%, about 2%, about 2.5%, about 3%, about 4%, or about 5%.

A level of 4-(1-(4-methoxyphenyl)-2-phenylbut-1-en-1-yl)phenol present in a compound, such as a compound synthesized as illustrated in SCHEME 1, may be reduced or removed using one or more purification methods described herein. Reduction of a level of 4-(1-(4-methoxyphenyl)-2-phenylbut-1-en-1-yl)phenol in a compound (e.g., a compound of Formula (III) or a compound of Formula (IV)) may comprise ethyl acetate fractional crystallization (e.g., as described in EXAMPLE 3). Reduction of a level of 4-(1-(4-methoxyphenyl)-2-phenylbut-1-en-1-yl)phenol in a compound may comprise acetone recrystallization, (e.g., as described in EXAMPLE 3). Reduction of a level of 4-(1-(4-methoxyphenyl)-2-phenylbut-1-en-1-yl)phenol in a compound may comprise THF recrystallization, (e.g., as described in EXAMPLE 3). In some embodiments, all three crystallization steps may be applied.

Prior to purification, 4-(1-(4-methoxyphenyl)-2-phenylbut-1-en-1-yl)phenol may be present in a compound (e.g., a compound of Formula (III) or a compound of Formula (IV)) at a level of greater than about 0.1%, about 0.15%, about 0.2%, about 0.25%, about 0.3%, about 0.4%, about 0.5%, about 0.6%, about 0.7%, about 0.8%, about 0.9%, about 1%, about 1.5%, about 2%, about 2.5%, about 3%, about 4%, or about 5%. Following purification, 4-(1-(4-methoxyphenyl)-2-phenylbut-1-en-1-yl)phenol may be present in a compound (e.g., a compound of Formula (IV)) at a level of no more than about 0.1%, about 0.15%, about 0.2%, about 0.25%, about 0.3%, about 0.4%, about 0.5%, about 0.6%, about 0.7%, about 0.8%, about 0.9%, about 1%, about 1.5%, about 2%, about 2.5%, about 3%, about 4%, or about 5%.

4-(1-(4-methoxyphenyl)-2-phenylbut-1-en-1-yl)phenol impurities in a compound, such as a compound of Formula (IV), may be identified by an analytical method, such as HPLC or LC-MS. In some embodiments, 4-(1-(4-methoxyphenyl)-2-phenylbut-1-en-1-yl)phenol may have a relative retention time (RRT) of about 1.53, relative to (Z)-endoxifen, as measured by HPLC and LC-MS.

Reduction of 4-(1-(4-isopropoxyphenyl)-2-phenylbut-1-en-1-yl)phenol

A level of 4-(1-(4-isopropoxyphenyl)-2-phenylbut-1-en-1-yl)phenol may be reduced in a compound described herein (e.g., a compound of Formula (III) or a compound of Formula (IV)) using one or more purification methods described herein. In some embodiments, 4-(1-(4-isopropoxyphenyl)-2-phenylbut-1-en-1-yl)phenol may form during one or more steps of a reaction of SCHEME 1. For example, 4-(1-(4-isopropoxyphenyl)-2-phenylbut-1-en-1-yl)phenol may form via substitution of a compound of Formula (III) in the presence of 2-propanol, as illustrated in SCHEME 4:

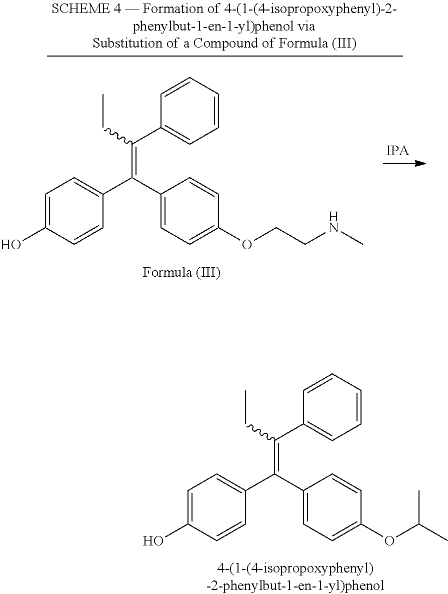 [SMILES: CCC(=C(c1ccc(O)cc1)c1ccc(OCCNC)cc1)c1ccccc1 ; CCC(=C(c1ccc(O)cc1)c1ccc(OC(C)C)cc1)c1ccccc1]

Prior to purification, 4-(1-(4-isopropoxyphenyl)-2-phenylbut-1-en-1-yl)phenol may be present in a compound described herein at a concentration above a permissible concentration. In some embodiments, 4-(1-(4-isopropoxyphenyl)-2-phenylbut-1-en-1-yl)phenol may be present in a compound formed during one or more steps of a reaction of SCHEME 1. For example, 4-(1-(4-isopropoxyphenyl)-2-phenylbut-1-en-1-yl)phenol may be present in a compound of Formula (I), a compound of Formula (II), a compound of Formula (III), and/or a compound of Formula (IV) at a concentration above a permissible concentration prior to purification. A permissible concentration of 4-(1-(4-isopropoxyphenyl)-2-phenylbut-1-en-1-yl)phenol in a compound may be not more than about 1%. In some embodiments, a permissible concentration of 4-(1-(4-isopropoxyphenyl)-2-phenylbut-1-en-1-yl)phenol in a compound may be not more than about 0.1%, about 0.15%, about 0.2%, about 0.25%, about 0.3%, about 0.4%, about 0.5%, about 0.6%, about 0.7%, about 0.8%, about 0.9%, about 1%, about 1.5%, about 2%, about 2.5%, about 3%, about 4%, or about 5%.

A level of 4-(1-(4-isopropoxyphenyl)-2-phenylbut-1-en-1-yl)phenol present in a compound, such as a compound synthesized as illustrated in SCHEME 1, may be reduced or removed using one or more purification methods described herein. Reduction of a level of 4-(1-(4-isopropoxyphenyl)-2-phenylbut-1-en-1-yl)phenol in a compound (e.g., a compound of Formula (III) or a compound of Formula (IV)) may comprise ethyl acetate fractional crystallization (e.g., as described in EXAMPLE 3). Reduction of a level of 4-(1-(4-isopropoxyphenyl)-2-phenylbut-1-en-1-yl)phenol in a compound may comprise acetone recrystallization, (e.g., as described in EXAMPLE 3). Reduction of a level of 4-(1-(4-isopropoxyphenyl)-2-phenylbut-1-en-1-yl)phenol in a compound may comprise THE recrystallization, (e.g., as described in EXAMPLE 3). In some embodiments, all three crystallization steps may be applied.

Prior to purification, 4-(1-(4-isopropoxyphenyl)-2-phenylbut-1-en-1-yl)phenol may be present in a compound (e.g., a compound of Formula (III) or a compound of Formula (IV)) at a level of greater than about 0.1%, about 0.15%, about 0.2%, about 0.25%, about 0.3%, about 0.4%, about 0.5%, about 0.6%, about 0.7%, about 0.8%, about 0.9%, about 1%, about 1.5%, about 2%, about 2.5%, about 3%, about 4%, or about 5%. Following purification, 4-(1-(4-isopropoxyphenyl)-2-phenylbut-1-en-1-yl)phenol may be present in a compound (e.g., a compound of Formula (IV)) at a level of no more than about 0.1%, about 0.15%, about 0.2%, about 0.25%, about 0.3%, about 0.4%, about 0.5%, about 0.6%, about 0.7%, about 0.8%, about 0.9%, about 1%, about 1.5%, about 2%, about 2.5%, about 3%, about 4%, or about 5%. [0258]4-(1-(4-isopropoxyphenyl)-2-phenylbut-1-en-1-yl)phenol impurities in a compound, such as a compound of Formula (IV), may be identified by an analytical method, such as HPLC or LC-MS. In some embodiments, 4-(1-(4-isopropoxyphenyl)-2-phenylbut-1-en-1-yl)phenol may have a relative retention time (RRT) of about 1.66, relative to (Z)-endoxifen, as measured by HPLC and LC-MS.

### Reduction of 1-(4-(2-hydroxyethoxy)phenyl)-1,2-bis(4-hydroxyphenyl)-2-(4-(2-(methylamino)ethoxy)phenyl)ethane-1,2-diol

A level of 1-(4-(2-hydroxyethoxy)phenyl)-1,2-bis(4-hydroxyphenyl)-2-(4-(2-(methylamino)ethoxy)phenyl)ethane-1,2-diol may be reduced in a compound described herein (e.g., a compound of Formula (III) or a compound of Formula (IV)) using one or more purification methods described herein. 1-(4-(2-hydroxyethoxy)phenyl)-1,2-bis(4-hydroxyphenyl)-2-(4-(2-(methylamino)ethoxy)phenyl)ethane-1,2-diol may be present as one or more possible stereoisomers. For example, 1-(4-(2-hydroxyethoxy)phenyl)-1,2-bis(4-hydroxyphenyl)-2-(4-(2-(methylamino)ethoxy)phenyl)ethane-1,2-diol may be present as (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or combinations thereof. In some embodiments, 1-(4-(2-hydroxyethoxy)phenyl)-1,2-bis(4-hydroxyphenyl)-2-(4-(2-(methylamino)ethoxy)phenyl)ethane-1,2-diol may form during one or more steps of a reaction of SCHEME 1. For example, 1-(4-(2-hydroxyethoxy)phenyl)-1,2-bis(4-hydroxyphenyl)-2-(4-(2-(methylamino)ethoxy)phenyl)ethane-1,2-diol may form via substitution of a compound of Formula (I) in the presence of acid followed by condensation with a compound of Formula (II) in the presence of Zn/TiCl₄, as illustrated in SCHEME 5.

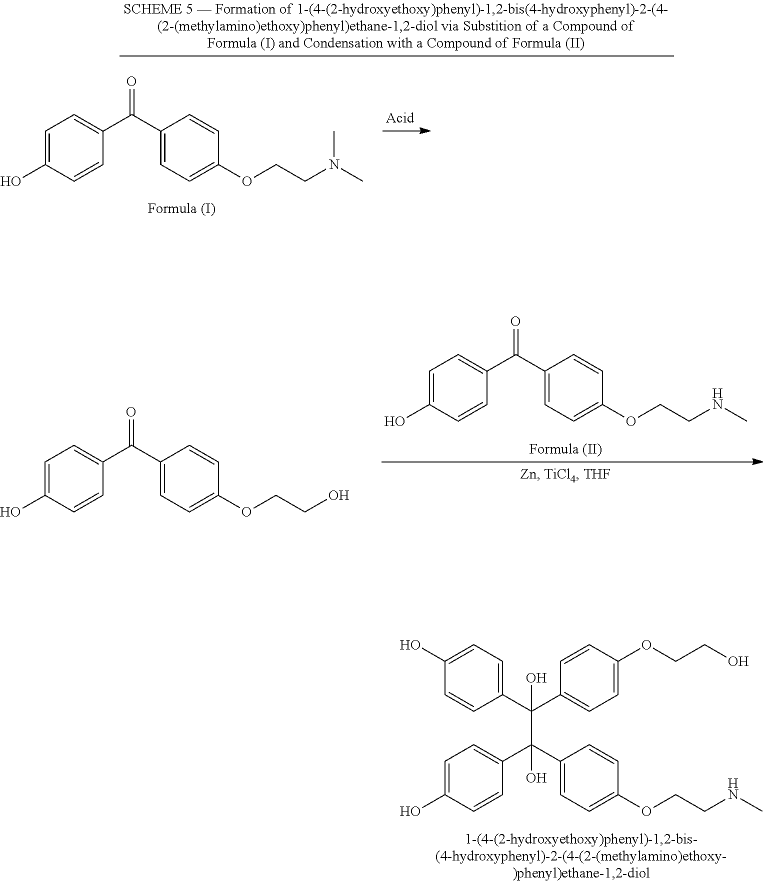 [SMILES: CN(C)CCOc1ccc(C(=O)c2ccc(O)cc2)cc1 ; O=C(c1ccc(O)cc1)c1ccc(OCCO)cc1 ; CNCCOc1ccc(C(=O)c2ccc(O)cc2)cc1 ; CNCCOc1ccc(C(O)(c2ccc(O)cc2)C(O)(c2ccc(O)cc2)c2ccc(OCCO)cc2)cc1]

In another example, 1-(4-(2-hydroxyethoxy)phenyl)-1,2-bis(4-hydroxyphenyl)-2-(4-(2-(methylamino)ethoxy)phenyl)ethane-1,2-diol may form via condensation of a compound of Formula (II) in the presence of Zn/TiCl₄ followed by substitution in the presence of acid, as illustrated in SCHEME 6:

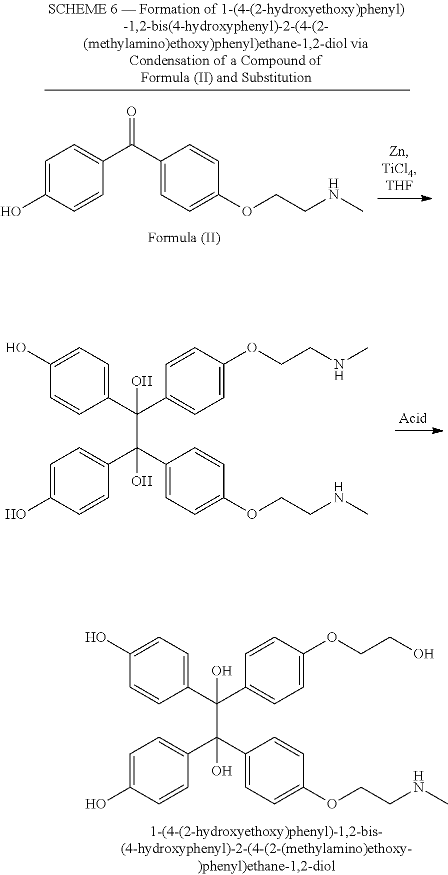 [SMILES: CNCCOc1ccc(C(=O)c2ccc(O)cc2)cc1 ; CNCCOc1ccc(C(O)(c2ccc(O)cc2)C(O)(c2ccc(O)cc2)c2ccc(OCCNC)cc2)cc1 ; CNCCOc1ccc(C(O)(c2ccc(O)cc2)C(O)(c2ccc(O)cc2)c2ccc(OCCO)cc2)cc1]

Prior to purification, (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof may be present in a compound described herein at a concentration above a permissible concentration. In some embodiments, (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof may be present in a compound formed during one or more steps of a reaction of SCHEME 1. For example, (1S,2R)-1-(4-(2-hydroxyethoxy)phenyl)-1,2-bis(4-hydroxyphenyl)-2-(4-(2-(methylamino)ethoxy)-phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof may be present in a compound of Formula (I), a compound of Formula (II), a compound of Formula (III), and/or a compound of Formula (IV) at a concentration above a permissible concentration prior to purification. A permissible concentration of (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof in a compound may be not more than about 1%. In some embodiments, a permissible concentration of (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof in a compound may be not more than about 0.1%, about 0.15%, about 0.2%, about 0.25%, about 0.3%, about 0.4%, about 0.5%, about 0.6%, about 0.7%, about 0.8%, about 0.9%, about 1%, about 1.5%, about 2%, about 2.5%, about 3%, about 4%, or about 5%.

A level of (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof present in a compound, such as a compound synthesized as illustrated in SCHEME 1, may be reduced or removed using one or more purification methods described herein. Reduction of a level of (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof in a compound (e.g., a compound of Formula (III) or a compound of Formula (IV)) may comprise ethyl acetate fractional crystallization (e.g., as described in EXAMPLE 3). Reduction of a level of (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)-phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof in a compound may comprise acetone recrystallization, (e.g., as described in EXAMPLE 3). Reduction of a level of (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof in a compound may comprise THE recrystallization, (e.g., as described in EXAMPLE 3). In some embodiments, all three crystallization steps may be conducted.

Prior to purification, (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof may be present in a compound (e.g., a compound of Formula (III) or a compound of Formula (IV)) at a level of greater than about 0.1%, about 0.15%, about 0.2%, about 0.25%, about 0.3%, about 0.4%, about 0.5%, about 0.6%, about 0.7%, about 0.8%, about 0.9%, about 1%, about 1.5%, about 2%, about 2.5%, about 3%, about 4%, or about 5%. Following purification, (1S,2R)-1-(4-(2-hydroxyethoxy)phenyl)-1,2-bis(4-hydroxyphenyl)-2-(4-(2-(methylamino)ethoxy)phenyl)ethane-1,2-diol; (1R,2S)-1-(4-(2-hydroxyethoxy)phenyl)-1,2-bis(4-hydroxyphenyl)-2-(4-(2-(methylamino)ethoxy)phenyl)ethane-1,2-diol; (1S,2S)-1-(4-(2-hydroxyethoxy)phenyl)-1,2-bis(4-hydroxyphenyl)-2-(4-(2-(methylamino)ethoxy)phenyl)ethane-1,2-diol; (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof may be present in a compound (e.g., a compound of Formula (IV)) at a level of no more than about 0.1%, about 0.15%, about 0.2%, about 0.25%, about 0.3%, about 0.4%, about 0.5%, about 0.6%, about 0.7%, about 0.8%, about 0.9%, about 1%, about 1.5%, about 2%, about 2.5%, about 3%, about 4%, or about 5%. [0264]1-(4-(2-hydroxyethoxy)phenyl)-1,2-bis(4-hydroxyphenyl)-2-(4-(2-(methylamino)ethoxy)phenyl)ethane-1,2-diol impurities in a compound, such as a compound of Formula (IV), may be identified by an analytical method, such as HPLC or LC-MS. In some embodiments, 1-(4-(2-hydroxyethoxy)phenyl)-1,2-bis(4-hydroxyphenyl)-2-(4-(2-(methylamino)ethoxy)phenyl)ethane-1,2-diol may have a relative retention time (RRT) of about 1.76, 1.87, or 1.90, depending on the stereoisomer, relative to (Z)-endoxifen, as measured by HPLC and LC-MS.

### Endoxifen Free Base Compositions

In one aspect, the present disclosure provides stable (Z)-endoxifen free base or salts thereof, and compositions comprising (Z)-endoxifen free base or salts thereof. In some embodiments, the pharmaceutical composition comprises endoxifen predominantly as (Z)-endoxifen free base.

In certain embodiments, compositions may comprise endoxifen as at least 0.1%, at least 0.2%, at least 0.3%, at least 0.4%, at least 0.5%, at least 1%, at least 5%, at least 10%, at least 20%, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, at least 99.5%, at least 99.99%, or 100% of (Z)-endoxifen free base wt/wt of total endoxifen in the composition. In at least one composition, the composition comprises ≥90% of (Z)-endoxifen free base wt/wt of the total endoxifen in the composition. In another embodiment, the composition comprises ≥95% of (Z)-endoxifen free base wt/wt of the total endoxifen in the composition. In yet another embodiment, the compositions comprise ≥96%, ≥97%, ≥98%, ≥99%, or ≥99.5% of (Z)-endoxifen free base wt/wt of the total endoxifen in the composition.

In other embodiments, compositions comprising endoxifen comprise 0.01% to 20%, 0.05% to 15%, or 0.1% to 10% of (Z)-endoxifen wt/wt or w/v of the composition. In at least one embodiment, the compositions comprising endoxifen comprise 0.01% to 20% of (Z)-endoxifen wt/wt or w/v of the composition. In various other embodiments, the compositions comprising endoxifen comprise 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 10%, or 20% of (Z)-endoxifen wt/wt of the composition.

In an aspect, the compositions comprising (Z)-endoxifen further comprises (E)-endoxifen. In some embodiments, the endoxifen in the composition has a ratio of (E)-endoxifen to (Z)-endoxifen (E/Z-ratio) of 1:99; 5:95; 10:90, 15:85; 20:80, 25:75; 30:70; 40:70, 45:55; 50:50; 55:45; 60:40; 65:45; and 70:30. In other embodiments, compositions comprise endoxifen having E/Z-ratio ranging from 10:90 to 70:30. In still other embodiments, compositions comprise endoxifen having E/Z-ratio ranging from 45:55 to 55:45.

Unless specifically referred to by the prefix (Z), (E) or (E/Z), endoxifen used generally without a prefix is used herein to include to any or all endoxifen isoforms.

### Endoxifen Salt Compositions

In some embodiments, the present disclosure provides compositions comprising salts of endoxifen. In some embodiments, the present disclosure provides compositions comprising pharmaceutically acceptable salts of endoxifen. Provided herein in certain embodiments are compositions comprising 1%, 5%, 10%, 20%, 25%, 30%, 40%, 50%, 60%, 70%, 80%, 90%, 95%, 96%, 97%, 98%, 99%, 99.5%, 99.99% or 100% of endoxifen salt.

In some embodiments, the salt is selected from the group consisting of arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, triethiodide, benzathine, clemizole, chloroprocaine, choline, diethylamine, diethanolamine, ethylenediamine, meglumine, piperazine, procaine, aluminum, barium, bismuth, lithium, magnesium, potassium, and zinc. In some embodiments, the salt is endoxifen gluconate. Endoxifen gluconate can be selected from the group consisting of (Z)-endoxifen D-gluconate, (E)-endoxifen D-gluconate, (Z)-endoxifen L-gluconate, (E)-endoxifen L-gluconate or a combination thereof.

In some embodiments, a composition comprising endoxifen gluconate is comprised of 10% to 100% of (Z)-endoxifen D-gluconate on a wt/wt basis of total endoxifen gluconate in the composition. In some embodiments, a composition comprising endoxifen gluconate is comprised of 10% to 100% of (Z)-endoxifen L-gluconate on a wt/wt basis of total endoxifen in the composition.

In other embodiments, a composition comprising endoxifen gluconate is comprised of 10%, 20%, 30%, 40%, 50%, 60%, 65%, 70%, 75%, 80%, 85%, 90%, 91%, 92%, 93%, 94%, 95%, 96%, 97%, 98%, 99%, 99.5%, 99.75%, 99.99%, or 100% of (Z)-endoxifen D-gluconate or (Z)-endoxifen L-gluconate with respect to total endoxifen gluconate. In some embodiments, the compositions comprise at least 80%, at least 85%, at least 90%, at least 91%, at least 92%, at least 93%, at least 94%, at least 95%, at least 96%, at least 97%, at least 98%, at least 99%, at least 99.5%, or at least 99.99% of (Z)-endoxifen D-gluconate, (Z)-endoxifen L-gluconate or a combination thereof.

Provided herein in some embodiments are compositions comprising (Z)-endoxifen D-gluconate and (E)-endoxifen D-gluconate. (Z)-endoxifen D-gluconate and (E)-endoxifen D-gluconate may be present in the compositions at ratios ranging from 10:90 to 99:1 wt/wt or v/v respectively. In some embodiments, the ratio of (Z)-endoxifen D-gluconate to (E)-endoxifen D-gluconate is (wt/wt or v/v) 10:90 to 99:1 (e.g., 45:55, 50:50, 60:40, 70:30, 80: 20, 90:10; 91:9; 92:8; 93:7; 94:8; 95:5, 96:4, 97:3, 98:2, 99:1, 99.5:0.5, or 99.99:0.01) respectively. In certain embodiments, the ratio of (Z)-endoxifen D-gluconate to (E)-endoxifen D-gluconate (wt/wt or v/v) is 90:10; 91:9; 92:8; 93:7; 94:8; 95:5, 96:4, 97:3, 98:2, 99:1, 99.5:0.5, or 99.99:0.01. One of skill in the art will recognize that other combinations of endoxifen gluconate isomers are encompassed in the present disclosure.

In some embodiments, a composition comprising endoxifen gluconate comprises 0.01%, 0.05%, 0.01%, 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%, 17%, 18%, 19% or 20% endoxifen gluconate (wt/wt) or (w/v) of the composition. In some embodiments, a composition comprising endoxifen gluconate comprises 0.01%, 0.05%, 0.01%, 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%, 17%, 18%, 19% or 20% (Z)-endoxifen gluconate (wt/wt) or (w/v) of the composition.

The compounds and compositions of the present disclosure can be administered to a subject in need thereof by any route known in the art, including without limitation, oral, parenteral, topical, and intraductal delivery. Accordingly, compositions disclosed herein are formulated to be compatible with the intended route of administration.

In some embodiments, the compositions comprising endoxifen further comprise an excipient. Such an excipient can be compatible with the intended route of administration.

### Polymorphic Forms of Endoxifen

The present disclosure provides compositions of various polymorphic crystalline form of endoxifen and the characterization thereof. These polymorphic crystalline forms can have enhanced stabilities, dissolution rates, biological activities, or combinations thereof, relative to other forms of endoxifen (e.g., dissolved endoxifen or amorphous endoxifen). In certain aspects disclosed herein, the present disclosure provides a composition comprising a crystalline form of a compound of Formula (III), wherein the crystalline form of the compound of Formula (III) comprises: (a) Form IV as characterized by an x-ray powder diffraction pattern comprising major peaks at 4.7±0.3° 2θ, 23.3±0.3° 2θ, and 13.6±0.3° 2θ; (b) Form V as characterized by x-ray powder diffraction pattern comprising major peaks at 12.5±0.3° 2θ, 19.6±0.3° 2θ, and 8.9±0.3° 2θ; (c) Form VI as characterized by x-ray powder diffraction pattern comprising major peaks at 9.9±0.3° 2θ, 13.4±0.3° 2θ, and 13.7±0.3° 2θ; (d) Form VII as characterized by x-ray powder diffraction pattern comprising major peaks at 20.0±0.3° 2θ, 22.6±0.3° 2θ, and 10.6±0.3° 2θ; (e) Form VIII as characterized by x-ray powder diffraction pattern comprising major peaks at 4.8±0.3° 2θ, 18.9±0.3° 2θ, and 9.5±0.3° 2θ; (f) Form IX as characterized by x-ray powder diffraction pattern comprising major peaks at 19.0±0.3° 2θ, 12.9±0.3° 2θ, and 15.9±0.3° 2θ; (g) Form X as characterized by x-ray powder diffraction pattern comprising major peaks at 7.2±0.3° 2θ, 14.3±0.3° 2θ, 18.7±0.3° 2θ, 21.5±0.3° 2θ, and 22.7±0.3° 2θ; (h) Form XI as characterized by x-ray powder diffraction pattern comprising major peaks at 14.0±0.3° 2θ, 17.7±0.3°2θ, 11.9±0.3°2θ, 18.4±0.3°2θ, 23.9±0.3°2θ, 17.3±0.3°2θ, 21.8±0.3°2θ, 20.8±0.3°2θ, and 23.0±0.3°2θ; (i) Form XII as characterized by x-ray powder diffraction pattern comprising major peaks at 12.5±0.3°2θ, 15.6±0.3°2θ, and 19.0±0.3°2θ; (1) Form XIV as characterized by x-ray powder diffraction pattern comprising major peaks at 11.6±0.3°2θ, 21.3±0.3°2θ, and 19.3±0.3°2θ; (k) Form XV as characterized by x-ray powder diffraction pattern comprising major peaks at 9.8±0.3°2θ, 4.7±0.3°20, and 14.0±0.3°2θ; (1) Form XIX as characterized by x-ray powder diffraction pattern comprising major peaks at 4.7±0.3°2θ, 23.6±0.3°2θ, and 18.9±0.3°2θ; or (m) a combination thereof.

In some cases, at least about 80%, at least about 90%, at least about 95%, at least about 98%, or at least about 99% by weight of the compound of Formula (III) in the composition is the (Z)-isomer. In some cases, the compound of Formula (III) is a compound of Formula (IV).

In some cases, at least about 50%, at least about 60%, at least about 70%, at least about 80%, at least about 90%, at least about 95%, at least about 98%, or at least about 99% of the compound of Formula (III) is Form IV, Form V, Form VI, Form VII, Form VIII, Form IX, Form X, Form XI, Form XII, Form XIII, Form XIV, Form XV, Form XVI, Form XVII, Form XVIII, Form XIX, or a combination thereof. In some cases, at least about 80%, at least about 90%, at least about 95%, at least about 98%, or at least about 99% by weight of the compound of Formula (III) in the composition is the (Z)-isomer. In some cases, at least about 50%, at least about 60%, at least about 70%, at least about 80%, at least about 90%, at least about 95%, at least about 98%, or at least about 99% of the compound of Formula (III) is Form IV, Form V, Form VI, Form VII, Form VIII, Form IX, Form X, Form XI, Form XII, Form XIII, Form XIV, Form XV, Form XVI, Form XVII, Form XVIII, or Form XIX. In some cases, at least about 50%, at least about 60%, at least about 70%, at least about 80%, at least about 90%, at least about 95%, at least about 98%, or at least about 99% of the compound of Formula (III) is in a single crystalline form (e.g., is Form IV, Form V, Form VI, Form VII, Form VIII, Form IX, Form X, Form XI, Form XII, Form XIII, Form XIV, Form XV, Form XVI, Form XVII, Form XVIII, or Form XIX).

In some cases, the crystalline form comprises Form IV, Form V, Form VI, Form VII, Form VIII, Form IX, Form XII, Form XIV, Form XV, Form XIX, or a combination thereof. In some cases, the crystalline form comprises at least about 50%, at least about 60%, at least about 70%, at least about 80%, at least about 90%, at least about 95%, at least about 98%, or at least about 99% of Form IV, Form V, Form VI, Form VII, Form VIII, Form IX, Form XII, Form XIV, Form XV, Form XIX, or the combination thereof. In some cases, the crystalline form comprises Form IV, Form VII, or a combination thereof. In some cases, the crystalline form at least about 50%, at least about 60%, at least about 70%, at least about 80%, at least about 90%, at least about 95%, at least about 98%, or at least about 99% of Form IV, Form VII, or the combination thereof.

In some cases, the crystalline form has a greatest dimension of at least about 500 nm, at least about 800 nm, at least about 1 μm, at least about 1.5 μm, at least about 2.5 μm, at least about 5 μm, at least about 10 μm, at least about 25 μm, at least about 50 μm, at least about 100 μm, at least about 200 μm, or at least about 500 μm (e.g., is collected on a 500 μm filter).

In some aspects, the polymorphic crystalline forms were characterized by x-ray power diffraction (XRPD) analysis. In some aspects, the polymorphic crystalline forms were characterized by differential scanning calorimetry (DSC). In some aspects, the polymorphic crystalline forms were characterized by thermogravimetric analysis (TGA) and evolved gas analysis (EGA).

### Form IV of the Compound of Formula (III)

A composition can comprise Form IV of the compound of Formula (III). In some cases, at least about 50%, at least about 60%, at least about 70%, at least about 80%, at least about 90%, at least about 95%, at least about 98%, or at least about 99% of the compound of Formula (III) is Form IV.

The crystalline form of Form IV of the compound of Formula (III) can be characterized by an XRPD pattern comprising major peaks at 4.7±0.3° 2θ, 23.3±0.3° 2θ, and 13.6±0.3° 2θ. In some cases, the XRPD pattern further comprises at least one peak, at least two peaks, at least three peaks, or at least four peaks selected from 23.8±0.3° 2θ, 14.2±0.3° 2θ, 22.5±0.3° 2θ, or 15.7±0.3° 2θ. In some cases, the XRPD pattern further comprises at least one peak, at least two peaks, or at least three peaks selected from 7.1±0.3° 2θ, 20.2±0.3° 2θ, or 9.5±0.3° 2θ. In some cases, the XRPD pattern further comprises at least one peak, comprises at least two peaks, at least three peaks, at least four peaks, at least five peaks, or at least six peaks selected from 7.1±0.3°2θ, 9.5±0.3°2θ, 14.2±0.3°2θ, 15.7±0.3°2θ, 20.2±0.3°2θ, 22.5±0.3°2θ, and 23.8±0.3°2θ.

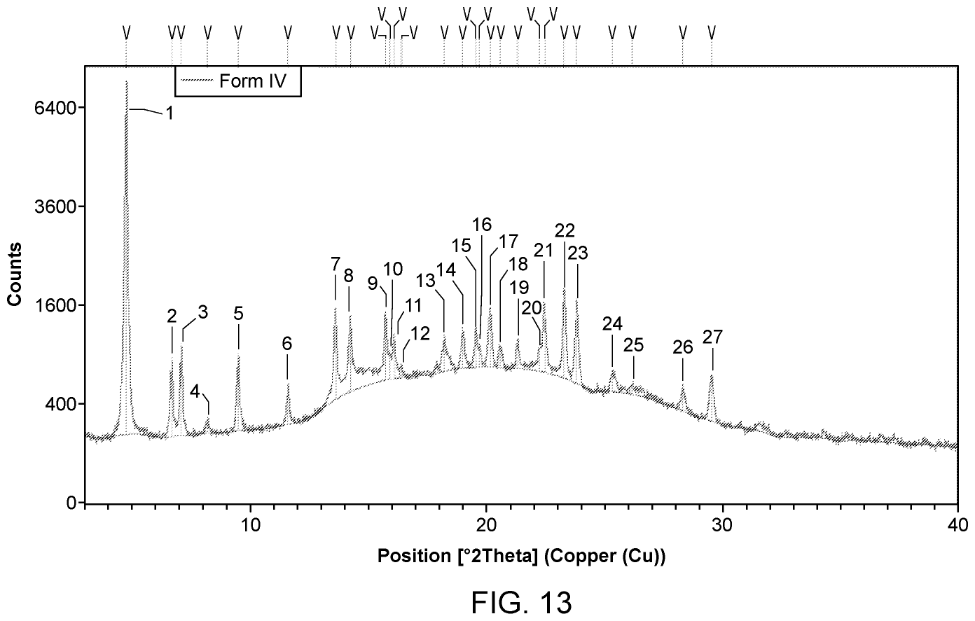

In some cases, the crystalline form of the compound of Formula (III) is characterized by an x-ray diffraction pattern substantially as set forth in FIG. 13.

In some cases, the crystalline form of the compound of Formula (III) comprises between about 1% and about 30% solvent by weight as determined by TGA analysis. In some cases, the crystalline form loses solvent upon heating to a temperature between 70° C. and 130° C. as determined by TGA analysis. In some cases, the solvent comprises 2-propanol, heptane, or a combination thereof.

### Form V of the Compound of Formula (III)

A composition can comprise Form V of the compound of Formula (III). In some cases, at least about 50%, at least about 60%, at least about 70%, at least about 80%, at least about 90%, at least about 95%, at least about 98%, or at least about 99% of the compound of Formula (III) is Form V.

The crystalline form of Form V of the compound of Formula (III) can be characterized by an XRPD pattern comprising major peaks at 12.5±0.3° 2θ, 19.6±0.3° 2θ, and 8.9±0.3° 2θ. In some cases, the XRPD pattern further comprises at least one peak, at least two peaks, at least three peaks, or at least four peaks selected from 21.7±0.3° 2θ, 20.8±0.3° 2θ, 19.8±0.3° 2θ, or 16.0±0.3°2θ. In some cases, the XRPD pattern further comprises at least one peak, at least two peaks, or at least three peaks selected from 22.0±0.3° 2θ, 13.5±0.3° 2θ, or 14.4±0.3°2θ. In some cases, the XRPD pattern further comprises at least one peak, comprises at least two peaks, at least three peaks, at least four peaks, at least five peaks, or at least six peaks selected from 21.7±0.3°2θ, 20.8±0.3°2θ, 19.8±0.3°2θ, 16.0±0.3°2θ, 22.0±0.3° 2θ, 13.5±0.3° 2θ, and 14.4±0.3° 2θ.

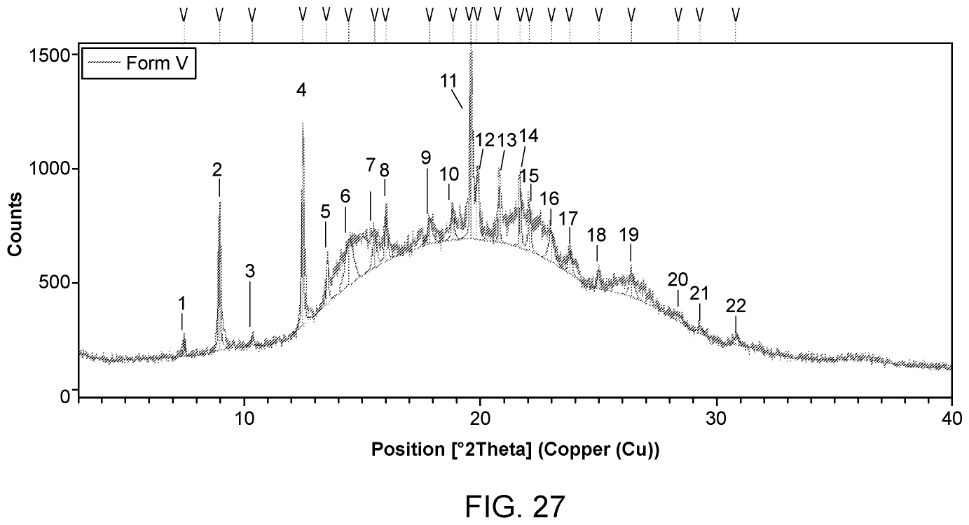

In some cases, the crystalline form of the compound of Formula (III) is characterized by an x-ray diffraction pattern substantially as set forth in FIG. 27.

### Form VI of the Compound of Formula (III)

A composition can comprise Form VI of the compound of Formula (III). In some cases, at least about 50%, at least about 60%, at least about 70%, at least about 80%, at least about 90%, at least about 95%, at least about 98%, or at least about 99% of the compound of Formula (III) is Form VI.

The crystalline form of Form VI of the compound of Formula (III) can be characterized by an XRPD pattern comprising major peaks at 9.9±0.3° 2θ, 13.4±0.3° 2θ, and 13.7±0.3° 2θ. In some cases, the XRPD pattern further comprises at least one peak, at least two peaks, at least three peaks, or at least four peaks selected from 17.6±0.3° 2θ, 18.6±0.3° 2θ, 17.3±0.3° 2θ, or 21.8±0.3° 2θ. In some cases, the XRPD pattern further comprises at least one peak, at least two peaks, or at least three peaks selected from 10.2±0.3° 2θ, 19.5±0.3° 2θ, or 14.2±0.3° 2θ. In some cases, the XRPD pattern further comprises at least one peak, comprises at least two peaks, at least three peaks, at least four peaks, at least five peaks, or at least six peaks selected from 17.6±0.3° 2θ, 18.6±0.3° 2θ, 17.3±0.3° 2θ, 21.8±0.3° 2θ, 10.2±0.3° 2θ, 19.5±0.3° 2θ, or 14.2±0.3° 2θ.

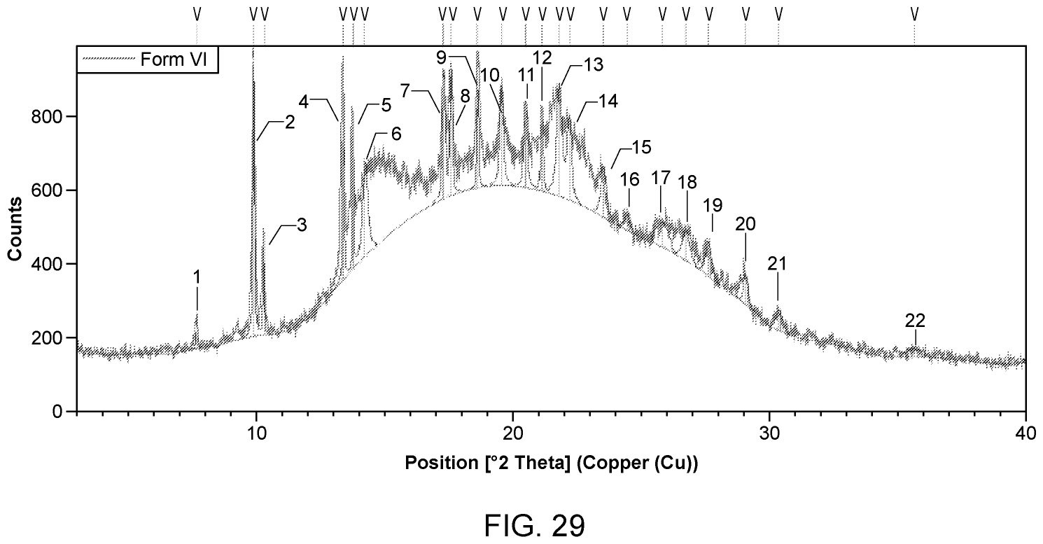

In some cases, the crystalline form of the compound of Formula (III) is characterized by an x-ray diffraction pattern substantially as set forth in FIG. 29.

### Form VII of the Compound of Formula (III)

A composition can comprise Form VII of the compound of Formula (III). In some cases, at least about 50%, at least about 60%, at least about 70%, at least about 80%, at least about 90%, at least about 95%, at least about 98%, or at least about 99% of the compound of Formula (III) is Form VII.

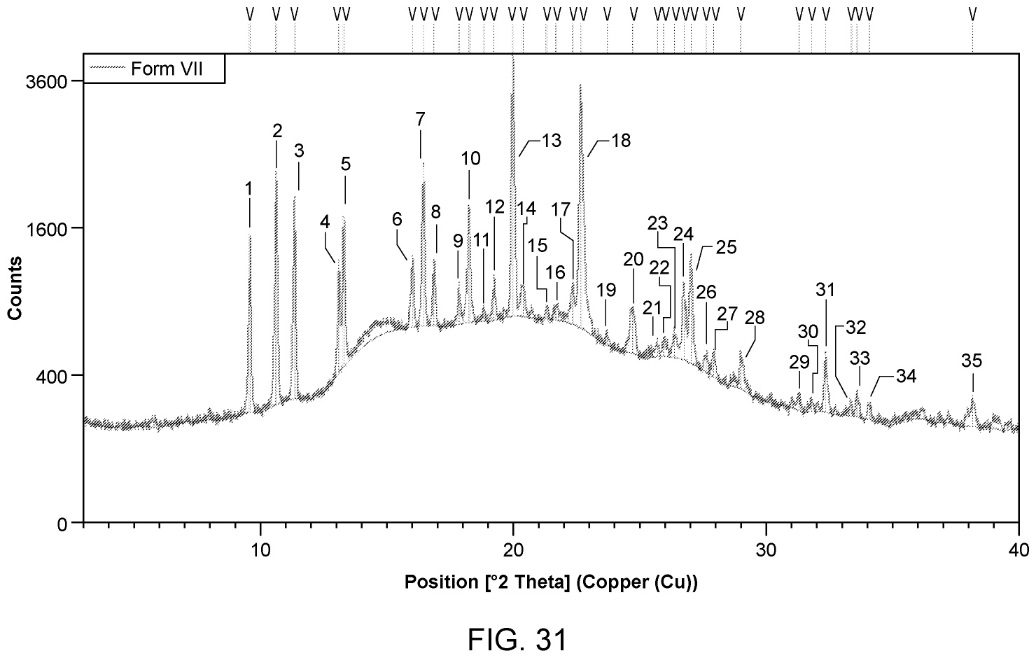

The crystalline form of Form VII of the compound of Formula (III) can be characterized by an XRPD pattern comprising major peaks at 20.0±0.3° 2θ, 22.6±0.3° 2θ, and 10.6±0.3° 2θ. In some cases, the XRPD pattern further comprises at least one peak, at least two peaks, at least three peaks, or at least four peaks selected from 11.4±0.3° 2θ, 16.4±0.3° 2θ, 9.6±0.3° 2θ, or 13.3±0.3° 2θ. In some cases, the XRPD pattern further comprises at least one peak, at least two peaks, or at least three peaks selected from 18.2±0.3° 2θ, 13.1±0.3° 2θ, or 27.0±0.3° 2θ. In some cases, the XRPD pattern further comprises at least one peak, comprises at least two peaks, at least three peaks, at least four peaks, at least five peaks, or at least six peaks selected from 11.4±0.3° 2θ, 16.4±0.3° 2θ, 9.6±0.3° 2θ, 13.3±0.3° 2θ, 18.2±0.3° 2θ, 13.1±0.3° 2θ, or 27.0±0.3° 2θ. In some cases, the crystalline form of the compound of Formula (III) is characterized by an x-ray diffraction pattern substantially as set forth in FIG. 31.

### Form VIII of the Compound of Formula (III)

A composition can comprise Form VIII of the compound of Formula (III). In some cases, at least about 50%, at least about 60%, at least about 70%, at least about 80%, at least about 90%, at least about 95%, at least about 98%, or at least about 99% of the compound of Formula (III) is Form VIII.

The crystalline form of Form VIII of the compound of Formula (III) can be characterized by an XRPD pattern comprising major peaks at 4.8±0.3° 2θ, 18.9±0.3° 2θ, and 9.5±0.3° 2θ. In some cases, the XRPD pattern further comprises at least one peak, at least two peaks, at least three peaks, or at least four peaks selected from 23.7±0.3° 2θ, 21.9±0.3° 2θ, 21.2±0.3° 2θ, or 12.9±0.3° 2θ. In some cases, the XRPD pattern further comprises at least one peak, at least two peaks, or at least three peaks selected from 25.0±0.3° 2θ, 21.5±0.3° 2θ, or 16.4±0.3° 2θ. In some cases, the XRPD pattern further comprises at least one peak, comprises at least two peaks, at least three peaks, at least four peaks, at least five peaks, or at least six peaks selected from 23.7±0.3° 2θ, 21.9±0.3° 2θ, 21.2±0.3° 2θ, 12.9±0.3° 2θ, 25.0±0.3° 2θ, 21.5±0.3° 2θ, or 16.4±0.3° 2θ.

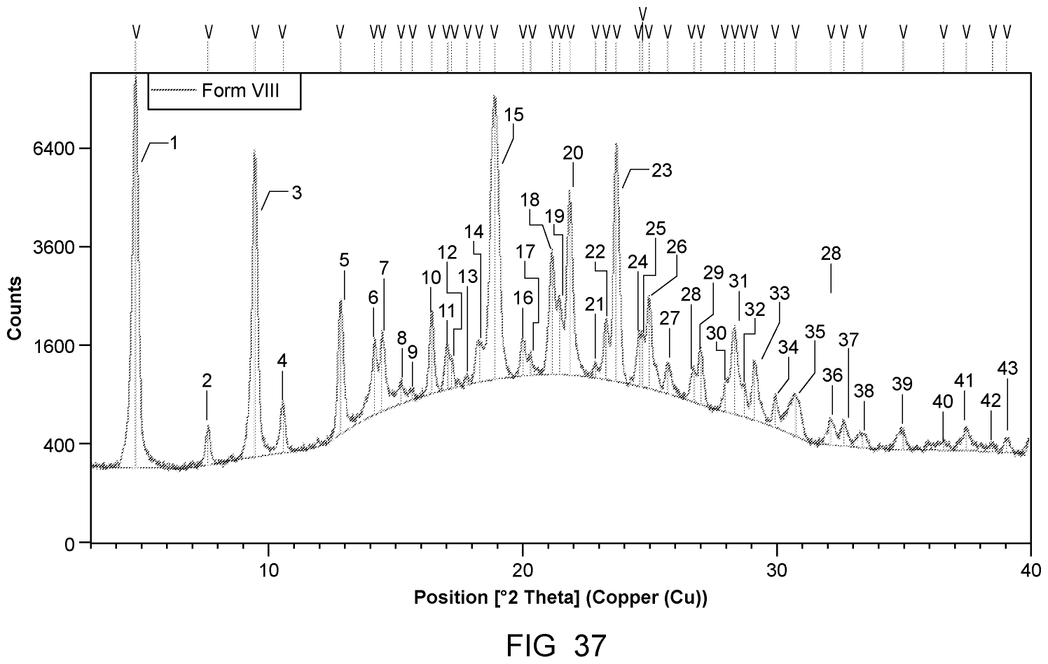

In some cases, the crystalline form of the compound of Formula (III) is characterized by an x-ray diffraction pattern substantially as set forth in FIG. 37.

### Form IX of the Compound of Formula (III)

A composition can comprise Form IX of the compound of Formula (III). In some cases, at least about 50%, at least about 60%, at least about 70%, at least about 80%, at least about 90%, at least about 95%, at least about 98%, or at least about 99% of the compound of Formula (III) is Form IX.

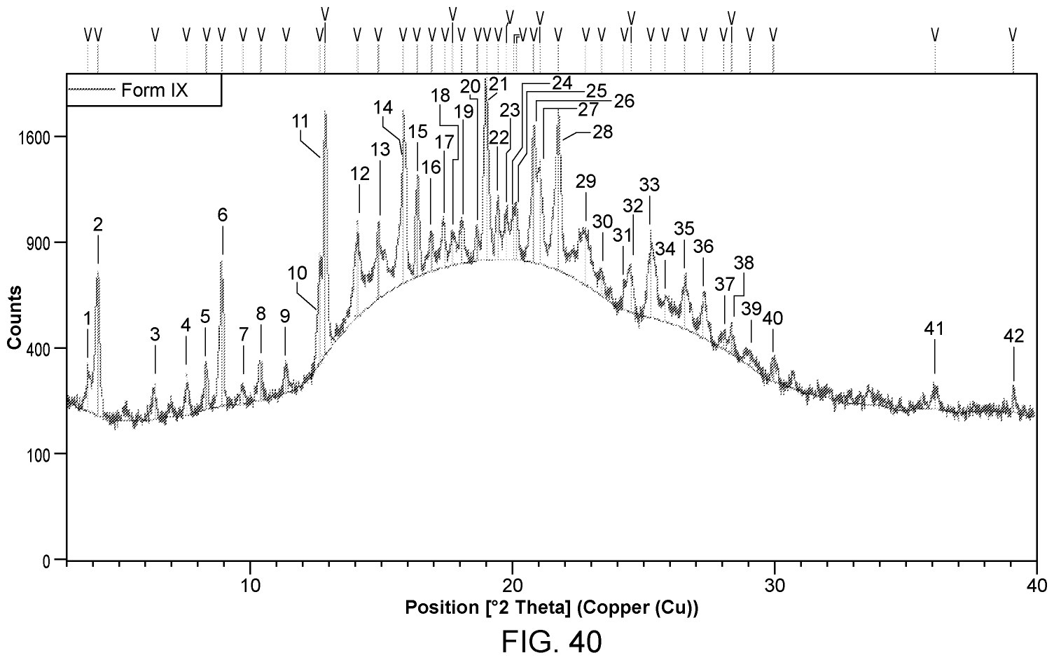

The crystalline form of Form IX of the compound of Formula (III) can be characterized by an XRPD pattern comprising major peaks at 19.0±0.3° 2θ, 12.9±0.3° 2θ, and 15.9±0.3° 2θ. In some cases, the XRPD pattern further comprises at least one peak, at least two peaks, at least three peaks, or at least four peaks selected from 21.7±0.3° 2θ, 20.8±0.3° 2θ, 21.1±0.3° 2θ, or 8.9±0.3° 2θ. In some cases, the XRPD pattern further comprises at least one peak, at least two peaks, or at least three peaks selected from 16.4±0.3° 2θ, 4.2±0.3° 2θ, or 12.7±0.3° 2θ. In some cases, the XRPD pattern further comprises at least one peak, comprises at least two peaks, at least three peaks, at least four peaks, at least five peaks, or at least six peaks selected from 21.7±0.3° 2θ, 20.8±0.3° 2θ, 21.1±0.3° 2θ, 8.9±0.3° 2θ, 16.4±0.3° 2θ, 4.2±0.3° 2θ, or 12.7±0.3° 2θ. In some cases, the crystalline form of the compound of Formula (III) is characterized by an x-ray diffraction pattern substantially as set forth in FIG. 40.

### Form X of the Compound of Formula (III)

A composition can comprise Form X of the compound of Formula (III). In some cases, at least about 50%, at least about 60%, at least about 70%, at least about 80%, at least about 90%, at least about 95%, at least about 98%, or at least about 99% of the compound of Formula (III) is Form X.

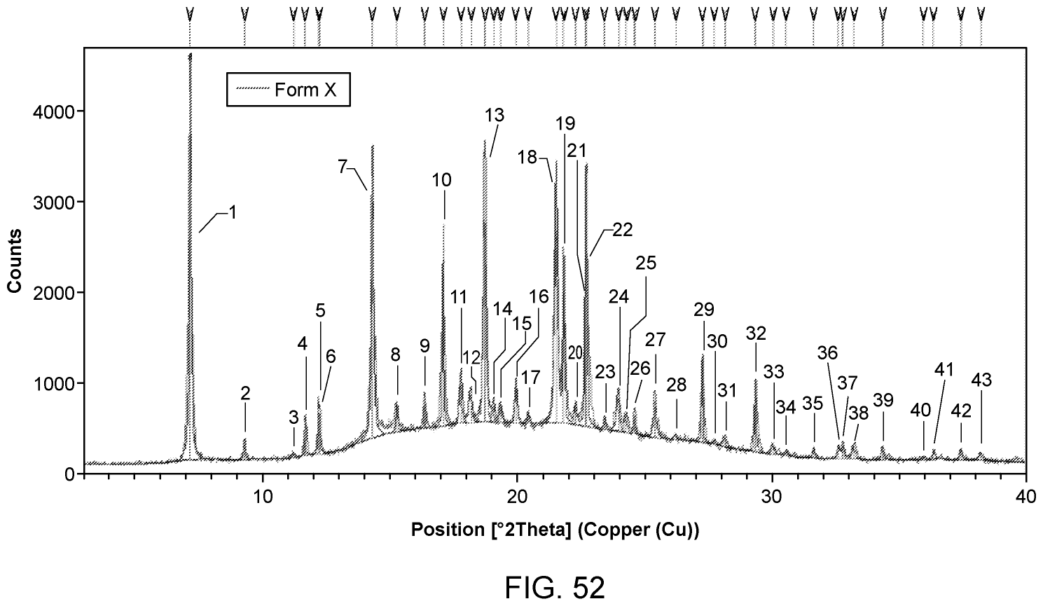

The crystalline form of Form X of the compound of Formula (III) can be characterized by an XRPD pattern comprising major peaks at 7.2±0.3° 2θ, 14.3±0.3° 2θ, 18.7±0.3° 2θ, 21.5±0.3° 2θ, and 22.7±0.3° 2θ. In some cases, the XRPD pattern further comprises a peak at 17.1±0.3° 2θ. In some cases, the XRPD pattern further comprises at least one peak, at least two peaks, or at least three peaks selected from 21.8±0.3° 2θ, 27.3±0.3° 2θ, or 29.4±0.3° 2θ. In some cases, the crystalline form of the compound of Formula (III) is characterized by an x-ray diffraction pattern substantially as set forth in FIG. 52.

### Form XI of the Compound of Formula (III)

A composition can comprise Form XI of the compound of Formula (III). In some cases, at least about 50%, at least about 60%, at least about 70%, at least about 80%, at least about 90%, at least about 95%, at least about 98%, or at least about 99% of the compound of Formula (III) is Form XI.

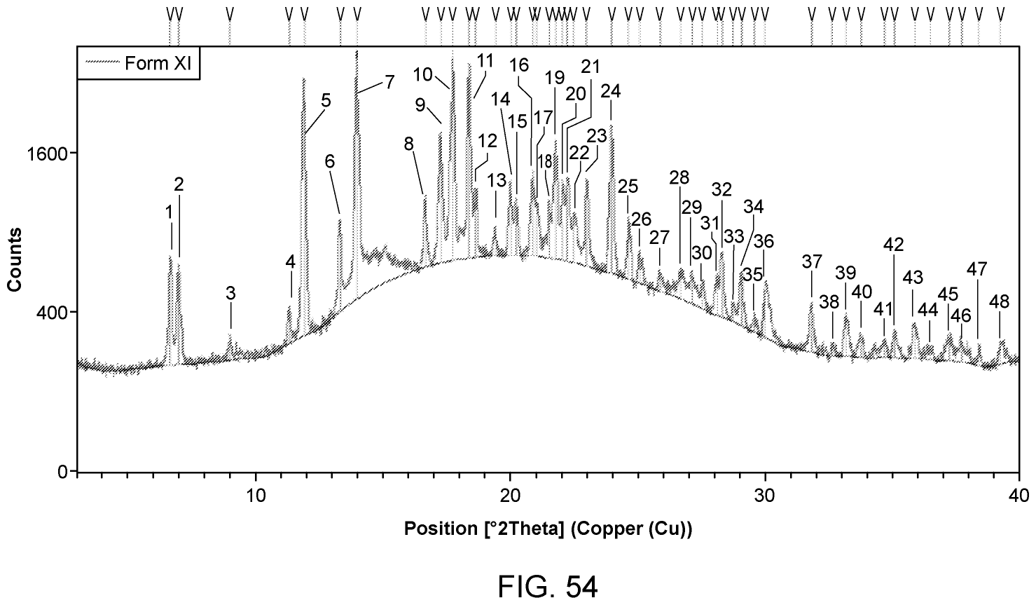

The crystalline form of Form XI of the compound of Formula (III) can be characterized by an XRPD pattern comprising major peaks at 14.0±0.3° 2θ, 17.7±0.3° 2θ, 11.9±0.3° 2θ, 18.4±0.3° 2θ, 23.9±0.3° 2θ, 17.3±0.3° 2θ, 21.8±0.3° 2θ, 20.8±0.3° 2θ, and 23.0±0.3° 2θ. In some cases, the XRPD pattern further comprises at least one peak or at least two peaks selected from 22.2±0.3° 20 or 16.6±0.3° 2θ. In some cases, the crystalline form of the compound of Formula (III) is characterized by an x-ray diffraction pattern substantially as set forth in FIG. 54.

### Form XII of the Compound of Formula (III)

A composition can comprise Form XII of the compound of Formula (III). In some cases, at least about 50%, at least about 60%, at least about 70%, at least about 80%, at least about 90%, at least about 95%, at least about 98%, or at least about 99% of the compound of Formula (III) is Form XII.

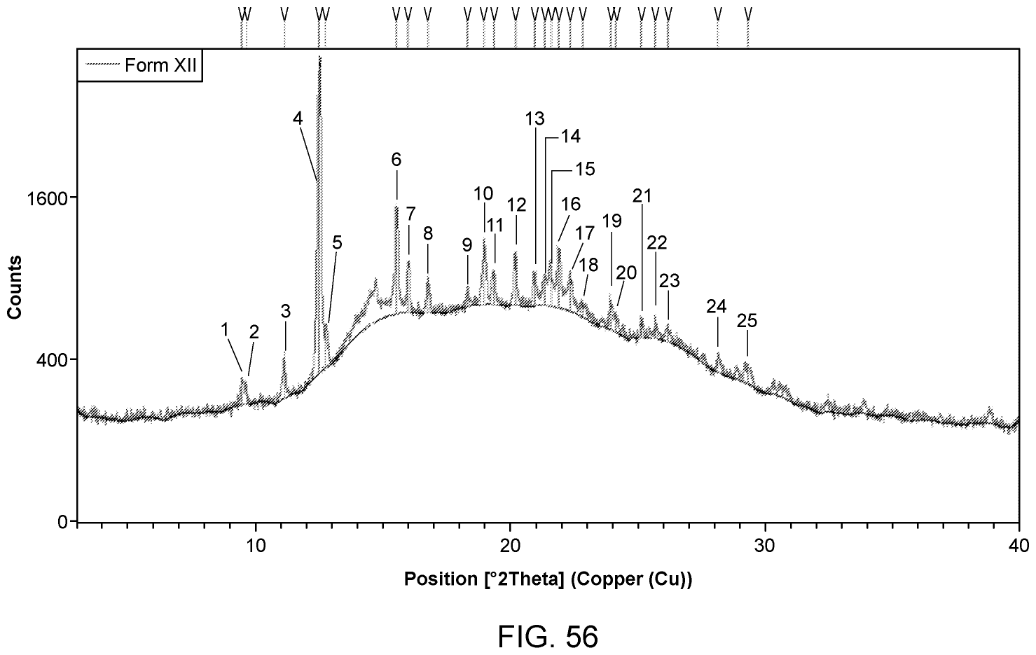

The crystalline form of Form XII of the compound of Formula (III) can be characterized by an XRPD pattern comprising major peaks at 12.5±0.3° 2θ, 15.6±0.3° 2θ, and 19.0±0.3° 2θ. In some cases, the XRPD pattern further comprises at least one peak, at least two peaks, at least three peaks, or at least four peaks selected from 21.9±0.3° 2θ, 20.2±0.3° 2θ, 16.0±0.3° 2θ, or 21.6±0.3° 2θ. In some cases, the XRPD pattern further comprises at least one peak, at least two peaks, or at least three peaks selected from 22.4±0.3° 2θ, 16.8±0.3° 2θ, or 12.8±0.3° 2θ. In some cases, the XRPD pattern further comprises at least one peak, comprises at least two peaks, at least three peaks, at least four peaks, at least five peaks, or at least six peaks selected from 21.9±0.3° 2θ, 20.2±0.3° 2θ, 16.0±0.3° 2θ, 21.6±0.3° 2θ, 22.4±0.3° 2θ, 16.8±0.3° 2θ, or 12.8±0.3° 2θ. In some cases, the crystalline form of the compound of Formula (III) is characterized by an x-ray diffraction pattern substantially as set forth in FIG. 56.

### Form XIV of the Compound of Formula (III)

A composition can comprise Form XIV of the compound of Formula (III). In some cases, at least about 50%, at least about 60%, at least about 70%, at least about 80%, at least about 90%, at least about 95%, at least about 98%, or at least about 99% of the compound of Formula (III) is Form XIV.

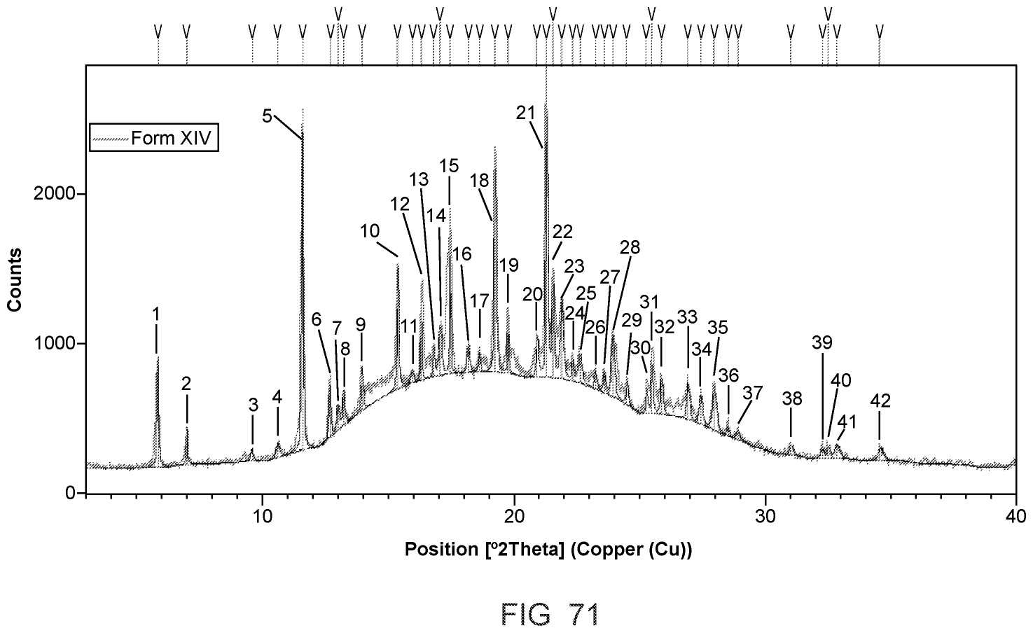

The crystalline form of Form XIV of the compound of Formula (III)can be characterized by an XRPD pattern comprising major peaks at 11.6±0.3° 2θ, 21.3±0.3° 2θ, and 19.3±0.3° 2θ. In some cases, the XRPD pattern further comprises at least one peak, at least two peaks, at least three peaks, or at least four peaks selected from 17.5±0.3° 2θ, 15.4±0.3° 2θ, 21.6±0.3° 2θ, or 5.8±0.3° 2θ. In some cases, the XRPD pattern further comprises at least one peak, at least two peaks, or at least three peaks selected from 16.3±0.3° 2θ, 21.9±0.3° 2θ, or 23.9±0.3° 2θ. In some cases, the XRPD pattern further comprises at least one peak, comprises at least two peaks, at least three peaks, at least four peaks, at least five peaks, or at least six peaks selected from 17.5±0.3° 2θ, 15.4±0.3° 2θ, 21.6±0.3° 2θ, 5.8±0.3° 2θ, 16.3±0.3° 2θ, 21.9±0.3° 2θ, or 23.9±0.3° 2θ. In some cases, the crystalline form of the compound of Formula (III) is characterized by an x-ray diffraction pattern substantially as set forth in FIG. 71.

### Form XV of the Compound of Formula (IH)

A composition can comprise Form XV of the compound of Formula (III). In some cases, at least about 50%, at least about 60%, at least about 70%, at least about 80%, at least about 90%, at least about 95%, at least about 98%, or at least about 99% of the compound of Formula (III) is Form XV.

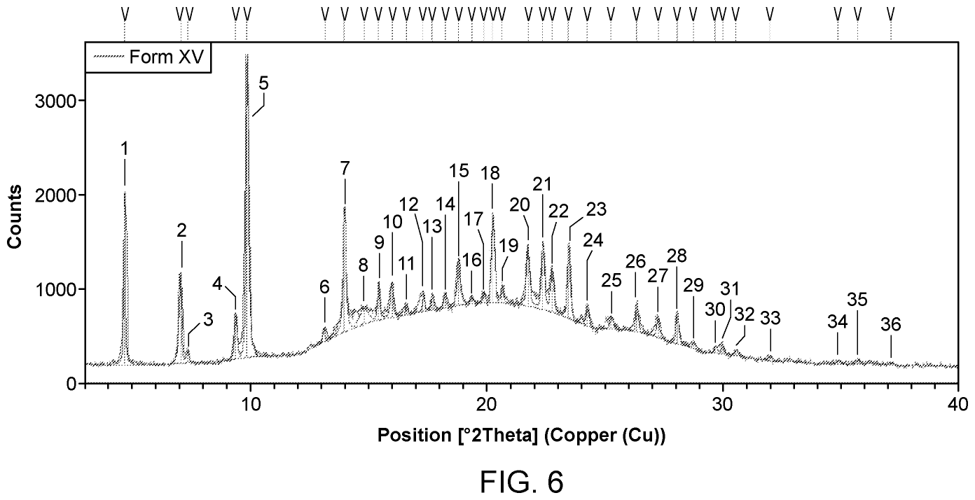

The crystalline form of Form XV of the compound of Formula (III) can be characterized by an XRPD pattern comprising major peaks at 9.8±0.3° 2θ, 4.7±0.3° 2θ, and 14.0±0.3° 2θ. In some cases, the XRPD pattern further comprises at least one peak, at least two peaks, at least three peaks, or at least four peaks selected from 20.2±0.3° 2θ, 7.1±0.3° 2θ, 23.4±0.3° 2θ, or 22.4±0.3° 2θ. In some cases, the XRPD pattern further comprises at least one peak, at least two peaks, or at least three peaks selected from 21.7±0.3° 2θ, 22.7±0.3° 2θ, or 18.8±0.3° 2θ. In some cases, the XRPD pattern further comprises at least one peak, comprises at least two peaks, at least three peaks, at least four peaks, at least five peaks, or at least six peaks selected from 20.2±0.3° 2θ, 7.1±0.3° 2θ, 23.4±0.3° 2θ, 22.4±0.3° 2θ, 21.7±0.3° 2θ, 22.7±0.3° 2θ, or 18.8±0.3° 2θ. In some cases, the crystalline form of the compound of Formula (III) is characterized by an x-ray diffraction pattern substantially as set forth in FIG. 6.

### Form XIX of the Compound of Formula (III)

A composition can comprise Form XIX of the compound of Formula (III). In some cases, at least about 50%, at least about 60%, at least about 70%, at least about 80%, at least about 90%, at least about 95%, at least about 98%, or at least about 99% of the compound of Formula (III) is Form XIX.

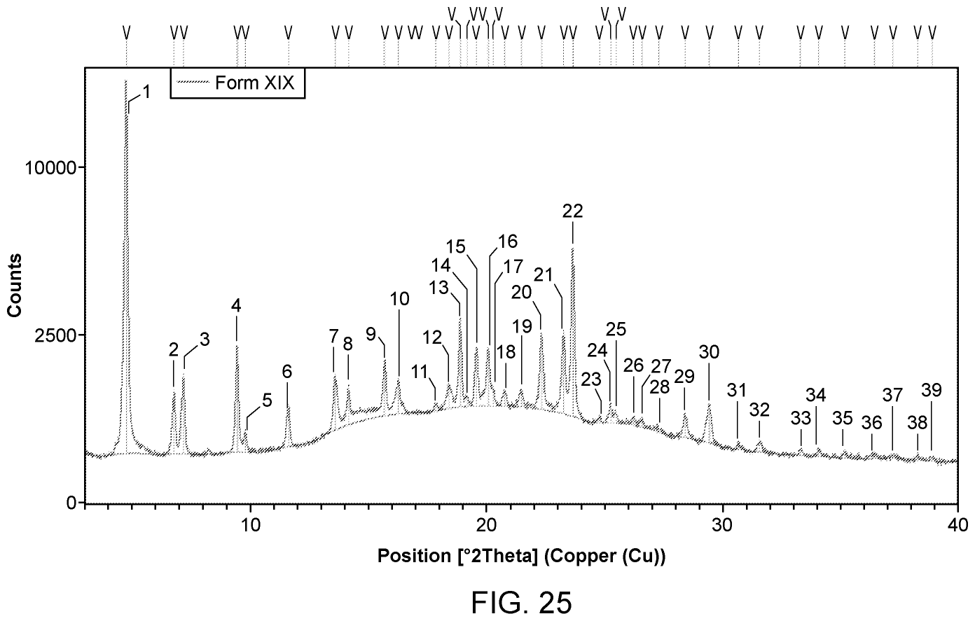

The crystalline form of Form XIX of the compound of Formula (III) can be characterized by an XRPD pattern comprising major peaks at 4.7±0.3° 2θ, 23.6±0.3° 2θ, and 18.9±0.3° 2θ. In some cases, the XRPD pattern further comprises at least one peak, at least two peaks, at least three peaks, or at least four peaks selected from 9.4±0.3° 2θ, 23.3±0.3° 2θ, 22.3±0.3° 2θ, or 20.1±0.3° 2θ. In some cases, the XRPD pattern further comprises at least one peak, at least two peaks, or at least three peaks selected from 19.6±0.3° 2θ, 7.1±0.3° 2θ, or 15.7±0.3° 2θ. In some cases, the XRPD pattern further comprises at least one peak, comprises at least two peaks, at least three peaks, at least four peaks, at least five peaks, or at least six peaks selected from 9.4±0.3° 2θ, 23.3±0.3° 2θ, 22.3±0.3° 2θ, 20.1±0.3° 2θ, 19.6±0.3° 2θ, 7.1±0.3° 2θ, or 15.7±0.3° 2θ. In some cases, the crystalline form of the compound of Formula (III) is characterized by an x-ray diffraction pattern substantially as set forth in FIG. 25.

### Form XVI of the Compound of Formula (III)

A composition can comprise Form XVI of the compound of Formula (III). In some cases, at least about 50%, at least about 60%, at least about 70%, at least about 80%, at least about 90%, at least about 95%, at least about 98%, or at least about 99% of the compound of Formula (III) is Form XVI. In some cases, the crystalline form of the compound of Formula (III) is Form XVI and may be present in compositions comprising Form XV, Form IX, Form XII, Form XIV, or combinations thereof.

### Form XVII of the Compound of Formula (III)

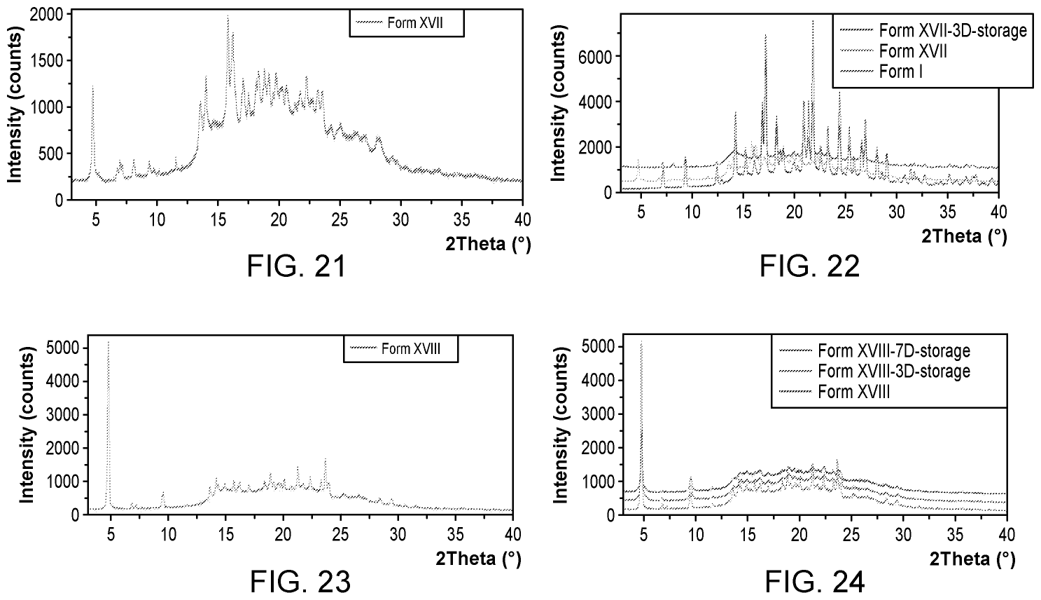

A composition can comprise Form XVII of the compound of Formula (III). In some cases, at least about 50%, at least about 60%, at least about 70%, at least about 80%, at least about 90%, at least about 95%, at least about 98%, or at least about 99% of the compound of Formula (III) is Form XVII. In some cases, the crystalline form of the compound of Formula (III) is Form XVII and is characterized by an x-ray diffraction pattern substantially as set forth in FIG. 21.

### Form XVIII of the Compound of Formula (III)

A composition can comprise Form XVIII of the compound of Formula (III). In some cases, at least about 50%, at least about 60%, at least about 70%, at least about 80%, at least about 90%, at least about 95%, at least about 98%, or at least about 99% of the compound of Formula (III) is Form XVIII. In some cases, the crystalline form of the compound of Formula (III) is Form XVIII and is characterized by an x-ray diffraction pattern substantially as set forth in FIG. 23.

### Endoxifen Crystallizations

In an aspect, the present disclosure provides methods for generating crystalline forms of the compound of Formula (III). The methods can produce single crystalline forms, or mixtures of forms (e.g., a mixture of Form I and Form V). The method can generate a single form of the compound of Formula (III) with at about least 50%, at about least 60%, at least about 70%, at least about 80%, at least about 90%, at least about 95%, at least about 98%, or at least about 99% purity (e.g., generate 99% pure Form IV of the compound of Formula (III)).

In some cases, the crystalline form of the compound of Formula (III) is collected through filtration. In some cases, the filtration collects solid material which has a greatest dimension of at least about 500 nm, at least about 800 nm, at least about 1 μm, at least about 1.5 μm, at least about 2.5 μm, at least about 5 μm, at least about 10 μm, at least about 25 μm, at least about 50 μm, at least about 100 μm, at least about 200 μm, or at least about 500 μm. In some cases, the crystalline form is dried following the collection.

### Methods of Generating Form I

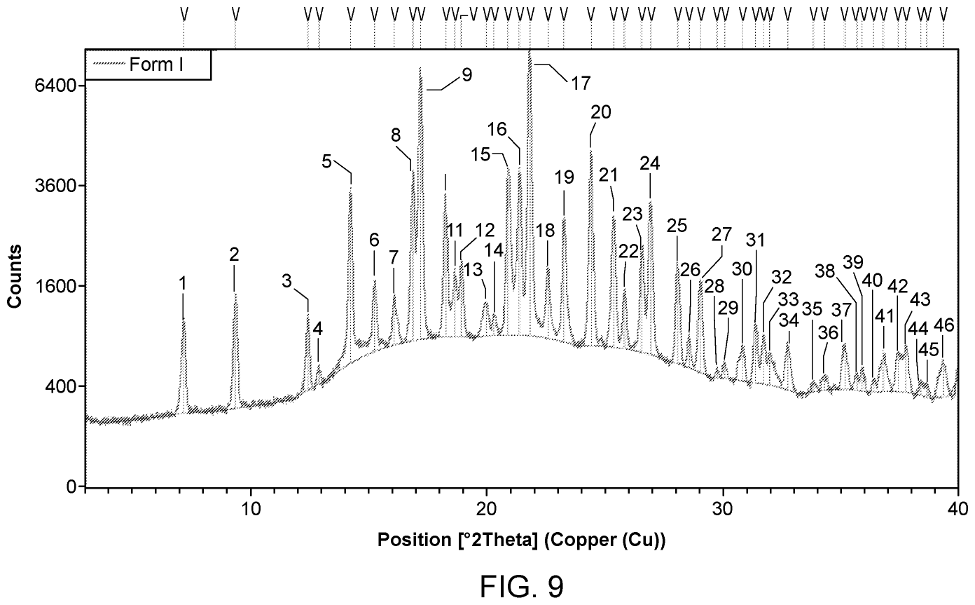

Aspects of the present disclosure provide methods of generating Form I of the compound of Formula (III). Form I may be characterized by an XRPD pattern comprising one or more peaks selected from 21.8±0.3° 2θ, 17.2±0.3° 2θ, 24.2±0.3° 2θ, 16.9±0.3° 2θ, 21.4±0.3° 2θ, 20.9±0.3° 2θ, 14.2±0.3° 2θ, 18.2±0.3° 2θ, 26.9±0.3° 2θ, or 25.4±0.3° 2θ. In some embodiments, Form I may be characterized by an x-ray diffraction pattern substantially as set forth in FIG. 9. In some cases, the methods comprise converting another solid form of the compound of Formula (III) to Form I. In particular instances, a method of generating a crystalline form of a compound of Formula (III) can comprise incubating a solid form of the compound of Formula (III) in a solvent system in which at least a portion of the solid form of the compound of Formula (III) remains solid; converting the portion of the solid form of the compound of Formula (III) to the crystalline form; and collecting the crystalline form. In some cases, the crystalline form of the compound of Formula (III) is Form I. In some cases, the incubating is for between about 3 and about 120 hours, between about 3 and about 20 hours, between about 8 and about 50 hours, or between about 30 and about 120 hours.

In some cases, the solid form of the compound of Formula (III) comprises at least about 50%, at least about 60%, at least about 70%, at least about 80%, at least about 90%, at least about 95%, at least about 98%, or at least about 99% isomeric purity prior to the incubating. In some cases, the solid form of the compound of Formula (III) comprises at least about 50%, at least about 60%, at least about 70%, at least about 80%, at least about 90%, at least about 95%, at least about 98%, or at least about 99% isomeric purity prior to the incubating. In some cases, at least about 50%, at least about 60%, at least about 70%, at least about 80%, at least about 90%, at least about 95%, at least about 98%, or at least about 99% of the compound of Formula (III) is the (Z)-isomer prior to the incubating.

In some cases, the crystalline form of the compound of Formula (III) comprises at least about 50%, at least about 60%, at least about 70%, at least about 80%, at least about 90%, at least about 95%, at least about 98%, or at least about 99% isomeric purity. In some cases, at least about 50%, at least about 60%, at least about 70%, at least about 80%, at least about 90%, at least about 95%, at least about 98%, or at least about 99% of the compound of Formula (III) of the crystalline form is the (Z)-isomer.

In some cases, the solid form of the compound of Formula (III) comprises at least one of Forms IV-XIX. In some cases, at least about 50%, at least about 60%, at least about 70%, at least about 80%, at least about 90%, at least about 95%, at least about 98%, or at least about 99% of the solid form of the compound of Formula (III) is one or more of Forms IV-XIX. In some cases, at least about 50%, at least about 60%, at least about 70%, at least about 80%, at least about 90%, at least about 95%, at least about 98%, or at least about 99% of the solid form of the compound of Formula (III) is one of Forms IV-XIX. In some cases, the solid form of the compound of Formula (III) is at least about 25%, at least about 50%, at least about 75%, at least about 90%, at least about 95%, or at least about 99% amorphous. In some cases, the solid form of the compound of Formula (III) is at most about 25%, at most about 50%, at most about 75%, at most about 90%, at most about 95%, or at most about 99% amorphous. In some cases, the compound of Formula (III) is Form IV. In some cases, the compound of Formula (III) is Form V. In some cases, the compound of Formula (III) is Form VI. In some cases, the compound of Formula (III) is Form VII. In some cases, the compound of Formula (III) is Form VIII. In some cases, the compound of Formula (III) is Form IX. In some cases, the compound of Formula (III) is Form X. In some cases, the compound of Formula (III) is Form XI. In some cases, the compound of Formula (III) is Form XII. In some cases, the compound of Formula (III) is Form XIII. In some cases, the compound of Formula (III) is Form XIV. In some cases, the compound of Formula (III) is Form XV. In some cases, the compound of Formula (III) is Form XVI. In some cases, the compound of Formula (III) is Form XVII. In some cases, the compound of Formula (III) is Form XVIII. In some cases, the compound of Formula (III) is Form XIX.

In some cases, between about 0.1% and about 75%, between about 0.1% and about 10%, between about 0.1% and about 25%, between about 1% and about 25%, between about 5% and about 50%, between about 20% and about 50%, or between about 25% and about 75% of the solid form of the compound of Formula (III) dissolves in the solvent system. In some cases, the solvent system comprises a temperature between about 5° C. and about 110° C., between about 5° C. and about 90° C., between about 5° C. and about 25° C., between about 10° C. and about 60° C., between about 25° C. and about 50° C., between about 15° C. and about 50° C., between about 15° C. and about 70° C., between about 25° C. and about 40° C., or between about 35° C. and about 60° C. In some cases, the incubating is performed for between about 0.1 and about 700 hours, between about 1 and about 360 hours, between about 3 and about 360 hours, between about 3 and about 36 hours, between about 6 and about 72 hours, between about 12 and about 150 hours, between about 24 and about 150 hours, between about 48 and about 240 hours, or between about 100 and about 700 hours. In some cases, the solvent system comprises between about 5 and about 10000 mg/ml, between about 5 and about 1000 mg/ml, between about 5 and about 300 mg/ml, between about 5 and about 50 mg/ml, between about 20 and about 300 mg/ml, between about 50 and about 300 mg/ml, between about 100 and about 1000 mg/ml, or between about 300 and about 10000 mg/ml of the solid form of the compound of Formula (III) during the incubating.

In some cases, the solvent system comprises 2-propanol, acetonitrile, acetone, butyl acetate, butyl methyl ether, dimethylformamide, ethanol, ethyl acetate, water, heptane, methanol, methyl isobutyl ketone, tetrahydrofuran, toluene, or a combination thereof. In some cases, the solvent system comprises a single solvent. In some cases, the solvent system comprises a plurality of solvents. In some cases, the solvent system comprises two solvents in a ratio of between about 99:1 and about 1:1, between about 19:1 and about 1:1, between about 19:1 and 5:1, between about 8:1 and about 1:1, between about 4:1 and about 1:1, or between about 2:1 and about 1:1. In some cases, the solvent system comprises less than about 25%, less than about 10%, less than about 5%, less than about 2%, less than about 1%, or less than about 0.25% water.

### Methods of Generating Form IV

Aspects of the present disclosure provide methods for generating Form IV of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises: dissolving the compound of Formula (III) in 2-propanol, and evaporating at least a portion of the 2-propanol; dissolving the compound of Formula (III) in a solvent system comprising 2-propanol and tetrahydrofuran at a first temperature, and cooling the solvent system to a second temperature; dissolving the compound of Formula (III) in a solvent system comprising heptane and 2-propanol at a first temperature, and cooling the solvent system to a second temperature; dissolving the compound of Formula (III) in a solvent system comprising 2-propanol and ethyl acetate at a first temperature, and cooling the solvent system to a second temperature; dissolving the compound of Formula (III) in tetrahydrofuran, and combining the solvent system with water; dissolving the compound of Formula (III) in a solvent system comprising 2-propanol and tetrahydrofuran, and evaporating at least a portion of the solvent system; dissolving the compound of Formula (III) in acetone, and evaporating at least a portion of the acetone; dissolving the compound of Formula (III) in a solvent system comprising acetone and tetrahydrofuran, and evaporating at least a portion of the solvent system; or a combination thereof, thereby producing the crystalline form of the compound of Formula (III). In some cases, a method for producing a crystalline form of a compound of Formula (III) comprises: dissolving the compound of Formula (III) in 2-propanol, and evaporating at least a portion of the 2-propanol; dissolving the compound of Formula (III) in a solvent system comprising 2-propanol and tetrahydrofuran at a first temperature, and cooling the solvent system to a second temperature; dissolving the compound of Formula (III) in a solvent system comprising heptane and 2-propanol at a first temperature, and cooling the solvent system to a second temperature; dissolving the compound of Formula (III) in a solvent system comprising 2-propanol and ethyl acetate at a first temperature, and cooling the solvent system to a second temperature; dissolving the compound of Formula (III) in tetrahydrofuran, and combining the solvent system with water; dissolving the compound of Formula (III) in a solvent system comprising 2-propanol and tetrahydrofuran, and evaporating at least a portion of the solvent system; dissolving the compound of Formula (III) in a solvent system comprising acetone and tetrahydrofuran, and evaporating at least a portion of the solvent system; or a combination thereof, thereby producing the crystalline form of the compound of Formula (III). In some cases, the crystalline form of the compound of Formula (III) is Form IV.

In some cases, the cooling is at a rate of between about 0.02° C./minute and about 10° C./minute, between about 0.02° C./minute and about 1° C./minute, between about 2° C./minute and about 10° C./minute. In some cases, the first temperature is between about 20° C. and about 100° C., and the second temperature is between about −20° C. and about 25° C. In some cases, the first temperature is between about 20° C. and about 50° C., between about 40° C. and about 70° C., between about 60° C. and about 100° C., or between about 20° C. and about 100° C. In some cases, the second temperature is between about −30° C. and about 30° C., between about −30° C. and about 0° C., or between about 0° C. and about 25° C. In some cases, the evaporating removes between about 10% and about 70%, between about 20% and about 50%, or between about 10% and about 35% of the solvent system. In some cases, the solvent or solvent-system is at least about 80%, at least about 90%, at least about 95%, at least about 98%, or at least about 99% pure (e.g., a 2-propanol solution comprises at least about 80%, at least about 90%, at least about 95%, at least about 98%, or at least about 99% 2-propanol).

### Methods of Generating Form V

Aspects of the present disclosure provide methods for generating Form V of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises dissolving the compound of Formula (III) in acetonitrile at a first temperature, and cooling the acetonitrile to a second temperature, thereby producing the crystalline form of the compound of Formula (III). In some cases, the cooling is at a rate of between about 0.02° C./minute and about 10° C./minute, between about 0.02° C./minute and about 1° C./minute, between about 2° C./minute and about 10° C./minute. In some cases, the first temperature is between about 20° C. and about 100° C., and the second temperature is between about −20° C. and about 25° C. In some cases, the first temperature is between about 20° C. and about 50° C., between about 40° C. and about 70° C., between about 60° C. and about 100° C., or between about 20° C. and about 100° C. In some cases, the second temperature is between about −30° C. and about 30° C., between about −30° C. and about 0° C., or between about 0° C. and about 25° C. In some cases, the acetonitrile is at least about 80%, at least about 90%, at least about 95%, at least about 98%, or at least about 99% pure.

### Methods of Generating Form VI

Aspects of the present disclosure provide methods for generating Form VI of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises storing Form V at a temperature of between about 10° C. and about 100° C., thereby producing the crystalline form of the compound of Formula (III). In some cases, the temperature is between about 10° C. and about 50° C., or between about 20° C. and about 50° C. In some cases, the Form V of the compound of Formula (III) is dry during storage. In some cases, the crystalline form of the compound of Formula (III) is Form VI. In some cases, the solvent or solvent-system is at least about 80%, at least about 90%, at least about 95%, at least about 98%, or at least about 99% pure.

### Methods of Generating Form VII

Aspects of the present disclosure provide methods for generating Form VII of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of Formula (III) comprises dissolving the compound of Formula (III) in ethyl acetate, and adding the ethyl acetate to heptane; dissolving the compound of Formula (III) in toluene, and evaporating at least a portion of the toluene; dissolving the compound of Formula (III) in toluene, and adding the toluene to heptane; dissolving the compound of Formula (III) in a solvent system comprising heptane and toluene, and heating the solvent system to between about 20° C. and about 100° C.; or a combination thereof, thereby producing the crystalline form of the compound of Formula (III). In some cases, the solvent system is heated to between about 20° C. and about 50° C. In some cases, the solvent system is heated to between about 50° C. and about 150° C. In some cases, the evaporating removes between about 10% and about 70%, between about 20% and about 50%, or between about 10% and about 35% of the toluene. In some cases, the crystalline form of the compound of Formula (III) is Form VII. In some cases, the solvent or solvent-system is at least about 80%, at least about 90%, at least about 95%, at least about 98%, or at least about 99% pure.

### Methods of Generating Form VIII

Aspects of the present disclosure provide methods for generating Form VIII of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of Formula (III) comprises dissolving the compound of Formula (III) in toluene at a first temperature and cooling the solvent system to a second temperature; dissolving the compound of Formula (III) in toluene, adding butyl methyl ether to the toluene to form a solvent system, and evaporating at least a portion of the solvent system; or a combination thereof, thereby producing the crystalline form of the compound of Formula (III). In some cases, the crystalline form of the compound of Formula (III) is Form VIII. In some cases, the cooling is at a rate of between about 0.02° C./minute and about 10° C./minute, between about 0.02° C./minute and about 1° C./minute, between about 2° C./minute and about 10° C./minute. In some cases, the first temperature is between about 20° C. and about 100° C., and the second temperature is between about −20° C. and about 25° C. In some cases, the first temperature is between about 20° C. and about 50° C., between about 40° C. and about 70° C., between about 60° C. and about 100° C., or between about 20° C. and about 100° C. In some cases, the second temperature is between about −30° C. and about 30° C., between about −30° C. and about 0° C., or between about 0° C. and about 25° C. In some cases, the evaporating removes between about 10% and about 70%, between about 20% and about 50%, or between about 10% and about 35% of the solvent system. In some cases, the toluene is at least about 80%, at least about 90%, at least about 95%, at least about 98%, or at least about 99% pure.

### Methods of Generating Form IX

Aspects of the present disclosure provide methods for generating Form IX of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of Formula (III) comprises dissolving the compound of Formula (III) in a solvent system comprising ethyl acetate, adding heptane to the solvent system, and incubating the solvent system at a temperature of between about 20° C. and about −20° C.; dissolving the compound of Formula (III) in a solvent system comprising butyl acetate, adding heptane to the solvent system; dissolving the compound of Formula (III) in ethyl acetate, adding the ethyl acetate to butyl methyl ether to form a solvent system, and evaporating at least a portion of the solvent system; dissolving the compound of Formula (III) in ethyl acetate, adding butyl methyl ether to the ethyl acetate to form a solvent system, and evaporating at least a portion of the solvent system; dissolving the compound of Formula (III) in acetone, combining the acetone with butyl methyl ether to form a solvent system, and evaporating at least a portion of the solvent system; a combination thereof, thereby producing the crystalline form of the compound of Formula (III). In some cases, the crystalline form of the compound of Formula (III) is Form IX. In some cases, the evaporating removes between about 10% and about 70%, between about 20% and about 50%, or between about 10% and about 35% of the solvent system. In some cases, the acetone, ethyl acetate, and/or butyl acetate is at least about 80%, at least about 90%, at least about 95%, at least about 98%, or at least about 99% pure.

### Methods of Generating Form X

Aspects of the present disclosure provide methods for generating Form X of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises dissolving the compound of Formula (III) in 4-methyl-2-pentanone at a first temperature and cooling the 4-methyl-2-pentanone to a second temperature, thereby producing the crystalline form of the compound of Formula (III). In some cases, the crystalline form of the compound of Formula (III) is Form X. In some cases, the cooling is at a rate of between about 0.02° C./minute and about 10° C./minute, between about 0.02° C./minute and about 1° C./minute, between about 2° C./minute and about 10° C./minute. In some cases, the first temperature is between about 20° C. and about 50° C., between about 40° C. and about 70° C., between about 60° C. and about 100° C., or between about 20° C. and about 100° C. In some cases, the second temperature is between about −30° C. and about 30° C., between about −30° C. and about 0° C., or between about 0° C. and about 25° C. In some cases, the 4-methyl-2-pentanone is at least about 80%, at least about 90%, at least about 95%, at least about 98%, or at least about 99% pure.

### Methods of Generating Form XII

Aspects of the present disclosure provide methods for generating Form XII of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises: dissolving less than about 100 micrograms of the compound of Formula (III) in tetrahydrofuran, adding the tetrahydrofuran to acetone to form a solvent system, and evaporating at least a portion of the solvent system, thereby producing the crystalline form of the compound of Formula (III). In some cases, the crystalline form of the compound of Formula (III) is Form XII. In some cases, the evaporating removes between about 10% and about 70%, between about 20% and about 50%, or between about 10% and about 35% of the solvent system. In some cases, the tetrahydrofuran and/or the acetone are at least about 80%, at least about 90%, at least about 95%, at least about 98%, or at least about 99% pure.

### Methods of Generating Form XIII

Aspects of the present disclosure provide methods for generating Form XIII of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises: dissolving greater than about 100 micrograms of the compound of Formula (III) in tetrahydrofuran, adding the tetrahydrofuran to acetone to form a solvent system, and evaporating at least a portion of the solvent system, thereby producing the crystalline form of the compound of Formula (III). In some cases, the crystalline form of the compound of Formula (III) is Form XIII. In some cases, the evaporating removes between about 10% and about 70%, between about 20% and about 50%, or between about 10% and about 35% of the solvent system. In some cases, the tetrahydrofuran and/or the acetone are at least about 80%, at least about 90%, at least about 95%, at least about 98%, or at least about 99% pure.

### Methods of Generating Form XIV

Aspects of the present disclosure provide methods for generating Form XIV of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises dissolving the compound of Formula (III) in ethyl acetate, and adding the ethyl acetate to heptane, thereby producing the crystalline form of the compound of Formula (III). In some cases, the crystalline form of the compound of Formula (III) is Form XV. In some cases, the ethyl acetate and/or heptane are at least about 80%, at least about 90%, at least about 95%, at least about 98%, or at least about 99% pure.

### Methods of Generating Form XVI

Aspects of the present disclosure provide methods for generating Form XVI of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises: dissolving the compound of Formula (III) in ethyl acetate at a first temperature, combining the ethyl acetate with a heptane to form a solvent system, and cooling the solvent system to a second temperature, thereby producing the crystalline form of the compound of Formula (III). In some cases, the crystalline form of the compound of Formula (III) is Form XVI. In some cases, the cooling is at a rate of between about 0.02° C./minute and about 10° C./minute, between about 0.02° C./minute and about 1° C./minute, between about 2° C./minute and about 10° C./minute. In some cases, the first temperature is between about 20° C. and about 100° C., and the second temperature is between about −20° C. and about 25° C. In some cases, the first temperature is between about 20° C. and about 50° C., between about 40° C. and about 70° C., between about 60° C. and about 100° C., or between about 20° C. and about 100° C. In some cases, the second temperature is between about −30° C. and about 30° C., between about −30° C. and about 0° C., or between about 0° C. and about 25° C. In some cases, the ethyl acetate is at least about 80%, at least about 90%, at least about 95%, at least about 98%, or at least about 99% pure. In some cases, the heptane is at least about 80%, at least about 90%, at least about 95%, at least about 98%, or at least about 99% pure.

### Methods of Generating Form XVII

Aspects of the present disclosure provide methods for generating Form XVII of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises dissolving the compound of Formula (III) in ethanol, and evaporating at least a portion of the ethanol, thereby producing the crystalline form of the compound of Formula (III). In some cases, the crystalline form of the compound of Formula (III) is Form XVII. In some cases, the evaporating removes between about 10% and about 70%, between about 20% and about 50%, or between about 10% and about 35% of the ethanol. In some cases, the ethanol is at least about 80%, at least about 90%, at least about 95%, at least about 98%, or at least about 99% pure.

### Methods of Generating Form XVIII

Aspects of the present disclosure provide methods for generating Form XVIII of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of Form (III) comprises dissolving the compound of Formula (III) in tetrahydrofuran, and evaporating at least a portion of the tetrahydrofuran, thereby producing the crystalline form of the compound of Formula (III). In some cases, the crystalline form of the compound of Formula (III) is Form XVIII. In some cases, the evaporating removes between about 10% and about 70%, between about 20% and about 50%, or between about 10% and about 35% of the tetrahydrofuran. In some cases, the tetrahydrofuran is at least about 80%, at least about 90%, at least about 95%, at least about 98%, or at least about 99% pure.

### Methods of Generating Form XIX

Aspects of the present disclosure provide methods for generating Form XIX of the compound of Formula (III). In some cases, at least about 80%, at least about 90%, at least about 95%, at least about 98%, or at least about 99% by weight of the compound of Formula (III) in the composition is the (Z)-isomer. In some cases, a method for producing a crystalline form of a compound of Formula (III) comprises: dissolving the compound of Formula (III) in acetone at a first temperature, combining the acetone with heptane to form a solvent system, and cooling the solvent system to a second temperature; dissolving the compound of Formula (III) in tetrahydrofuran at a first temperature, combining the tetrahydrofuran with heptane to form a solvent system, and cooling the solvent system to a second temperature; or a combination thereof; thereby producing the crystalline form of the compound of Formula (III). In some cases, the crystalline form of the compound of Formula (III) is Form XIX. In some cases, the cooling is at a rate of between about 0.02° C./minute and about 10° C./minute, between about 0.02° C./minute and about 1° C./minute, between about 2° C./minute and about 10° C./minute. In some cases, the first temperature is between about 20° C. and about 100° C., and the second temperature is between about −20° C. and about 25° C. In some cases, the first temperature is between about 20° C. and about 50° C., between about 40° C. and about 70° C., between about 60° C. and about 100° C., or between about 20° C. and about 100° C. In some cases, the second temperature is between about −30° C. and about 30° C., between about −30° C. and about 0° C., or between about 0° C. and about 25° C. In some cases, the acetone is at least about 80%, at least about 90%, at least about 95%, at least about 98%, or at least about 99% pure. In some cases, the heptane is at least about 80%, at least about 90%, at least about 95%, at least about 98%, or at least about 99% pure. In some cases, the tetrahydrofuran is at least about 80%, at least about 90%, at least about 95%, at least about 98%, or at least about 99% pure.

### Industrially Scalable Processes

In an aspect, the present disclosure provides industrially scalable processes for manufacturing (Z)-endoxifen and salts thereof. An industrially scalable process may be used to produce up to about 1 kg, up to about 2 kg, up to about 3 kg, up to about 4 kg, or up to about 5 kg of (Z)-endoxifen in a single batch. In some embodiments, an industrially scalable process may be used to produce from about 500 g to about 5 kg of (Z)-endoxifen in a single batch. In some embodiments, an industrially scalable process may be used to produce at least about 500 g, at least about 750 g, at least about 1 kg, at least about 2 kg, at least about 3 kg, at least about 4 kg, or at least about 5 kg of (Z)-endoxifen in a single batch.

In an aspect, the present disclosure relates to an industrially scalable process for manufacturing (Z)-endoxifen, comprising the steps of: (a) reacting a mixture of (E)-endoxifen and (Z)-endoxifen, compounds of Formula (III), to 6N HCL (1:1 to 1:5 wt/wt) in EtOAc (1:1 to 1:20 wt/wt), (b) neutralizing with 8N NaOH (1:1 to 1:20 wt/wt); (c) washing one or more times with EtOAc (1:1 to 1:10 wt/wt); (d) extracting with 20% NaCl (1:1 to 1:5 wt/wt); (e) reacting with activated carbon (1:0.01 to 1:1), optionally followed by crystallization using EtOAc, e.g., to recover and/or remove residual compounds of Formula (III) and/or (E)-endoxifen; (f) recrystallizing in a first solvent of acetone (1:0.1 to 1:10 wt/wt) and a second solvent of IPA (1:0.1 to 1:10 wt/wt); (g) recrystallizing in a first solvent of THF (1:0.1 to 1:10 wt/wt) and a second solvent of IPA (1:0.1 to 1:10 wt/wt); wherein the wt/wt is with respect to the mixture of (E)-endoxifen and (Z)-endoxifen, e.g., compounds of Formula (III). For example, the industrially scalable process for manufacturing (Z)-endoxifen may comprise the steps described in EXAMPLE 3.

In an aspect, the present disclosure relates to an industrially scalable process for manufacturing a mixture of (E)-endoxifen and (Z)-endoxifen (compounds of Formula (III)), comprising: (a) reacting the compound of Formula (II) with propiophenone in THE (4.4 wt/wt); (b) preparing a solution of TiCl₄ (1.4 wt/wt) and Zn (0.9 wt/wt) in THE (8.9 wt/wt); and (c) reacting the compound of Formula (II) of step (a) with TiCl₄ and Zn in THE from step (b) to form a mixture of (E)-endoxifen and (Z)-endoxifen; wherein wt/wt is with respect to the compound of Formula (II). For example, the industrially scalable process for manufacturing (Z)-endoxifen may comprise the steps described in EXAMPLE 2.

In an aspect, the present disclosure relates to an industrially scalable process for manufacturing a mixture of (E)-endoxifen and (Z)-endoxifen, compounds of Formula (III), from Formula (II) as starting material, comprising one or more steps of: (a) extraction with 25% ammonium chloride (1:20 wt/wt) and silica (Celite) (1:1 wt/wt); (b) one or more washes with THF (1:1 to 1:5 wt/wt); (c) one or more washes with 20% sodium chloride (1:3 wt/wt); (c) distillation with EtOAc (1:4.5 wt/wt); and (d) crystallization with (1:2 v/v) EtOAc/n-heptane (1:3.8 wt/wt); wherein the wt/wt is with respect to the compound of Formula (II).

In an aspect, the present disclosure relates to an industrially scalable process for manufacturing a mixture of (E)-endoxifen and (Z)-endoxifen, compounds of Formula (III), further comprising one or more steps of: (a) extraction one or more times with 40% K₂CO₃ (1:2 wt/wt); (b) extraction with 1N NaOH (1:10 wt/wt) and MeTHF (1:1 to 1:10 wt/wt); (c) two more extractions with MeTHF (1:1 to 1:20 wt/wt); (d) extraction with 20% sodium chloride (1:5 wt/wt); (e) distillation with IPA (1:4.5 wt/wt); and (f) crystallization with (1:2.7 v/v) IPA/n-heptane (1:3.4 wt/wt); wherein the wt/wt is with respect to the compound of Formula (II).

In an aspect, the present disclosure relates to an industrially scalable process for manufacturing the compound of Formula (II), comprising the steps of: (a) reacting the compound of Formula (I) (1 equiv.) with DIPEA (3 wt/wt) in THE (4.9 wt/wt); (b) adding 1-chloroethyl chloroformate (3.3 wt/wt); (c) distilling one to five times with methanol (4.0 wt/wt); (d) distilling with methanol (3.2 wt/wt); (e) reacting with methanol (3.2 wt/wt)/6N HCl (4 wt/wt); and (f) neutralizing with 8N NaOH (5 wt/wt); and wherein wt/wt is with respect to the compound of Formula (I). For example, the industrially scalable process for manufacturing (Z)-endoxifen may comprise the steps described in EXAMPLE 1.

In an aspect, the present disclosure relates to (Z)-endoxifen, (E)-endoxifen, compounds of Formula (III), and the compound of Formula (II), and salts thereof, prepared according to processes described herein. In an aspect, the present disclosure relates to (Z)-endoxifen prepared according to a process disclosed herein, wherein the (Z)-endoxifen is stable at ambient temperature for at least 6 months, at least 9 months, at least 12 months, or at least 18 months.

In another aspect, the (Z)-endoxifen free base prepared in accordance with the processes disclosed herein have <1% impurity. In still another aspect, the (E)/(Z)-endoxifen free base prepared in accordance with the processes disclosed herein has <1% impurity.

In various embodiments, the (Z)-endoxifen free base prepared in accordance with the processes disclosed herein has <1% impurity, e.g., less than 25 ppm mesityl oxide, and is stable at ambient temperature for at least 6 months, at least 9 months, at least 12 months, or at least 18 months.

### Methods of Use

Compounds of Formulas (I), (II), (III), and (IV), endoxifen salts (e.g., pharmaceutically acceptable salts) disclosed herein, and compositions comprising them may be used in the manufacture of medicaments for use in the treatment of a subject in need thereof, for example, subjects having or at risk of having a hormone-dependent breast disorder, a hormone-dependent reproductive tract disorder, or both. In various instances, the compound used for such treatments is (Z)-endoxifen of Formula (IV) prepared using the methods described herein, e.g., those of Example 1-Example 3

The compositions of the present disclosure may be used as a primary therapy, as a part of a neo-adjuvant therapy (to primary therapy), or as part of adjuvant therapy regimen, where the intention is to ameliorate or cure a subject having or at risk of having a hormone-dependent breast disorder, hormone-dependent reproductive tract disorder, or both.

In certain embodiments, the disorder is a hormone-dependent breast disorder. In other embodiments, the disorder is hormone-dependent reproductive tract disorder. In still other embodiments, the subject has both a hormone-dependent breast disorder and a hormone-dependent reproductive tract disorder. In some embodiments, the hormone dependent disorder is a benign breast disorder, hyperplasia, atypia, atypical ductal hyperplasia, atypical lobular hyperplasia, increased breast density, gynecomastia, DCIS, LCIS, breast cancer, endometrial cancer, ovarian cancer, uterine cancer, cervical cancer, vaginal cancer or vulvar cancer.

In some embodiments, the breast disorder is increased breast density. For example, the breast disorder is a class B (formerly Class II), class C (formerly class III) or class D (formerly class IV) breast density.

In some embodiments, the hormone-dependent breast disorder or hormone-dependent reproductive tract disorder is precocious puberty. In other embodiments, the hormone-dependent breast disorder or hormone-dependent reproductive tract disorder is McCune-Albright Syndrome.

In some embodiments, the breast disorder is gynecomastia. In some embodiments, gynecomastia is presented secondarily to an underlying disease. Accordingly, in some embodiments, the subject also has underlying disease selected from the group consisting of prostate cancer, cirrhosis and liver disease, male hypogonadism, hyperthyroidism, renal failure and in patients undergoing hemodialysis, or type I diabetes mellitus. In certain embodiments, the subject has prostate cancer as the underlying disease, wherein the subject has or is at risk of having gynecomastia.

In certain embodiments, the breast cancer is DCIS, LCIS, ILC, IDC, MIC, inflammatory breast cancer, ER-positive (ER+) breast cancer, HER2+ breast cancer, adenoid cystic (adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma. In at least one embodiment, a single breast cancer tumor may be a combination of the foregoing or be a mixture of invasive and in situ cancer.

The present disclosure contemplates the use of the compounds and compositions disclosed herein at various stages in tumor development and progression, including the treatment of advanced and/or aggressive neoplasms, i.e., overt disease in a subject that is not amenable to cure by local modalities of treatment such as surgery or radiotherapy, metastatic disease, or locally advanced disease. Accordingly, in some embodiments, the breast cancer is a pre-cancer, an early-stage cancer, a non-metastatic cancer, a pre-metastatic cancer, or a locally advanced cancer. In at least one embodiment, the breast disorder is metastatic cancer. In some embodiments, the subject further has prostate cancer.

Current choice for therapeutics for such disorders remains tamoxifen, despite serious adverse effects, poor patient compliance and resistance to the drug due to low plasma endoxifen levels seen subjects. Such subjects may have low endoxifen levels upon dosing with tamoxifen for any number of reasons, such as having CYP gene mutations, for example, in CYP2D6, CYP3A4, or CYP2C9, making them unable to metabolize tamoxifen to its active metabolite, endoxifen, or low or dysfunctional estrogen receptor preventing (or decreasing) sufficient tamoxifen uptake, for other reasons yet to be identified. Reported therapeutic levels of plasma tamoxifen in subjects dosed with 20 mg of oral tamoxifen is ≥30 nM ((Lyon et al. Genet Med. 2012 December; 14(12):990-1000). Notwithstanding the mechanism underlying the low plasma endoxifen in a subject, the compositions of the present disclosure are useful for any condition wherein a subject has low endoxifen or the subject has or is at a risk of having hormone-dependent breast disorder or hormone-dependent reproductive tract disorder. Therefore, the compositions of the present disclosure can be particularly important in the treatment of tamoxifen-resistant, hormone-dependent breast disorders or hormone-dependent reproductive tract disorders.

Provided herein in certain embodiments are patient populations for whom the pharmaceutical compositions are particularly useful. The compositions of the present disclosure are also particularly important in the treatment of tamoxifen-refractory subjects with hormone-dependent breast disorders or hormone-dependent reproductive tract disorders. Accordingly, in some embodiments, the compositions disclosed herein are useful for the treatment of tamoxifen refractory or tamoxifen resistant subjects having or at risk of having hormone-dependent breast disorders, hormone-dependent reproductive tract disorders, or both. In some embodiments, compositions comprising an endoxifen salt, such as endoxifen gluconate, administered to such subject at the doses disclosed herein, will be advantageous.

Drug interactions between tamoxifen and selective serotonin reuptake inhibitors (SSRI) drugs like Prozac and Paxil (paroxetine) may occur and may be detrimental to breast cancer subjects. The SSRI drugs reduce or stop liver metabolism of tamoxifen to endoxifen in subjects on SSRI drugs. Thus, provided herein in certain embodiments are patient populations being treated or to be treated with SSRI drugs that would be benefitted by treatment with compositions of the present disclosure.

Compositions disclosed herein administered orally maintain the subject's plasma endoxifen at steady state levels greater than 30 nM, for example, at levels ranging from 30 nM to 80 nM or at levels ranging from 30 nM to 300 nM. In some embodiments, the plasma steady state endoxifen levels are maintained at >40 nM. Maintenance of such a plasma endoxifen at steady state levels greater than 30 nM is advantageous in that the likelihood of recurrence (relapse) of hormone-dependent breast disorders or hormone-dependent reproductive tract disorders, particularly breast cancer, at plasma endoxifen levels lower than 30 nM is reduced. It is particularly advantageous for subjects that are poor metabolizers of tamoxifen (with plasma endoxifen levels lower than 16 nM), intermediate metabolizers of tamoxifen (with plasma endoxifen levels lower than 27 nM) to be dosed with a composition disclosed herein. It also advantageous for subjects being treated or to be treated with antidepressant drugs such as SSRI drugs such as citalopram (Celexa), escitalopram (Lexapro), fluoxetine (Prozac), paroxetine (Paxil, Pexeva), sertraline (Zoloft), vilazodone (Viibryd) and the like, for example, a subject having or likely to have depression.

Whether a subject is tamoxifen-refractory may be determined by dosing a subject with an initial dosage of tamoxifen and determining the subject's plasma endoxifen steady state level. Plasma endoxifen steady state levels in a subject dosed with tamoxifen serves as a biomarker for the tamoxifen-refractory subjects. The plasma endoxifen levels (acute and/or steady state) may be determined by obtaining from the subject a test sample, which may be blood sample, collected from the subject after dosing the subject with tamoxifen. Plasma or serum may be obtained from blood samples for testing the biomarker endoxifen levels. The initial dosage may comprise administering tamoxifen daily for at least 1 day, 2 days, 3 days, 15 days, 1 week, 2 weeks, 4 weeks, 1 month, 2 months, 3 months, 4 months, 5 months, or 6 months. The subject may also be administered with a first composition comprising tamoxifen daily for at least 1 day, 2 days, 3 days, 15 days, 1 week, 2 weeks, 4 weeks, 1 month, 2 months, 3 months, 4 months, 5 months, 6 months, 1 year, 2 years, 3 years, 4 years, 5 years, or 10 years.

A subject's plasma endoxifen steady state level may be determined by measuring endoxifen in a test sample. The subject's plasma endoxifen steady state levels are compared to a reference plasma endoxifen level. For the purposes of the present disclosure, the reference plasma level is 30 nM. If the subject's plasma endoxifen level is determined to be lower than 30 nM, then the subject is defined as tamoxifen-refractory. Such a tamoxifen-refractory subject who has or who may be at risk of having a hormone-dependent breast disorder or hormone-dependent reproductive tract disorder is treated by administering to the subject an oral composition comprising (Z)-endoxifen or a salt thereof disclosed herein, or a polymorphic form of endoxifen disclosed herein. In some embodiments, the composition administered to such a subject comprises (Z)-endoxifen free base. In other embodiments, the composition administered to such a subject comprises endoxifen gluconate selected from the group consisting of (Z)-endoxifen D-gluconate, (Z)-endoxifen L-gluconate, (E)-endoxifen D-gluconate, (E)-endoxifen L-gluconate, or a combination thereof. In other embodiments, the composition comprising endoxifen is endoxifen HCl or endoxifen citrate. The present disclosure also contemplates that a subject's plasma endoxifen levels are tracked or monitored periodically or as necessary. If required, a subject who has been administered an initial dosage of tamoxifen may have his or her plasma endoxifen steady state levels adjusted by administering a composition comprising endoxifen on an ongoing basis based on the test results.

In some embodiments, the subject's tamoxifen-refractory status may be determined by determining the subject's tamoxifen-metabolites profile which is compared with a reference tamoxifen-metabolite profile as seen in control or normal subjects. Subjects with low plasma endoxifen levels in subject's tamoxifen-metabolite profile as compared to the reference tamoxifen-metabolite profile are administered an oral composition comprising endoxifen or a salt thereof. Such compositions may comprise synthetically prepared endoxifen.

The plasma endoxifen may be measured by any of method known in the art. The levels of plasma endoxifen in test sample may be determined based on subject's genes, DNA, RNA, protein, tamoxifen-metabolite profile or a combination thereof. The tamoxifen-metabolites profile can include at least tamoxifen, 4-OHT, N-desmethyltamoxifen, and/or endoxifen. In some embodiments, the level of plasma endoxifen and/or tamoxifen-metabolite profile in the test sample is measured by High Performance Liquid Chromatography (HPLC), Gas Chromatography Mass Spectrometry (GC-MS), Liquid Chromatography Mass spectrometry (LC-MS), Liquid Chromatography Tandem Mass spectrometry (LC-MS/MS), immunohistochemistry (IHC), polymerase chain reaction (PCR), quantitative PCR (qPCR), and the like. In some embodiments, the tamoxifen-metabolites profile is predicted based on the subject's genetic composition. In some embodiments, the subject's CYP genotype includes, without limitation, analysis of CYP2D6, CYP3A4, CYP2C9 genes. In some embodiments, subject's estrogen receptor levels may be analyzed. In other embodiments, the determination of plasma endoxifen may be done by a third-party laboratory.

Accordingly, provided herein are methods of maintaining in a subject in need thereof a plasma endoxifen a level greater than 30 nM by administering to the subject a composition comprising endoxifen or a salt thereof. In some embodiments, the subject's plasma endoxifen level is maintained at a steady state level greater than 30 nM. In some embodiments, the subject's plasma endoxifen levels are maintained at a steady state level ranging from 30 nM to 300 nM (for example, from 30 nM to 200 nM, or from 30 nm to 80 nM). In some embodiments, the subject's plasma endoxifen levels are maintained at a steady state level >40 nM.

In another aspect, the subjects may have their test samples tested for their biomarker profile that may be indicative or monitoring a hormone-dependent breast disorder, a hormone-dependent reproductive tract disorder, or both. Such biomarkers are known in the art and include, by way of non-limiting examples, biomarkers such as CYP2D6, BRCA-1, BRCA-2, ER, PR, Her2, uPA, PAI, Tf, p53, Ki67, cytokeratins, cancer tumor antigens, and other biomarkers measured by Mammaprint, OncotypeDx, PAM50, EndoxPredict, MammoStrat, and other diagnostic and predictive tests. A subject with biomarker profile indicating that the subject has or is at risk of having a hormone-dependent breast disorder, a hormone-dependent reproductive tract disorder, or both can be administered a composition disclosed herein. In one aspect, the present disclosure provides a method of treating a subject having or at risk of having a hormone-dependent breast disorder, a hormone-dependent reproductive tract disorder, or both, comprising determining a subject's tamoxifen-refractory or tamoxifen-resistant status and administering to the subject a composition described herein.

In some aspects, provided herein are methods of treating a tamoxifen-refractory or tamoxifen-resistant subject, the method comprising administration to the subject a composition comprising endoxifen, or a salt or polymorph thereof.

In some embodiments disclosed herein are methods of treating a tamoxifen-refractory subject having or at risk for having a hormone-sensitive breast disorder, a hormone-sensitive reproductive tract disorder, or both, the method comprising administration to the subject an oral composition comprising endoxifen, or a salt thereof, wherein the subject has a plasma endoxifen level of less than 30 nM, less than 25 nM, less than 20 nM, less than 15 nM, less than 10 nM, less than 5 nM or less than 1 nM. In certain embodiments, the endoxifen salt is endoxifen gluconate, endoxifen HCl, or endoxifen citrate. In other embodiments, an oral solid dosage form comprising at least 90% (Z)-endoxifen or a salt thereof is administered.

Also provided herein are methods of treating a tamoxifen-refractory subject, the method comprising: (a) determining or having determined plasma endoxifen level in a test sample obtained from the subject; (b) comparing or having compared or having determined the level of plasma endoxifen in the test sample with a reference plasma endoxifen level; (c) determining or having determined a reduced level of plasma endoxifen in the test sample as compared to the reference plasma endoxifen level; and (d) administering a composition comprising endoxifen or a salt or polymorph thereof to the subject. The administration of a composition comprising endoxifen or a salt or polymorph thereof maintains the levels of plasma endoxifen in the subject at steady state levels greater than 30 nM. In some embodiments, the levels of plasma endoxifen in the subject are maintained at steady state levels ranging from 30 nM to 80 nM.

Provided herein are methods of treating a subject having or at risk of having a hormonal dependent breast disorder or a hormonal dependent reproductive tract disorder, the method comprising: (a) administering to the subject a first composition comprising tamoxifen; (b) determining or having determined the level of plasma endoxifen in a test sample obtained from the subject; (c) determining or having determined reduced level of plasma endoxifen in test sample as compared to a reference level of plasma endoxifen; and (d) administering an oral composition disclosed herein to the subject. The subject may be administered with the first composition comprising tamoxifen daily for at least 1 day, 2 days, 3 days, 15 days, 1 week, 2 weeks, 4 weeks, 1 month, 2 months, 3 months, 4 months, 5 months, 6 months, 1 year, 2 years, 3 years, 4 years, 5 years or 10 years. In some embodiments, administration of the oral composition comprising endoxifen or a salt or polymorph thereof maintains the subject's plasma endoxifen at levels greater than 30 nM. In other embodiments, administration of the oral composition comprising endoxifen or a salt or polymorph thereof maintains the subject's plasma endoxifen at levels ranging from 30 nM to 300 nM (e.g., from 30 nM to 200 nM, or from 30 nM to 80 nM). In some embodiments, the subject is administered an oral composition comprising (Z)-endoxifen D-gluconate, (Z)-endoxifen L-gluconate, (E)-endoxifen D-gluconate, (E)-endoxifen L-gluconate, or a combination thereof. In other embodiments, the oral composition comprising endoxifen salt is endoxifen HCl or endoxifen citrate.

Provided herein are methods of treating a subject with a hormone-dependent breast disorder or hormone-dependent reproductive tract disorder, the method comprising: (a) dosing the subject with a first composition comprising tamoxifen; (b) determining or having determined the subject's tamoxifen-metabolites profile in a test sample obtained from the subject; (c) determining a reduced level of subject's plasma endoxifen based on the subject's tamoxifen-metabolites profile to compared to a level of reference plasma endoxifen in a reference tamoxifen-metabolites profile; and (d) administering an oral composition comprising endoxifen or a salt or polymorph thereof to the subject. In certain embodiments, the composition comprising endoxifen is endoxifen gluconate, endoxifen HCl, or endoxifen citrate.

Provided herein are methods for adjusting plasma endoxifen levels in a subject being treated for hormone-dependent breast disorder or hormone-dependent reproductive tract disorder who has one or more CYP2D6 or CYP3A4 mutations or has previously administered with initial dosage of tamoxifen, and who has a plasma endoxifen level less than reference plasma endoxifen level, the method comprising: (a) measuring the subject's plasma endoxifen level after initial dosage of tamoxifen; (b) comparing the subject's plasma endoxifen levels to the reference plasma endoxifen level; (c) administering an oral composition comprising endoxifen or a salt or polymorph thereof to the subject to maintain the subject's plasma endoxifen level at levels greater than 30 nM. In some embodiments, administration of the oral composition comprising endoxifen or a salt or polymorph thereof maintains the subject's plasma endoxifen at levels ranging from 30 nM to 300 nM (e.g., from 30 nM to 200 nM, or from 30 nM to 80 nM). In some embodiments, the subject's plasma endoxifen level is maintained at a steady state level. The subject may be administered with initial dosage of tamoxifen daily for at least 1 day, 2 days, 3 days, 15 days, 1 week, 2 weeks, 4 weeks, 1 month, 2 months, 3 months, 4 months, 5 months, or 6 months.

Provided herein are methods for adjusting the level of plasma endoxifen in a subject being treated for hormone-dependent breast disorder or hormone-dependent reproductive tract disorder who has been previously administered an initial dosage of tamoxifen and who has a level of plasma endoxifen less than 30 nM, the method comprising: (a) measuring the level of plasma tamoxifen-metabolite endoxifen of the subject after the initial dosage of tamoxifen; (b) comparing the plasma level of tamoxifen-metabolite endoxifen to the reference level for normal tamoxifen-metabolite endoxifen level; (c) administering an adjusting dosage of a composition comprising synthetically prepared endoxifen, wherein the dosage of synthetically prepared endoxifen is sufficient to maintain the subject's plasma endoxifen at a level greater than 30 nM. In some embodiments, administration of the second composition comprising a synthetically prepared endoxifen maintains the subject's plasma endoxifen at levels ranging from 30 nM to 300 nM (e.g., from 30 nM to 200 nM, from 30 nM to 80 nM). In some embodiments, the steps (a) to (c) may be repeated until the subject exhibits a desired plasma level of endoxifen.

In an aspect, the present disclosure contemplates a method of treating a subject having or at risk of having a hormone-dependent breast disorder, a hormone-dependent reproductive tract disorder, or both, the method comprising resection of breast tissue of the subject or administering radiotherapy to the subject and administering an oral composition comprising endoxifen or a salt or polymorph thereof disclosed herein. In another aspect, the present disclosure contemplates a method of treating a subject having or at risk of having a hormone-dependent breast disorder, a hormone-dependent reproductive tract disorder, or both, the method comprising administering an oral composition disclosed herein prior to resection of breast tissue of the subject or administering radiotherapy to the subject.

Dosage to be administered to a subject will be usually in a unit dosage form. Examples of ranges for endoxifen in each dosage unit form are from 0.01 mg to 200 mg. Dosage shall generally be an effective amount and equivalent, on a molar basis, of the pharmacologically active (Z)-form produced by a dosage formulation upon metabolic release of the active free drug to achieve its desired pharmacological and physiological effects. In some embodiments, the compositions comprising endoxifen or an endoxifen salt or polymorph are administered to the subject at a dose of 0.01 mg to 200.0 mg. In other embodiments, the oral compositions comprising endoxifen or an endoxifen salt or polymorph are administered to the subject at a dose of 1 mg to 200.0 mg. In some embodiments, the oral compositions comprising endoxifen or an endoxifen salt or polymorph are administered to the subject at a dose of 0.01 mg, 0.05 mg, 0.1 mg, 0.25 mg, 0.5 mg, 0.75 mg, 1.0 mg, 1.5 mg, 2.0 mg, 4.0 mg, 6 mg, 8 mg, 10 mg, 20 mg, 40 mg, 50 mg, 100 mg or 200 mg per unit dose. In certain embodiments, the oral compositions comprising at least 95%, 97%, or at least 99% (Z)-endoxifen (wt/wt) of endoxifen are administered at a dose of 1 mg, 2.0 mg, 4.0 mg 6 mg, 8 mg, 10 mg, 20 mg, 40 mg, 50, 100 mg or 200 mg per unit dose. In some embodiments, the compositions comprising endoxifen gluconate are administered at a dose ranging from 0.01 to 20 mg. In some embodiments, a composition comprising (Z)-endoxifen D-gluconate is administered at 0.5 mg, 1 mg, 2 mg 4.0 mg, 6 mg, 8 mg, 10 mg, 20 mg, 40 mg, 50 mg, 100 mg, and 200 mg per unit dose. In some embodiments, a composition comprising 1 mg of (Z)-endoxifen D-gluconate is administered. In other embodiments, a composition comprising 1 mg of (Z)-endoxifen L-gluconate is administered. In yet other embodiments, a composition comprising 2 mg of (Z)-endoxifen D-gluconate and (E)-endoxifen D-gluconate is administered. In certain embodiments, an oral composition comprising at least 90% of a polymorph, such as polymorphic Form I, Form IV, Form V, Form VI, Form VII, Form VIII, Form IX, Form X, Form XI, Form XII, Form XIII, Form XIV, Form XV, Form XVI, Form XVII, Form XVIII, or Form XIX, of endoxifen (wt/wt) is administered at a dose of 1 mg, 2.0 mg, 4.0 mg 6 mg, 8 mg, 10 mg, 20 mg, 40 mg, 50, 100 mg or 200 mg per unit dose. In some embodiments, a composition comprising a polymorphic form, such as Form I, Form IV, Form V, Form VI, Form VII, Form VIII, Form IX, Form X, Form XI, Form XII, Form XIII, Form XIV, Form XV, Form XVI, Form XVII, Form XVIII, or Form XIX, of endoxifen is administered at a dose ranging from 0.01 to 20 mg.

Breast cancer growth rate studies have shown, using mammographic screening of subjects with breast cancer, that the breast cancer growth rate in the 25^{th} percentiles of women ages 50 to 59 indicate an unmet need for fast exposure of the subject to therapeutics (Weeden-Fekjaer et al. Breast Cancer Research200810:R41). Rapid absorption and bioavailability of the anti-cancer therapeutics such as endoxifen that can further reduce the cancer growth rate is highly desirable.

In one aspect, rapid achievement of maximal and steady state plasma levels of endoxifen is a particular aspect of the present disclosure. The present disclosure provides that administration of compositions disclosed herein to a subject achieves a maximal plasma level of endoxifen ranging within 2 to 30 hours, within 3 to 20 hours, within 2 to 10 hours or within 4 to 8 hours after administration of the compositions. Accordingly, in some embodiments, time to maximal (peak) plasma level of endoxifen ranges from 2 to 10 hours after administration of the compositions. In some embodiments, the time to maximal plasma level of endoxifen ranges from 4 to 8 hours after administration of composition disclosed herein.

Rapid achievement of steady-state plasma levels of endoxifen is also highly desirable. Plasma levels of endoxifen in a subject administered with a composition disclosed herein comprising (Z)-endoxifen or a salt or polymorph thereof rapidly achieves steady state. Steady state plasma levels can be achieved from day 7 to day 21. In some embodiments, the steady state plasma levels can be achieved by day 7 following daily administration of a composition disclosed herein.

In an aspect, the present provides that circulating endoxifen released from compositions disclosed herein can be cleared faster than tamoxifen. Terminal elimination half-life of tamoxifen is said to be 5-7 days (Jordan C. Steroids. 2007 November; 72(13): 829-842) and peak concentration time of tamoxifen is approximately 5 hours post-dose. Endoxifen released from a composition disclosed herein can have a terminal elimination half-life ranging from 30 to 60 hours, significantly lower than tamoxifen. In some embodiments, the mean half-life ranges from 40 to 53 hours. Mean Ratio of AUC₂₄ₕᵣ (Day 21)/AUC_{0-inf} (Day 1) typically ranges from 0.7 to 1.2 for compositions comprising 1 mg to 4 mg (Z)-endoxifen or a salt or polymorph thereof. Thus, accumulation of endoxifen released from the compositions disclosed herein do not significantly vary over continued treatment.

In another aspect, the present disclosure provides that administration of compositions disclosed herein achieve absorption of endoxifen that is therapeutically effective.

Area under Curve AUC₍₀₋₂₄ₕᵣ₎ (“AUC₂₄ₕᵣ”) describes the total exposure of the subject to a drug from time of dosing (0 hr) over a 24-hour period. Compositions comprising (Z)-endoxifen or a salt thereof typically achieve mean (AUC₂₄ₕᵣ) of 150 hr*ng/mL to 600 hr*ng/mL on Day 1 of initial (first) dose of compositions comprising 1 mg to 4 mg of (Z)-endoxifen. Compositions comprising (Z)-endoxifen or salts thereof typically achieve mean AUC₂₄ₕᵣ of 400 hr*ng/mL to 2500 hr*ng/mL on Day 21 of initial (first) dose of compositions comprising 1 mg to 4 mg of (Z)-endoxifen.

AUC_{(0-inf)} (“AUC_{0-inf}”), a time-averaged concentration of drug circulating in the body fluid analyzed (normally plasma, blood or serum), describes the total exposure of the subject to a drug. The present provides that the exposure of subjects to endoxifen (AUC_{0-inf}) can be dose proportional. In some embodiments, AUC_{0-inf} ranges from 200 hr*ng/mL to 10000 hr*ng/mL. In other embodiments, the AUC_{0-inf} ranges from 300 hr*ng/mL to 8000 hr*ng/mL. In certain embodiments, the AUC_{0-inf} ranges from 400 hr*ng/mL to 6000 hr*ng/mL over the dosing range of 1 mg to 4 mg of (Z)-endoxifen.

A healthcare professional, such as an attending physician, may adjust the dosing regimen based on the pharmacokinetic profile of the composition in the subject.

In one aspect, the compositions of the disclosure can be used alone or in a combination therapy. For example, compositions disclosed herein may be used in combination with one or more therapeutic agents as part of primary therapy, neoadjuvant therapy, or an adjuvant therapy. It is an aspect of the present disclosure that the compositions of the disclosure can be used in combination with other therapies such as surgery and radiation as neo-adjuvant or adjuvant therapy. Combinations of the compositions may act to improve the efficacy of the therapeutic agents, and therefore can be used to improve standard cancer therapies. For example, when a subject has prostate cancer and is on bicalutamide or enzalutamide therapy for the treatment of prostate cancer, the subject is likely to develop gynecomastia a result of the therapy. The compositions disclosed herein can be administered as a combination therapy to the subject having prostate cancer in order to prevent and/or treat gynecomastia. As another example, a subject with ER+/Her2+ positive breast cancer would be on a combination therapy with trastuzumab or other oncology drugs such as anti-neoplastics or immunotherapy, and a composition disclosed herein can be used to treat such a subject with ER+/Her2+ positive breast cancer. Accordingly, in some embodiments, the compositions further comprise bicalutamide, enzalutamide or anticancer drugs such as trastuzumab, antineoplastics such as capecitabine (Xeloda), carboplatin (Paraplatin), cisplatin (Platinol), cyclophosphamide (Neosar), docetaxel (Docefrez, Taxotere), doxorubicin (Adriamycin), pegylated liposomal doxorubicin (Doxil), epirubicin (Ellence), fluorouracil (5-FU, Adrucil), gemcitabine (Gemzar), methotrexate (multiple brand names), paclitaxel (Taxol), protein-bound paclitaxel (Abraxane), vinorelbine (Navelbine), eribulin (Halaven), ixabepilone (Ixempra), and ATP-cassette binding protein inhibitors.

In another aspect, a composition disclosed herein may comprise therapeutic agents that increase bioavailability of endoxifen in a subject. P-glycoprotein (P-gp, ABCB1) is a highly efficient drug efflux pump expressed in brain, liver, and small intestine, but also in cancer cells, that affects pharmacokinetics and confers therapy resistance for many anticancer drugs. Accordingly, in some embodiments, the compositions further comprise inhibitors of ATP-binding cassette (ABC family) transporters, such as inhibitors of breast cancer resistance protein (BCRP protein) and P-gp. Several inhibitors of BCRP protein and P-Gp are known in the art. For example, inhibitors of BCRP protein include cyclosporine, omeprazole, pantoprazole, saquinavir, and tacrolimus.

Non-limiting examples of P-gp inhibitors include first generation inhibitors such as Verapamil, cyclosporin A, reserpine, quinidine, yohimbine, tamoxifen and toremifene, second generation inhibitors such as Dexverapamil, dexniguldipine, valspodar (PSC 833), and Dofequidar fumarate (MS-209), third generation P-gp inhibitors such as Cyclopropyldibenzosuberane zosuquidar (LY335979), laniquidar (R101933), mitotane (NSC-38721), biricodar (VX-710), elacridar (GF120918/GG918), ONT-093, tariquidar (XR9576), and HM30181 and anti-P-gp monoclonal antibodies such as MRK-16).

The present disclosure additionally provides for therapeutic kits containing one or more of the compositions for use in the treatment of a subject having or at risk of having a hormone-dependent breast disorder, a hormone-dependent reproductive tract disorder, or both. The kits of the present disclosure may include an oral composition disclosed herein, a sealed container for housing the composition, and instructions for use of the orally administered composition. In an aspect, the kits of the present disclosure can include a second therapeutic agent. Such a second therapeutic agent may be bicalutamide, enzalutamide or an anticancer drug such as trastuzumab, antineoplasitcs such as capecitabine (Xeloda), carboplatin (Paraplatin), cisplatin (Platinol), cyclophosphamide (Neosar), docetaxel (Docefrez, Taxotere), doxorubicin (Adriamycin), pegylated liposomal doxorubicin (Doxil), epirubicin (Ellence), fluorouracil (5-FU, Adrucil), gemcitabine (Gemzar), methotrexate (multiple brand names), paclitaxel (Taxol), protein-bound paclitaxel (Abraxane), vinorelbine (Navelbine), eribulin (Halaven), ixabepilone (Ixempra), and ATP-binding cassette (ABC transporter) inhibitors such as P-gp inhibitors.

### Oral Formulations

In some embodiments, a pharmaceutical composition of the present disclosure is formulated for oral delivery. Compositions intended for oral use may be prepared in solid or fluid unit dosage forms. For example, a compound of Formula (IV) comprising (Z)-endoxifen prepared and purified as described herein may be formulated for oral delivery. In at least some embodiments, the compositions are formulated for oral delivery as tablets, caplets, capsules, pills, powders, troches, elixirs, suspensions, syrups, wafers, chewing gums, dragees, lozenges, and the like.

In some embodiments, the oral dosage forms are solid oral dosage forms such as tablets, caplets, and capsules. In some embodiments, the capsule is a hard capsule or a soft capsule. In other embodiments, the capsule is a gelatin capsule, gelatin-free capsule, a “cap-in-cap” capsule, alginate capsule, hydroxypropylmethyl cellulose (HPMC) capsule, a polyvinyl alcohol (PVA) capsule, a Hypromellose capsule, or a starch capsule. An example of an oral dosage form of (Z)-endoxifen may comprise a compound of Formula (IV) encapsulated in an enteric resistant delayed release Hypromellose capsule.

Exemplary, non-limiting compositions are provided below. As mentioned above, percentages (%) refer to amounts by weight based upon the total weight of the composition (wt/wt). The sum of the different components of the composition adds up to 100% (wt/wt) of the total composition. The at least 90% (≥90%) (Z)-endoxifen free base refers to the percent weight of (Z)-endoxifen isomer as compared to the total weight of endoxifen in any composition.

In some embodiments, a compound of Formula (IV) with a purity of at least 90%, 91%, 92%, 93%, 94%, 95%, 96%, 97%, 98%, or 99%, as described herein, may be prepared as an oral formulation. The compound of Formula (IV) may be prepared as described herein, for example in SCHEME 1 and EXAMPLE 1-EXAMPLE 3.

In an aspect, the present disclosure relates to compositions comprising (Z)-endoxifen free base or a salt thereof prepared according to any of the methods disclosed herein.

In another aspect, the present disclosure relates to compositions comprising (Z)-endoxifen free base or a salt thereof prepared according to any of the methods disclosed herein, the (Z)-endoxifen is at least 90% (Z)-endoxifen free base wt/wt of total endoxifen in the composition.

In still another aspect, the compositions further comprise (E)-endoxifen, wherein the ratio of (E)-endoxifen to (Z)-endoxifen (E/Z-ratio) is 1:99; 5:95; 10:90, 15:85; 20:80, 25:75; 30:70; 40:70, 45:55; 50:50; 55:45; 60:40; 65:45; or 70:30.

In a further aspect, the compositions further comprise (E)-endoxifen, wherein the E/Z-ratio ranges from 45:55 to 55:45 or is approximately 1:1.

In an aspect, the compositions comprise (Z)-endoxifen or a salt or polymorph thereof prepared according to any of the methods disclosed herein, wherein the (Z)-endoxifen is stable at ambient temperature for at least 6 months, at least 9 months, at least 12 months or at least 18 months.

In another aspect, the compositions comprise (Z)-endoxifen or salts thereof prepared according to any of the methods disclosed herein: wherein the compositions have aerobic bacterial plate count of not more than 20,000 g/mL; wherein water content of the compositions is not more than 1.0% as tested by Method Ic of the USP 921; wherein the water activity (Aw) of the compositions is less than 0.9; wherein the residue on ignition is not more than 0.1% as tested by a method of USP 281; wherein a heavy metal is not more than 20 ppm as tested by Method II of USP 231; and/or wherein methanol is NMT 3000 ppm, ethanol is NMT 5000 ppm, tetrahydrofuran is NMT 720 ppm, acetone is NMT 5000 ppm, isopropanol is NMT 5000 ppm, ethyl acetate is NMT 5000 ppm; n-Heptane is NMT 5000 ppm, acetonitrile is NMT 410 ppm, MeTHF is NMT 520 ppm, zinc is NMT 130 ppm, benzene is NMT 2 ppm, and mesityl oxide is NMT 25 ppm, as tested by a validated HPLC method.

In still another aspect, the compositions comprising (Z)-endoxifen or salts thereof prepared according to any of the methods disclosed herein can comprise 0.01 mg to 200 mg (Z)-endoxifen or a salt thereof per unit dose. In yet another aspect, the compositions comprising (Z)-endoxifen or salts thereof prepared according to any of the methods disclosed herein comprise 1 mg to 80 mg of (Z)-endoxifen or a salt thereof per unit dose. In a further aspect, the compositions comprising (Z)-endoxifen or salts thereof prepared according to any of the methods disclosed herein comprise 0.01% to 20% (wt/wt) of endoxifen or a salt thereof.

In another aspect, the present disclosure relates to a composition formulated for oral administration comprising: 1 mg to 200 mg of endoxifen per unit dose; and wherein the composition is stable for at least 6 months. In some aspects, the composition formulated for oral administration comprises from 1 mg to 80 mg of endoxifen per unit dose, from 2 mg to 80 mg per unit dose, from 4 mg to 80 mg per unit dose, or from 4 mg to 40 mg per unit dose. In some aspects, the composition formulated for oral administration comprises about 1 mg, about 2 mg, about 4 mg, about 6 mg, about 8 mg, about 10 mg, about 20 mg, about 40 mg, or about 80 mg per unit dose.

In a still further aspect, the present disclosure relates to compositions comprising (Z)-endoxifen or salts thereof prepared according to any of the methods disclosed herein, wherein the composition comprises 0.1% to 10% (wt/wt) of (Z)-endoxifen or a salt thereof. In some cases, the (Z)-endoxifen or salt thereof is solid. Solid (Z)-endoxifen of a composition disclosed herein can be amorphous, polycrystalline, or of a single crystalline form.

In another aspect, the present disclosure relates to compositions comprising (Z)-endoxifen or salts thereof prepared according to any of the methods disclosed herein further comprising one or more excipient.

In yet another aspect, the present disclosure relates to compositions comprising (Z)-endoxifen or salts thereof prepared according to any of the methods disclosed herein, further comprising one or more excipient, wherein the excipient is a binder, a filler, a disintegrating agent, a lubricant, a glidant, a control release agent, an enteric coating agent, a film forming agent, a plasticizer, a sweetening agent, a flavoring agent, or a combination thereof.

In still another aspect, the present disclosure relates to compositions comprising (Z)-endoxifen or salts thereof prepared according to any of the methods disclosed herein, further comprising one or more excipient, wherein the excipients are about 0.1% to about 99% wt/wt of the composition.

In a further aspect, the present disclosure relates to a composition comprising (Z)-endoxifen or salts thereof prepared according to any of the methods disclosed herein, wherein the composition comprises one or more control release agents selected from the group consisting of acid-insoluble polymers, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, waxes, including synthetic waxes, microcrystalline waxes, paraffin wax, carnauba wax, and beeswax, polyethoxylated castor oil derivatives, hydrogenated oils, glyceryl mono-, di- tribenates, glyceryl monostearate, glyceryl distearate, long chain alcohols, such as stearyl alcohol, cetyl alcohol, polyethylene glycol, and mixtures thereof.

In one aspect, the present disclosure relates to a composition comprising (Z)-endoxifen or salts thereof prepared according to any of the methods disclosed herein, wherein the composition is formulated for oral, parenteral, topical, and intraductal delivery.

In another aspect, the present disclosure relates to a composition comprising (Z)-endoxifen or salts thereof prepared according to any of the methods disclosed herein, wherein the composition formulated for oral delivery is a tablet, a caplet, a capsule, a pill, a powder, a troche, an elixir, a suspension, a syrup, a wafer, a chewing gum, a dragee, and a lozenge.

In yet another aspect, the present disclosure relates to compositions comprising (Z)-endoxifen or salts thereof prepared according to any of the methods disclosed herein, wherein the compositions formulated for oral delivery are tablets formulated as enteric tablets, caplets formulated as enteric caplets, and capsule formulated as enteric capsules. For example, the composition comprising (Z)-endoxifen may be formulated for oral delivery in an enteric capsule comprising Hypromellose (e.g., CAPSUGEL® DRCAPS™). In some embodiments, the composition may be formulated as an enteric resistant delayed release Hypromellose capsule.

In yet another aspect, the present disclosure relates to compositions comprising (Z)-endoxifen or salts thereof prepared according to any of the methods disclosed herein, wherein the compositions formulated for oral delivery are tablets formulated as delayed-release tablets, caplets formulated as delayed-release caplets, or capsules formulated as delayed-release capsules.

In yet another aspect, the present disclosure relates to a composition comprising (Z)-endoxifen or salts thereof prepared according to any of the methods disclosed herein, wherein the composition is formulated for oral delivery, and wherein as tested by a dissolution method, the composition releases in stomach: less than 10% of endoxifen in 2 hours after administration; less than 20% of endoxifen in 2 hours after administration; or less than 30% of endoxifen in 2 hours after administration.

In still another aspect, the present disclosure relates to a composition comprising (Z)-endoxifen or salts thereof prepared according to any of the methods disclosed herein, wherein the composition is formulated for oral delivery, and wherein as tested by a dissolution method, the composition releases in stomach: less than 10% of (Z) endoxifen in 2 hours after administration; or less than 20% of (Z) endoxifen in 2 hours after administration. In some aspects, a composition comprising (Z)-endoxifen or salts thereof prepared according to any of the methods disclosed herein, wherein the composition is formulated for oral delivery, releases less than 20% of (Z)-endoxifen in the stomach in 2 hours after administration.

In another aspect, the present disclosure relates to a composition comprising (Z)-endoxifen or salts thereof prepared according to any of the methods disclosed herein, wherein the composition is formulated for oral delivery, and wherein as tested by a dissolution method, the composition releases at least 20%, at least 40%, at least 50%, at least 60%, at least 70%, or at least 80% of endoxifen in the intestines.

In another aspect, the present disclosure relates to a composition comprising (Z)-endoxifen or salts thereof prepared according to any of the methods disclosed herein, wherein the composition is formulated for oral delivery, and wherein as tested by a dissolution method, the composition releases in the intestines at least 40%, at least 50%, at least 60%, at least 70%, at least 80%, or at least 90% endoxifen in 3.5 hours after administration.

In another aspect, the present disclosure relates to a composition comprising (Z)-endoxifen or salts thereof prepared according to any of the methods disclosed herein, wherein the composition is formulated for oral delivery, and wherein as tested by a dissolution method, the composition releases in the intestines at least 20%, at least 40%, at least 50%, at least 60%, at least 70%, or at least 80% of endoxifen in 3 hours after administration.

In another aspect, the present disclosure relates to a composition comprising (Z)-endoxifen or salts thereof prepared according to any of the methods disclosed herein, wherein the composition is formulated for oral delivery, and wherein as tested by a dissolution method, the composition releases in the intestines at least 20%, at least 40%, at least 50%, at least 60%, at least 70%, or at least 80% of endoxifen in 4 hours after administration. In some aspects, a composition comprising (Z)-endoxifen or salts thereof prepared according to any of the methods disclosed herein, wherein the composition is formulated for oral delivery, releases at least 80% of (Z)-endoxifen in the intestines in 2 hours, 3 hours, 3.5 hours, 4 hours, or 8 hours after administration.

In another aspect, the present disclosure relates to a composition comprising (Z)-endoxifen or salts thereof prepared according to any of the methods disclosed herein, wherein the composition is formulated for oral delivery, and wherein as tested by a dissolution method, the composition releases in small intestine: at least 10% of endoxifen after 4 hours after administration; at least 30% of endoxifen after 6 hours after administration; at least 40% of endoxifen after 7 hours after administration; at least 50% of endoxifen after 8 hours after administration; at least 50% of endoxifen after 2 hours after administration; at least 60% of endoxifen after 2 hours after administration; at least 70% of endoxifen after 2 hours after administration; at least 80% of endoxifen after 2 hours after administration; or at least 90% of endoxifen after 2 hours after administration.

In another aspect, the present disclosure relates to a composition comprising (Z)-endoxifen or salts thereof prepared according to any of the methods disclosed herein, wherein the composition is formulated for oral delivery, and wherein as tested by a dissolution method, the composition is formulated to release at least 80% of endoxifen after 3.5 hours after administration in the colon.

In another aspect, the present disclosure relates to a composition formulated for oral administration comprising: 1 mg to 200 mg of endoxifen per unit dose; wherein the endoxifen is at least 90% (Z)-endoxifen free base; and wherein: the composition has an aerobic bacterial plate count of not more than 20,000 g/ml; water content of the (Z)-endoxifen is not more than 1% as determined by Method Ic of the USP 921; residue on ignition of the (Z)-endoxifen is not more than 0.10% as tested by a method of USP 281; or a heavy metal is not more than 20 ppm as tested by Method II of USP 231.

In another aspect, the present disclosure relates to a composition formulated for oral administration comprising: 1 mg to 200 mg of endoxifen per unit dose, wherein the composition is formulated as an enteric tablet, an enteric caplet, and an enteric capsule; wherein as determined by a dissolution method, the composition is formulated to release in small intestine: at least 25% of endoxifen after 4 hours after administration; at least 30% of endoxifen after 6 hours after administration; at least 40% of endoxifen after 7 hours after administration; or at least 50% of endoxifen after 8 hours after administration; or wherein as determined by a dissolution method, the composition is formulated to release at least 50% of endoxifen in the colon.

In another aspect, the present disclosure relates to an oral solid dosage form composition comprising: 1 mg to 200 mg of endoxifen per unit dose; wherein the endoxifen is at least 90% (Z)-endoxifen free base; and wherein the composition has an aerobic bacterial plate count of not more than 20,000 g/ml; water content of the (Z)-endoxifen is not more than 1% as determined by Method Ic of the USP 921; residue on ignition of the (Z)-endoxifen is not more than 0.10% as tested by a method of USP 281; or a heavy metal is not more than 20 ppm as tested by Method II of USP 231; and wherein the composition is formulated as an enteric tablet, an enteric caplet and an enteric capsule; and wherein the endoxifen is neat; or wherein the composition further comprises an excipient selected from the group consisting of a binder, a filler, a disintegrating agent, a lubricant, a glidant, a control release agent, an enteric coating agent, a film forming agent, a plasticizer, a colorant, a sweetening agent, and a flavoring agent, or a combination thereof.

In another aspect, the present disclosure relates to an enteric capsule, comprising (Z)-endoxifen free base prepared by a process described herein.

In another aspect, the present disclosure relates to an enteric capsule comprising 1 mg to 200 mg of neat endoxifen per unit dose: wherein the endoxifen is at least 90% (Z)-endoxifen free base; and wherein the composition has an aerobic bacterial plate count of not more than 20,000 g/ml; water content of the (Z)-endoxifen is not more than 1% as determined by Method Ic of the USP 921; residue on ignition of the (Z)-endoxifen is not more than 0.10% as tested by a method of USP 281; and a heavy metal is not more than 20 ppm as tested by Method II of USP 231.

In another aspect, the present disclosure relates to a composition comprising (Z)-endoxifen or a salt or polymorph thereof prepared by any of the processes described herein, wherein the endoxifen salt is selected from the group consisting of arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, Teoclate, triethiodide, benzathine, clemizole, chloroprocaine, choline, diethylamine, diethanolamine, ethylenediamine, meglumine, piperazine, procaine, aluminum, barium, bismuth, lithium, magnesium, potassium, and zinc.

In still another aspect, the present disclosure relates to a composition comprising (Z)-endoxifen or a salt or polymorph thereof prepared by any of the processes described herein for the treatment and prevention of a hormone-dependent breast disorder, a hormone-dependent reproductive tract disorder, or both.

In still another aspect, the present disclosure relates to a composition comprising (Z)-endoxifen or a salt or polymorph thereof prepared by any of the processes described herein for the treatment and prevention of a hormone-dependent breast disorder, a hormone-dependent reproductive tract disorder, or both, wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer.

In still another aspect, the present disclosure relates to a composition comprising (Z)-endoxifen or a salt or polymorph thereof prepared by any of the processes described herein for the treatment and prevention of a hormone-dependent breast disorder, a hormone-dependent reproductive tract disorder, or both, wherein the hormone-dependent breast disorder is breast cancer.

In still another aspect, the present disclosure relates to a composition comprising (Z)-endoxifen or a salt or polymorph thereof prepared by any of the processes described herein for the treatment and prevention of a hormone-dependent breast disorder, a hormone-dependent reproductive tract disorder, or both, wherein the hormone-dependent breast disorder is breast cancer and wherein the breast cancer is a pre-cancer, an early stage cancer, a non-metastatic cancer, a pre-metastatic cancer, a locally advanced cancer, or a metastatic cancer.

In still another aspect, the present disclosure relates to a composition comprising (Z)-endoxifen or a salt or polymorph thereof prepared by any of the processes described herein for the treatment and prevention of a hormone-dependent breast disorder, a hormone-dependent reproductive tract disorder, or both, wherein the hormone-dependent breast disorder or the hormone-dependent reproductive tract disorder is tamoxifen-refractory or tamoxifen resistant.

In still another aspect, the present disclosure relates to a composition comprising (Z)-endoxifen or a salt or polymorph thereof prepared by any of the processes described herein, wherein the composition is administered to a subject in need thereof.

In still another aspect, the present disclosure relates to a composition comprising (Z)-endoxifen or a salt or polymorph thereof prepared by any of the processes described herein, wherein the composition is administered to a subject in need thereof and wherein the subject has or is at risk of having a hormone-dependent breast disorder, a hormone-dependent reproductive tract disorder, or both.

In still another aspect, the present disclosure relates to a composition comprising (Z)-endoxifen or a salt or polymorph thereof prepared by any of the processes described herein, wherein the composition is administered to a subject in need thereof, wherein the subject's hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer.

In still another aspect, the present disclosure relates to a composition comprising (Z)-endoxifen or a salt or polymorph thereof prepared by any of the processes described herein, wherein the composition is administered to a subject in need thereof, wherein the subject has breast cancer and wherein breast cancer is a pre-cancer, an early stage cancer, a non-metastatic cancer, a pre-metastatic cancer, a locally advanced cancer, or a metastatic cancer.

In still another aspect, the present disclosure relates to a composition comprising (Z)-endoxifen or a salt or polymorph thereof prepared by any of the processes described herein, wherein the composition is administered to a subject in need thereof, wherein the subject has prostate cancer and wherein the subject has or is about to initiate prostate cancer therapy.

In still another aspect, the present disclosure relates to a composition comprising (Z)-endoxifen or a salt or polymorph thereof prepared by any of the processes described herein, wherein the composition is administered to a subject in need thereof, wherein the subject has prostate cancer, and wherein the subject has or is at risk of having gynecomastia.

In still another aspect, the present disclosure relates to a composition comprising (Z)-endoxifen or a salt or polymorph thereof prepared by any of the processes described herein, wherein the composition is administered to a subject that has or is at risk of having a hormone-dependent breast disorder, a hormone-dependent reproductive tract disorder, or both, and wherein the subject's hormone-dependent breast disorder or the hormone-dependent reproductive tract disorder is tamoxifen-refractory or tamoxifen resistant.

In still another aspect, the present disclosure relates to a composition comprising (Z)-endoxifen or a salt or polymorph thereof prepared by any of the processes described herein, wherein the composition is administered to the subject at a unit dose of 1.0 mg, 1.5 mg, 2.0 mg, 4 mg, 5 mg, 10 mg, 20 mg, 25 mg, 40 mg, 50 mg, 80 mg, 120 mg, 160 mg, or 200 mg of (Z)-endoxifen.

In still another aspect, the present disclosure relates to a composition comprising (Z)-endoxifen or a salt or polymorph thereof prepared by any of the processes described herein, wherein the composition is administered to the subject at a dose of 1.0 mg, 1.5 mg, 2.0 mg, 4 mg, 5 mg, 10 mg, 20 mg, 25 mg, 40 mg, 50 mg, 80 mg, 120 mg, 160 mg, or 200 mg of (Z)-endoxifen.

In still another aspect, the present disclosure relates to a composition comprising (Z)-endoxifen or a salt or polymorph thereof prepared by any of the processes described herein, wherein the composition is administered to the subject once a day, twice a day, thrice a day, four times a day, every other day, twice a week, weekly, fortnightly, twice a month, monthly, quarterly, once every six months, or annually.

In still another aspect, the present disclosure relates to a composition comprising (Z)-endoxifen or a salt or polymorph thereof prepared by any of the processes described herein, wherein administration of the composition orally maintains the subject's plasma endoxifen at a level greater than 30 nM.

In still another aspect, the present disclosure relates to a composition comprising (Z)-endoxifen or a salt or polymorph thereof prepared by any of the processes described herein, wherein the composition is administered to a subject as combination therapy.

In still another aspect, the present disclosure relates to a composition comprising (Z)-endoxifen or a salt or polymorph thereof prepared by any of the processes described herein, wherein the composition is administered as a primary therapy, a neo-adjuvant therapy, or an adjuvant therapy.

In still another aspect, the present disclosure relates to a composition comprising (Z)-endoxifen or a salt or polymorph thereof prepared by any of the processes described herein, wherein the composition is administered to a subject either alone in combination with a second therapeutic agent.

In still another aspect, the present disclosure relates to a composition comprising (Z)-endoxifen or a salt or polymorph thereof prepared by any of the processes described herein, wherein the composition is administered to a subject either alone in combination with a second therapeutic agent, and wherein the second therapeutic agent is bicalutamide, enzalutamide, or an anticancer drug such as trastuzumab, antineoplastic such as capecitabine (Xeloda), carboplatin (Paraplatin), cisplatin (Platinol), cyclophosphamide (Neosar), docetaxel (Docefrez, Taxotere), doxorubicin (Adriamycin), PEGylated liposomal doxorubicin (Doxil), epirubicin (Ellence), fluorouracil (5-FU, Adrucil), gemcitabine (Gemzar), methotrexate (multiple brand names), paclitaxel (Taxol), protein-bound paclitaxel (Abraxane), vinorelbine (Navelbine), eribulin (Halaven), ixabepilone (Ixempra), or inhibitors of ATP-binding cassette transporters.

In still another aspect, the present disclosure relates to a method of treating a subject in need thereof comprising administering a composition prepared according to any one of the processes disclosed herein.

In still another aspect, the present disclosure relates to a method of treating a subject in need thereof comprising administering a composition comprising (Z)-endoxifen or a salt or polymorph thereof prepared according to any one of the processes disclosed herein.

In still another aspect, the present disclosure relates to a method of treating a subject in need thereof comprising administering a composition comprising (Z)-endoxifen or a salt or polymorph thereof prepared according to any one of the processes disclosed herein, wherein the subject has or is at risk of having a hormone-dependent breast disorder, a hormone-dependent reproductive tract disorder, or both.

In still another aspect, the present disclosure relates to a method of treating a subject in need thereof comprising administering a composition comprising (Z)-endoxifen or a salt or polymorph thereof prepared according to any one of the processes disclosed herein, wherein the subject has or is at risk of having a hormone-dependent breast disorder, a hormone-dependent reproductive tract disorder, or both, wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer.

In still another aspect, the present disclosure relates to a method of treating a subject in need thereof comprising administering a composition comprising (Z)-endoxifen or a salt or polymorph thereof prepared according to any one of the processes disclosed herein, wherein the subject has breast cancer.

In still another aspect, the present disclosure relates to a method of treating a subject in need thereof comprising administering a composition comprising (Z)-endoxifen or a salt or polymorph thereof prepared according to any one of the processes disclosed herein, wherein the subject has breast cancer, and wherein the breast cancer is a pre-cancer, an early stage cancer, a non-metastatic cancer, a pre-metastatic cancer, a locally advanced cancer, or a metastatic cancer.

In still another aspect, the present disclosure relates to a method of treating a subject in need thereof comprising administering a composition comprising (Z)-endoxifen or a salt or polymorph thereof prepared according to any one of the processes disclosed herein, wherein the subject has prostate cancer and wherein the subject further has or is at risk of having gynecomastia.

In still another aspect, the present disclosure relates to a method of treating a subject in need thereof comprising administering a composition comprising (Z)-endoxifen or a salt or polymorph thereof prepared according to any one of the processes disclosed herein, wherein the subject has tamoxifen-refractory or tamoxifen resistant hormone-dependent breast disorder or hormone-dependent reproductive tract disorder.

In still another aspect, the present disclosure relates to a method of treating a subject in need thereof comprising administering a composition comprising (Z)-endoxifen or a salt or polymorph thereof prepared according to any one of the processes disclosed herein, wherein the composition is administered to the subject at a unit dose of 1.0 mg, 1.5 mg, 2.0 mg, 4 mg, 5 mg, 6 mg, 8 mg, 10 mg, 20 mg, 25 mg, 40 mg, 50 mg, 80 mg, 100 mg, 120 mg, 160 mg, or 200 mg.

In still another aspect, the present disclosure relates to a method of treating a subject in need thereof comprising administering a composition comprising (Z)-endoxifen or a salt or polymorph thereof prepared according to any one of the processes disclosed herein, wherein the composition is administered once a day, twice, a day, thrice a day, four times a day, every other day, twice a week, weekly, fortnightly, twice a month, monthly, quarterly, once every six months, or annually.

In still another aspect, the present disclosure relates to a method of treating a subject in need thereof comprising administering a composition comprising (Z)-endoxifen or a salt or polymorph thereof prepared according to any one of the processes disclosed herein, wherein administration of the composition orally maintains the subject's plasma endoxifen at a steady state level greater than 30 nM.

In still another aspect, the present disclosure relates to a method of treating a subject in need thereof comprising administering a composition comprising (Z)-endoxifen or a salt or polymorph thereof prepared according to any one of the processes disclosed herein, wherein administration of the composition orally achieves the subject's plasma endoxifen at a steady state level greater than 30 nM by day 14 after the administration of the first dose (on day 1).

In still another aspect, the present disclosure relates to a method of treating a subject in need thereof comprising administering a composition comprising (Z)-endoxifen or a salt or polymorph thereof, wherein administration of the composition orally achieves the subject's plasma endoxifen at a steady state level greater than 30 nM by day 14 after the administration of the first dose (on day 1).

In still another aspect, the present disclosure relates to a method of treating a subject in need thereof comprising administering a composition comprising (Z)-endoxifen or a salt or polymorph thereof prepared according to any one of the processes disclosed herein, wherein administration of the composition orally achieves a time to maximal plasma levels of endoxifen in the subject from 2 to 10 hours after administration (post-dose).

In still another aspect, the present disclosure relates to a method of treating a subject in need thereof comprising administering a composition comprising (Z)-endoxifen or a salt or polymorph thereof, wherein administration of the composition orally achieves a time to maximal plasma levels of endoxifen in the subject from 2 to 10 hours after administration (post-dose).

In still another aspect, the present disclosure relates to a method of treating a subject in need thereof comprising administering a composition comprising (Z)-endoxifen or a salt or polymorph thereof, wherein the mean terminal elimination half-life of endoxifen in the subject ranges from 30 to 60 hours post dose.

In still another aspect, the present disclosure relates to a method of treating a subject in need thereof comprising administering a composition comprising (Z)-endoxifen or a salt or polymorph thereof, wherein the mean terminal elimination half-life of endoxifen in the subject ranges from 40 to 55 hours post dose.

In another aspect, the present disclosure relates to a method of treating a subject having or at risk of having a hormone-dependent breast disorder, a hormone-dependent reproductive tract disorder, or both, the method comprising administering an oral composition comprising (Z)-endoxifen or a salt or polymorph thereof, wherein administration of the composition achieves: a mean half-life of endoxifen in the subject ranging from 30 hours to 60 hours after administration; a time to maximum plasma levels of endoxifen ranging from 2 hours to 10 hours after administration; and a steady state plasma level of endoxifen greater than 30 nM.

In another aspect, the present disclosure relates to a method of treating a subject having or at risk of having a hormone-dependent breast disorder or a hormone-dependent reproductive tract disorder selected from the group consisting of benign breast disorders, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, and vulvar cancer, the method comprising administering an oral composition comprising (Z)-endoxifen or a salt or polymorph thereof, wherein administration of the composition achieves: a mean half-life of endoxifen in the subject ranging from 30 hours to 60 hours after administration; a time to maximum plasma levels of endoxifen ranging from 2 hours to 10 hours after administration; and a steady state plasma level of endoxifen greater than 30 nM.

In another aspect, the present disclosure relates to a method of treating a subject having or at risk of having a hormone-dependent breast disorder or a hormone-dependent reproductive tract disorder selected from the group consisting of benign breast disorders, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer and vulvar cancer, the method comprising administering an oral composition comprising (Z)-endoxifen or a salt or polymorph thereof formulated as an enteric tablet, an enteric caplet, an enteric capsule, a delayed-release tablet, a delayed-release caplet, or a delayed-release capsule, wherein administration of the composition achieves: a mean half-life of endoxifen in the subject ranging from 30 hours to 60 hours after administration; a time to maximum plasma levels of endoxifen ranging from 2 hours to 10 hours after administration; and a steady state plasma level of endoxifen greater than 30 nM.

In another aspect, the present disclosure relates to a method of treating a subject having or at risk of having a hormone-dependent breast disorder or a hormone-dependent reproductive tract disorder selected from the group consisting of benign breast disorders, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, and vulvar cancer, the method comprising administering an oral composition comprising (Z)-endoxifen or a salt or polymorph thereof formulated as an enteric tablet, an enteric caplet, an enteric capsule, a delayed-release tablet, a delayed-release caplet, or a delayed-release capsule, wherein administration of the composition achieves: a mean half-life of endoxifen in the subject ranging from 30 hours to 60 hours after administration; a time to maximum plasma levels of endoxifen ranging from 2 hours to 10 hours after administration; and a steady state plasma level of endoxifen greater than 30 nM; and wherein at least 50%, at least 60%, at least 70%, at least 80% or at least 90% of the endoxifen is released in the intestines.

In another aspect, the present disclosure relates to a method of treating a subject having or at risk of having a hormone-dependent breast disorder or a hormone-dependent reproductive tract disorder selected from the group consisting of benign breast disorders, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, and vulvar cancer, the method comprising administering an oral composition comprising 0.01 mg to 200 mg of (Z)-endoxifen or a salt or polymorph thereof formulated as an enteric tablet, an enteric caplet, an enteric capsule, a delayed-release tablet, a delayed-release caplet, or a delayed-release capsule, wherein administration of the composition achieves: a mean half-life of endoxifen in the subject ranging from 30 hours to 60 hours after administration; a time to maximum plasma levels of endoxifen ranging from 2 hours to 10 hours after administration; and a steady state plasma level of endoxifen greater than 30 nM; and wherein at least 50%, at least 60%, at least 70%, at least 80% or at least 90% of the endoxifen is released in the intestines.

In another aspect, the present disclosure relates to a method of treating a subject having or at risk of having a hormone-dependent breast disorder or a hormone-dependent reproductive tract disorder selected from the group consisting of benign breast disorders, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, and vulvar cancer, the method comprising administering daily an oral composition comprising 0.01 mg to 200 mg of (Z)-endoxifen or a salt or polymorph thereof formulated as an enteric tablet, an enteric caplet, an enteric capsule, a delayed-release tablet, a delayed-release caplet, or a delayed-release capsule, wherein administration of the composition achieves: a mean half-life of endoxifen in the subject ranging from 30 hours to 60 hours after administration; a time to maximum plasma levels of endoxifen ranging from 2 hours to 10 hours after administration; and a steady state plasma level of endoxifen greater than 30 nM.

In another aspect, the present disclosure relates to a method of treating a subject having or at risk of having a hormone-dependent breast disorder or a hormone-dependent reproductive tract disorder selected from the group consisting of benign breast disorders, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, and vulvar cancer, the method comprising administering daily an oral composition comprising 0.01 mg to 200 mg of (Z)-endoxifen or a salt or polymorph thereof formulated as an enteric tablet, an enteric caplet, an enteric capsule, a delayed-release tablet, a delayed-release caplet, or a delayed-release capsule, wherein administration of the composition achieves: a mean half-life of endoxifen in the subject ranging from 30 hours to 60 hours after administration; a time to maximum plasma levels of endoxifen ranging from 2 hours to 10 hours after administration; and a steady state plasma level of endoxifen greater than 30 nM; and wherein at least 50%, at least 60%, at least 70%, at least 80% or at least 90% of the endoxifen is released in the intestines.

In another aspect, the present disclosure relates to a method of treating a subject having or at risk of having a hormone-dependent breast disorder or a hormone-dependent reproductive tract disorder selected from the group consisting of benign breast disorders, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, and vulvar cancer, the method comprising administering daily an oral composition comprising 0.01 mg to 200 mg of (Z)-endoxifen or a salt or polymorph thereof formulated as an enteric tablet, an enteric caplet, an enteric capsule, a delayed-release tablet, a delayed-release caplet, or a delayed-release capsule, wherein administration of the composition achieves: a mean half-life of endoxifen in the subject ranging from 30 hours to 60 hours after administration; a time to maximum plasma levels of endoxifen ranging from 2 hours to 10 hours after administration; and a steady state plasma level of endoxifen greater than 30 nM; wherein the mean area under the curve extrapolated to time infinity (AUC0-inf) is 200 hr*ng/mL to 10000 hr*ng/mL, 300 hr*ng/mL to 8000 hr*ng/mL, 400 hr*ng/mL to 6000 hr*ng/mL or 700 hr*ng/mL to 6000 hr*ng/mL; and wherein at least 50%, at least 60%, at least 70%, at least 80% or at least 90% of the endoxifen is released in the intestines.

In still another aspect, the present disclosure relates to a method of treating a subject in need thereof comprising administering a composition comprising (Z)-endoxifen or a salt or polymorph thereof prepared according to any one of the processes disclosed herein, wherein the method is a primary therapy, a neo-adjuvant therapy, or an adjuvant therapy.

In still another aspect, the present disclosure relates to a method of treating a subject in need thereof comprising administering a composition comprising (Z)-endoxifen or a salt or polymorph thereof prepared according to any one of the processes disclosed herein, wherein a composition is administered to a subject as combination therapy.

In still another aspect, the present disclosure relates to a method of treating a subject in need thereof comprising administering a composition comprising (Z)-endoxifen or a salt or polymorph thereof prepared according to any one of the processes disclosed herein, wherein a composition is administered to a subject either alone in combination with a second therapeutic agent.

In still another aspect, the present disclosure relates to a method of treating a subject in need thereof comprising administering a composition comprising (Z)-endoxifen or a salt or polymorph thereof prepared according to any one of the processes disclosed herein, wherein a composition is administered to a subject with a second therapeutic agent selected from the group consisting of bicalutamide, enzalutamide, or an anticancer drug such as trastuzumab, antineoplastics such as capecitabine (Xeloda), carboplatin (Paraplatin), cisplatin (Platinol), cyclophosphamide (Neosar), docetaxel (Docefrez, Taxotere), doxorubicin (Adriamycin), PEGylated liposomal doxorubicin (Doxil), epirubicin (Ellence), fluorouracil (5-FU, Adrucil), gemcitabine (Gemzar), methotrexate (multiple brand names), paclitaxel (Taxol), protein-bound paclitaxel (Abraxane), vinorelbine (Navelbine), eribulin (Halaven), ixabepilone (Ixempra), and ATP-cassette binding protein transport inhibitors.

In still another aspect, the present disclosure relates to a use for the treatment and prevention of a hormone-dependent breast disorder, a hormone-dependent reproductive tract disorder, or both, in a subject, the use comprising: dosing the subject with a first composition comprising tamoxifen; determining the subject's tamoxifen-metabolites profile in a test sample obtained from the subject; determining a reduced level of subject's plasma endoxifen based on subject's tamoxifen-metabolites profile as to compared to a level of plasma endoxifen in a reference tamoxifen-metabolites profile; and administering to the subject a composition of the present disclosure.

In still another aspect, the present disclosure relates to a use for the treatment and prevention of a hormone-dependent breast disorder, a hormone-dependent reproductive tract disorder, or both, in a subject, the use comprising: dosing the subject with a first composition comprising tamoxifen; determining the subject's tamoxifen-metabolites profile in a test sample obtained from the subject; determining a reduced level of subject's plasma endoxifen based on subject's tamoxifen-metabolites profile as to compared to a level of plasma endoxifen in a reference tamoxifen-metabolites profile; and administering to the subject a composition of the present disclosure; wherein the composition is administered orally in a dose sufficient to maintain the subject's plasma endoxifen at a steady state level greater than 30 nM; wherein the tamoxifen-metabolites profile comprises a panel of at least tamoxifen, 4-hydroxytamoxifen, n-desmethyltamoxifen, or endoxifen; wherein the hormone-dependent breast disorder or the hormone-dependent reproductive tract disorder is a benign breast disorder, hyperplasia, atypia, atypical ductal hyperplasia, atypical lobular hyperplasia, increased breast density, gynecomastia, DCIS, LCIS, breast cancer, precocious puberty, McCune-Albright Syndrome, endometrial cancer, ovarian cancer, uterine cancer, cervical cancer, vaginal cancer, or vulvar cancer; wherein the hormone-dependent breast disorder is a pre-cancer, an early stage cancer, a non-metastatic cancer, a pre-metastatic cancer, a locally advanced cancer, or metastatic cancer; wherein the hormone-dependent breast disorder is a non-metastatic cancer; wherein the subject has prostate cancer, and wherein the subject further has or is at risk of having gynecomastia; or wherein the hormone-dependent breast disorder or hormone-dependent reproductive tract disorder is tamoxifen-refractory or tamoxifen resistant.

In still another aspect, the present disclosure relates to a kit for treating a subject having or at risk of having a hormone-dependent breast disorder or hormone dependent reproductive tract disorder, in a subject in need thereof comprising: (a) a composition of the present disclosure; and (b) a sealed container for housing the composition; and c) instructions for use of the orally administered composition.

In still another aspect, the present disclosure relates to a kit for treating a subject having or at risk of having a hormone-dependent breast disorder or hormone dependent reproductive tract disorder, in a subject in need thereof comprising: (a) a composition of the present disclosure; and (b) a sealed container for housing the composition; and c) instructions for use of the orally administered composition, wherein the kit comprises a second therapeutic agent selected from the group consisting of bicalutamide, enzalutamide and anticancer drugs such as trastuzumab, antineoplastics such as capecitabine (Xeloda), carboplatin (Paraplatin), cisplatin (Platinol), cyclophosphamide (Neosar), docetaxel (Docefrez, Taxotere), doxorubicin (Adriamycin), PEGylated liposomal doxorubicin (Doxil), epirubicin (Ellence), fluorouracil (5-FU, Adrucil), gemcitabine (Gemzar), methotrexate (multiple brand names), paclitaxel (Taxol), protein-bound paclitaxel (Abraxane), vinorelbine (Navelbine), eribulin (Halaven), ixabepilone (Ixempra), and ATP-cassette binding protein transport inhibitors.

In still another aspect, the present disclosure relates to a method of administering a composition prepared in accordance with any of the processes described herein to a subject in need thereof in accordance with instructions for use comprised in a kit comprising the composition.

### Excipients

In some embodiments, an oral composition comprising (Z)-endoxifen or a salt thereof further comprises one or more excipients. In some embodiments, an oral composition comprising endoxifen or a polymorph thereof further comprises one or more excipients. Accordingly, compositions designed for oral administration can be made with an inert or active excipient or with an edible carrier as disclosed herein.

In various embodiments, the composition provided herein comprises from about 1% to about 99.99%, about 5% to about 95%, about 5% to about 90%, about 10% to about 80%, about 15% to about 70%, about 20% to about 60%, from about 30% to about 95%, from about 50% to about 90%, from about 60% to about 90%, from about 60% to about 80%, or from about 70% to about 80% by weight of one or more excipients. In certain embodiments, the composition provided herein comprises about 99.99%, about 95%, about 90%, about 85%, about 80%, about 75%, about 70%, about 65%, about 60%, about 55%, or about 50% by weight of one or more excipients. In certain embodiments, the composition provided herein comprises about 99.99%, about 99%, about 98%, about 97%, about 96%, about 95%, about 94%, about 93%, about 92%, about 91%, about 90%, about 89%, about 88%, about 87%, about 86%, or about 85% by weight of one or more excipients. In certain embodiments, the composition provided herein comprises about 85%, about 84%, about 83%, about 82%, about 80%, about 79%, about 78%, about 77%, about 76%, about 75%, about 74%, about 73%, about 72%, about 71%, about 70%, about 69%, about 68%, about 67%, about 66%, or about 65% by weight of one or more excipients. In certain embodiments, the composition provided herein comprises about 55%, about 54%, about 53%, about 52%, about 51%, about 50%, about 49%, about 48%, about 47%, about 46%, or about 45% by weight of one or more excipients. In certain embodiments, the composition provided herein comprises about 30%, about 29%, about 28%, about 27%, about 26%, about 25%, about 24%, about 23%, about 22%, about 21%, or about 20% by weight of one or more excipients.

Examples of excipients that can be used in the compositions formulated for oral administration are provided herein and can include, but are not limited to, one or more of bulking agents, binders, fillers, disintegrating agents, lubricants, glidants, control release agents, enteric coatings, film-forming agents, plasticizers, colorants, sweeteners, flavoring agents and the like, or any combination thereof.

Binders suitable for use in the pharmaceutical compositions provided herein include, but are not limited to, sucrose, starches such as corn starch, potato starch, or starches such as starch paste, pregelatinized starch, and starch 1500, PEG 6000, methocel, walocel HM, Luvitec, Luvicaparolactam, Avicel, SMCC, UNIPURE, gelatin, natural and synthetic gums such as acacia, sodium alginate, alginic acid, other alginates, tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, polyvinyl pyrrolidone, hydroxypropyl methyl cellulose, (e.g., Nos. 2208, 2906, 2910), microcrystalline cellulose, and mixtures thereof. Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. In some embodiments, the binder is a mixture of microcrystalline cellulose and sodium carboxymethyl cellulose. Suitable anhydrous or low moisture excipients or additives include AVICEL PH 103 and Starch 1500 LM.

Examples of fillers suitable for use in the pharmaceutical compositions provided herein include, but are not limited to, talc, calcium carbonate (e.g., granules or powder), sugars such as dextrose, sucrose, lactose, a salt such as calcium carbonate, calcium phosphate, sodium carbonate, sodium phosphate, starches, microcrystalline cellulose, powdered cellulose, cellulosic bases such as methyl cellulose, carboxymethyl cellulose dextrates, kaolin, mannitol, silicic acid, sorbitol, starch, pregelatinized starch, and mixtures thereof.

One or more binder or filler in compositions is typically present in from about 10% to about 99% (wt/wt) of the composition or the dosage form. In some embodiments, binders and/or fillers in a composition comprise about 15% to 99%, about 20% to 60%, about 25% to 55%, about 30% to 50%, about 35% to 60%, about 50% to 99% (wt/wt) of the composition.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. In some embodiments, the disintegrant is deep in the oral solid dosage form to delay disintegration. The amount of disintegrant used varies based upon the type of formulation, and is readily discernible to those of ordinary skill in the art.

Typical compositions comprise from 0.5% to 15% (wt/wt) of disintegrant. In some embodiments, compositions comprise from 1% to 5% (wt/wt) of disintegrant in the composition. In another embodiment, the disintegrant is 1% to 25%, 2% to 20%, 5% to 15%, 8% to 12%, or about 10% (wt/wt) of the composition.

Disintegrants that can be used in the pharmaceutical compositions provided herein include, but are not limited to, agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, magnesium stearate or potassium stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), Q7-9120 (Dow Corning), and mixtures thereof. If used at all, lubricants are typically used in an amount of less than 1% (wt/wt) of the compositions or dosage forms into which they are incorporated. In yet another embodiment, the lubricant is 0.1% to 3%, such as 0.5% to 1% (wt/wt), of the composition.

Plasticizers may be added to control the softness or pliability of oral dosage forms such as shell of a capsule, caplet or a tablet and thus, may improve the mechanical properties of the pH-sensitive materials of the coatings on the oral dosage forms. Suitable plasticizers, include, without limitation, petroleum oils (for e.g., a paraffinic process oil, a naphthenic process oil, and an aromatic process oil), squalene, squalane, plant oils, (e.g., olive oil, camelia oil, castor oil, tall oil, and a peanut oil), silicon oils, dibasic acid esters, (e.g., dibutyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, triethyl citrate, triacetin, diethylene glycol, polyethylene glycols, polypropylene glycol, phthalates, sorbitol, glycol salicylate, crotamiton, and glycerin or mixtures thereof. The amount of plasticizer may vary depending upon the chemical composition of the pharmaceutical preparation. In one embodiment, the at least one plasticizer is sorbitol, dimethyl isosorbide, or a glycerol. In another embodiment, the plasticizer is 1% to 10%, such as 3% to 5% (wt/wt), of the composition.

Examples of glidants include, but are not limited to, colloidal silicone dioxide, cellulose, calcium phosphate, di or tri-basic and the like.

As an example of sweeteners or sweetening agents include sucrose, saccharin, dextrose, maltose, sugar substitutes, aspartame, xylitol, mannitol, cyclamate, sucralose, maltitol, sorbitol, acesulfame K and the like.

Examples of flavoring agents include peppermint, methyl salicylate, peppermint, spearmint, methyl salicylate, raspberry, red berry, strawberry, pineapple, orange, cherry and the like.

Compositions formulated for oral delivery as disclosed herein, for example, tablets, caplets, and capsules, may be coated with one or more enteric coating agent, control release agent or film forming agent to control or delay disintegration and absorption of the compositions comprising endoxifen or salts thereof in the gastrointestinal tract and thereby provide a sustained action over a longer period of time. Accordingly, in some embodiments, the tablet can be an enteric tablet, the caplet can be an enteric caplet, or the capsule can be an enteric capsule. The enteric tablets, enteric caplets, or enteric capsules of the present disclosure may be prepared by techniques known in the art.

Pharmaceutical preparations disclosed herein may comprise a control release agent. Examples of control release agent suitable for use include, without limitation, pH-dependent polymers, acid-insoluble polymers, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, waxes, including synthetic waxes, microcrystalline waxes, paraffin wax, carnauba wax, and beeswax; polyethoxylated castor oil derivatives, hydrogenated oils, glyceryl mono-, di- tribenates, glyceryl monostearate, glyceryl distearate, long chain alcohols, such as stearyl alcohol, cetyl alcohol, and polyethylene glycol; and mixtures thereof. In some embodiments, a time delay material such as glyceryl monostearate or glyceryl distearate may be used. In other embodiments, the controlled release reagent is a digestible waxy substance such as hard paraffin wax.

In some embodiments, compositions may comprise one or more of pH-dependent polymers such as acid insoluble polymers. The pH-dependent polymers become increasingly permeable above pH 5.0 but are impermeable at pH below 5.0 whereas acid insoluble polymers become soluble in neutral to weakly alkaline conditions. Such control release polymers target upper small intestines and colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers (commercially available under the tradename EUDRAGIT® L and EUDRAGIT® S from Rohm America Inc., Piscataway, NJ as a powder or a 30% aqueous dispersion; or under the tradename EASTACRYL®, from Eastman Chemical Co., Kingsport, TN, as a 30% dispersion). Additional examples include EUDRAGIT® L100-55, EUDRAGIT® L30D-55, EUDRAGIT® L100, EUDRAGIT® L100 12,5, EUDRAGIT® S100, EUDRAGIT® S12,5, EUDRAGIT® FS 30D, EUDRAGIT® E100, EUDRAGIT® E 12,5, and EUDRAGIT® PO.

In at least one embodiment, the composition comprises EUDRAGIT® L100-55. EUDRAGIT® RS and RL and EUDRAGIT®NE and NM are also useful polymers for the purpose of this disclosure. In some embodiments, the composition comprises EUDRAGIT® L30D 55. In another embodiment, the preparation comprises EUDRAGIT® FS 30D. One of skill in the art will recognize that at least some acid insoluble polymers listed herein will also be biodegradable.

For time delay or delayed-release pharmaceutical preparations of oral dosage forms, glyceryl monostearate, glyceryl distearate, and acid-insoluble polymers, for example polymethacrylate pH-sensitive polymer-based coatings can be used, (e.g., as coating material, such as enteric coating agents, for enteric coating of capsules, caplets, and tablets). Commercial sources for delayed-release oral dosage forms are available, for example DRCAPS™ made of hypromellose (HPMC) from CAPSUGEL®, USA. Such delayed-release oral dosage forms are acid-resistant and can resist acidity as seen in stomach for at least 30 min, such as for at least 1 hour, for at least 1.5 hour, or for at least 2 hours. Such delayed release oral dosage forms can release at least 40%, at least 50%, at least 60%, at least 70%, at least 80% or at least 90% of the endoxifen or salts thereof in the intestines (small intestines, large intestine/colon etc.).

In an aspect of the present disclosure, the enteric tablets, enteric caplets, and enteric capsules may be uncoated. Hard uncoated capsules with enteric capability using intrinsically enteric capsule technology (for example, ENTRINSIC™ Drug Delivery available from CAPSUGEL®) are suitable for the purpose of the present disclosure.

In various embodiments, the enteric tablet is a hard tablet made with free-flowing powder of (Z)-endoxifen or a salt thereof. In various embodiments, the enteric capsule is a capsule made with free-flowing powder of (Z)-endoxifen or a salt thereof. In various embodiments, the enteric tablet is a hard tablet made with free-flowing powder of endoxifen or a polymorph thereof. In various embodiments, the enteric capsule is a capsule made with free-flowing powder of endoxifen or a polymorph thereof.

In some embodiments, the enteric capsule is a non-animal based capsule, such as a hypromellose capsule (for example, commercially available self-gelling VCAPS®, VCAPS® Plus, VCAPS® enteric, other enteric capsules made using Xcellodose, ENCODE colonic delivery technology, and ENTRINSICm drug delivery technology from CAPSUGEL®). Other technologies known in the art and available commercially (for example, QUALICAPS®, USA, Nutrascience, USA, etc.) for the formulating enteric forms of oral solid dosage forms can also be utilized. In at least one embodiment, the capsule is an API-in-capsule, meaning that the (Z)-endoxifen free base or salts thereof is filled neat into the capsule. In such API-in-capsule oral dosage forms, the active ingredient, (Z)-endoxifen or salts thereof can be free flowing powders or micronized powders. When the dosage form is a capsule, in at least one embodiment, the capsule can be a seamless capsule or a banded capsule.

Rapid absorption and bioavailability of the anti-cancer therapeutics such as endoxifen that can further reduce the cancer growth rate are highly desirable. In an aspect, the present disclosure provides that the compositions are formulated for certain pharmacokinetic (PK) properties.

In one aspect, rapid achievement of maximal and steady state plasma levels of endoxifen is a particular aspect of the present disclosure. The present disclosure provides compositions that achieve a maximal plasma level of endoxifen ranging within 2 to 30 hours, within 3 to 20 hours, within 2 to 10 hours or within 4 to 8 hours after administration of the compositions.

Accordingly, in some embodiments, time to maximal (peak) plasma level of endoxifen ranges from 2 to 10 hours after administration of the composition. In some embodiments, the time to maximal plasma level of endoxifen ranges from 4 to 8 hours after administration of a composition disclosed herein.

Rapid achievement of steady-state plasma levels of endoxifen is also highly desirable, and a composition of the present disclosure may provide a plasma level of endoxifen in a subject administered the composition comprising a polymorphic form of endoxifen, (Z)-endoxifen or a salt thereof that rapidly achieves steady state. Steady state plasma levels can be achieved from day 7 to day 21. In some embodiments, the steady state plasma levels can be achieved by day 7 upon daily administration of a composition disclosed herein.

In an aspect, the present disclosure provides that circulating endoxifen released from a composition disclosed herein can be cleared faster than tamoxifen. Terminal elimination half-life of tamoxifen may be 5-7 days (Jordan C. Steroids. 2007 November; 72(13): 829-842) and peak concentration time of tamoxifen may be approximately 5 hours post-dose. Endoxifen released from a composition disclosed herein can have a terminal elimination half-life ranging from 30 to 60 hours, significantly lower than tamoxifen. In some embodiments, the mean half-life ranges from 40 to 53 hours. The mean ratio of AUC₂ₕᵣ (Day 21)/AUC_{0-inf} (Day1) typically ranges from 0.7 to 1.2 for compositions comprising 1 mg to 4 mg (Z)-endoxifen, or a salt thereof. Thus, accumulation of endoxifen released from a composition disclosed herein does not significantly vary over continued treatment.

In another aspect, a composition described herein achieves absorption of endoxifen that is therapeutically effective.

Area under Curve AUC₍₀₋₂₄ₕᵣ₎ (“AUC₂₄ₕᵣ”) describes the total exposure of the subject to a drug from time of dosing (0 hr) over a 24-hour period. Compositions comprising (Z)-endoxifen or a salt thereof typically achieve mean (AUC₂₄ₕᵣ) of 150 hr*ng/mL to 600 hr*ng/mL on Day 1 of initial (first) dose of a composition comprising 1 mg to 4 mg of (Z)-endoxifen. Compositions comprising (Z)-endoxifen or a salt thereof typically achieve mean AUC₂₄ₕᵣ of 400 hr*ng/mL to 2500 hr*ng/mL on Day 21 of initial (first) dose of compositions comprising 1 mg to 4 mg of (Z)-endoxifen.

AUC_{0-inf} (“AUC_{0-inf}”), a time-averaged concentration of drug circulating in the body fluid analyzed (normally plasma, blood or serum), describes the total exposure of the subject to a drug. The present disclosure provides that the exposure of a subject to endoxifen (AUC_{0-inf}) can be dose proportional. In some embodiments, AUC_{0-inf} ranges from 200 hr*ng/mL to 10000 hr*ng/mL. In other embodiments, the AUC_{0-inf} ranges from 300 hr*ng/mL to 8000 hr*ng/mL. In certain embodiments, the AUC_{0-inf} ranges from 400 hr*ng/mL to 6000 hr*ng/mL over the dosing range of 1 mg to 4 mg of (Z)-endoxifen.

Dissolution of the oral dosage forms disclosed herein is tested by the dissolution tests according to the current methods of USP 711. In some embodiments, the oral dosage forms disclosed herein are protected from the acidic environment of the stomach and do not dissolve for at least 2 hours, at least 3 hours, at least 4 hours, at least 5 hours, 6 hours, at least 7 hours or at least 8 hours. In at least one embodiment, the oral dosage forms do not release endoxifen for at least 6 hours. In another embodiment, the oral dosage forms do not release endoxifen or a salt thereof for at least 2 hours.

In other embodiments, less than 10% of (Z)-endoxifen in a composition comprising endoxifen or a salt thereof disclosed herein is released in the stomach after 2 hours after administration; or wherein less than 40% of (Z)-endoxifen is released in the stomach after 4 hours of administration; or less than 50% of (Z)-endoxifen is released in the stomach after 6 hours of administration, as tested by a method of USP 711.

In another embodiment, a composition disclosed herein releases in the stomach less than 10% of (Z)-endoxifen in 2 hours after administration, less than 40% of (Z)-endoxifen in 4 hours after administration; and less than 50% of (Z)-endoxifen in 6 hours after administration, as tested by a method of USP 711.

In yet another embodiment, the composition is formulated to release in the small intestine, such that at least 10% of endoxifen is released after 4 hours after administration; or at least 30% of endoxifen is released after 6 hours after administration; or at least 40% of endoxifen is released after 7 hours after administration; or at least 50% of endoxifen is released after 8 hours after administration, as tested by a method of USP 711.

In a further embodiment, as determined by a method of USP 711, the composition is formulated to release in the colon at least 50% of endoxifen after 8 hours after administration.

In still further embodiments, the composition is formulated to release in the colon at least 20% of endoxifen after 4 hours after administration; at least 40% of endoxifen after 6 hours after administration; at least 60% of endoxifen after 7 hours after administration; or at least 80% of endoxifen after 8 hours after administration.

An oral dosage form can be of any shape suitable for oral administration, such as spherical (0.05-5 mL), oval (0.05-7 mL), ellipsoidal, pear (0.3-5 mL), cylindrical, cubic, regular and/or irregular shaped. An oral dosage form may be of any size suitable for oral administration, for example, size 0, size 2, and the like.

One of skill in the art will further recognize that compositions disclosed herein may comprise one or more of the excipients known in the art and disclosed herein in any combination appropriate for a desired formulation or preparation. Additional excipients may generally be found in Remington's The Science and Practice of Pharmacy, Meade Publishing Co., United States Pharmacopeia/National Formulary. One of skill in the art will be able to select suitable excipients necessary for the preparation of the formulations and appropriate dosage forms compatible with the route of administration based on his or her skill and knowledge in the art and the disclosures made herein. In all cases, the ultimate dosage form should be sterile and stable under the conditions of manufacture and storage.

For formulations of the solid dosage compositions disclosed herein, as the water activity (Aw) is less than 0.75, testing Total Aerobic Plate Count (TAC) and USP indicator organism is typically not necessary. The publication, “Microbial Bioburden on Oral Solid Dosage Form,” by Jose E. Martinez, Pharmaceutical Technology, February 2002, pages 58 to 70, is hereby incorporated by reference in its entirety.

Furthermore, since formulations of the compositions disclosed herein also have water activity of less than 0.75, then detailed microbial testing is typically not necessary. TAC is an estimation of the total viable aerobic microbes present in a sample of raw material, in-process material, or finished product. Samples are analyzed in accordance with the most current USP 39 <61>, “Microbiological Examination of Nonsterile Products: Microbial Enumeration Tests.”

Acceptable TAC for oral solid dosage forms (OSDFs) are established for the formulation of the inventive compositions in terms of alert and action levels, which could be 1000 cfu g/mL, and 10,000 cfu g/mL, respectively. A TAC that is 20,000 cfu g/mL may be considered unacceptable.

For other formulations, such as liquid or fluid formulations with water activity of less than 0.75, Tests for Specified Microorganisms (S. aureus, Ps. aeruginosa, Salmonella, C. albicans, Clostridia, E. coli and Bile Tolerant Gram negative bacteria) in compliance with USP Guidelines Chapter 62 may not need to be performed.

### Abbreviations Used Herein

ACN/PPW: Acetonitrile/Process Purified Water
NaHCO₃: Sodium hydrocarbonate
HCl: hydrochloric acid
THF: Tetrahydrofuran
MeTHF: 2-Methyltetrahydrofuran
CH₂Cl₂: Dicholoromethane
EtOH: Ethanol
MeOH: Methanol
EtOAc: Ethyl acetate
IPA: Isopropyl alcohol; isopropanol
TFA: Trifluoroacetic acid
TCA: Trichloroacetic acid
PPW: Process Purified Water
IPA/PPW: Isopropyl alcohol/Process Purified Water
CV: Coefficient of Variance
Tₘₐₓ: time to maximum concentration
Cₘₐₓ: maximum concentration,
AUC₀₋₂₄ₕ: (“AUC24 hr”) area under the concentration-time curve from time 0 to 24 hours following drug administration
Kel: terminal elimination rate constant, and volume of
t_{1/2}: terminal half-life
C_{L/F}: terminal clearance
V_{d/F}: distribution.
AUC_{0-inf}: (“AUC_{0-inf}”) Area under the concentration-time curve from time 0 to infinity
2PR: 2-propanol
ACN: acetonitrile
ACT: acetone
BAC: butyl acetate
BME: butyl methyl ether
DMF: dimethylformamide
ETA: ethyl acetate
ETH: ethanol
H2O: water
HEP: heptane
MET: methanol
MIBK: methyl isobutyl ketone (4-methyl-2-pentanone)
TOL: toluene
DSC: differential scanning calorimetry
EGA: evolved gas analysis
EVHT: evaporation at high temperature
EVRT: evaporation at room temperature
GRAD: gradient precipitation
HT: high temperature
PAD: precipitation by antisolvent addition to a saturated solution
PAL: precipitation by saturated solution addition to an antisolvent
SLRT: slurry experiment at room temperature
SLHT: slurry experiment at high temperature
TGA: thermogravimetric analysis

Compounds are described using standard nomenclature. Unless otherwise defined, all technical and scientific terms used herein have the same meaning as is commonly understood by one of skill in the art to which this invention belongs.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the terms “a,” “an,” and “the” include plural reference unless the context dictates otherwise.

As used herein, the terms “active pharmaceutical ingredient”, “active ingredient”, “API,” “drug,” “active,” “actives” or “therapeutic agent” may be used interchangeably to refer to the pharmaceutically active compound(s) in a pharmaceutical composition. This is in contrast to other ingredients in the compositions, such as excipients, which are substantially or completely pharmaceutically inert. A suitable API in accordance with the present disclosure is one where there is or likely may be patient compliance issues for treating a certain disease, condition, or disorder. The therapeutic agent as used herein includes the active compound and its salts, prodrugs, and metabolites. As used herein the term “drug” means a compound intended for use in diagnosis, cure, mitigation, treatment, and/or prevention of disease in man or other animals.

As used herein, “adjuvant therapy” refers to a therapy that follows a primary therapy and that is administered to subjects at risk of relapsing. Adjuvant systemic therapy in case of breast cancer or reproductive tract cancer, for example with tamoxifen, usually begins soon after primary therapy to delay recurrence, prolong survival or cure a subject.

As used herein, the term “tamoxifen” refers to (Z)-2-[4-(1,2-diphenyl-1-butenyl)phenoxy]-N,N-dimethylethanamine. Tamoxifen can also refer to the E-isomer or a combination of the E-isomer and the Z-isomer.

As used herein, the terms “4-hydroxytamoxifen,” “afimoxifene,” and “4-OHT” used interchangeably refer to 4-1-[4-[2-(dimethylamino)ethoxy]phenyl]-2-phenylbut-1-enyl]phenol, and constitutes an active metabolite of tamoxifen. 4-OHT can refer to the Z-isomer, E-isomer or a combination thereof.

As used herein, the term “endoxifen” refers to 4-hydroxy-N-desmethyl-tamoxifen. It is a secondary active metabolite of tamoxifen.

Embodiments that reference throughout this specification to “a compound”, such as compounds of Formula (I), Formula (II), Formula (III) and Formula (IV), include the polymorphic, salt, free base, co-crystal, and solvate forms of the formulas and/or compounds disclosed herein. Thus, the appearances of the phrases “a compound”, “compound of Formula (I)”, “compound of Formula (II)”, “compounds of Formula (III)” and “compound of Formula (IV)” include Form I of the compound of Formula (IV), Forms IV-XIX of the compounds of Formula (III), the free base of the compound of Formula (IV), the free base of the compounds of Formula (III), and/or the gluconate salts as described herein.

The terms “crystalline form”, “polymorph” and “Form” may be used interchangeably herein, and are meant to include all crystalline and amorphous forms of the compound, including, for example, polymorphs, pseudopolymorphs, salts, solvates, hydrates, unsolvated polymorphs (including anhydrates), conformational polymorphs, and amorphous forms, as well as mixtures thereof, unless a particular crystalline or amorphous form is referred to. Compound of the present disclosure include crystalline and amorphous forms of those compounds, including, for example, polymorphs, pseudopolymorphs, salts, solvates, hydrates, unsolvated polymorphs (including anhydrates), conformational polymorphs, and amorphous forms of the compounds, as well as mixtures thereof.

In some embodiments, the solution of (Z)-endoxifen is cooled from a temperature between 20° C. and 100° C. to a temperature in the range of −20° C. and 25° C.

In some embodiments, a slurry of (Z)-endoxifen is heated to a temperature in the range of 20° C. to 50° C.

In some embodiments, the solution of (Z) endoxifen may be cooled from 20° C. to −0.20° C. prior to storage.

The term “substantially as shown in” when referring, for example, to an XRPD pattern, includes a pattern that is not necessarily identical to those depicted herein, but that falls within the limits of experimental error or deviations when considered by one of ordinary skill in the art. The relative intensities of XRPD peaks can vary, depending upon the particle size, the sample preparation technique, the sample mounting procedure and the particular instrument employed. Moreover, instrument variation and other factors can affect the two theta (2θ) values. Accordingly, when a specified two theta angle is provided, it is to be understood that the specified two theta angle can vary by the specified value±0.5°, such as ±0.4°, ±0.3°, +0.2°, or ±0.1°. As used herein, “major peak” can refer to an XRPD peak with a relative intensity greater than 30%, such as greater than 35%. Alternatively, or in addition thereto, “major peak” can refer to an XRPD peak which is among the ten most intense XRPD peaks within an XRPD pattern. Relative intensity is calculated as a ratio of the peak intensity of the peak of interest versus the peak intensity of the largest peak in the XRPD pattern.

All compounds disclosed herein are further understood to include all possible isotopes of atoms occurring in the compounds. Isotopes include those atoms having the same atomic number but different mass numbers. By way of example, and without limitation, isotopes of hydrogen include tritium and deuterium and isotopes of carbon include ¹¹C, ¹³C and ¹⁴C.

As used herein and in the claims, the terms “comprising,” “containing,” and “including” are inclusive, open-ended and do not exclude additional unrecited elements, compositional components or method steps. Accordingly, the terms “comprising” and “including” encompass the more restrictive terms “consisting of” and “consisting essentially of.”

As used herein, the term “combination therapy” refers to the use of a composition described herein in combination with one or more additional treatment. Treatment in combination therapy can be any treatment such as any prophylactic agent, therapeutic agent (such as chemotherapy), radiotherapy, surgery and the like. The combination can refer to inclusion of a therapeutic or prophylactic agent in a same composition as a composition disclosed herein (for example, in the same capsule, tablet, ointment, etc.) or in separate compositions (for example, in 2 separate capsules). The separate compositions may be in a different dosage form. The use of the terms “combination therapy” and “in combination with” does not restrict the order in which a composition described herein and prophylactic and/or therapeutic agent and/or treatment are administered to a subject in need thereof. Compositions of the present disclosure can be administered prior to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year before), concomitant with, or subsequent to (e.g., 1 minute (min), 5 min, 15 min, 30 min, 45 min, 1 hour (h), 2 h, 4 h, 6 h, 8 h, 10 h, 12 h, 24 h, 36 h, 48 h, 72 h, 96 h, 1 week (wk), 2 wk, 3 wk, 4 wk, 5 wk, 6 wk, 8 wk, 12 wk, 6 months (m), 9 m, or 1 year after) administration of one or more prophylactic and/or therapeutic agent and/or treatment to a subject in thereof. Combination therapy as used herein can also refer to treatment of a subject having a single disease or multiple diseases, for example, prostate cancer in men and gynecomastia.

As used herein, the term “test sample” means sample of blood obtained from a subject. It is to be understood that when blood sample is obtained from a subject, subject's blood is used for determining the subject's endoxifen levels and/or other biomarkers that may be measured or tested. As used herein “plasma endoxifen” is used to refer to endoxifen levels in the subject's test sample, whether the test is conducted on whole blood, plasma, or serum.

As used herein, the term “dosage form” means the form in which the compounds or compositions of the present disclosure are delivered to a patient.

As used herein, the term “pharmaceutically acceptable” or “pharmacologically acceptable” means materials, compositions, or vehicles that are compatible with other ingredients of the formulation and that they do not substantially produce adverse reactions, e.g., toxic, allergic, or immunological reactions, when administered to a subject. They may be approved by a regulatory agency, e.g., of the U.S. Federal or state government or listed in the U.S. pharmacopeia or other generally recognized pharmacopeia for use in animals, and more particularly in humans.

As used herein, the term “pharmaceutically acceptable carrier” or “carrier” means a pharmaceutically acceptable material, composition, or vehicle, such as a liquid or solid filler, diluent, excipient, solvent, or encapsulating material, involved in carrying or transporting one or more of the compounds of the present disclosure from one tissue, organ, or portion of the body or across the skin.

As used herein, the term “pharmaceutically acceptable salt” refers to any salt (e.g., obtained by reaction with an acid or a base) of a compound of the present disclosure that is physiologically tolerated in a subject (e.g., a mammal, and/or in vivo, ex-vivo, in vitro cells, tissues, or organs). A “salt” of a compound of the present disclosure may be derived from inorganic or organic acids and bases. Suitable anion salts include, arecoline, besylate, bicarbonate, bitartarate, butylbromide, citrate, camysylate, gluconate, glutamate, glycollylarsanilate, hexylresorcinate, hydrabamine, hydrobromide, hydrochloride, hydroxynapthanoate, isethionate, malate, mandelate, mesylate, methylbromide, methylbromide, methylnitrate, methylsulfate, mucate, napsylate, nitrate, pamaoate (Embonate), pantothenate, phosphate/diphosphate, polygalacuronate, salicylate, stearate, sulfate, tannate, teoclate, fatty acid anions, and triethiodide.

Suitable cations include benzathine, clemizole, chloroprocaine, choline, diethylamine, diethanolamine, ethylenediamine, meglumine, piperazine, procaine, aluminum, barium, bismuth, lithium, magnesium, potassium, and zinc.

For the purposes of this application, the salts of the compounds of the present disclosure are contemplated to be pharmaceutically acceptable for therapeutic uses. However, salts of acids and bases that are non-pharmaceutically acceptable may also be useful, for example, in the preparation or purification of a pharmaceutically acceptable compound.

As used herein, the term “pharmaceutical composition” means a combination of the active agent (e.g., an active pharmaceutical compound or ingredient, API) with a carrier, inert or active (e.g., a phospholipid), making the compositions especially suitable for diagnostic or therapeutic uses in vitro, in vivo, or ex vivo.

As used herein “primary therapy” refers to a first line of treatment upon initial diagnosis of a hormone-dependent breast disorder, a hormone-dependent reproductive tract disorder, or both in a subject. Exemplary primary therapies may involve surgery, a wide range of chemotherapies, and radiotherapy.

As used herein, the terms “subject,” “patient,” “participant,” and “individual,” may be used interchangeably herein and refer to a mammal such as a human. Mammals also include pet animals such as dogs, cats, laboratory animals, such as rats, mice, and farm animals such as cows and horses. Unless otherwise specified, a mammal may be of any gender or sex.

As used herein, the term “tamoxifen refractory” refers to a subject that has been dosed daily with tamoxifen for at least 2 days and has a level of plasma endoxifen of less than 30 nM (e.g., less than 20 nM, less than 25 nM, or less than 30 nM). As used herein, the term “tamoxifen resistance” refers to two classes of resistance: (a) de novo resistance, i.e., non-responsiveness to tamoxifen therapy from the beginning of the treatment, or (b) acquired resistance, i.e., non-responsiveness to tamoxifen therapy after initial responsiveness or tamoxifen-dependent growth/stimulated growth while continuing to express estrogen receptors (Minsun Chang. Biomol. Ther. 20(3), 256-267 (2012)). The acquired resistance to tamoxifen may develop as early as 3 m to 1 year to as late as 5 to 10 years. As used herein, the term “reference plasma endoxifen level” refers to a value of 30 nM.

As used herein, the term “unit dosage form” refers to physically discrete units suitable for unitary dosages for subjects, each unit containing a predetermined quantity of active material calculated to produce the desired therapeutic effect, in association with a suitable pharmaceutical excipient.

It is specifically understood that any numerical value cited herein includes all values from the lower value to the upper value, i.e., all possible combination of numerical values between the lowest value and the highest value enumerated are to be considered to be expressly stated in this application and the endpoint of all ranges are included within the range and independently combinable. For example, if a concentration range or beneficial range is stated as 1% to 50%, it is intended that values such as 2% to 40%, 10% to 30%, or 1% to 3% etc., are expressly enumerated in this specification. It is also to be understood that if a concentration or dose is stated as a specific value such as 1 mg or 10 mg, it is intended that it is intended to include 10% variation. As another example, a stated concentration of 20% is intended to include values±10%. Yet another example, if a ratio of 1:10 to 10:1 is stated, then it is intended that ratios such as 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1, from 1:1 to 2:1 or from 2:5 to 3:5 etc. are specifically intended. There are only some examples of what is specifically intended. Unless specified otherwise, the values of the constituents or components of the compositions are expressed in weight percent of each ingredient in the component.

All methods described herein can be performed in a suitable order unless otherwise indicated or otherwise clearly contradicted by context. The use of any and all examples, or exemplary language (e.g., “such as” and “the like”) is intended merely to illustrate the invention and does not pose a limitation on the scope of the invention unless otherwise claimed. No language in the specification should be construed as any indicating any non-claimed element as essential to practice of the invention as used herein.

As used herein, the terms “hormone-dependent breast disorder,” “hormone-dependent reproductive tract disorder,” “hormone-dependent breast and reproductive tract disorder” each and collectively include, without limitation, any breast or reproductive tract (gynecologic) disorder that is related to or is sensitive to high estrogen or normal estrogen levels that need to be reduced, disorders with estrogen-receptor positive (ER+) and/or progesterone-receptor positive (PR+) disorders, for example, breast disorders, endometriosis, uterine fibroids (also called leiomyomas) etc. Reproductive tract disorders include endometrial, ovarian, cervical, uterus, vaginal, and vulvar cancers. The terms “estrogen-related disorder” and “estrogen-receptor related disorder” may be used interchangeably to refer to the foregoing hormone dependent disorders. The disorders may be presented primarily or secondarily to an underlying disease, for example, prostate cancer or other disorders such as liver diseases. Hormone-dependent breast and reproductive tract disorder include, for example, McCune-Albright syndrome, which is a disorder caused by a mutation in the GNAS gene affecting bones, skin, and several hormone-producing (endocrine) tissues, often resulting in abnormal scar-like (fibrous) tissue in their bones, a condition called polyostotic fibrous dysplasia, hyperthyroidism in individuals carrying such mutations, and in girls often resulting in precocious puberty.

As used herein, “breast disorder” means any aberration or a constellation of aberrations in the breast. Such aberration may be proliferative, non-proliferative, benign or malignant. Breast disorders include benign lesions of the breast (e.g., hyperplasia), increased breast density, gynecomastia, mastalgia, and breast cancer. Benign breast lesions include, but are not limited to, hyperplasia, atypia, ductal hyperplasia, lobular hyperplasia, atypical ductal hyperplasia (ADH), and atypical lobular hyperplasia (ALH). While not cancerous, ADH and ALH may be indicative of a predisposition for breast cancer.

As used herein, “breast cancer” means any malignant tumor of breast cells. Breast cancer may be at any stage of breast cancer, including stages of a pre-cancer, an early-stage cancer, a non-metastatic cancer, a pre-metastatic cancer, a locally advanced cancer, and a metastatic cancer. There are several types of breast cancer. Exemplary breast cancers include, but are not limited to, ductal carcinoma in situ (DCIS), lobular carcinoma in situ (LCIS), invasive (or infiltrating) lobular carcinoma (ILC), invasive (or infiltrating) ductal carcinoma (IDC), microinvasive breast carcinoma (MIC), inflammatory breast cancer, ER-positive (ER+) breast cancer, ER-negative (ER−) breast cancer, HER2+ breast cancer, triple negative breast cancer (TNBC), adenoid cystic (adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma. A single breast cancer tumor can be a combination of these types or be a mixture of invasive and in situ cancer.

### EXAMPLES

The invention is further illustrated by the following non-limiting examples.

### Example 1

### Preparation of [4-[2-(methylamino)ethoxy]phenyl](4-hydroxyphenyl)methanone) (Formula

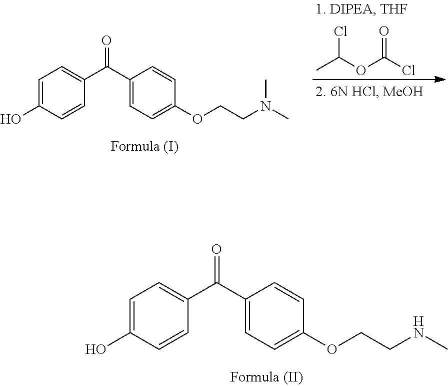

This example describes preparation of [4-[2-(methylamino)-ethoxy]phenyl](4-hydroxyphenyl)methanone) (Formula (II)).

A suitable reactor was charged with starting material [4-[2-(dimethylamino)ethoxy]-phenyl](4-hydroxyphenyl)methanone, the compound of Formula (I), (1.0 Kg, 1.0 equiv.), N-Ethyldiisopropylamine (1.8 Kg, 4.0 equiv., 1.8 wt.) and Tetrahydrofuran (10.0 L, 8.9 Kg, 10.0 vol., 8.9 wt.) under N₂ atmosphere. The mixture was heated to NLT 60° C. and 1-Chloroethyl chloroformate (2.0 Kg, 4.0 equiv., 2.0 wt.) was added. The mixture was heated to reflux and stirred for NLT 12 hr. The mixture was concentrated until the volume reached about 5 vol. Methanol (5.0 L, 4.0 Kg, 5.0 vol., 4.0 wt.) was then added slowly and the resulting mixture was again concentrated until the volume reached 5 L. Methanol (2.0 L, 1.6 Kg, 2.0 vol., 1.6 wt.) and 6N HCl (4.0 L, 4.0 Kg, 4.0 vol., 4.0 wt.) were added and the mixture was heated to reflux. The mixture was stirred at reflux for NLT 12 hr. After reaction completion, the mixture was concentrated until the volume reached −4 vol. The mixture was cooled to ambient temperature and 8N NaOH (NLT 5.0 L, 5.0 Kg, 5.0 vol., 5.0 wt.) was added slowly until the mixture turned from suspension to a clear solution and a pH 13. Ethyl acetate (4.0 L, 3.6 Kg, 4.0 vol., 3.6 wt.) was added for extraction. After phase separation, 6N HCl (NLT 0.6 L, 0.6 Kg, 0.6 vol., 0.6 wt.) was added to the aqueous phase until the pH was 8-10. The resulting aqueous mixture was cooled to 0° C.±5° C. and stirred for NLT 2 hr. The mixture was then filtered and washed with purified water (NLT 3.0 L, 3.0 Kg, 3.0 vol., 3.0 wt.) and ethyl acetate (NLT 2 L, 1.8 Kg, 2 vol., 1.8 wt.). The wet cake was dried at NMT 60° C. under reduced pressure to afford (4-hydroxyphenyl)(4-(2-(methylamino)ethoxy)phenyl)methanone, the compound of Formula (II) (˜0.7 Kg). The expected yield of this reaction was NLT 70%, based on the compound of Formula (I).

### Example 2

### Preparation of E/Z-endoxifen mixture (Formula III) in a McMurry Reaction

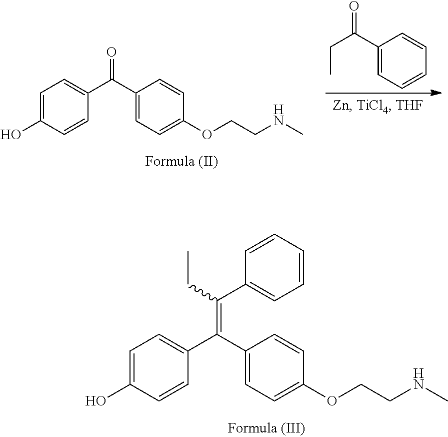

This example describes preparation of E/Z-endoxifen mixture in a McMurry reaction.

A suitable reactor was charged with Tetrahydrofuran (10.0 L, 8.9 Kg, 10.0 vol., 8.9 wt.) and Zinc powder (0.96 Kg, 4.0 equiv., 0.96 wt.) under N₂ atmosphere. Titanium (IV) chloride (1.4 Kg, 2.0 equiv., 1.4 wt.) was added slowly while maintaining the internal temperature at NMT 20° C. The reaction was heated to NLT 60° C. and stirred for NLT 2 hr. A suspension of (4-hydroxyphenyl)(4-(2-(methylamino)ethoxy)phenyl)methanone, the compound of Formula (II) obtained from EXAMPLE 1, (1.0 Kg, 1.0 equiv.) and propiophenone (0.74 Kg, 1.5 equiv., 0.74 wt.) in Tetrahydrofuran (8.0 L, 7.1 Kg, 8.0 vol., 7.1 wt.) was heated to NLT 60° C., added to the above mixture for NLT 1 hr and stirred at NLT 60° C. for NLT 8 hr. The mixture was concentrated to ˜10 vol. and MeTHF (10.0 L, 8.6 Kg, 10.0 vol., 8.6 wt.) was added. The mixture was concentrated to ˜10 vol. and another amount of MeTHF (10.0 L, 8.6 Kg, 10.0 vol., 8.6 wt.) was added. The mixture was concentrated to ˜10 vol. and cooled to NMT 30° C. 1N HCl solution (10.0 L, 10.0 Kg, 10.0 vol., 10.0 wt.) was added at NMT 30° C. and stirred for NLT 30 min.

The above solution was filtered, and the phases were let to separate. The aqueous layer was extracted 2 times with MeTHF (10.0 L, 8.6 Kg, 10.0 vol., 8.6 wt.). The combined organic layer was concentrated at NMT 40° C. to ˜10 vol. and extracted with 40% K₂CO₃ solution (8.0 L, 11.4 Kg, 8.0 vol., 11.4 wt.). The mixture was filtered and washed with MeTHF (NLT 4.0 L, 3.4 Kg, 4.0 vol., 3.4 wt.). The filtrate was concentrated to ˜22 vol, settled for phase separation and the organic layer was extracted with 1N NaOH solution (10.0 L, 10.0 Kg, 10.0 vol., 10.0 wt.). Sodium chloride (1.0 Kg, 1.0 wt.) was added to the aqueous layer and then extracted 3 times with MeTHF (NLT 10.0 L, 8.6 Kg, 10.0 vol., 8.6 wt.). The combined organic layer was concentrated until the volume reached ˜15 vol. The mixture was extracted with 20% NaCl solution (5.0 L, 5.0 Kg, 5.0 vol., 5.0 wt.). After phase separation, the organic layer was concentrated until no distillate was present. To the mixture was added Acetone (10.0 L, 7.9 Kg, 10.0 vol., 7.9 wt.) followed by concentration until no distillate was present. Acetone (10.0 L, 7.9 Kg, 10.0 vol., 7.9 wt.) was added and the mixture was concentrated until the volume reached ˜6 vol. The mixture was heated to NLT 50° C. and Acetonitrile (3.0 L, 2.4 Kg, 3.0 vol., 2.4 wt.) was added for NLT 1 hr at NLT 50° C. The mixture was concentrated until the volume reached ˜6 vol., stirred at NLT 50° C. for NLT 1 hr, cooled to NMT 5° C. and stirred at NMT 5° C. for NLT 12 hr. The mixture was filtered and washed with pre-cooled Acetonitrile (NLT 2.0 L, 1.6 Kg, 2.0 vol., 1.6 wt.). The wet cake was dried at NMT 70° C. under reduced pressure to afford a mixture of (Z)-endoxifen and (E)-endoxifen, (E/Z)-4-[1-[4-[2-(Methylamino)ethoxy]phenyl]-2-phenyl-1-buten-1-yl]-phenol, compounds of Formula (III), (˜0.6 Kg). The expected yield of this reaction was NLT 30% based on the compound of Formula (II).

### Example 3

### Enrichment and Preparation of (Z)-Endoxifen Free Base

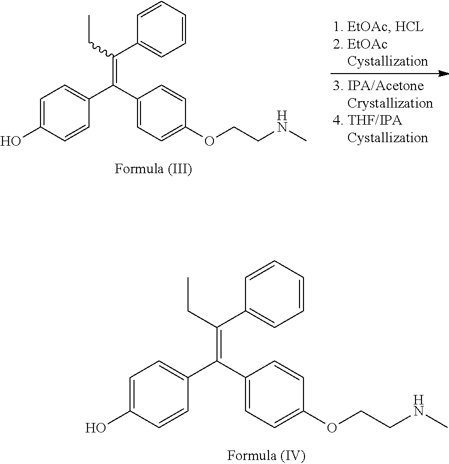 [SMILES: CCC(=C(c1ccc(O)cc1)c1ccc(OCCNC)cc1)c1ccccc1 ; CC/C(=C(\c1ccc(O)cc1)c1ccc(OCCNC)cc1)c1ccccc1]

This example describes the enrichment and preparation of (Z)-endoxifen free base using multiple steps of (re-)crystallization comprising ethyl acetate (EtOAc) crystallization, IPA/acetone recrystallization, and THF/IPA recrystallization.

A suitable reactor was charged with a mixture of (Z)-endoxifen and (E)-endoxifen, (E/Z)-4-[1-[4-[2-(Methylamino)ethoxy]phenyl]-2-phenyl-1-buten-1-yl]-phenol, compounds of Formula (III), produced in EXAMPLE 2 or obtained commercially, (1.0 Kg, 1.0 wt.) and ethyl acetate (10.0 L, 9.0 Kg, 10.0 vol., 9.0 wt.). 6N HCl solution (4.0 L, 4.0 Kg, 4.0 vol., 4.0 wt.) was added slowly while maintaining the temperature at NMT 10° C. The mixture was heated to 60±5° C. and stirred for NLT 6 hr. 8N NaOH solution (around 8.0 L, 8.0 Kg, 8.0 vol., 8.0 wt.) was added slowly until the pH of the mixture was NLT 12 while maintaining the temperature at NMT 10° C. The mixture was warmed to NLT 20° C. and settled for phase separation. The aqueous layer was washed with ethyl acetate (5.0 L, 4.5 Kg, 5 vol., 4.5 wt.). The combined organic layer was washed with 20% NaCl solution (3.0 L, 3.0 Kg, 3.0 vol., 3.0 wt.). The organic layer was filtered through Activated carbon (0.05 Kg, 0.05 wt.)/Celite bed and washed with Ethyl acetate (5.0 L, 4.5 Kg, 5.0 vol., 4.5 wt.). The filtrate was concentrated until the volume reached 13 vol. (13 L).

Ethyl acetate (7.0 L, 6.3 Kg, 7.0 vol., 6.3 wt.) was added and the mixture was concentrated until the volume reached 13 vol (13 L). The mixture was cooled to NMT 5° C., then heated to 50±5° C. and then cooled again to NMT 5° C. The mixture was filtered and washed with ethyl acetate (1.5 L, 1.5 Kg, 1.5 vol., 1.3 wt.). The wet cake was dried at NMT 90° C. to afford a mixture of (Z)-endoxifen and (E)-endoxifen (recovery). The filtrate was collected and concentrated until no distillate was present.

Acetone (10.0 L, 7.9 Kg, 10.0 vol., 7.9 wt.) was added to the filtrate and the mixture was concentrated until no distillate was present. Another volume of acetone (10.0 L, 7.9 Kg, 10.0 vol., 7.9 wt.) was added and the mixture was concentrated at NLT 30° C. until the volume reached ˜3 vol. 2-propanol (IPA, 1.5 L, 1.2 Kg, 1.5 vol., 1.2 wt.) was then added at NLT 40° C. and the mixture was concentrated at NLT 40° C. until the volume reached 3 vol. The mixture was cooled to 0±5° C. and stirred at 0±5° C. for NLT 6 hr. The mixture was filtered and washed with pre-cooled IPA (NLT 2 L, 1.6 Kg, 2 vol., 1.6 wt.). The wet cake was dried at NMT 80° C. to afford crude (Z)-endoxifen, the crude compound of Formula (IV).

A suitable reactor was charged with the crude (Z)-endoxifen and THF. The mixture was heated to 50±5° C. The solution was concentrated until the volume reached about 3 vol (3 L). IPA was added and the mixture was concentrated at NLT 40° C. until the volume reached about 8 vol. IPA was added and the mixture was concentrated at NLT 40° C. until the volume reached 7 vol. The mixture was cooled to NMT 5° C. and stirred at NMT 5° C. for NLT 6 hr. The mixture was filtered and washed with pre-cooled 2-Propanol. The wet cake was dried at NMT 80° C. to afford purified (Z)-Endoxifen, a compound of Formula (IV) (˜0.26 Kg). The expected yield based on the compound of Formula (III) was NLT 18%.

(Z)-Endoxifen prepared by this method was analyzed for its appearance and impurities content, of which an overview is shown in TABLE 1 below, and demonstrating that the methods described herein (e.g., mesityl oxide-controlled methods) can furnish highly pure (Z)-Endoxifen with significantly reduced amounts of mesityl oxide compared to conventional methods:

TABLE 1
Analytical Results of (Z)-Endoxifen Produced herein

| Method Description | Acceptance Criteria |
|---|---|
| Appearance | White to Beige Powder |
| Identification: FT-IR | Conform with Standard |
| Assay: HPLC (dried basis, % w/w) | 94%-102% |
| Identification: HPLC | Retention time and peak shape |
| | conform with standard |
| (Z)-Endoxifen Purity: HPLC (% w/w) | NLT 96% |
| Related Substances: HPLC (% w/w) | |
| (E)-Endoxifen | NMT 3% |
| Max. individual impurity | NMT 0.3% |
| Total impurities | NMT 4% |
| Water content: KF | NMT 1.0% |

Residual Solvents: Headspace GC

| | |
|---|---|
| Methanol | NMT 3000 ppm |
| Ethanol | NMT 5000 ppm |
| Acetone | NMT 5000 ppm |
| Isopropanol (IPA) | NMT 5000 ppm |
| Acetonitrile | NMT 410 ppm |
| Ethyl Acetate | NMT 3000 ppm |
| Tetrahydrofuran | NMT 720 ppm |
| 2-Methyltetrahydrofuran | NMT 520 ppm |
| n-Heptane | NMT 5000 ppm |

Titanium, Zinc, Benzene, and Mesityl Oxide content:

| | |
|---|---|
| Titanium (Ti) | NMT 32.5 ppm |
| Zinc (Zn) | NMT 130 ppm |
| Benzene | NMT 2 ppm |
| Mesityl Oxide | NMT 25 ppm |

The synthetic step described in this EXAMPLE comprises one or more of the following differences compared to conventional syntheses of endoxifen: (i) concentration of acetone was conducted at NLT 30° C., and eliminating the step of stirring the mixture at 50±5° C. for NLT 2 hours was surprisingly found to reduce and/or prevent the generation of mesityl oxide; (ii) following the acetone/2-propanol crystallization, a THF/2-propanol crystallization was added in the purification process to reduce and/or remove mesityl oxide; (iii) in order to have a stable crystallization rate of (E)-Endoxifen in EtOAc crystallization step, the filtrate is concentrated until the volume reached 13 vol, followed by addition of 7 vol. of Ethyl acetate and concentration until the volume reached again 13 vol.; and (iv) in order to create (E)-Endoxifen seed in the EtOAc crystallization and to thereby speed up (E)-Endoxifen precipitation, the reaction mixture is cooled to NMT 5° C. first to produce seed prior to heating, and heating at reflux is changed to 50±5° C. to prevent increase generation of impurity at higher temperature.

Furthermore, it was demonstrated that the herein described processes using an ethyl acetate crystallization to enrich the mother liquor with (Z)-endoxifen followed by acetone/IPA and THF/IPA recrystallizations to furnish highly pure (Z)-endoxifen are superior to conventional processes using, e.g., (multiple) acetone recrystallization(s) only. TABLES 2 and 3 below show that (Z)-endoxifen produced from methods using acetone recrystallization only is unstable:

TABLE 2
Stability of (Z)-Endoxifen in acetone at 40° C.

| | | | | | | | | | | | | | |
|---|---|---|---|---|---|---|---|---|---|---|---|---|---|
| | | | | | 15.40 | 16.25 | | | | | | | |
| | RT | 3.14 | 4.25 | 14.68 | E-form | Z-form | 17.69 | 20.47 | 20.80 | 21.56 | 21.83 | 22.68 | 22.76 |
| Time | RRT | 0.19 | 0.26 | 0.90 | 0.95 | 1.00 | 1.09 | 1.26 | 1.28 | 1.33 | 1.34 | 1.39 | 1.40 |

| | | | | | | | | | | | | |
|---|---|---|---|---|---|---|---|---|---|---|---|---|
| T₀ | 0.16 | 0.12 | 0.04 | 0.65 | 98.28 | 0.08 | 0.03 | 0.07 | 0.08 | 0.07 | 0.03 | 0.06 |
| 4 hr | 0.15 | 0.57 | 0.04 | 0.78 | 97.28 | 0.08 | 0.04 | 0.15 | 0.08 | 0.10 | 0.10 | 0.08 |
| 20 hr | 0.11 | 2.00 | 0.04 | 1.03 | 95.35 | 0.08 | 0.05 | 0.21 | 0.08 | 0.08 | 0.09 | 0.01 |
| 24 hr | 0.13 | 2.81 | 0.04 | 1.14 | 94.73 | 0.09 | 0.05 | 0.17 | 0.09 | 0.01 | 0.07 | 0.02 |
| 28 hr | 0.11 | 3.14 | 0.04 | 1.26 | 93.99 | 0.1 | 0.06 | 0.19 | 0.09 | 0.01 | 0.09 | 0.02 |

TABLE 3
Stability of (Z)-Endoxifen in acetone at reflux (56° C.)

| | | | | | | | | | | | | | |
|---|---|---|---|---|---|---|---|---|---|---|---|---|---|
| | | | | 14.58 | 15.13 | | | | | | | | |
| | RT | 4.37 | 13.84 | E-form | Z-form | 20.32 | 20.51 | 21.68 | 22.88 | 27.15 | 27.21 | 27.45 | 27.88 |
| Time | RRT | 0.28 | 0.91 | 0.96 | 1.00 | 1.34 | 1.35 | 1.43 | 1.51 | 1.79 | 1.80 | 1.81 | 1.84 |

| | | | | | | | | | | | | |
|---|---|---|---|---|---|---|---|---|---|---|---|---|
| T₀ | 0.05 | 0.04 | 0.30 | 98.67 | 0.04 | 0.12 | 0.15 | 0.03 | 0.03 | 0.05 | 0.17 | 0.04 |
| 3 hr | 0.96 | 0.05 | 0.54 | 96.67 | 0.37 | 0.06 | 0.16 | 0.13 | 0.03 | 0.05 | 0.24 | 0.04 |
| 19 hr | 5.74 | 0.04 | 1.01 | 91.92 | 0.12 | 0.04 | 0.14 | 0.09 | 0.02 | 0.04 | 0.22 | 0.04 |
| 27 hr | 7.93 | 0.04 | 1.53 | 89.05 | 0.09 | 0.10 | 0.15 | 0.06 | 0.03 | 0.04 | 0.22 | 0.04 |
| 43 hr | 12.64 | 0.04 | 5.33 | 80.36 | 0.13 | 0.05 | 0.13 | 0.05 | 0.04 | 0.05 | 0.15 | 0.04 |

RT: Retention time (min)
RRT: Relative retention time

### Example 4

### Oral Compositions of (Z)-Endoxifen

This example describes oral compositions of (Z)-endoxifen.

≥95% (Z)-endoxifen free base was prepared as described herein in EXAMPLE 3. White to beige powder is formulated as stable free-flowing powder and filled neat into a capsule as a Drug-in-Capsule (DIC, also called API-in-capsule, AIC) oral solid dosage form. 1 mg, 2 mg, 4 mg, 10 mg, 40 mg, and 80 mg of endoxifen which was at least 95% (Z)-endoxifen was filled neat in an enteric resistant delayed release Hypromellose capsule. (Z)-endoxifen free base in the enteric resistant delayed release Hypromellose capsule is released in predominantly in the intestines (upper GI and colon) and is protected from the acidic environment in the stomach.

### Example 5

### Recrystallization of (Z)-Endoxifen to Yield Form I

This example describes crystallization conditions for generating Form I of (Z)-endoxifen (Formula (IV)), as well as physical characterizations of this polymorphic form. A (Z)-endoxifen free base composition with ≥95% enantiomeric purity was prepared as described in EXAMPLE 3. Form I was then generated in a high temperature acetonitrile slurry containing 100 mg/ml (Z)-endoxifen.

### XRPD Characterization

Polymorphic Form I exhibited an x-ray powder diffraction (XRPD) pattern shown in FIG. 9 with peaks 1-46 corresponding to the peak numbers (No.) in TABLE 4. Peaks in FIG. 9 and TABLE 4 are provided as relative intensities (Rel. Int.) standardized against the peak at 4.7° 2θ (the peak with the highest intensity). The ten largest peaks by measure of Rel. Int. identified in the XRPD of Form I, were at 21.8, 17.2, 24.2, 16.9, 21.4, 20.9, 14.2, 18.2, 26.9, and 25.4° 2θ, corresponding to peak numbers (No.) 17, 9, 20, 8, 16, 15, 5, 10, 24, 21, respectively. The three largest peaks by measure of Rel. Int. were at 21.8, 17.2, and 24.4° 2θ. Form I was isolated from the majority of recrystallization experiments performed during the polymorph crystallization experiments suggesting its stable nature when compared to other polymorphic forms of (Z)-endoxifen.

TABLE 4
X-Ray Powder Diffraction Peak Table for Form I

| | Pos. | d-spacing | Rel. Int. | Height | Backgr. |
|---|---|---|---|---|---|
| No. | (°2Th.) | (Å) | (%) | (cts) | (cts) |
| 1 | 7.1477 | 12.36765 | 13.28 | 896.63 | 211 |
| 2 | 9.3456 | 9.46337 | 18.47 | 1246.95 | 241 |
| 3 | 12.4014 | 7.13753 | 11.55 | 779.43 | 367.14 |
| 4 | 12.872 | 6.87762 | 2.48 | 167.67 | 415.2 |
| 5 | 14.2157 | 6.23044 | 43.31 | 2923.98 | 601.57 |
| 6 | 15.2345 | 5.816 | 14.18 | 957.28 | 721.45 |
| 7 | 16.0511 | 5.52188 | 9.22 | 622.18 | 791.11 |
| 8 | 16.8589 | 5.25909 | 46.11 | 3112.61 | 841.89 |
| 9 | 17.1664 | 5.16557 | 90.53 | 6111.27 | 857 |
| 10 | 18.2345 | 4.86532 | 37.34 | 2520.7 | 885 |
| 11 | 18.6207 | 4.76529 | 14.48 | 977.8 | 887 |
| 12 | 18.8986 | 4.69582 | 17.01 | 1148.36 | 884.14 |
| 13 | 19.9551 | 4.44954 | 7.23 | 487.8 | 895 |
| 14 | 20.2973 | 4.37529 | 4.07 | 274.63 | 903 |
| 15 | 20.899 | 4.25066 | 45.73 | 3087.1 | 908 |
| 16 | 21.3655 | 4.1589 | 46.07 | 3110.3 | 906 |
| 17 | 21.7978 | 4.07739 | 100 | 6750.74 | 898.22 |
| 18 | 22.5716 | 3.93932 | 15.33 | 1034.79 | 868 |
| 19 | 23.2575 | 3.82467 | 30.32 | 2046.56 | 825 |
| 20 | 24.3974 | 3.64849 | 54.73 | 3694.8 | 784 |
| 21 | 25.356 | 3.5127 | 31.52 | 2127.78 | 767 |
| 22 | 25.8183 | 3.45084 | 11.59 | 782.23 | 750.17 |
| 23 | 26.5611 | 3.35599 | 23.56 | 1590.7 | 711 |
| 24 | 26.9179 | 3.31231 | 36.9 | 2491.19 | 686 |
| 25 | 28.0567 | 3.1804 | 20.97 | 1415.7 | 596 |
| 26 | 28.5534 | 3.1262 | 4.85 | 327.39 | 555.66 |
| 27 | 29.0311 | 3.07584 | 17.68 | 1193.37 | 511.89 |
| 28 | 29.7352 | 3.00459 | 1.21 | 81.71 | 464 |
| 29 | 30.0504 | 2.97379 | 2.08 | 140.74 | 458.96 |
| 30 | 30.79 | 2.90402 | 5.32 | 359 | 438 |
| 31 | 31.3621 | 2.85235 | 9.98 | 673.71 | 423.79 |
| 32 | 31.7008 | 2.82264 | 7.44 | 502.49 | 414 |
| 33 | 31.9692 | 2.79955 | 4.31 | 290.99 | 405 |
| 34 | 32.7255 | 2.73655 | 6.41 | 432.65 | 367 |
| 35 | 33.8097 | 2.65124 | 1.03 | 69.64 | 355 |
| 36 | 34.27 | 2.61668 | 1.93 | 130 | 367 |
| 37 | 35.13 | 2.55457 | 6.09 | 411 | 372 |
| 38 | 35.65 | 2.51849 | 2.04 | 138 | 365 |
| 39 | 35.8906 | 2.50216 | 2.87 | 193.81 | 359 |
| 40 | 36.3592 | 2.47098 | 1.43 | 96.21 | 351 |
| 41 | 36.78 | 2.44367 | 5.47 | 369 | 355 |
| 42 | 37.38 | 2.40581 | 5.42 | 366 | 354 |
| 43 | 37.73 | 2.3843 | 6.38 | 431 | 348 |
| 44 | 38.3486 | 2.34725 | 1.44 | 97.16 | 326.14 |
| 45 | 38.6094 | 2.33199 | 1.41 | 94.88 | 315 |
| 46 | 39.31 | 2.29203 | 4.87 | 329 | 318 |

### Stability Analysis

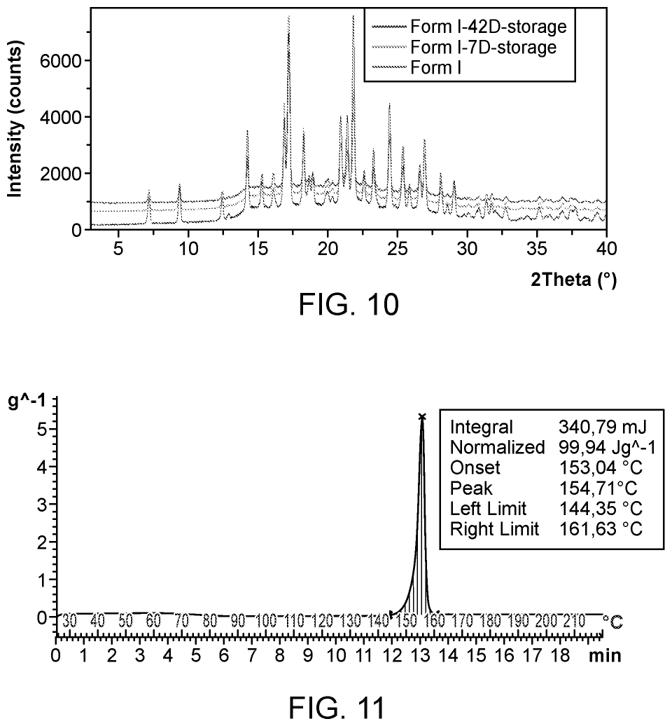

Form I samples were collected for XR-PD analysis following 0, 7, and 42 days of storage, the results of which are shown in FIG. 10. In this figure, XR-PD following 0 days (Form I), 7 days (Form J-71D-storage), and 42 days (Form J-42D-storage) of storage are displayed. Minimal changes were observed in the XR-PD time course, indicating that polymorphic Form I is stable under ambient conditions.

### DSC Characterization

FIG. 11 provides a DSC profile of Form I performed with a scanning rate of 10K/min. Form I DSC exhibits an endothermic peak at 155° C. (onset: 153° C., Delta H: approx. 99.9 J/g) likely due to Form I melting. No other features were discernible in the DSC profile, suggesting minimal solvent release from Form I upon heating.

### TGA and EGA Analyses

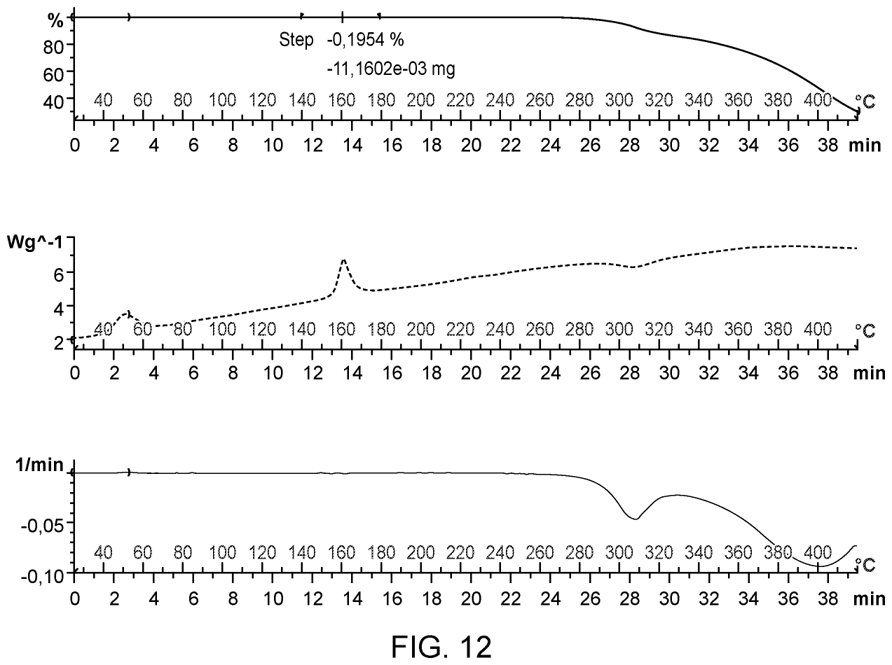

The TGA profile of Form I is shown in the top panel of FIG. 12 with the heat-flow profile in the middle panel of FIG. 12, and the first derivative of the TGA profile in the bottom pane of FIG. 12. The TGA profile of Form I in FIG. 12 showed no weight loss between 25° C.-140° C. A weight loss of 0.2% was detected between 140° C. and 180° C., potentially ascribable to solvent release. Degradation of Form I took place above 270° C. The heat-flow profile included two main peaks around 50° C. and 160° C. The first derivative of the TGA profile indicated a greatest decline in the TGA profile occurred around 400° C., and that weight loss occurred around 320° C.

### Example 6

### Recrystallization of (Z)-Endoxifen to Yield Polymorphic Form IV

This example describes multiple crystallization conditions for generating Form IV of (Z)-endoxifen (Formula (IV)), as well as physical characterizations of this form. A (Z)-endoxifen free base composition with ≥95% enantiomeric purity was prepared as described herein in EXAMPLE 3 recrystallization. Some solutions in the following example were precipitated by cooling of the solution from a temperature between 50-100° C. to between 10-25° C. at a rate ranging from −0.2 to −0.5° C./minute resulting in a precipitate.

### Form IV Recrystallization (1)-2-Propanol Evaporation

Form IV was generated by dissolving (Z)-endoxifen powder in 2-propanol at a concentration of 10 mg/ml. The solution was concentrated through room temperature evaporation of the 2-propanol, yielding the crystalline solid of polymorphic Form IV.

### Form IV Recrystallization (2)-2-Propanol THF Precipitation

In a further recrystallization, Form IV was generated by dissolving (Z)-endoxifen powder in 2-propanol/THF 95/5 (v/v) solvent system at a concentration of 40 mg/ml. The solution was then cooled yielding the crystalline solid of polymorphic Form IV.

### Form IV Recrystallization (3)-2-Propanol/Heptane

In a separate recrystallization, Form IV was obtained by dissolving (Z)-endoxifen powder in a heptane/2-propanol 50/50 (v/v) solvent system at a concentration of 40 mg/ml. The solution was then cooled, yielding the crystalline solid of polymorphic Form IV.

### Form IV Recrystallization (4)-2-Propanol/Ethyl Acetate

In a further recrystallization, Form IV was generated by dissolving (Z)-endoxifen powder in an ethyl acetate/2-propanol 50/50 (v/v) solvent system at a concentration of 100 mg/ml. The solution was then cooled, yielding the crystalline solid of polymorphic Form IV.

### Form IV Recrystallization (5)-Antisolvent-Driven Crystallization In THF

A further recrystallization was performed by precipitating from a saturated THF solution by slow addition of water, yielding the crystalline solid of polymorphic Form IV.

### Form IV Recrystallization (6)-Water Bath Suspension

In a further recrystallization utilizing a THF solvent/water antisolvent system, Form IV was obtained by adding a (Z)-endoxifen saturated THF solution to water, precipitating the crystalline solid of polymorphic Form IV.

### Form IV Recrystallization (7)-2-Propanol THF Slurry

Form IV was also isolated from a room-temperature slurry of amorphous (Z)-endoxifen. Amorphous (Z)-endoxifen was dissolved in a 2-propanol/THF 63/37 (v/v) solvent system, and then evaporated at room temperature to yield the crystalline solid of polymorphic Form IV.

### Form IV Recrystallization (8)-Acetone Evaporation

Polymorphic Form IV was also generated through recrystallization in acetone. For this recrystallization, (Z)-endoxifen powder was acetone at a concentration of 10 mg/ml. The solution was then evaporated yielding a crystalline solid composed primarily of polymorphic Form IV and minor traces of polymorphic Form I.

### Form IV Recrystallization (9)-Acetone THF Evaporation

Polymorphic Form IV was also isolated by dissolving (Z)-endoxifen powder in an acetone/THF 60/40 (v/v) solvent system at a concentration of 40 mg/ml. The solution was then evaporated at room temperature yielding a crystalline solid composed primarily of polymorphic Form IV and minor traces of polymorphic Form I.

### Recrystallizations Which Generated Form IV as a Minor Component

A number of crystallizations disclosed herein generate a minor amount of Form IV of (Z)-endoxifen.

A minor amount of polymorphic Form IV was generated during a 2-Propanol recrystallization which primarily yielded Form I. In this recrystallization, (Z)-endoxifen was dissolved in 2-Propanol at a concentration of 20 mg/ml. The solution was then precipitated by slow cooling, yielding the crystalline solid of polymorphic Form I and with minor traces of polymorphic Form IV.

A minor amount of polymorphic Form IV was also observed in an amorphous (Z)-endoxifen composition produced during crystallization in a cooled acetone/2-propanol system. In this recrystallization, (Z)-endoxifen powder was dissolved in an acetone/2-Propanol 67/33 (v/v) solvent system at a concentration of 40 mg/ml. The solution was slowly cooled to −20° C. and incubated for 1 day, yielding amorphous (Z)-endoxifen and with minor traces of polymorphic Form IV, as evidenced by an XRPD peak at 4.8° 20.

Trace polymorphic Form IV was generated from a high temperature (50° C.-100° C.) heptane/2-Propanol 50/50 (v/v) slurry prepared with 100 mg/ml (Z)-endoxifen in a high temperature slurry yielding the crystalline solid of polymorphic Form I with minor traces of polymorphic Form IV, as evidenced by an XRPD peak at 4.8° 20.

### XRPD Characterization

Polymorphic Form IV exhibited an x-ray powder diffraction (XRPD) pattern shown in FIG. 13 with peaks 1-27 corresponding to the peak numbers (No.) in TABLE 5. Peaks in FIG. 13 and TABLE 5 are provided as relative intensities (Rel. Int.) standardized against the peak at 4.7° 2θ (the peak with the highest intensity). The ten largest peaks by measure of relative intensity (Rel. Int.) identified in the XRPD of Form IV, were at 4.7, 23.3, 13.6, 23.8, 14.2, 22.5, 15.7, 7.1, 20.2, and 9.5° 20, corresponding to peak numbers (No.) 1, 22, 7, 23, 8, 21, 9, 3, 17, and 5, respectively. The three largest peaks by measure of Rel. Int. were at 4.7, 23.3, and 13.6° 2θ.

TABLE 5
X-Ray Powder Diffraction Peak Table for Form IV

| | Pos. | | Rel. | Height | Backgr. |
|---|---|---|---|---|---|
| No. | (°2θ) | d-spacing (Å) | Int. (%) | (cts]) | (cts) |
| 1 | 4.7426 | 18.63286 | 100 | 7538.64 | 192.26 |
| 2 | 6.6743 | 13.24369 | 9.36 | 705.4 | 183 |
| 3 | 7.0843 | 12.47821 | 11.36 | 856.3 | 188 |
| 4 | 8.18 | 10.80904 | 0.99 | 75 | 200 |
| 5 | 9.483 | 9.32657 | 9.4 | 708.84 | 215.3 |
| 6 | 11.6054 | 7.62528 | 4.27 | 321.8 | 257 |
| 7 | 13.6081 | 6.50723 | 15.08 | 1136.59 | 440.81 |
| 8 | 14.2405 | 6.21965 | 12.36 | 931.95 | 510.05 |
| 9 | 15.7357 | 5.63186 | 11.76 | 886.73 | 620 |
| 10 | 15.9064 | 5.57178 | 4.51 | 339.86 | 628.64 |
| 11 | 16.0882 | 5.50923 | 7.08 | 533.7 | 636 |
| 12 | 16.3919 | 5.40784 | 1.92 | 145.05 | 646 |
| 13 | 18.22 | 4.86917 | 5.57 | 420 | 711 |
| 14 | 19.0036 | 4.67013 | 6.52 | 491.7 | 746 |
| 15 | 19.5559 | 4.53945 | 6.41 | 483.57 | 758.59 |
| 16 | 19.7095 | 4.50442 | 3.52 | 265.72 | 761 |
| 17 | 20.1613 | 4.4045 | 10.3 | 776.15 | 761 |
| 18 | 20.596 | 4.31251 | 3.23 | 243.59 | 754.4 |
| 19 | 21.3322 | 4.16531 | 4.51 | 340.25 | 743 |
| 20 | 22.2517 | 3.99523 | 3.71 | 279.45 | 711 |
| 21 | 22.4576 | 3.95906 | 11.85 | 893.15 | 699.24 |
| 22 | 23.306 | 3.81682 | 15.29 | 1152.43 | 637.4 |
| 23 | 23.8251 | 3.73483 | 14.21 | 1071.5 | 586.49 |
| 24 | 25.34 | 3.51488 | 2.73 | 206 | 510 |
| 25 | 26.1808 | 3.40387 | 1.41 | 106.63 | 486 |
| 26 | 28.3295 | 3.1504 | 2.82 | 212.22 | 349.05 |
| 27 | 29.5451 | 3.02349 | 5.2 | 391.96 | 274.49 |

### Stability Analysis

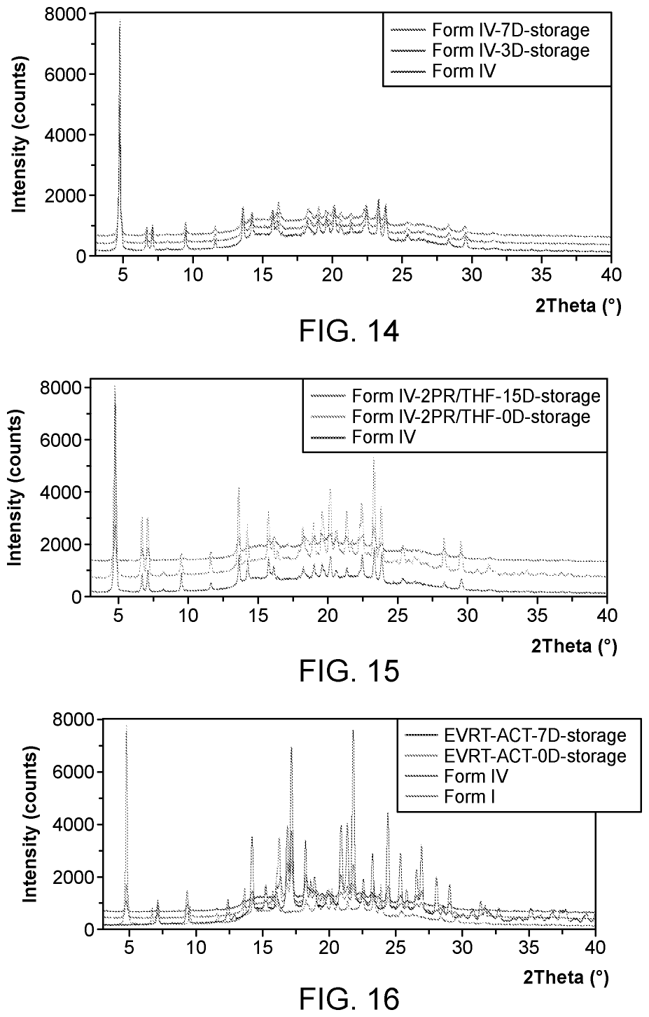

The stability of polymorphic Form IV was evaluated over 7 days of storage at room temperature in a sealed vial. Portions of the Form IV samples were collected for XRPD analysis following 0 (Form IV), 3 (Form IV-3D3-storage), and 7 days (Form IV-7D3-storage) of storage, the results of which are shown in FIG. 14. Minimal changes were observed in the XRPD time course, indicating that polymorphic Form IV is stable under ambient conditions.

The stability of polymorphic Form IV was also evaluated following crystallization from a room temperature 2-propanol/THF 95/5 (v/v) solvent system containing 40 mg/ml (Z)-endoxifen. XPRD was collected immediately after recovery and following 15 days storage at room temperature in a sealed vial. FIG. 15 provides the resultant XRPD patterns following 15 days storage (Form IV-2PR/THF-15D-storage) and 0 days storage (Form IV-2PR/THF-0D-storage), as well as an XRPD spectrum of Form IV (Form IV) obtained from 2-propanol recrystallization. While the sample maintained a Form IV XRPD pattern following 15 days storage, its XRPD spectrum also exhibited a new signal at 16.1° 2θ.

Since the room temperature evaporation in acetone yielded a mixture of polymorphic Form I and Form IV XRPD patterns with a very intense unassigned signal at 16.1° 2θ, the sample was re-measured following 7 days storage in a sealed vial. From top to bottom, FIG. 16 provides XRPD patterns of the acetone-generated polymorphs following 7 (EVRT-ACT-7D-storage) and 0 days of storage (EVRT-ACT-0D-storage), a Form IV spectrum (Form IV), and a Form I (Form I) spectrum, respectively. No significant changes were detected in the acetone-recrystallized generated samples, indicating ambient stabilities of Forms I and IV polymorphic mixture.

### DSC Characterization

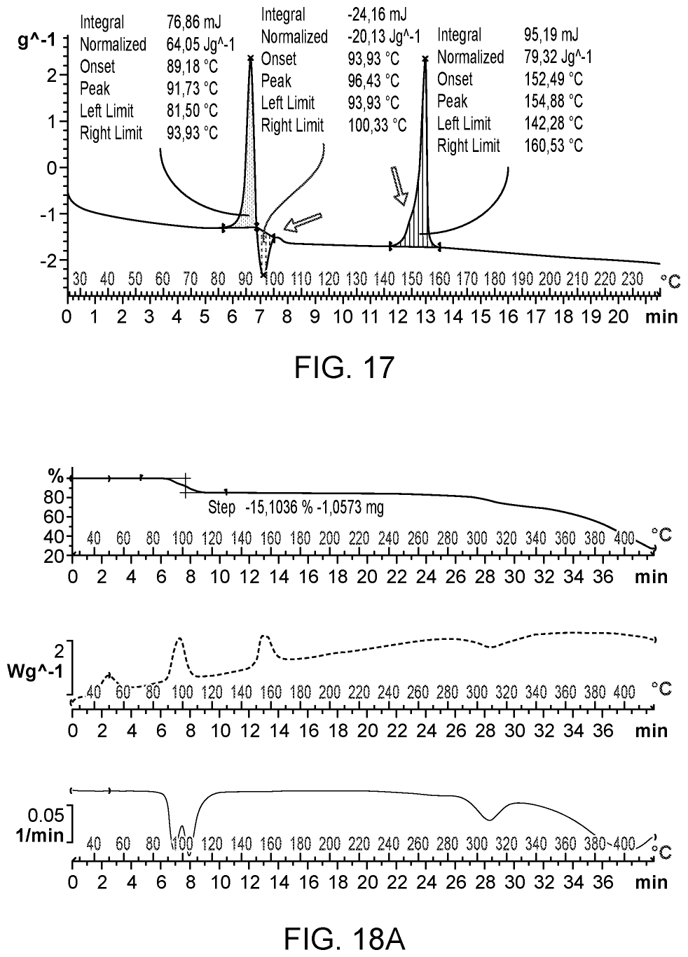

A DSC profile of polymorphic Form IV is shown in FIG. 17. Form IV exhibited an endothermic peak at 92° C. (onset at 89° C.) ascribable to solvent release, an exothermic peak at 96° C. (onset at 94° C.) ascribable to a recrystallization process, and an endothermic peak at 155° C. (onset at 153° C.) ascribable to Form I melting. The mild hump in the 155° C. endothermic peak may be due to trace Form VII melting.

### TGA and EGA Analyses

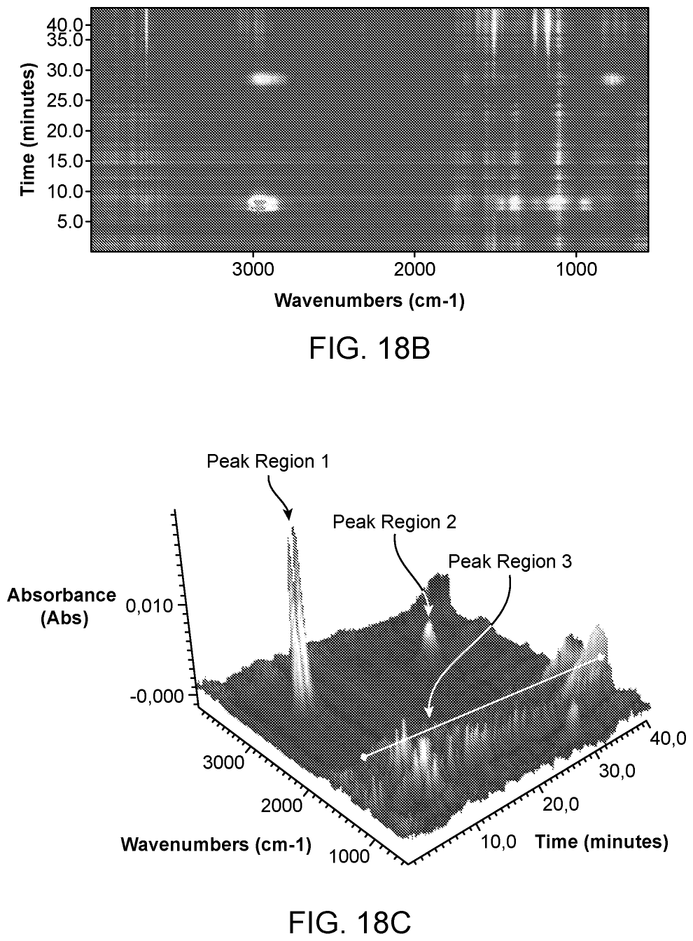

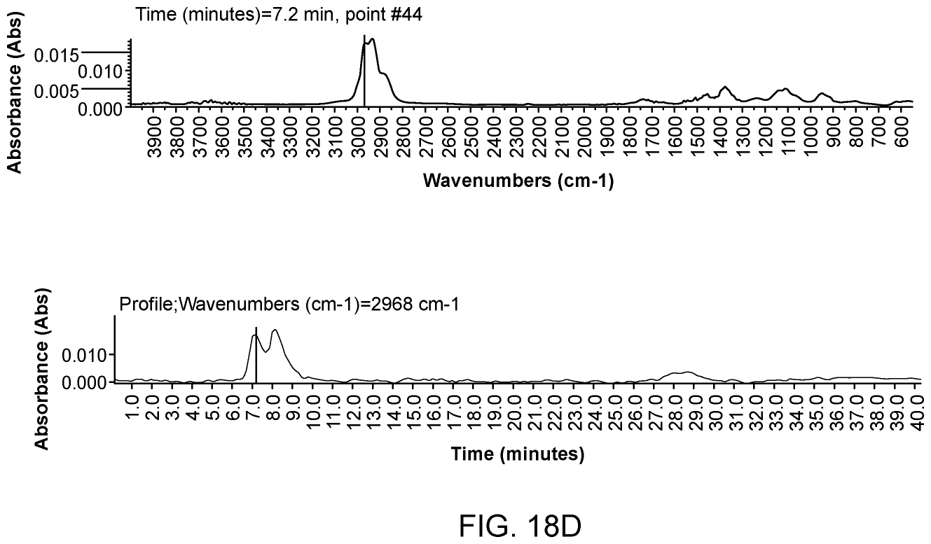

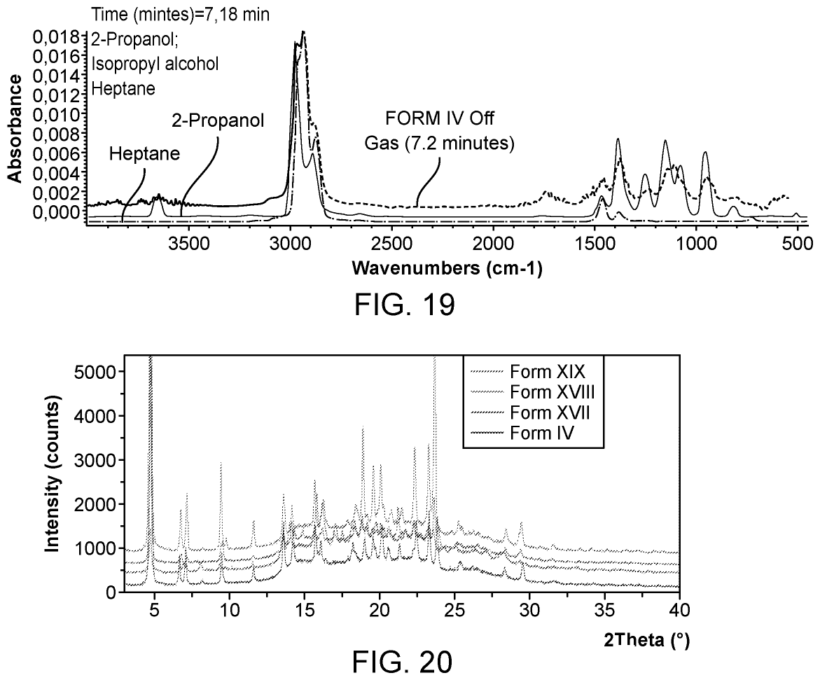

TGA and EGA analyses of polymorphic Form IV are shown in FIG. 18 A-FIG. 18D. FIG. 18A summarizes TGA (top), heat flow (middle), and 1^{st} derivative (bottom) profiles of Form IV. The TGA analyses exhibited two consecutive weight losses totaling 15.1% between 70° C. and 130° C., corresponding to 2-propanol and heptane release, respectively. Degradation of polymorphic Form IV occurred above 270° C. These assignments were confirmed by 2D and 3D gas evolution analysis shown in FIG. 18B and FIG. 18C. FIG. 18B shows the 2D gas evolution analysis of Form IV, specifically showing a strong absorbance at around 3000 wavenumbers (cm⁻¹) and approximately 7 minutes. The absorbance in wavenumbers (cm⁻¹) over time is also represented in 3D in FIG. 18C again showing the large absorbance peak at around 3000 wavenumbers (cm⁻¹) and 7 minutes (Peak Region 1); an absorbance peak at around 3000 wavenumbers (cm⁻¹) and 28 minutes (Peak Region 2); and several other absorbance peaks including peaks from 0-40 minutes in the range of 100-2000 wavenumbers (cm⁻¹) (Peak Region 3). The kinetic profiles in FIG. 18D profile the spectra of the gas evolution at approximately 7 minutes (FIG. 18D, top), and the absorbance over time at 2908 wavenumbers (cm⁻¹) (FIG. 18D, bottom). Similarly, to the 2D and 3D gas evolution analysis, the kinetic profiles in FIG. 18D exhibit that the gas evolution at approximately 7 minutes has a strong absorbance at around 2970 wavenumbers (cm⁻¹) (FIG. 18D, top), and there are multiple absorbance peaks at 2908 wavenumbers (cm⁻¹) over time, notably around approximately 7, 8.4, and 28.5 minutes (FIG. 18D, bottom). The gases evolved during the EGA at approximately 7 minutes were identified by FT-IR analysis (FIG. 19) comparing the FT-IR spectra of the gas evolved at approximately 7 minutes in the EGA experiment and to the reference FT-IR spectra of 2-propanol and heptane. Specifically, the EGA sample at approximately 7 minutes shows similar peaks to heptane at around 2800-3000 wavenumbers (cm⁻¹) and 1350-1500 wavenumbers (cm⁻¹) and to 2-propanol at around 2800-3000 wavenumbers (cm⁻¹) and in the region of 800-1500 2800-3000 wavenumbers (cm⁻¹).

### Example 7

Polymorphs of (Z)-Endoxifen with XRPD Patterns Similar to That of Form IV

This example covers the production, isolation, and characterization of Forms XVII, XVIII, and XIX. These forms have XRPD patterns which are similar to that of Form IV and appear to differ primarily by solvent content. XRPD patterns of each of these forms is shown in FIG. 20 and show the similarity between their XRPD patterns and that of Form IV.

### Isolation of Form XVII

Form XVII was isolated by slowly evaporating a 10 mg/ml ethanol solution of (Z)-endoxifen. XRPD of the resultant Form XVII crystals is shown in FIG. 21.

### Stability of Form XVII

Form XVII was further assessed after 3 days storage in a sealed vial at room temperature. As shown in FIG. 22, a complete conversion of Form XVII (Form XVII) into Form I (Form I) was observed in the XRPD spectrum of Form XVII after 3 days of storage (Form XVII-3D-storage), along with the formation of an unassigned peak at 8.2° 20 (top line).

### Isolation of Form XVIII

Form XVIII was obtained by slowly evaporating a 10 mg/ml THF solution of (Z)-endoxifen at room temperature. XRPD of Form XVIII is shown in FIG. 23.

### Stability of Form XVIII

Form XVIII was further reassessed by XRPD following 3 and 7 days storage at room temperature in a sealed vial. The data from these analyses are summarized in FIG. 24, which (from top to bottom) provides the sample following 7 days storage (Form XVIII-7D-storage), the sample following 3 days storage (Form XVIII-3D-storage), and the sample immediately following crystallization (Form XVIII). As shown in this figure, Form XVIII underwent partial amorphization over 7 days of storage.

### Isolation of Form XIX

Form XIX crystals were obtained through multiple heptane crystallizations. In a first recrystallization, Form XIX was obtained following 1 day of −20° C. incubation of (Z)-endoxifen-saturated acetone added to heptane. A similar Form XIX crystallization was performed by adding heptane to (Z)-endoxifen saturated acetone and incubating for 1 day at −20° C. Form XIX was also generated through heptane addition to (Z)-endoxifen-saturated THF, and through (Z)-endoxifen-saturated THE addition to heptane.

### XRPD Characterization of Form XIX

Form XIX exhibited the XRPD pattern shown in FIG. 25. with peaks 1-39 corresponding to the peak numbers (No.) in TABLE 6. Peaks in FIG. 25 and TABLE 6 are provided as relative intensities (Rel. Int.) standardized against the peak at 4.7° 2θ (the peak with the highest intensity). The ten largest peaks by measure of Rel. Int. identified in the XRPD of Form XIX, were at 4.7, 23.6, 18.9, 9.4, 23.3, 22.3, 20.1, 19.6, 7.1, and 15.7° 2θ, corresponding to peak numbers (No.) 1, 22, 13, 4, 21, 20, 16, 15, 3, and 9, respectively. The three largest peaks by measure of Rel. Int. were at 4.7, 23.6, and 18.9° 2θ.

TABLE 6
X-Ray Powder Diffraction Peak Table for Form XIX

| | Pos. | | Rel. | | Backgr. |
|---|---|---|---|---|---|
| No. | (°2Th.) | d-spacing | Int. (%) | Height (cts) | (cts) |
| 1 | 4.725 | 18.70221 | 100 | 15823.52 | 219 |
| 2 | 6.7405 | 13.1138 | 5.88 | 930.9 | 215 |
| 3 | 7.1434 | 12.37515 | 8.14 | 1288.8 | 216 |
| 4 | 9.4223 | 9.38652 | 12.23 | 1935.69 | 231 |
| 5 | 9.7719 | 9.05149 | 1.58 | 250.37 | 231 |
| 6 | 11.5822 | 7.64047 | 3.68 | 582.46 | 283 |
| 7 | 13.5873 | 6.51715 | 6.39 | 1011.6 | 478.45 |
| 8 | 14.1388 | 6.26414 | 4.32 | 683.41 | 546.88 |
| 9 | 15.67 | 5.65531 | 7.21 | 1140.64 | 681 |
| 10 | 16.2462 | 5.45602 | 4.04 | 639.36 | 707.62 |
| 11 | 17.8438 | 4.97096 | 0.79 | 125.71 | 757.38 |
| 12 | 18.3914 | 4.82418 | 2.74 | 433.04 | 798 |
| 13 | 18.8781 | 4.70089 | 13.57 | 2146.99 | 823 |
| 14 | 19.1656 | 4.63101 | 1.17 | 184.82 | 834 |
| 15 | 19.5633 | 4.53775 | 8.2 | 1298.08 | 843 |
| 16 | 20.0566 | 4.42724 | 8.23 | 1302.49 | 847 |
| 17 | 20.2342 | 4.3888 | 2.58 | 408.5 | 847 |
| 18 | 20.7525 | 4.28033 | 1.65 | 261.77 | 841 |
| 19 | 21.459 | 4.14099 | 2.28 | 361.12 | 821.1 |
| 20 | 22.3216 | 3.98287 | 11.25 | 1780.22 | 780.84 |
| 21 | 23.2503 | 3.82584 | 12.01 | 1900.49 | 706.97 |
| 22 | 23.6488 | 3.76226 | 31.64 | 5007.18 | 666.12 |
| 23 | 24.7846 | 3.59237 | 0.46 | 73.06 | 584 |
| 24 | 25.2359 | 3.52914 | 1.77 | 279.57 | 578 |
| 25 | 25.4567 | 3.49903 | 1.25 | 198 | 573 |
| 26 | 26.2232 | 3.39847 | 0.75 | 119.12 | 538 |
| 27 | 26.5818 | 3.35343 | 0.55 | 86.68 | 512.82 |
| 28 | 27.3067 | 3.26603 | 0.34 | 54.24 | 452.33 |
| 29 | 28.4035 | 3.14236 | 1.89 | 298.81 | 381.65 |
| 30 | 29.435 | 3.03455 | 3.29 | 520.73 | 323 |
| 31 | 30.6588 | 2.91615 | 0.33 | 52.85 | 259 |
| 32 | 31.5598 | 2.83493 | 0.63 | 99.95 | 232 |
| 33 | 33.3055 | 2.69022 | 0.33 | 51.75 | 201.45 |
| 34 | 34.0734 | 2.63132 | 0.38 | 59.56 | 194 |
| 35 | 35.1636 | 2.55221 | 0.33 | 52.85 | 179 |
| 36 | 36.4223 | 2.46684 | 0.22 | 34.93 | 177 |
| 37 | 37.2204 | 2.41576 | 0.14 | 21.82 | 179 |
| 38 | 38.2505 | 2.35304 | 0.28 | 43.76 | 165 |
| 39 | 38.8729 | 2.31679 | 0.18 | 27.81 | 158 |

### Stability of Form XIX

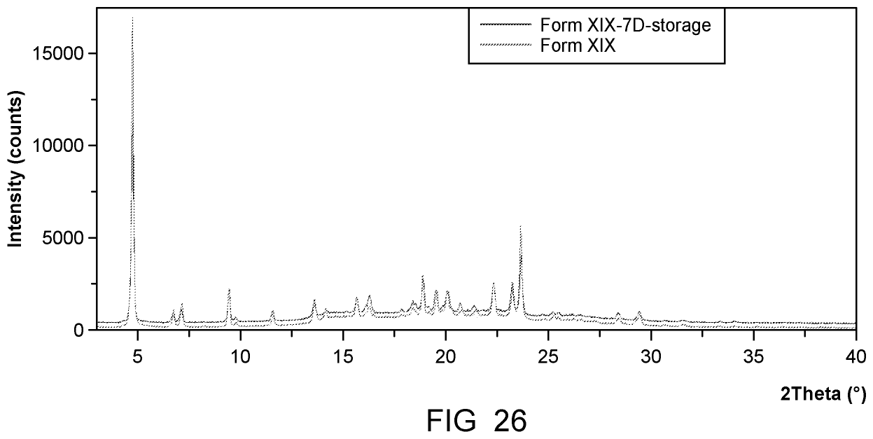

Form XIX room temperature stability was evaluated with XRPD. XRPD of a Form XIX sample immediately following crystallization (Form XIX) and following 7 days of storage (Form XIX-7D-storage) are shown in FIG. 26. No significant difference was observed between the two spectra, suggesting that polymorphic Form XIX is stable under ambient conditions.

### Example 8

### Recrystallization of (Z)-Endoxifen to Yield Polymorphic Form V

This example describes recrystallization of (Z)-endoxifen (Formula (IV)) to yield a polymorphic Form V of (Z)-endoxifen (Formula (IV)), as well as physical characterizations of this form. A (Z)-endoxifen free base composition with ≥95% enantiomeric purity was prepared as described herein in EXAMPLE 3. Some solutions in the following example were precipitated by cooling of the solution from a temperature between 50-100° C. to between 10-25° C. at a rate ranging from −0.2 to −0.5° C./minute resulting in a precipitate.

### Form V Recrystallization (I)-Acetonitrile

To obtain crystals, a 20 mg/ml acetonitrile solution of (Z)-endoxifen was precipitated during slow cooling, yielding a crystalline solid termed polymorphic Form V.

### Precipitation Which Generated Form V as a Minor Component

Form V was also obtained in a mixture with Forms I and VI during a room temperature evaporation of acetonitrile containing 10 mg/ml (Z)-endoxifen. The resultant mixture of forms contained polymorphic Form V, Form I, and Form VI.

### XRPD

Polymorphic Form V exhibited an x-ray powder diffraction (XRPD) pattern shown in FIG. 27 with peaks 1-22 corresponding to the peak numbers (No.) in TABLE 7. Peak heights are provided as relative intensities (Rel. Int.), calculated by dividing each peak height (Height (cts)) by the largest peak height. The ten largest peaks by measure of Rel. Int. identified in the XRPD of Form V, were at 12.5, 19.6, 8.9, 21.7, 20.8, 19.8, 16.0, 22.0, 13.5, and 14.4° 2θ, corresponding to peak numbers (No.) 4, 11, 2, 14, 13, 12, 8, 15, 5, and 6, respectively. The three largest peaks by measure of Rel. Int. were at 12.5, 19.6, and 8.9° 2θ.

TABLE 7
X-Ray Powder Diffraction Peak Table for Form V

| | Pos. | d-spacing | Rel. | | Backgr. |
|---|---|---|---|---|---|
| No. | (°2Th.) | (Å) | Int. (%) | Height (cts) | (cts) |
| 1 | 7.4409 | 11.88091 | 12.3 | 105.46 | 174.99 |
| 2 | 8.938 | 9.89396 | 72.5 | 621.49 | 201.87 |
| 3 | 10.3252 | 8.56762 | 4.54 | 38.94 | 220.56 |
| 4 | 12.4519 | 7.10874 | 100 | 857.23 | 309.76 |
| 5 | 13.4847 | 6.56647 | 21.73 | 186.25 | 405.22 |
| 6 | 14.4218 | 6.14186 | 21.31 | 182.7 | 487.19 |
| 7 | 15.4895 | 5.72081 | 18.51 | 158.7 | 562.94 |
| 8 | 15.986 | 5.54424 | 28.98 | 248.46 | 591.95 |
| 9 | 17.8232 | 4.97666 | 11.9 | 102 | 665.59 |
| 10 | 18.7945 | 4.7216 | 16.56 | 141.99 | 683.85 |
| 11 | 19.5952 | 4.53043 | 99.12 | 849.65 | 686.87 |
| 12 | 19.8391 | 4.4753 | 34.73 | 297.72 | 685.64 |
| 13 | 20.7535 | 4.28014 | 36.35 | 311.64 | 674.18 |
| 14 | 21.6595 | 4.10311 | 36.45 | 312.47 | 649.85 |
| 15 | 22.0384 | 4.0334 | 24.36 | 208.82 | 635.35 |
| 16 | 22.971 | 3.87172 | 15.25 | 130.73 | 588.2 |
| 17 | 23.7642 | 3.74426 | 11.87 | 101.71 | 535.87 |
| 18 | 24.9912 | 3.56314 | 9.92 | 85.06 | 470.08 |
| 19 | 26.3691 | 3.38 | 11.69 | 100.21 | 433.58 |
| 20 | 28.3314 | 3.1502 | 3.35 | 28.68 | 329.59 |
| 21 | 29.2654 | 3.05174 | 3.95 | 33.88 | 273.06 |
| 22 | 30.8057 | 2.90258 | 6.2 | 53.11 | 223.71 |

### Stability Analyses

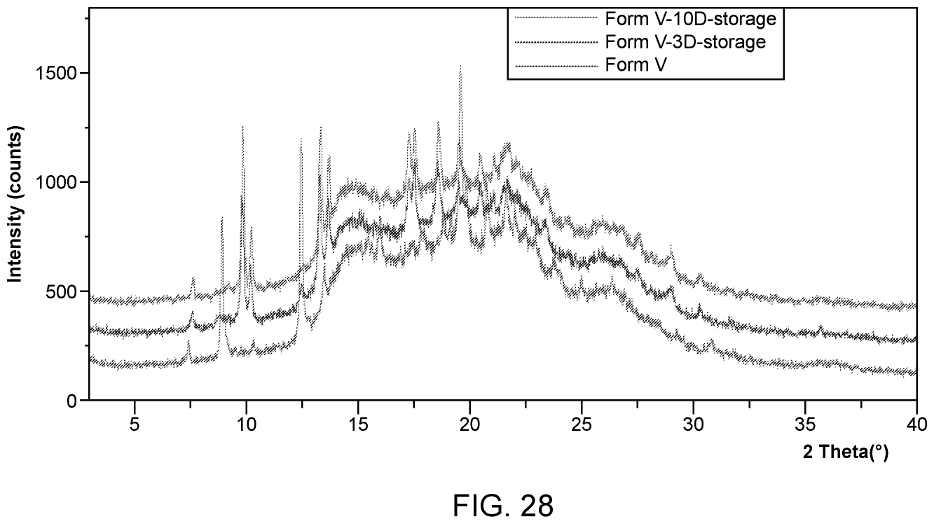

The stability of polymorphic Form V was over 10 days of storage at room temperature in a sealed vial. XR-PD was performed following 0, 3, and 10 days storage. The resulting XR-PD patterns are shown in FIG. 28 and show that Form V converted almost completely to Form VI after 3 days storage (Form V-3D3-storage), and completely to Form VI following 10 days storage (Form V-10D-storage).

### Example 9

### Purification of (Z)-Endoxifen to Yield Form VI

This example describes recrystallization of (Z)-endoxifen (Formula (IV)) to yield a polymorphic Form VI of (Z)-endoxifen, as well as physical characterizations of this polymorph. As described in EXAMPLE 8, Form V converted to Form VI during room temperature storage in a sealed vial, suggesting that Form VI might have a higher stability than Form V.

Additionally, traces of polymorphic Form VI were observed in a mixture of forms obtained through slow evaporation of an acetonitrile solution containing 10 mg/ml (Z)-endoxifen. This resultant mixture of forms was primarily composed of Forms I and V and contained traces of Form VI.

### XRPD Characterization

Polymorphic Form VI exhibited an x-ray powder diffraction (XRPD) pattern shown in FIG. 29 with peaks 1-22 corresponding to the peak numbers (No.) in TABLE 8. Peaks in FIG. 29 and TABLE 8 are provided as relative intensities (Rel. Int.) standardized against the peak at 9.9° 2θ (the peak with the highest intensity). The ten largest peaks by measure of Rel. Int. identified in the XRPD of Form VI, were at 9.9, 13.4, 13.7, 17.6, 18.6, 17.3, 21.8, 10.2, 19.5, and 14.2° 2θ, corresponding to peak numbers (No.) 2, 4, 5, 8, 9, 7, 13, 3, 10, and 6, respectively. The three largest peaks by measure of Rel. Int. were at 9.9, 13.4, and 13.7° 2θ.

TABLE 8
X-Ray Powder Diffraction Peak Table for Form VI

| | Pos. | | Rel. | | Backgr. |
|---|---|---|---|---|---|
| No. | (°2Th.) | d-spacing (Å) | Int. (%) | Height (cts) | (cts) |
| 1 | 7.6423 | 11.56827 | 11.9 | 90.01 | 167.71 |
| 2 | 9.8602 | 8.97065 | 100 | 756.67 | 201.11 |
| 3 | 10.2488 | 8.63133 | 35.82 | 271.07 | 205.01 |
| 4 | 13.3513 | 6.63178 | 75.44 | 570.87 | 353.9 |
| 5 | 13.7351 | 6.44733 | 56.86 | 430.22 | 383.31 |
| 6 | 14.2095 | 6.23315 | 30.64 | 231.88 | 417.36 |
| 7 | 17.2727 | 5.13402 | 44.15 | 334.05 | 574.08 |
| 8 | 17.5542 | 5.05231 | 46.76 | 353.83 | 582.59 |
| 9 | 18.5912 | 4.77277 | 44.76 | 338.68 | 604.45 |
| 10 | 19.5175 | 4.54831 | 33.81 | 255.8 | 611.01 |
| 11 | 20.4578 | 4.34133 | 30.39 | 229.94 | 605.53 |
| 12 | 21.0949 | 4.21163 | 27.86 | 210.82 | 595.61 |
| 13 | 21.768 | 4.0829 | 37.38 | 282.87 | 581.63 |
| 14 | 22.1619 | 4.0112 | 27.61 | 208.88 | 571.27 |
| 15 | 23.4955 | 3.78646 | 17.32 | 131.05 | 524.13 |
| 16 | 24.412 | 3.64635 | 5.21 | 39.44 | 488.53 |
| 17 | 25.7864 | 3.45503 | 8.09 | 61.23 | 443.42 |
| 18 | 26.7036 | 3.3384 | 10.44 | 79.03 | 406.5 |
| 19 | 27.5842 | 3.2338 | 11.86 | 89.73 | 364.42 |
| 20 | 29.0331 | 3.07563 | 11.7 | 88.53 | 287.09 |
| 21 | 30.3349 | 2.94655 | 6.77 | 51.26 | 217.36 |
| 22 | 35.6109 | 2.52117 | 2.15 | 16.3 | 147.39 |

### Stability Analyses

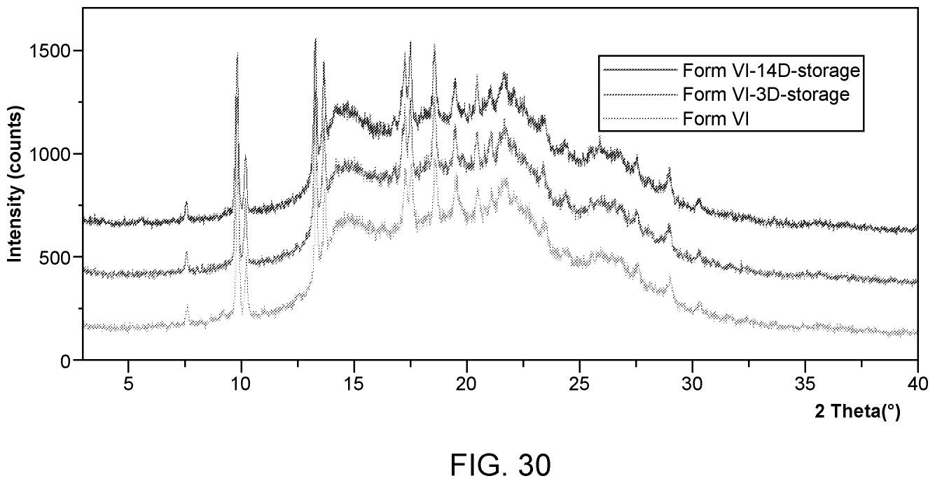

To evaluate Form VI stability, XRPD spectra of a Form VI sample were collected following 0, 3, and 14 days of room temperature storage in a sealed vial. The resulting XRPD patterns of the sample following 0 (Form VI), 3 (Form VI-3D-storage), and 14 days storage (Form VI-14D-storage) are shown in FIG. 30. Peak intensities were largely invariant over 14 days of storage, indicating that polymorphic Form VI is stable under ambient conditions.

### Example 10

### Recrystallization of (Z)-Endoxifen to Yield Polymorphic Form VII

This example describes multiple conditions for generating Form VII of (Z)-endoxifen (Formula (IV)), as well as physical characterizations of this form. A (Z)-endoxifen free base composition with ≥95% enantiomeric purity was prepared as described herein in EXAMPLE 3.

### Form VII Recrystallization (1)—Ethyl Acetate Heptane Precipitation

Form VII was generated by precipitation, in which a saturated ethyl acetate solution was slowly added to heptane, and then stored for one day at −20° C., yielding the crystalline solid of polymorphic Form VII.

### Form VII Recrystallization (2)—Toluene Evaporation

Form VII was also generated in an amorphous mixture by high temperature evaporation of a 10 mg/ml solution of (Z)-endoxifen in toluene, yielding the crystalline solid of polymorphic Form VII.

### Form VII Recrystallization (3)—Toluene Heptane Precipitation

Form VII was also generated in an amorphous mixture by dissolving (Z)-endoxifen in toluene at a concentration of 20 mg/ml, and then adding that solution to heptane, yielding the crystalline solid of polymorphic Form VII.

### Form VII Recrystallization (4)—Heptane Toluene Slurry

Polymorphic Form VII was also generated by generating a high temperature slurry of a heptane/toluene 50/50 (v/v) with 100 mg/ml (Z)-endoxifen. Crystallization of (Z)-endoxifen from this slurry yielded a crystalline solid primarily containing polymorphic Form VII and with small amounts of polymorphic Form I.

### Recrystallizations Which Generated Form VII as a Minor Component

A number of crystallizations disclosed herein generate a minor amount of Form VII of (Z)-endoxifen.

Polymorphic Form I was generated with minor traces of polymorphic Form VII by dissolving (Z)-endoxifen in a high temperature slurry in a heptane/toluene 50/50 (v/v) solvent system at concentration of 40 mg/ml yielding polymorphic Form I and minor traces of polymorphic Form VII as evidenced by low intensity XRPD peaks at 9.6, 10.6, and 11.3° 20.

Polymorphic Form I was generated with minor traces of polymorphic Form VII by first dissolving (Z)-endoxifen in DMF to saturation, and then precipitating (Z)-endoxifen by addition of the solution to water, yielding polymorphic Form I with minor traces of polymorphic Form VII as evidenced by XRPD peaks at 10.5, 13.3, 14.7, 18.0, and 23.8° 20.

Polymorphic Form IX was generated with minor traces of polymorphic Form VII by dissolving (Z)-endoxifen in acetone to saturation, and then precipitating (Z)-endoxifen by addition of butyl methyl ether to the solution, yielding polymorphic Form IX and with minor traces of polymorphic Form VII.

### XRPD Characterization

Polymorphic Form VII exhibited an x-ray powder diffraction (XRPD) pattern shown in FIG. 31 with peaks 1-35corresponding to the peak numbers (No.) in TABLE 9. Peaks in FIG. 31 and TABLE 9 are provided as relative intensities (Rel. Int.) standardized against the peak at 20.0° 20 (the peak with the highest intensity). The ten largest peaks by measure of Rel. Int. identified in the XRPD of Form VII, were at 20.0, 22.6, 10.6, 11.4, 16.4, 9.6, 13.3, 18.2, 13.1, and 27.0° 20, corresponding to peak numbers (No.) 13, 18, 2, 3, 7, 1, 5, 10, 4, and 25, respectively. The three largest peaks by measure of Rel. Int. were at 20.0, 22.6, and 10.6° 2θ.

TABLE 9
X-Ray Powder Diffraction Peak Table for Form VII

| | Pos. | | Rel. | Height | Backgr. |
|---|---|---|---|---|---|
| No. | (°2Th.) | d-spacing (Å) | Int. (%) | (cts) | (cts) |
| 1 | 9.5987 | 9.21444 | 40.34 | 1298.2 | 220 |
| 2 | 10.6299 | 8.3227 | 61.84 | 1989.94 | 253.99 |
| 3 | 11.3521 | 7.7948 | 51.74 | 1664.72 | 278 |
| 4 | 13.1069 | 6.7549 | 26.4 | 849.42 | 413.38 |
| 5 | 13.2935 | 6.66048 | 39.25 | 1263.07 | 448.71 |
| 6 | 16.0062 | 5.53727 | 18.64 | 599.78 | 700.62 |
| 7 | 16.4362 | 5.39338 | 50.87 | 1636.8 | 709 |
| 8 | 16.8585 | 5.25919 | 16.31 | 524.81 | 706 |
| 9 | 17.8381 | 4.97255 | 11.22 | 360.9 | 721 |
| 10 | 18.2346 | 4.86529 | 32.01 | 1029.98 | 735 |
| 11 | 18.8175 | 4.7159 | 2.94 | 94.47 | 739 |
| 12 | 19.2279 | 4.61616 | 10.09 | 324.76 | 751.79 |
| 13 | 19.9719 | 4.44584 | 100 | 3217.77 | 779 |
| 14 | 20.3518 | 4.36369 | 7.34 | 236.14 | 778 |
| 15 | 21.3019 | 4.17116 | 2.83 | 90.97 | 759.81 |
| 16 | 21.6689 | 4.10134 | 3.61 | 116.23 | 752 |
| 17 | 22.3164 | 3.98379 | 9.89 | 318.35 | 719 |
| 18 | 22.6452 | 3.92669 | 88 | 2831.71 | 690.48 |
| 19 | 23.697 | 3.75472 | 2.39 | 76.98 | 575.3 |
| 20 | 24.6791 | 3.60749 | 9.64 | 310.15 | 521 |
| 21 | 25.6547 | 3.47247 | 3.63 | 116.9 | 503 |
| 22 | 25.925 | 3.43687 | 3.7 | 118.97 | 504 |
| 23 | 26.3372 | 3.38401 | 5.24 | 168.59 | 497.28 |
| 24 | 26.7177 | 3.33667 | 17.15 | 551.89 | 481 |
| 25 | 26.9985 | 3.30261 | 24.93 | 802.19 | 462 |
| 26 | 27.5814 | 3.23412 | 3.56 | 114.51 | 411.86 |
| 27 | 27.8936 | 3.19863 | 4.94 | 158.99 | 385 |
| 28 | 28.9638 | 3.08284 | 6.17 | 198.65 | 322.62 |
| 29 | 31.2571 | 2.86169 | 2.25 | 72.34 | 226.29 |
| 30 | 31.7472 | 2.81862 | 1.54 | 49.51 | 224 |
| 31 | 32.2885 | 2.77259 | 9.44 | 303.88 | 222 |
| 32 | 33.31 | 2.68986 | 1.68 | 54 | 206 |
| 33 | 33.5546 | 2.67082 | 2.71 | 87.36 | 200.54 |
| 34 | 34.0251 | 2.63495 | 1.74 | 55.86 | 190 |
| 35 | 38.1169 | 2.36098 | 3.4 | 109.35 | 167 |

### Stability Analysis

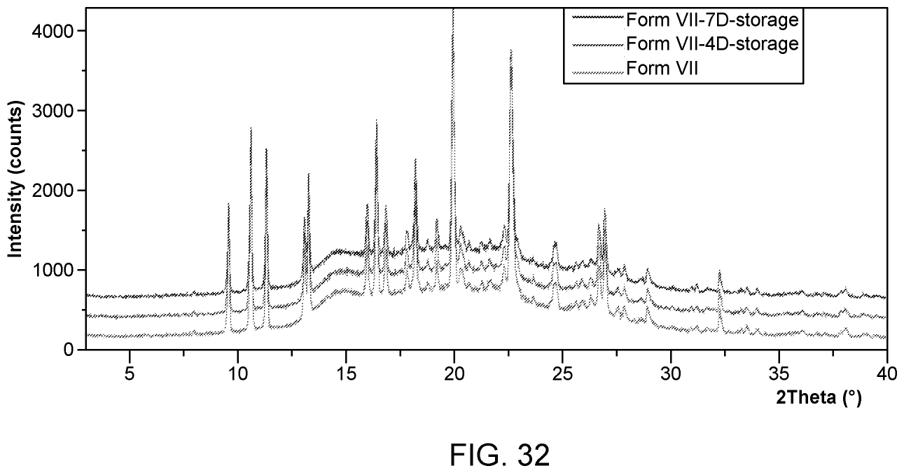

The stability of polymorphic Form VII was evaluated over 7 days of storage at room temperature in a sealed vial. Portions of the Form VII samples were collected for XRPD analysis over 7 days of storage, the results of which are shown in FIG. 32. In FIG. 32, XRPD following 0 days (Form VII), 3 days (Form VII-3D-storage), and 7 days of storage (Form VII-71D-storage) are displayed. Minimal changes were observed in the XRPD time course, indicating that polymorphic Form VII is stable under ambient conditions.

### DSC Characterization

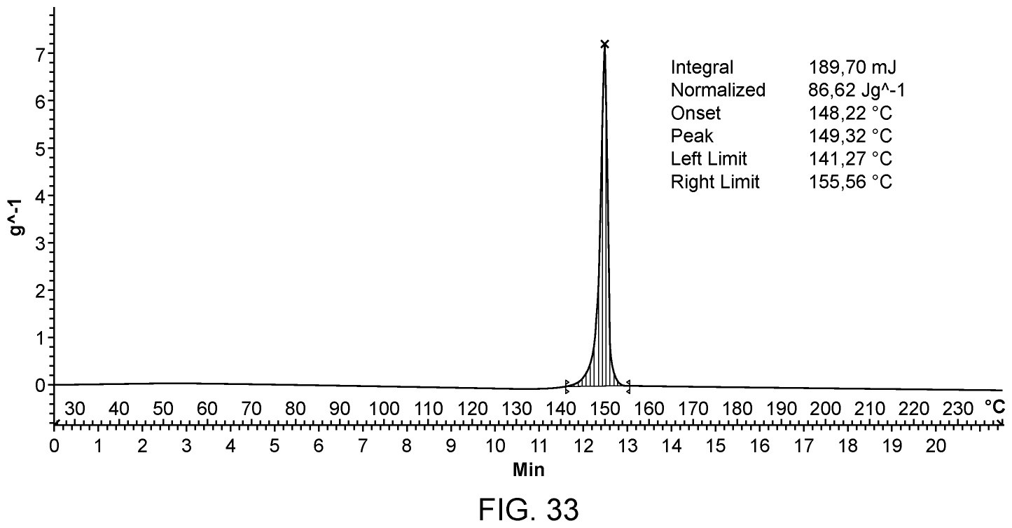

A DSC profile of polymorphic Form VII is shown in FIG. 33. Form VII exhibited an endothermic peak at 149° C. (onset at 148° C., Delta H: approx. 86.6 J/g), ascribable to Form VII melting.

### TGA Analysis

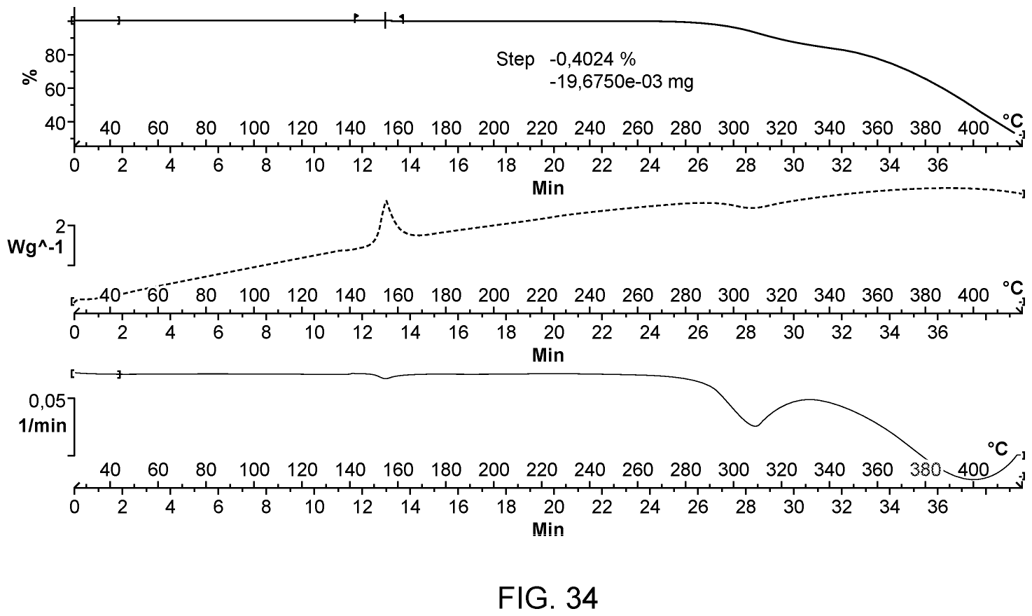

The TGA analysis of polymorphic Form VII is shown in FIG. 34. FIG. 34 summarizes TGA (top), heat flow (middle), and 1^{st} derivative (bottom) profiles of Form VII. The TGA analysis revealed a weight loss of 0.4% in total between 140° C.-165° C. potentially ascribable to solvent release. Degradation of polymorphic Form VII occurred above 270° C.

### Micro Scale-Up Experiments

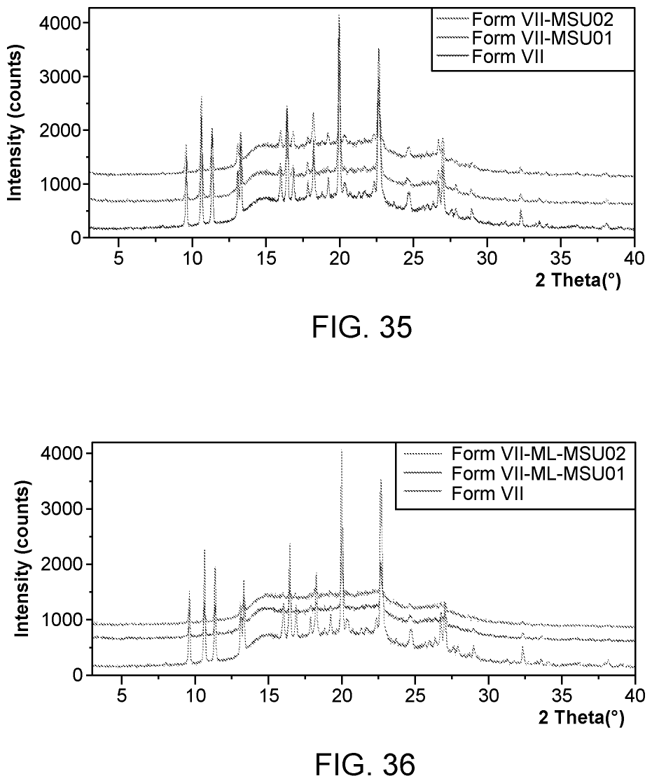

Two micro scale-up experiments (micro-scale experiments 1 and 2) of Form VII were attempted using the following procedure. The micro scale-up experiments 1 and 2 of Form VII were performed by adding (Z)-endoxifen ethyl acetate saturated solution to heptane, and then storing for one day at −20° C. Specifically, 1.2 ml of ethyl acetate saturated solution was added to 12 ml of heptane under stirring at room temperature to reach the solvent/antisolvent ratio of 1:10. No precipitation was observed. Then, the clear solution was stored at −20° C. for one day. The obtained white suspension was filtered with a Whatman 0.45 μm paper filter under vacuum and the collected powder was analyzed by XRPD (FIG. 35). In FIG. 35 the XRPD pattern of Form VII from the ethyl acetate/heptane precipitation (Form VII) is compared to the XRPD patterns of micro scale-up experiments 1 (Form VII-MSU01) and 2 (Form VII-MSU02) showing that the micro scale-up experiments led to the recovery of Form VII.

The mother liquors recovered from micro scale-up experiments of Form VII were left to evaporate at room temperature. FIG. 36 provides XRPD patterns of the crystals obtained from the ethyl acetate/heptane precipitation (Form VII), and the mother liquor recovered from micro scale up experiment 1 (Form VII-ML-MSU01) and 2 (Form VII-ML-MSU02) and confirms that Form VII was obtained from the solvent evaporation.

### Example 11

### Recrystallization of (Z)-Endoxifen to Yield Polymorphic Form VIII

This example describes multiple crystallization conditions for generating Form VIII of (Z)-endoxifen (Formula (IV)), as well as physical characterizations of this form. A (Z)-endoxifen free base composition with ≥95% enantiomeric purity was prepared as described herein in EXAMPLE 3. Some solutions in the following example were precipitated by cooling of the solution from a temperature between 50-100° C. to between 10-25° C. at a rate ranging from −0.2 to −0.5° C./minute resulting in a precipitate.

### Form VIII Recrystallization (1)—Toluene Precipitation

Form VIII was generated by precipitation from cooling the 100 mg/ml solution of (Z)-endoxifen in toluene, yielding the crystalline solid of polymorphic Form VIII.

### Form VIII Recrystallization (2)—Toluene Butyl Methyl Ether Precipitation and Evaporation

Form VIII was also generated by precipitation from a 20 mg/ml solution of (Z)-endoxifen in toluene. The solution was precipitated by addition of butyl methyl ether and evaporated at room temperature to yield the crystalline solid of polymorphic Form VIII.

### XRPD Characterization

Polymorphic Form VIII exhibited an x-ray powder diffraction (XRPD) pattern shown in FIG. 37 with peaks 1-43 corresponding to the peak numbers (No.) in TABLE 10. Peaks in FIG. 37 and TABLE 10 are provided as relative intensities (Rel. Int.) standardized against the peak at 4.8° 2θ (the peak with the highest intensity). The ten largest peaks by measure of Rel. Int. identified in the XRPD of Form VIII, were at 4.8, 18.9, 9.5, 23.7, 21.9, 21.2, 12.9, 25.0, 21.5, and 16.4° 2θ, corresponding to peak numbers (No.) 1, 15, 3, 23, 20, 18, 5, 26, 19, and 10, respectively. The three largest peaks by measure of Rel. Int. were at 4.8, 18.9, and 9.5° 2θ.

TABLE 10
X-Ray Powder Diffraction Peak Table for Form VIII

| | Pos. | | Rel. | | Backgr. |
|---|---|---|---|---|---|
| No. | (°2Th.) | d-spacing | Int. (%) | Height (cts) | (cts) |
| 1 | 4.7794 | 18.48958 | 100 | 11538.7 | 225 |
| 2 | 7.6356 | 11.57836 | 2.53 | 291.51 | 241 |
| 3 | 9.4752 | 9.33425 | 52.01 | 6000.82 | 294 |
| 4 | 10.5766 | 8.36456 | 4.03 | 465.14 | 322 |
| 5 | 12.8518 | 6.88841 | 16.93 | 1953.21 | 475.36 |
| 6 | 14.1862 | 6.24333 | 8.87 | 1023.03 | 659.62 |
| 7 | 14.4867 | 6.11448 | 9.79 | 1129.79 | 695.67 |
| 8 | 15.2286 | 5.81824 | 2.68 | 309.31 | 778.86 |
| 9 | 15.668 | 5.65602 | 1.04 | 120.23 | 823.8 |
| 10 | 16.4381 | 5.39276 | 11.11 | 1281.54 | 897.81 |
| 11 | 17.0519 | 5.2 | 5.41 | 624.36 | 951.19 |
| 12 | 17.23 | 5.14664 | 3.75 | 432.86 | 966 |
| 13 | 17.8057 | 4.98152 | 1.29 | 148.91 | 1010.57 |
| 14 | 18.3288 | 4.84051 | 5.26 | 606.9 | 1046 |
| 15 | 18.9092 | 4.69322 | 61.58 | 7105.22 | 1080 |
| 16 | 20.0243 | 4.43432 | 5.2 | 599.82 | 1127.43 |
| 17 | 20.3245 | 4.36949 | 2.83 | 326.35 | 1135.45 |
| 18 | 21.1605 | 4.19872 | 20.48 | 2362.68 | 1145 |
| 19 | 21.4658 | 4.13969 | 11.71 | 1350.87 | 1143 |
| 20 | 21.8678 | 4.06449 | 34.27 | 3953.77 | 1136 |
| 21 | 22.8691 | 3.88874 | 1.92 | 221.4 | 1098 |
| 22 | 23.2972 | 3.81824 | 8.2 | 946.67 | 1075 |
| 23 | 23.6975 | 3.75465 | 46.98 | 5420.66 | 1050.25 |
| 24 | 24.613 | 3.61702 | 7.47 | 862.5 | 986.7 |
| 25 | 24.72 | 3.60161 | 7.39 | 852.3 | 979 |
| 26 | 24.9993 | 3.562 | 13.15 | 1517.52 | 957.07 |
| 27 | 25.7311 | 3.46234 | 3.55 | 409.46 | 894 |
| 28 | 26.754 | 3.33224 | 4.08 | 471.23 | 795.6 |
| 29 | 27.0319 | 3.2986 | 6.48 | 747.16 | 767.81 |
| 30 | 27.9999 | 3.18672 | 3.25 | 375.19 | 688 |
| 31 | 28.3444 | 3.14878 | 10.64 | 1227.87 | 663.56 |
| 32 | 28.751 | 3.10517 | 3.14 | 362.6 | 633.9 |
| 33 | 29.1369 | 3.06492 | 6.49 | 748.71 | 603.31 |
| 34 | 29.9563 | 2.98292 | 3.06 | 352.81 | 531.37 |
| 35 | 30.77 | 2.90587 | 3.77 | 435 | 452 |
| 36 | 32.1302 | 2.78589 | 1.9 | 219.15 | 383 |
| 37 | 32.6595 | 2.74194 | 1.83 | 211.1 | 373 |
| 38 | 33.397 | 2.68306 | 1.02 | 117.78 | 359 |
| 39 | 34.9938 | 2.5642 | 1.39 | 160.46 | 344 |
| 40 | 36.57 | 2.45722 | 0.63 | 73 | 341 |
| 41 | 37.4786 | 2.39971 | 1.58 | 182.28 | 337 |
| 42 | 38.5 | 2.33836 | 0.62 | 72 | 330 |
| 43 | 39.0518 | 2.30659 | 0.87 | 100.73 | 324 |

### Stability Analysis

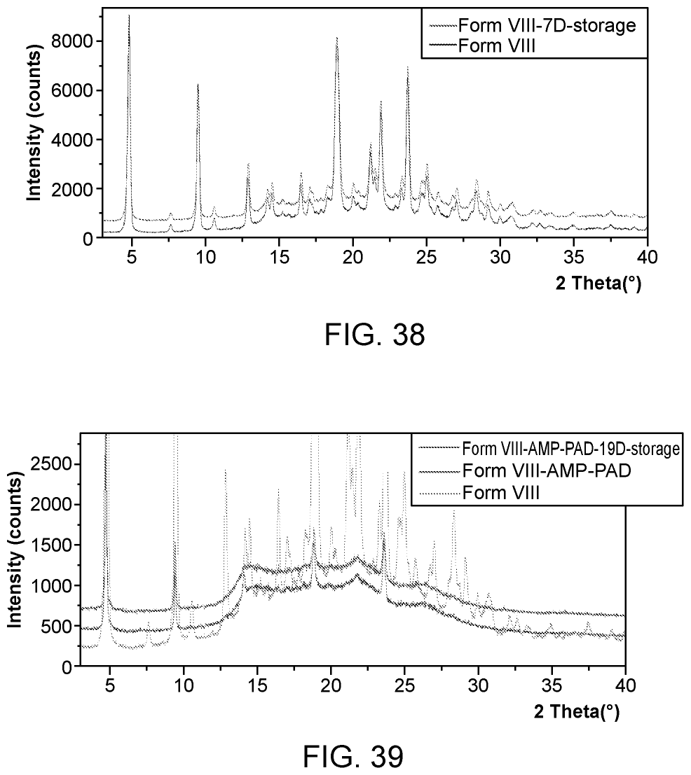

Form VIII stability over 7 days storage in a sealed vial at room temperature was evaluated with XRPD. The resulting XRPD patterns are shown in FIG. 38 and show no significant difference between Form VIII after 7 days of storage (Form VIII-7D-storage) and Form VIII with 0 days of storage (Form VIII) indicating polymorphic Form VIII's stability under ambient conditions.

Attributes of Form VIII were also observed as a minor component in amorphous (Z)-endoxifen generated by precipitation from a toluene/butyl methyl ether solvent/antisolvent mixture containing 20 mg/ml (Z)-endoxifen. The sample was remeasured after 19 days of storage in a sealed vial. XRPD patterns of the resultant solids shown in FIG. 39, which (from bottom to top) provides the XRPD pattern of Form VIII (Form VIII), the XRPD pattern of the mixture of Form VIII and amorphous (Z)-endoxifen achieved by antisolvent precipitation (Form VIII-AMP-PAD), and the same mixture of Form VIII and amorphous (Z)-endoxifen after storage for 19 days (Form VIII-AMP-PAD-19D-storage).

### Example 12

### Recrystallization of (Z)-Endoxifen to Yield Form IX

This example describes recrystallization of (Z)-endoxifen (Formula (IV)) to yield a polymorphic Form IX of (Z)-endoxifen. A (Z)-endoxifen free base composition with ≥95% enantiomeric purity was prepared as described herein in EXAMPLE 3.

### Form IX Recrystallization (1)—Ethyl Acetate Heptane Precipitation

Form IX was generated by dissolving (Z)-endoxifen powder in ethyl acetate at a concentration of 20 mg/ml. The solution was then precipitated by addition of heptane yielding the crystalline solid of polymorphic Form IX.

### Repeat Studies for Ethyl Acetate Heptane Precipitations

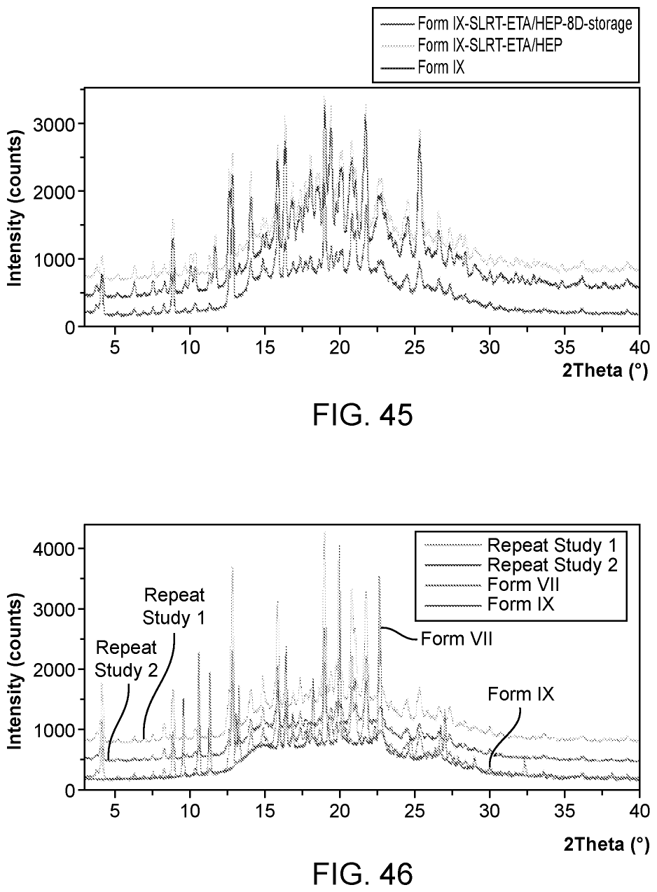

Variations of Form IX Recrystallization (1) were performed with multiple solvent volumes and filtration methods, referred to as Repeat Study 1-4. In Repeat Study 1, 20 mg of (Z)-endoxifen was added to 1 ml of ethyl acetate and stirring for one day. The suspension was then filtered at room temperature with a nylon filter (0.22 μm) and centrifuged at 4000 rpm for 3 minutes, recovering 800 μl of saturated solution. Heptane was added to the (Z)-endoxifen saturated ethyl acetate solution to reach a solvent/antisolvent ratio of 1:10. If no precipitate was observed, the clear solution was stored for one day at −20° C. The obtained precipitate was then filtered with a 0.45 μm paper filter under vacuum and the collected material was analyzed by XRPD. As shown in FIG. 46, Repeat Study 1 of Recrystallization (1) (Repeat Study 1) yielded polymorph mixture with Form IX (Form IX) as a primary component and minor amounts of Form VII (Form VII).

In Repeat Study 2 of Recrystallization (1), Form IX was generated by suspending 20 mg of (Z)-endoxifen in 1 ml of ethyl acetate and stirring for one day. The suspension was then filtered at room temperature with a nylon filter (0.22 μm) and centrifuged at 4000 rpm for 3 minutes, recovering 600 μl of saturated solution. Heptane was added to the (Z)-endoxifen saturated ethyl acetate solution to reach a solvent/antisolvent ratio of 1:10. If no precipitate was observed, the clear solution was stored for one day at −20° C. The obtained precipitate was then filtered with a 0.45 μm paper filter under vacuum and the collected material was analyzed by XRPD. As shown in FIG. 46, Repeat Study 2 of Recrystallization (1) (Repeat Study 2) yielded polymorph mixture with Form IX (Form IX) as a primary component and minor amounts of Form VII (Form VII).

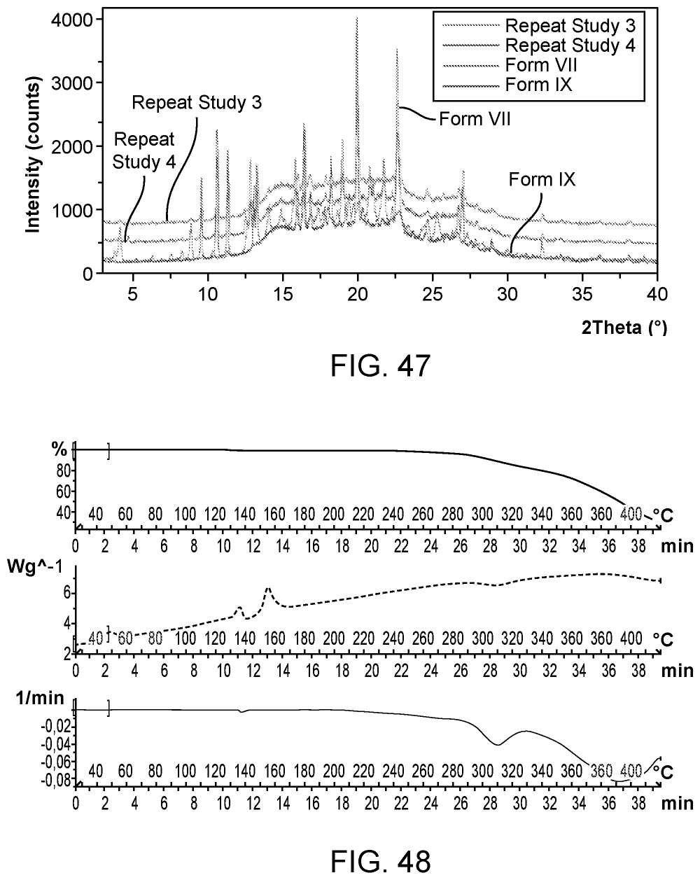

In Repeat Study 3 and Repeat Study 4, Form IX Recrystallization (1) were performed by suspending 20 mg of (Z)-endoxifen in 1 ml of ethyl acetate and stirring for one day. The suspension was then filtered at room temperature with a nylon filter (0.22 μm) and centrifuged at 4000 rpm for 3 minutes. After filtration, the solution appeared as an opalescent solution, so it was then re-filtered with a 0.45 μm filter, recovering 600 μl of saturated solution. Heptane was added to the (Z)-endoxifen saturated ethyl acetate solution to reach a solvent/antisolvent ratio of 1:10. If no precipitate was observed, the clear solution was stored for one day at −20° C. The obtained precipitate was then filtered with a 0.45 μm paper filter under vacuum and the collected material was analyzed by XRPD. As shown in FIG. 47, Repeat Study 3 (Repeat Study 3) and Repeat Study 4 (Repeat Study 4) of Recrystallization (1) yielded polymorph mixtures primarily composed of Form VII (Form VII) and containing minor amounts of Form IX (Form IX).

### Form IX Recrystallization (2)—Ethyl Acetate Heptane Precipitation by Antisolvent Addition

Form IX was also generated by addition of heptane to a (Z)-endoxifen saturated ethyl acetate solution, and storage of the resultant solution for one day at −20° C. It should be noted that this procedure is similar to the procedure for isolation of polymorphic Form VII, except for the antisolvent addition method. This isolation of Form IX also proved to be more crystalline than the above isolation method, so it was chosen as the XRPD pattern reference for Form IX (FIG. 40) discussed below.

### Form IX Recrystallization (3)—Butyl Acetate Heptane Precipitation

Form IX was also generated by dissolving (Z)-endoxifen powder in butyl acetate at a concentration of 20 mg/ml. The solution was then precipitated by addition of heptane to the solution yielding the crystalline solid of polymorphic Form IX.

### Form IX Recrystallization (4)—Ethyl Acetate Butyl Methyl Ether Precipitation and Evaporation

Form IX was also generated by precipitation of a saturated solution of (Z)-endoxifen in ethyl acetate by addition of the saturated solution to butyl methyl ether and subsequent evaporation at room temperature yielding the crystalline solid of polymorphic Form IX.

Form IX Recrystallization (5)—Ethyl Acetate Butyl Methyl Ether Precipitation and Evaporation by Antisolvent Addition

Form IX was also generated by precipitation of a saturated solution of (Z)-endoxifen in ethyl acetate by addition of butyl methyl ether to the saturated solution and subsequent evaporation at room temperature yielding the crystalline solid of polymorphic Form IX.

### Recrystallizations Which Generated Form IX and Uncharacterized Polymorphs

A number of crystallizations yielded Form IX in combination with other polymorphs not yet isolated or characterized. In a first of these crystallizations, a saturated solution of (Z)-endoxifen in acetone as added to butyl methyl ether, and then subsequently evaporated at room temperature yielding the crystalline solid of polymorphic Form IX with an unassigned signal at 3.5020.

A further crystallization involved addition of butyl methyl ether to a saturated solution of (Z)-endoxifen in acetone, followed by subsequent evaporation at room temperature to yield the crystalline solid of polymorphic Form IX with unassigned signals at 3.5 and 13.7°2θ, as well as a small amount of Form VII.

A further crystallization was performed by dissolving amorphous (Z)-endoxifen in an ethyl acetate/heptane 50/50 (v/v) solvent system to from a room temperature slurry, crystallization from which yielded the crystalline solid of polymorphic Form IX with unassigned signals at 10.1, 11.7, 16.8, 18.5, 19.4, and 23.1° 2θ.

### XRPD Characterization

Polymorphic Form IX exhibited an x-ray powder diffraction (XRPD) pattern shown in FIG. 40 with peaks 1-42 corresponding to the peak numbers (No.) in TABLE 11. Peaks in FIG. 40 and TABLE 11 are provided as relative intensities (Rel. Int.) standardized against the peak at 19.0° 2θ (the peak with the highest intensity). The ten largest peaks by measure of Rel. Int. identified in the XRPD of Form IX, were at 19.0, 12.9, 15.9, 21.7, 20.8, 21.1, 8.9, 16.4, 4.2, and 12.7020, corresponding to peak numbers (No.) 21, 11, 14, 28, 26, 27, 6, 15, 2, and 10, respectively. The three largest peaks by measure of Rel. Int. were at 19.0, 12.9, and 15.9° 2θ.

TABLE 11
X-Ray Powder Diffraction Peak Table for Form IX

| | Pos. | | Rel. | Height | Backgr. |
|---|---|---|---|---|---|
| No. | (°2Th.) | d-spacing (Å) | Int. (%) | (cts) | (cts) |
| 1 | 3.8 | 23.25239 | 8.14 | 149 | 198 |
| 2 | 4.1905 | 21.0866 | 29.24 | 535.16 | 187 |
| 3 | 6.35 | 13.91939 | 5.52 | 101 | 179 |
| 4 | 7.5868 | 11.65284 | 6.13 | 112.24 | 190 |
| 5 | 8.3258 | 10.62014 | 7.04 | 128.94 | 204.58 |
| 6 | 8.9096 | 9.92549 | 32.53 | 595.37 | 213 |
| 7 | 9.73 | 9.09038 | 3.59 | 65.78 | 219 |
| 8 | 10.3932 | 8.51169 | 6.37 | 116.53 | 229 |
| 9 | 11.3564 | 7.79186 | 5.69 | 104.14 | 252 |
| 10 | 12.657 | 6.99399 | 27.36 | 500.7 | 350.4 |
| 11 | 12.8641 | 6.88182 | 78.03 | 1428.09 | 378.41 |
| 12 | 14.1027 | 6.2801 | 26.42 | 483.63 | 533.27 |
| 13 | 14.8948 | 5.94786 | 20.84 | 381.43 | 613.48 |
| 14 | 15.8642 | 5.58652 | 58.62 | 1072.98 | 690.42 |
| 15 | 16.378 | 5.41241 | 32.28 | 590.82 | 722.8 |
| 16 | 16.9117 | 5.24279 | 10.35 | 189.4 | 750 |
| 17 | 17.3862 | 5.10074 | 14.25 | 260.89 | 770 |
| 18 | 17.7288 | 5.00294 | 8.79 | 160.91 | 781.88 |
| 19 | 18.0609 | 4.91169 | 13.21 | 241.73 | 791.09 |
| 20 | 18.6536 | 4.75695 | 10.99 | 201.2 | 804 |
| 21 | 18.9941 | 4.67243 | 100 | 1830.27 | 808 |
| 22 | 19.4401 | 4.56624 | 21.44 | 392.5 | 811 |
| 23 | 19.7884 | 4.48663 | 17.35 | 317.56 | 811 |
| 24 | 20.0294 | 4.4332 | 16.58 | 303.54 | 809 |
| 25 | 20.1661 | 4.40346 | 18.37 | 336.29 | 807.39 |
| 26 | 20.8231 | 4.26598 | 49.45 | 905.08 | 793 |
| 27 | 21.05 | 4.22051 | 34.33 | 628.3 | 786 |
| 28 | 21.7463 | 4.08693 | 55.42 | 1014.3 | 753 |
| 29 | 22.7606 | 3.90703 | 16.33 | 298.94 | 681.94 |
| 30 | 23.3914 | 3.80308 | 6.74 | 123.41 | 627.86 |
| 31 | 24.2521 | 3.67002 | 7.99 | 146.26 | 561.79 |
| 32 | 24.4952 | 3.63415 | 11.85 | 216.95 | 553 |
| 33 | 25.2934 | 3.52125 | 21.74 | 397.9 | 527 |
| 34 | 25.85 | 3.44668 | 5.67 | 103.81 | 509 |
| 35 | 26.5688 | 3.35505 | 12.15 | 222.39 | 480.12 |
| 36 | 27.3068 | 3.26602 | 10.27 | 187.92 | 442.32 |
| 37 | 28.051 | 3.18104 | 3.59 | 65.66 | 398.9 |
| 38 | 28.3519 | 3.14796 | 5.79 | 106.03 | 381.81 |
| 39 | 29.0582 | 3.07303 | 2.83 | 51.8 | 337.18 |
| 40 | 29.9729 | 2.9813 | 4.01 | 73.4 | 286.71 |
| 41 | 36.1303 | 2.48611 | 3.72 | 68.16 | 204 |
| 42 | 39.1102 | 2.30328 | 3.13 | 57.21 | 194 |

### Stability Analysis

The stability of Form IX generated by precipitation from an ethyl acetate/heptane solvent system (Form IX Recrystallization (1)) was evaluated over 7 days of storage at room temperature. Portions of the Form IX sample were collected following 0 and 7 days of storage in a sealed vial and analyzed by XRPD analysis to identify possible changes in the polymorph.

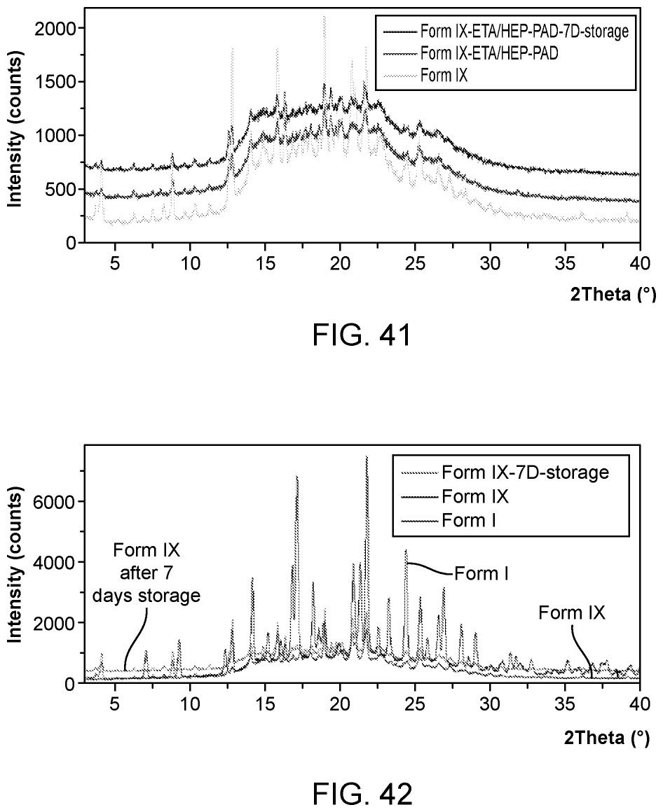

These XRPD patterns are displayed in FIG. 41, with 0 (Form IX-ETA/HEP-PAD) and 7 (Form IX-ETA/HEP-PAD-7D-storage) days storage compared against a reference pattern of Form IX generated by precipitation from ethyl acetate/heptane precipitation at −20° C. (Form IX). Minimal changes were observed in the XRPD time course indicating that, Form IX is stable under ambient conditions.

Form IX stability was separately characterized for crystals generated through the addition of (Z)-endoxifen saturated ethyl acetate to heptane (Form IX Recrystallization (2)), which yielded higher crystallinity Form IX than was afforded by heptane addition to (Z)-endoxifen saturated ethyl acetate. Form IX was collected following 0 days, 7 days (FIG. 42), and 13 days (FIG. 43) of storage in a sealed vial at room temperature and compared to an XRPD reference pattern of Form I. In FIG. 42, XRPD patterns of Form IX following 0 (Form IX) and 7 (Form IX-71D-storage) days of room temperature storage are compared against a reference XRPD pattern of Form I (Form I). In FIG. 43 XRPD patterns of Form IX following 0 (Form IX) and 13 (Form IX-13D-storage) days of storage in a sealed vial and compared to the reference XRPD pattern of Form I (Form I). As seen in these figures, Form IX generated with Recrystallization method (2) shows weak XRPD signals ascribable to small amounts of Form I following 7 days storage, and following 13 days.

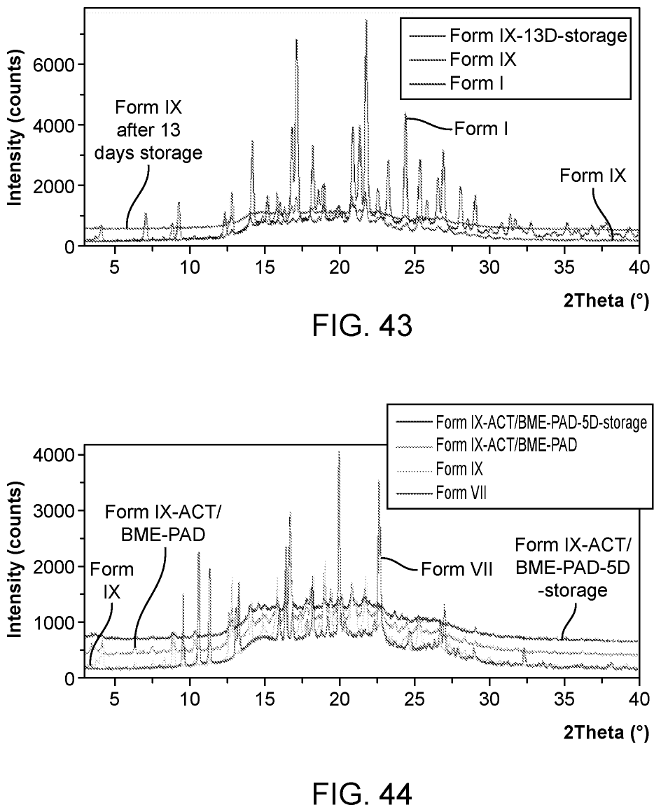

Form IX stability was further evaluated for crystals generated by precipitation from an acetone/butyl methyl ether (acetone/BME) solvent/antisolvent mixture (Form IX Recrystallization (4)). Portions of the Form IX sample were collected following 0 and 5 days of storage in a sealed vial at room temperature and compared to reference XRPD patterns of Forms VII and IX. In FIG. 44, XRPD patterns of the Form IX sample from Recrystallization (4) following 0 (Form IX-ACT/BME-PAD) and 5 days storage (Form IX-ACT/BME-PAD-5D-storage) are presented with the reference patterns of Form IX generated by recrystallization method (2) (Form IX) and Form VII (Form VII). As seen in the figure, this Form IX sample exhibited additional XRPD signals at 3.5, 14.6, and 16.7° 2θ, possibly indicating the presence of Form VII. The signals at 3.5, 14.6, and 16.7° 2θ persisted over 5 days of storage and were accompanied by a new signal at 9.1° 2θ, possibly indicating the presence of Form VII as well as partial amorphization of the sample.

Stability analyses were separately performed for Form IX crystals isolated from room temperature ethyl acetate/heptane 50/50 (v/v) slurries. XRPD was collected on portions of the Form IX sample were stored for 0 and 8 days of storage in a sealed vial at room temperature and compared against a Form IX reference pattern. FIG. 45 provides XRPD patterns of Form IX isolated from room temperature ethyl acetate/heptane 50/50 (v/v) slurries following 0 (Form IX-SLRT-ETA/HEP) and 8 (Form IX-SLRT-ETA/HEP-8D-storage) days storage along with a reference pattern of Form IX generated by Recrystallization (2) (Form IX). As seen in the figure, Form IX generated from the room temperature ethyl acetate/heptane slurries included unassigned XRPD signals at 10.1, 11.7, 16.8, 18.5, 19.4, and 23.1° 20. Further seen in the figure, these peaks persisted following 8 days of storage.

XRPD Characterization of Form IX Generated with Recrystallization (1) Variations

Some variations of Recrystallization (1) generated mixtures of polymorphic Forms VII and IX. As shown in FIG. 46, Repeat Study 1 (Repeat Study 1) and Repeat Study 2 (Repeat Study 2) of Recrystallization (1) yielded polymorph mixture with Form IX (Form IX) as a primary component and minor amounts of Form VII (Form VII). As shown in FIG. 47, Repeat Study 3 (Repeat Study 3) and Repeat Study 4 (Repeat Study 4) of Recrystallization (1) yielded polymorph mixtures primarily composed of Form VII (Form VII) and containing minor amounts of Form IX (Form IX).

### TGA Analysis

The TGA analysis for Form IX was performed on the sample obtained by variations 1 and 2 of Recrystallization (1). These results are shown in FIG. 48, which displays the TGA profile (top), the heat flow (middle), and the 1^{st} derivative of the TGA (bottom). As seen in this figure, the TGA analysis revealed a weight loss of 0.2% recorded between 125-145° C., potentially due to solvent release. Degradation of Form IX took place above 270° C.

### Microgram-Scale Crystallization

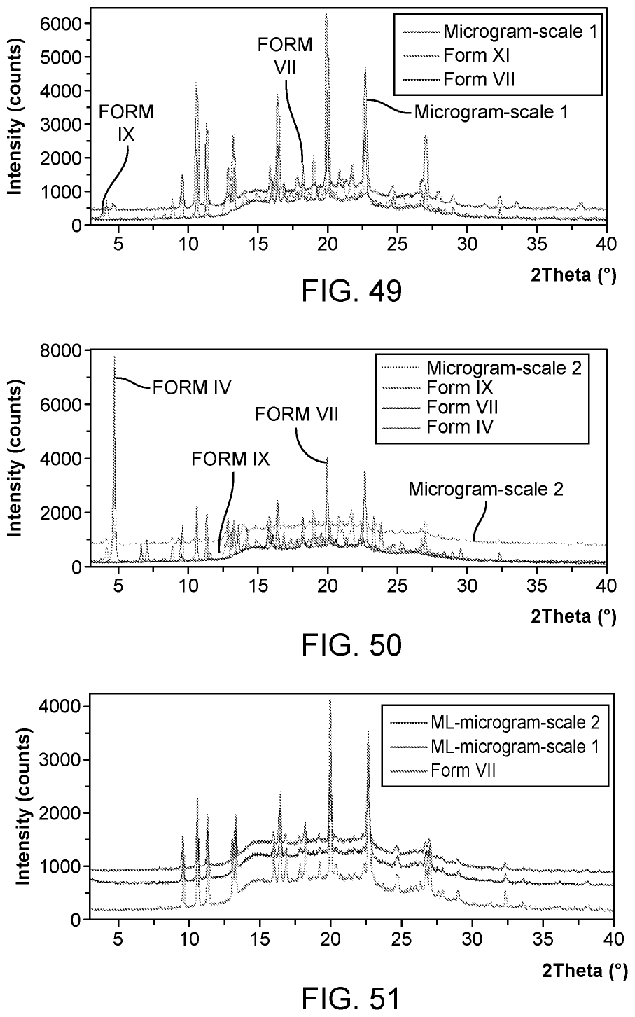

Two microgram-scale Form IX crystallizations were performed following the procedure of Form IX Recrystallization (1). 12 ml of heptane was added to 1.2 ml of a (Z)-endoxifen saturated ethyl acetate solution under stirring at room temperature to reach a solvent/antisolvent ratio of 1:10. No precipitation was observed. The resultant clear solution was then stored at −20° C. for one day, yielding a white suspension, which was collected by vacuum filtration with a 0.45 μm filter paper. XRPD of the resultant crystals of the first microgram-scale crystallization (Microgram-scale 1) is shown in FIG. 49 and the second microgram-scale crystallization (Microgram-scale 2) is shown in FIG. 50. As seen in these figures, the product primarily included Form VII with traces of Form IX and Form IV.

### Mother Liquor Evaporations of Form IX

Heptane/ethyl acetate mother liquors were recovered from both micro-scale crystallizations described above and evaporated at room temperature. The resulting solids were characterized by XRPD (ML-microgram-scale 1 and ML-microgram-scale 2) and compared to Form VII, as shown in FIG. 51.

### Example 13

### Recrystallization of (Z)-Endoxifen to Yield Form X

This example describes crystallization of (Z)-endoxifen (Formula (IV)) to yield polymorphic Form X of (Z)-endoxifen. An endoxifen composition containing ≥95% (Z)-endoxifen free base was prepared as described herein in EXAMPLE 3. Some solutions in the following example were precipitated by cooling of the solution from a temperature between 50-100° C. to between 10-25° C. at a rate ranging from −0.2 to −0.5° C./minute resulting in a precipitate.

Form X Recrystallization (1)—4-Methyl-2-Pentanone Precipitation Form X was generated by dissolving (Z)-endoxifen powder in 4-methyl-2-pentanone at a concentration of 100 mg/ml. The solution was then precipitated by cooling the solution and storage of the cooled solution for one day of storage at −20° C., yielding the crystalline solid of polymorphic Form X.

### XRPD Characterization

Polymorphic Form X exhibited an XRPD pattern shown in FIG. 52 with peaks 1-42 corresponding to the peak numbers (No.) in TABLE 12. Peaks in FIG. 52 and TABLE 12 are provided as relative intensities (Rel. Int.) standardized against the peak at 7.2° 2θ (the peak with the highest intensity). The ten largest peaks by measure of Rel. Int. identified in the XRPD of Form X, were at 7.2, 14.3, 18.7, 21.5, 22.7, 17.1, 22.7, 21.8, 27.3, and 29.4020, corresponding to peak numbers (No.) 1, 7, 13, 18, 22, 10, 21, 19, 29, and 32, respectively. The three largest peaks by measure of Rel. Int. were at 7.2, 14.3, and 18.7° 2θ.

TABLE 12
X-Ray Powder Diffraction Peak Table for Form X

| | Pos. | d-spacing | Rel. | Height | Backgr. |
|---|---|---|---|---|---|
| No. | (°2Th.) | (Å) | Int. (%) | (cts) | (cts) |
| 1 | 7.1892 | 12.28617 | 100 | 4079.6 | 151.11 |
| 2 | 9.3107 | 9.4909 | 5.16 | 210.58 | 153.53 |
| 3 | 11.2409 | 7.86517 | 1.14 | 46.51 | 181.68 |
| 4 | 11.6945 | 7.56112 | 9.46 | 386.07 | 201.11 |
| 5 | 12.2186 | 7.23793 | 9.11 | 371.47 | 220.62 |
| 6 | 12.2592 | 7.21406 | 10.49 | 428.08 | 222.31 |
| 7 | 14.3244 | 6.17826 | 71.01 | 2896.94 | 394.61 |
| 8 | 15.277 | 5.7951 | 7.38 | 300.91 | 465.28 |
| 9 | 16.3673 | 5.41145 | 8.56 | 349.12 | 500.68 |
| 10 | 17.1103 | 5.17808 | 36.07 | 1471.45 | 528.71 |
| 11 | 17.8055 | 4.97746 | 9.83 | 401.18 | 558.44 |
| 12 | 18.1848 | 4.87446 | 6.18 | 252.26 | 569.76 |
| 13 | 18.7378 | 4.73185 | 50.04 | 2041.31 | 576.28 |
| 14 | 19.0869 | 4.64609 | 3.46 | 140.99 | 573.11 |
| 15 | 19.3599 | 4.58117 | 3.64 | 148.7 | 568.47 |
| 16 | 19.9633 | 4.44405 | 8.1 | 330.45 | 557.06 |
| 17 | 20.4448 | 4.34045 | 2.08 | 84.96 | 554.26 |
| 18 | 21.5385 | 4.12247 | 46.74 | 1906.64 | 563.2 |
| 19 | 21.8363 | 4.06692 | 30.59 | 1247.85 | 558.21 |
| 20 | 22.298 | 3.98374 | 4.26 | 173.92 | 542.66 |
| 21 | 22.6877 | 3.91618 | 34.96 | 1426.11 | 525.6 |
| 22 | 22.7331 | 3.90847 | 41.09 | 1676.16 | 523.33 |
| 23 | 23.4485 | 3.7908 | 2.36 | 96.41 | 486.38 |
| 24 | 23.9683 | 3.70977 | 7.46 | 304.51 | 471.27 |
| 25 | 24.2747 | 3.66362 | 3.42 | 139.6 | 459.71 |
| 26 | 24.6237 | 3.61248 | 4.4 | 179.32 | 441.26 |
| 27 | 25.3992 | 3.50392 | 8.45 | 344.79 | 404.15 |
| 28 | 26.2102 | 3.39731 | 1.06 | 43.31 | 376.36 |
| 29 | 27.2705 | 3.26758 | 15.59 | 636.06 | 349.87 |
| 30 | 27.7298 | 3.21449 | 0.92 | 37.61 | 326.69 |
| 31 | 28.1584 | 3.16653 | 1.98 | 80.92 | 298.66 |
| 32 | 29.3526 | 3.04036 | 13.11 | 534.76 | 243.82 |
| 33 | 30.0321 | 2.9731 | 1.84 | 75.07 | 218.9 |
| 34 | 30.5655 | 2.92242 | 0.94 | 38.17 | 201.95 |
| 35 | 31.6336 | 2.82614 | 1.92 | 78.37 | 177.03 |
| 36 | 32.6048 | 2.74414 | 2.17 | 88.5 | 169.17 |
| 37 | 32.7713 | 2.73058 | 2.92 | 119.09 | 168.46 |
| 38 | 33.2301 | 2.69392 | 2.92 | 119.33 | 161.94 |
| 39 | 34.3393 | 2.60939 | 2.57 | 105 | 155.38 |
| 40 | 35.9558 | 2.4957 | 0.63 | 25.7 | 152.69 |
| 41 | 36.3391 | 2.47025 | 1.84 | 75.14 | 155.76 |
| 42 | 37.3895 | 2.40323 | 1.86 | 75.76 | 154.42 |
| 43 | 38.1793 | 2.35531 | 1.4 | 57.01 | 150.19 |

### Stability Analysis

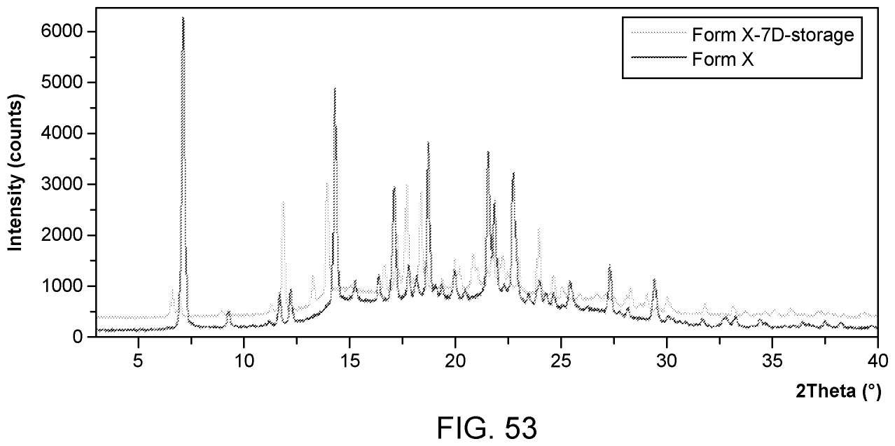

The stability of Form X was evaluated over 7 days of storage at room temperature in a sealed vial. Portions of the Form X sample were collected following 0 and 7 days of storage, the results of which are shown in FIG. 53. In this figure, the XR-PD patterns are displayed for Form X following 0 (Form X) and 7 days of storage (Form X-7D3-storage). As seen in the figure, Form X converted to a new form (hereinafter identified as Form XI) with a distinct XR-PD pattern after 7 days of storage in a sealed vial at room temperature. This conversion included the near complete loss of all Form X XR-PD signals, suggesting near complete conversion to Form XI.

### Example 14

### Recrystallization of (Z)-Endoxifen to Yield Form XI

This example describes crystallization of (Z)-endoxifen (Formula (III) e.g., Formula (IV)) to yield a polymorphic Form XI of (Z)-endoxifen. An endoxifen composition containing ≥95% (Z)-endoxifen free base was prepared as described herein in EXAMPLE 3. Some solutions in the following example were precipitated by cooling of the solution from a temperature between 50-100° C. to between 10-25° C. at a rate ranging from −0.2 to −0.5° C./minute resulting in a precipitate.

### Form XI Formation—Storage of Form X

Form XI was generated from Form X by storing the Form X for 7 days in a sealed vial at room temperature, as described in EXAMPLE 13.

Form XI Recrystallization—Precipitation from 4-Methyl-2-Pentanone

Form XI was also generated by precipitation during slow cooling of a 200 mg/ml (Z)-endoxifen solution in 4-methyl-2-pentanone, yielding the crystalline solid of Form XI.

### XRPD Characterization

FIG. 54 provides an x-ray powder diffraction spectrum of Form XI and includes peaks 1-48 corresponding to the peak numbers (No.) in TABLE 13. Peaks in FIG. 54 and TABLE 13 are provided as relative intensities (Rel. Int.) standardized against the peak at 14.0° 2θ (the peak with the highest intensity). The ten largest peaks by measure of Rel. Int. identified in the XRPD of Form XI, were at 14.0, 17.7, 11.9, 18.4, 23.9, 17.3, 21.8, 20.8, 23.0, and 22.2° 2θ, corresponding to peak numbers (No.) 7, 10, 5, 11, 24, 9, 19, 16, 23, and 21, respectively. The three largest peaks by measure of Rel. Int. were at 14.0, 17.7, and 11.9° 2θ.

TABLE 13
X-Ray Powder Diffraction Peak Table for Form XI

| | Pos. | d-spacing | | Height | Backgr. |
|---|---|---|---|---|---|
| No. | (°2Th.) | (Å) | Rel. Int. (%) | (cts) | (cts) |
| 1 | 6.6756 | 13.24114 | 16.37 | 530.34 | 183 |
| 2 | 7.0051 | 12.61917 | 15.18 | 491.96 | 185 |
| 3 | 9.0057 | 9.81981 | 3.16 | 102.23 | 199 |
| 4 | 11.3291 | 7.81057 | 4.88 | 158.04 | 263 |
| 5 | 11.904 | 7.43464 | 65.38 | 2118.12 | 294.4 |
| 6 | 13.311 | 6.65177 | 17.72 | 574.25 | 410.1 |
| 7 | 13.966 | 6.34125 | 100 | 3239.8 | 475.6 |
| 8 | 16.6618 | 5.32086 | 15.72 | 509.14 | 668.18 |
| 9 | 17.2548 | 5.13931 | 34.82 | 1128.18 | 694.48 |
| 10 | 17.7298 | 5.00266 | 65.96 | 2136.89 | 711 |
| 11 | 18.3734 | 4.82885 | 58.9 | 1908.39 | 727 |
| 12 | 18.6172 | 4.76616 | 17.04 | 552 | 732 |
| 13 | 19.3825 | 4.57968 | 6.34 | 205.28 | 741.25 |
| 14 | 19.982 | 4.44361 | 18.63 | 603.62 | 745 |
| 15 | 20.1975 | 4.39669 | 13.24 | 429.08 | 745 |
| 16 | 20.8444 | 4.26168 | 21.3 | 690.14 | 740 |
| 17 | 21.03 | 4.22448 | 12.8 | 414.8 | 737 |
| 18 | 21.4998 | 4.13322 | 13.92 | 450.9 | 727 |
| 19 | 21.7525 | 4.08577 | 31.57 | 1022.73 | 719.75 |
| 20 | 22.0376 | 4.03355 | 19.44 | 629.96 | 710.24 |
| 21 | 22.2315 | 3.99881 | 19.8 | 641.55 | 703.85 |
| 22 | 22.48 | 3.95516 | 11.55 | 374.1 | 694 |
| 23 | 22.9726 | 3.87145 | 21 | 680.46 | 672.74 |
| 24 | 23.9425 | 3.71677 | 39.3 | 1273.27 | 627 |
| 25 | 24.6308 | 3.61445 | 12.91 | 418.42 | 589.92 |
| 26 | 25.044 | 3.55574 | 6.27 | 203.24 | 565 |
| 27 | 25.8615 | 3.44517 | 3.23 | 104.79 | 517.85 |
| 28 | 26.6687 | 3.34269 | 5.07 | 164.24 | 475.13 |
| 29 | 27.087 | 3.29202 | 5.73 | 185.53 | 449.3 |
| 30 | 27.5142 | 3.24187 | 4.69 | 151.94 | 421 |
| 31 | 28.0666 | 3.17931 | 6.55 | 212.1 | 388.34 |
| 32 | 28.2747 | 3.15638 | 11.39 | 368.95 | 379 |
| 33 | 28.68 | 3.11269 | 2.59 | 84 | 358 |
| 34 | 29.0274 | 3.07622 | 8.83 | 286.05 | 339.26 |
| 35 | 29.5641 | 3.02159 | 1.92 | 62.29 | 309.59 |
| 36 | 29.9701 | 2.98157 | 8.77 | 284.15 | 286.99 |
| 37 | 31.7608 | 2.81744 | 6.16 | 199.65 | 228 |
| 38 | 32.5837 | 2.74814 | 1.2 | 39.01 | 214 |
| 39 | 33.1274 | 2.70427 | 5.11 | 165.47 | 213 |
| 40 | 33.7178 | 2.65826 | 2.33 | 75.64 | 208 |
| 41 | 34.6375 | 2.58975 | 1.88 | 60.83 | 209 |
| 42 | 35.0199 | 2.56235 | 3.22 | 104.4 | 208 |
| 43 | 35.7921 | 2.50882 | 4.35 | 140.88 | 204 |
| 44 | 36.4 | 2.4683 | 1.02 | 33 | 200 |
| 45 | 37.1831 | 2.4181 | 2.29 | 74.09 | 196 |
| 46 | 37.6624 | 2.38842 | 1.89 | 61.25 | 192 |
| 47 | 38.3357 | 2.34801 | 1.91 | 61.8 | 180 |
| 48 | 39.179 | 2.29939 | 2.41 | 78.04 | 185 |

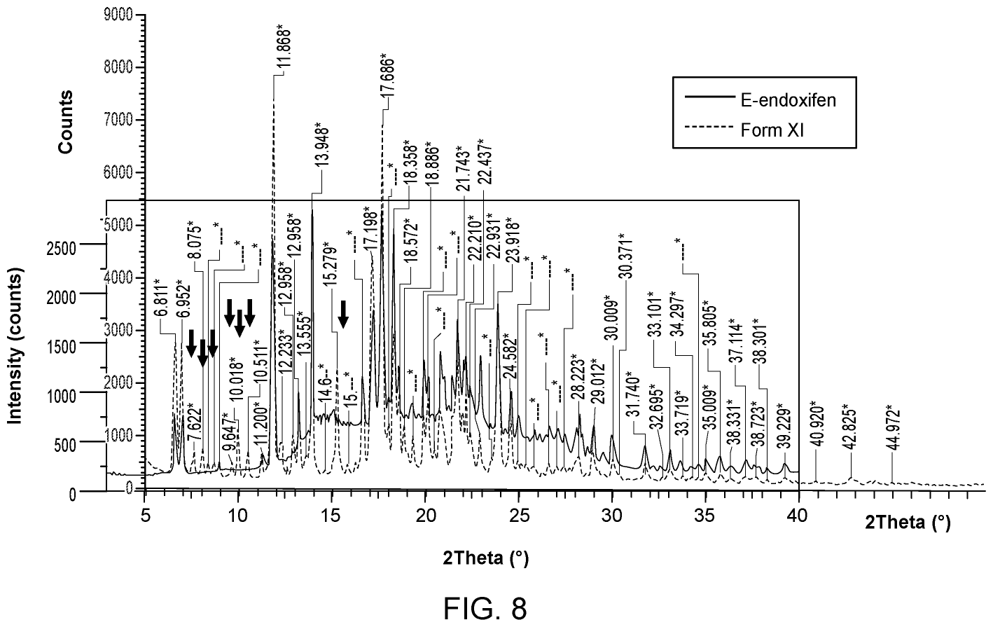

It was observed that Form XI shows some similarity to the XRPD pattern of (E)-endoxifen. FIG. 8 provides an overlay of Form XI and (E)-endoxifen XRPD spectra. The arrows indicate select peaks which are present in the (E)-endoxifen XRPD, and not in Form XI XRPD.

### Stability Analysis

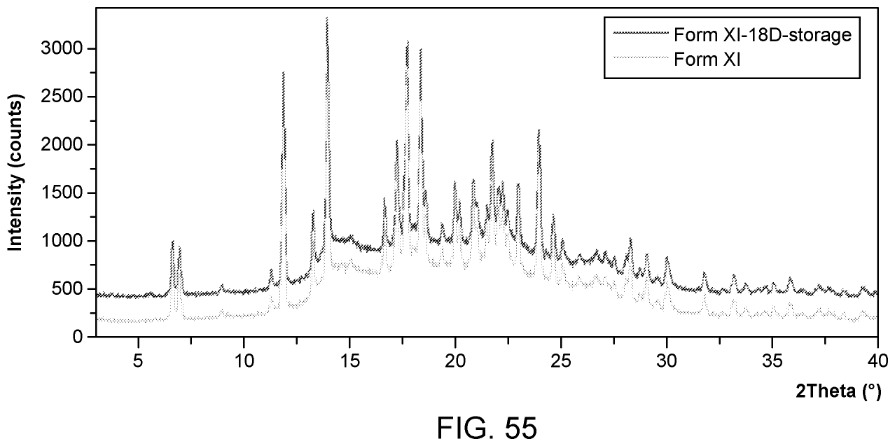

The stability of Form XI over 18 days of room temperature storage was evaluated with XRPD. Portions of a Form XI sample were collected for XRPD analysis following 0 (Form XI) and 18 days of storage (Form XI-18D-storage). The results of these analyses are shown in FIG. 55, in which the bottom XRPD pattern corresponds to Form XI following 0 days of storage and the top XRPD pattern corresponds to Form XI following 18 days of storage. Minimal changes were observed in the Form XI XRPD over the 18 days of storage, indicating that Form XI is stable under ambient conditions.

### Example 15

### Recrystallization of (Z)-Endoxifen to Yield Form XII

This example describes crystallization of (Z)-endoxifen (Formula (IV)) to yield polymorphic Form XII of (Z)-endoxifen. An endoxifen composition containing ≥95% (Z)-endoxifen free base was prepared as described in EXAMPLE 3 herein.

### Form XII Recrystallization (1)—THFAcetone Precipitation

Form XII was generated by precipitation of (Z)-endoxifen from a saturated THF solution by addition of the saturated solution to acetone, four days of storage in a sealed vial, and subsequent evaporation at room temperature yielding the crystalline solid of polymorphic Form XII. Further replicates of this method yielded small amounts of Form I and Form XIII mixed with Form XII. A similar microgram-scale method experiment utilizing 5 days storage in a sealed vial also yielded Form XII.

Recrystallizations Which Generated Form XII with Minor Traces of Other Forms

Form XII was also generated by a micro scale-up of Form XII Recrystallization (1), and yielded Form XII in mixture with polymorphic Form XIII, detailed further in EXAMPLE 16. Form XII was also generated by a room temperature evaporation of a precipitation by addition of acetone to a (Z)-endoxifen saturated THF solution with traces of Form I. Form XII was also generated by a room temperature evaporation of a precipitation by addition of THF to a (Z)-endoxifen saturated acetone solution with traces of Form XIII.

### XRPD Characterization

Polymorphic Form XII exhibited the X-ray powder diffraction (XRPD) pattern shown in FIG. 56 with peaks 1-25 corresponding to the peak numbers (No.) in TABLE 14. Peaks in FIG. 56 and TABLE 14 are provided as relative intensities (Rel. Int.) standardized against the peak at 12.5° 2θ (the peak with the highest intensity). The ten largest peaks by measure of Rel. Int. identified in the XRPD of Form XII, were at 12.5, 15.6, 19.0, 21.9, 20.2, 16.0, 21.6, 22.4, 16.8, and 12.8° 2θ, corresponding to peak numbers (No.) 4, 6, 10, 16, 12, 7, 15, 17, 8, and 5, respectively. The three largest peaks by measure of Rel. Int. were at 12.5, 15.6, and 19.0° 2θ.

TABLE 14
X-Ray Powder Diffraction Peak Table for Form XII

| | Pos. | d-spacing | Rel. | Height | Backgr. |
|---|---|---|---|---|---|
| No. | (°2Th.) | (Å) | Int. (%) | (cts) | (cts) |
| 1 | 9.49 | 9.3197 | 3.56 | 106 | 213 |
| 2 | 9.65 | 9.16553 | 2.52 | 75 | 214 |
| 3 | 11.142 | 7.94135 | 6.57 | 195.48 | 235 |
| 4 | 12.5243 | 7.06778 | 100 | 2973.87 | 340.43 |
| 5 | 12.7875 | 6.9229 | 7.7 | 229.05 | 365.75 |
| 6 | 15.5564 | 5.69634 | 29.38 | 873.81 | 658.64 |
| 7 | 16.0123 | 5.53518 | 11.9 | 353.86 | 669 |
| 8 | 16.7857 | 5.28187 | 8.57 | 255 | 671 |
| 9 | 18.332 | 4.83968 | 3.27 | 97.17 | 707 |
| 10 | 18.9869 | 4.6742 | 16.44 | 488.78 | 722 |
| 11 | 19.3512 | 4.58701 | 7.59 | 225.62 | 721 |
| 12 | 20.2086 | 4.39429 | 12.47 | 370.86 | 712 |
| 13 | 20.9622 | 4.23798 | 7.55 | 224.61 | 710 |
| 14 | 21.3527 | 4.16135 | 6.66 | 198 | 711 |
| 15 | 21.579 | 4.11823 | 10.47 | 311.31 | 708 |
| 16 | 21.9096 | 4.05682 | 15.13 | 449.83 | 698 |
| 17 | 22.3581 | 3.97646 | 8.68 | 258.13 | 674 |
| 18 | 22.85 | 3.89195 | 3.33 | 99.08 | 637 |
| 19 | 23.9402 | 3.71714 | 6.3 | 187.37 | 561.98 |
| 20 | 24.1334 | 3.68781 | 3.68 | 109.44 | 548 |
| 21 | 25.1408 | 3.54228 | 3.56 | 105.72 | 518 |
| 22 | 25.7083 | 3.46535 | 3.18 | 94.59 | 512 |
| 23 | 26.183 | 3.4036 | 2.51 | 74.75 | 495.7 |
| 24 | 28.1567 | 3.16934 | 2.83 | 84.13 | 342.33 |
| 25 | 29.3323 | 3.04494 | 2.55 | 75.95 | 289.77 |

### Stability Analysis

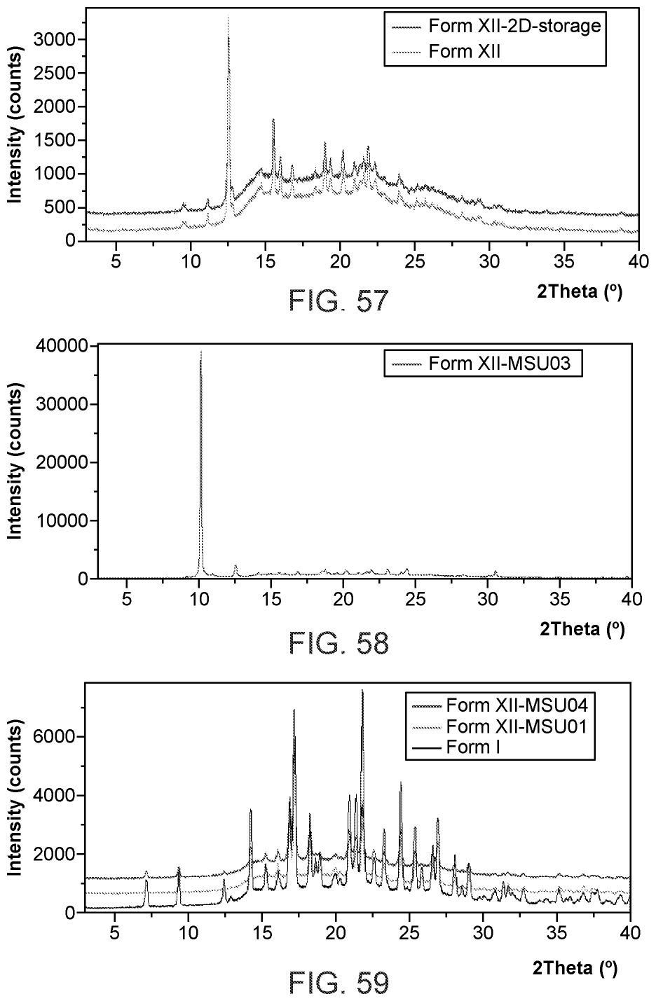

The stability of Form XII was evaluated over 2 days of storage in a sealed vial at room temperature. Portions of the Form XII sample were collected following 0 (Form XII) and 2 days of storage (Form XII-2D-storage) for XRPD analysis, the results of which are shown in FIG. 57. This figure shows XRPD of Form XII following 0 (Form XII) and 2 days (Form XII-2D-storage) storage. Minimal changes in the XRPD were observed following two days of storage, indicating that Form XII is stable under ambient conditions.

### Microgram Scale-Up Recrystallizations of Form XI

The Form XII Recrystallization (1) procedure was repeated at microgram scale. In total, 7 replicates were performed with varying starting volumes of (Z)-endoxifen saturated THE solutions and storage times. These recrystallizations generated a range of XRPD patterns including mixtures of different polymorphic forms, as shown in TABLE 15.

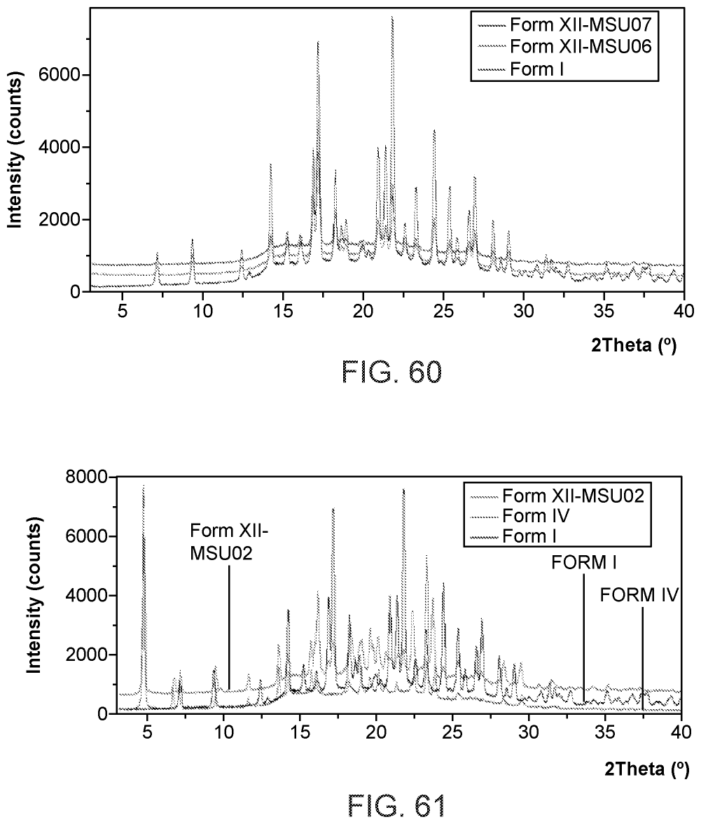

TABLE 15
Microgram Scale-Up Crystallizations

| | | Further | |
|---|---|---|---|
| | Procedure | Treatment Prior | |
| | (solvent/antisolvent is | to Evaporation at | |
| | acetone/tetrahydrofuran | Room | XRPD Results/ |
| Experiment | (THF)) | Temperature | Figure |
| Form XII | 30 μL of saturated solution | Storage at- | Form XII + signal at |
| Recrystallization | 1:10 solvent/antisolvent ratio | 20° C. 3 days | 10.1°2θ ascribable to |
| (1) | | | Form XIII |
| Micro Scale-Up | 100 μL of saturated solution | Storage at- | Form I/FIG. 59 |
| 1 of Form XII | 1:10 solvent/antisolvent ratio | 20° C. 1 day | |
| (Form XII- | | | |
| MSU01) | | | |
| Micro Scale-Up | 150 μL of saturated solution | Storage at- | Form I and Form IV/ |
| 2 of Form XII | 1:10 solvent/antisolvent ratio | 20° C. 1 day | FIG. 61 |
| (Form XII- | | | |
| MSU02) | | | |
| Micro Scale-Up | 200 μL of saturated solution | Storage at- | Form XII and Form |
| 3 of Form XII | 1:10 solvent/antisolvent ratio | 20° C. 1 day | XIII/FIG. 58 |
| (Form XII- | | | |
| MSU03) | | | |
| Micro Scale-Up | 200 μL of saturated solution | None | Form I/FIG. 59 |
| 4 of Form XII | 1:10 solvent/antisolvent ratio | | |
| (Form XII- | | | |
| MSU04) | | | |
| Micro Scale-Up | 500 μL of saturated solution | None | Form XIV |
| 5 of Form XII | 1:10 solvent/antisolvent ratio | | |
| (Form XII- | | | |
| MSU05) | | | |
| Micro Scale-Up | 200 μL of saturated solution | Storage at- | Form I/FIG. 60 |
| 6 of Form XII | 1:10 solvent/antisolvent ratio | 20° C. 1 day | |
| (Form XII- | | | |
| MSU06) | | | |
| Micro Scale-Up | 200 μL of saturated solution | Storage at- | Form I/FIG. 60 |
| 7 of Form XII | 1:10 solvent/antisolvent ratio | 20° C. 1 day | |
| (Form XII- | | | |
| MSU07) | | | |

XRPD of the 7 scale-up recrystallizations are summarized in FIG. 58-FIG. 61. As provided in FIG. 58, some scale-ups of Form XII exhibited XRPD patterns reflective of Form XII and Form XIII. FIG. 59 and FIG. 60 provide XRPD of scale-up recrystallizations which yielded Form I. FIG. 61 provides XRPD of a scale-up recrystallization Form XII (Form XII-MSU02) that exhibited a mixture of the reference XRPD pattern for Form I (Form I) and Form IV (Form IV).

### Example 16

### Purification of (Z)-Endoxifen to Yield a Mixture of Form XII and Form XIII

This example describes recrystallization of (Z)-endoxifen (Formula (IV)) to yield a polymorphic mixture of Form XII and Form XIII of (Z)-endoxifen. An endoxifen composition containing ≥95% (Z)-endoxifen free base was prepared as described herein in EXAMPLE 3.

### Form XII and Form XIII Recrystallization (1)—Form XII Micro Scale-Up

The mixture of Form XII and Form XIII was generated during a scaled-up (microgram-scale) crystallization for Form XII, described in EXAMPLE 15. Form XIII was observed as a minor component of a predominantly Form XII mixture generated by precipitation of (Z)-endoxifen from a saturated THE solution by addition of the saturated solution to acetone, four days of storage in a sealed vial, and subsequent evaporation at room temperature.

### XRPD Characterization

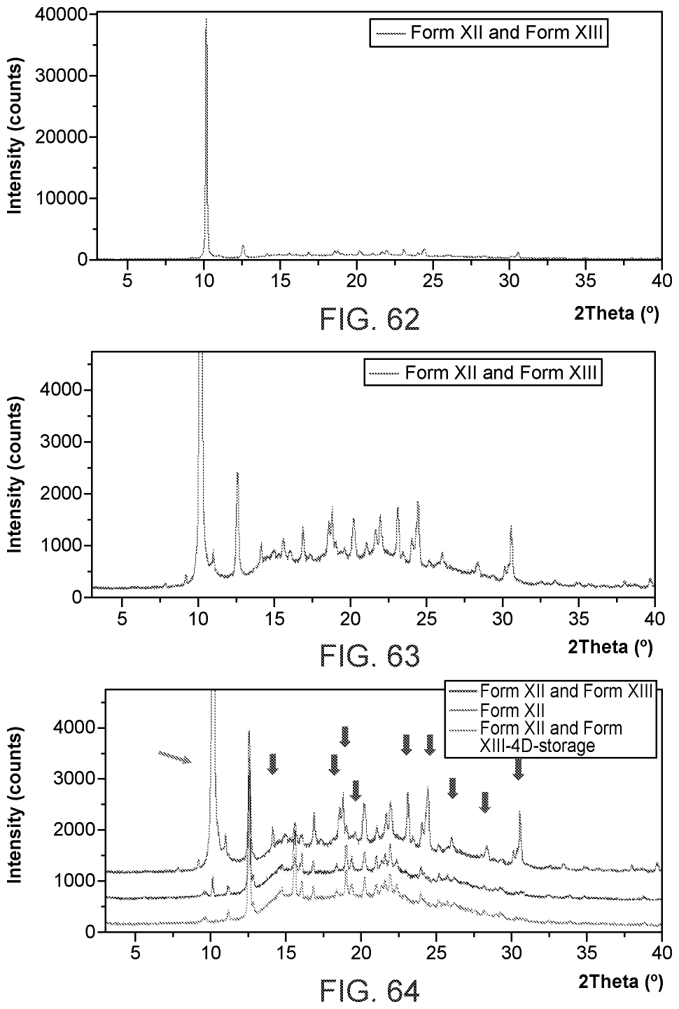

The XRPD pattern of the mixture of Form XII and Form XIII is seen in FIG. 62, with a magnification of the pattern seen in FIG. 63. As seen in the figure, Form XIII showed an intense peak at 10.1° 20 with the mixture also containing the pattern of Form XII.

### Stability Analysis

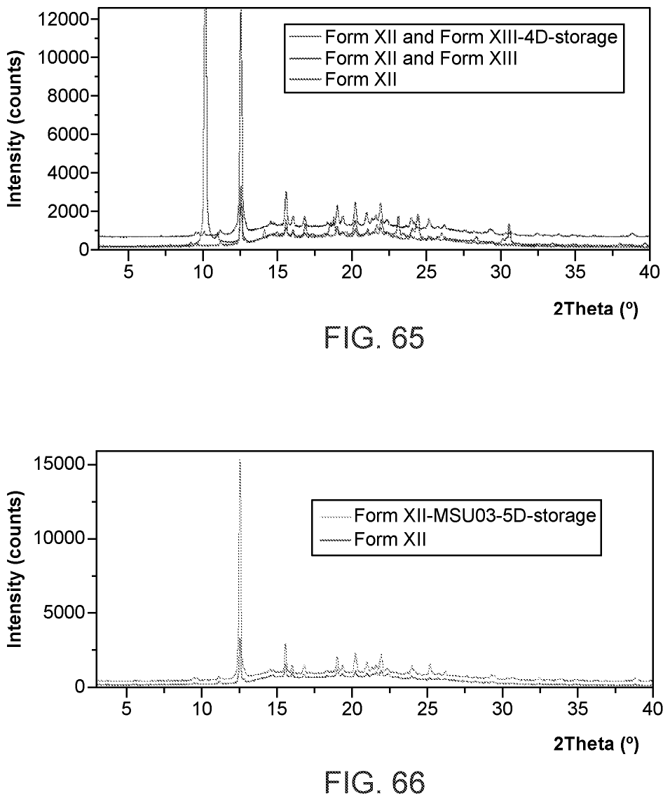

The stability of the mixture of Form XII and Form XIII generated Form XII Recrystallization (1) was evaluated over 4 days of storage in a sealed vial at room temperature. Portions of the sample were collected following 0 and 4 days of storage for XRPD analysis, the results of which are shown magnified in FIG. 64 and not magnified in FIG. 65. As seen in the figures, when compared to the XRPD pattern of the mixture of Form XII and Form XIII at 0 days (Form XII and Form XIII), and the reference pattern for Form XII (Form XII), the mixture exhibited conversion of Form XIII to Form XII following 4 days storage (Form XII and Form XIII-4D-storage), notably by the loss of the peak signal at 10.1° 2θ ascribable to Form XIII. After storage for an additional day (5 days total), shown in FIG. 66, the 5 day storage of the Micro Scale-Up 3 of Form XII experiment (Form XII-MSU03-5D-storage) resulted in conversion to only Form XII (Form XII).

### DSC Characterization

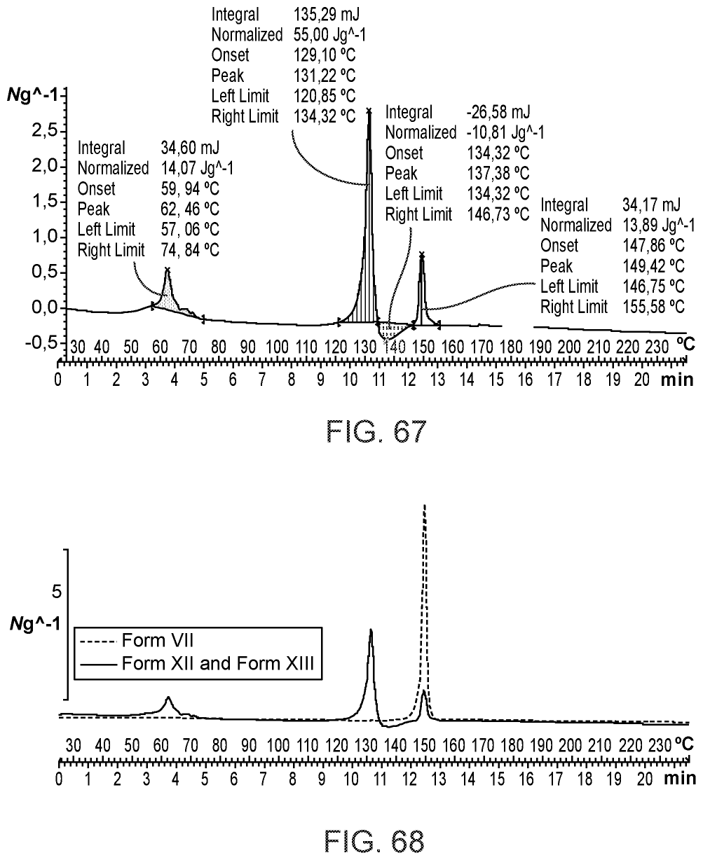

DSC characterization was performed for the mixture of polymorphic Form XII and Form XIII and showed several thermal events displayed in FIG. 67. As seen in the figure, there is a first endothermic event at 63° C. (onset at 60° C.), ascribable to solvent release, a second endothermic event at 131° C. (onset at 129° C.) possibly ascribable to Form IX melting, an exothermic event at 137° C. (onset at 134° C.) possibly consistent with recrystallization, and another endothermic event at 149° C. (onset 148° C.) possibly ascribable to Form VII melting. The DSC profile of the mixture of Form XII and Form XIII is further compared to the DSC profile of Form VII, as shown in FIG. 68. As seen in this figure, the DSC profile of the mixture of Form XII and Form XIII (bottom) and the DSC profile of Form VII (top) share a peak around 149° C. (onset at 148° C.) further confirming that this peak in the DSC profile might have been ascribable to Form VII melting.

### TGA Analysis

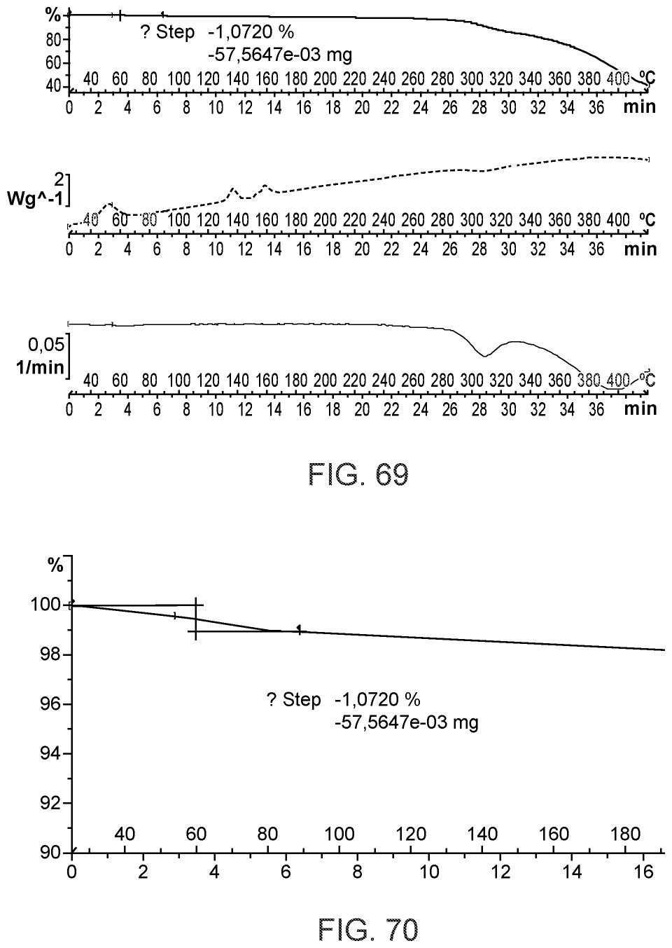

TGA analysis of the mixture of Form XII and Form XIII is shown in FIG. 69 and FIG. 70. As seen in FIG. 69, the TGA profile (top) showed a weight loss of 1.1% between 25-90° C., ascribable to solvent release. Degradation of the mixture of Form XII and Form XIII occurred above 270° C. FIG. 69 also shows the heat flow profile (middle) and the 1^{st} derivative of the TGA profile (bottom). FIG. 70 displays a magnification of the heat loss of 1.1% between 25-90° C.

### Example 17

### Purification of (Z)-Endoxifen to Yield Form XIV

This example describes recrystallization of (Z)-endoxifen (Formula (IV)) to yield a polymorphic Form XIV of (Z)-endoxifen. An endoxifen composition containing ≥95% (Z)-endoxifen free base was prepared as described herein in EXAMPLE 3.

### Form XIV Recrystallization—Micro Scale-Up of Form XII

Form XIV was generated by a microgram scale-up of Form XII Recrystallization (1) by precipitation from a (Z)-endoxifen saturated THE solution added to acetone and subsequently evaporated at room temperature as described in EXAMPLE 15.

### XRPD Characterization

Polymorphic Form XIV exhibited an x-ray powder diffraction (XRPD) pattern shown in FIG. 71 with peaks 1-42 corresponding to the peak numbers (No.) in TABLE 16. Peaks in FIG. 71 and TABLE 16 are provided as relative intensities (Rel. Int.) standardized against the peak at 11.6° 2θ (the peak with the highest intensity). The ten largest peaks by measure of Rel. Int. identified in the XRPD of Form XIV, were at 11.6, 21.3, 19.3, 17.5, 15.4, 21.6, 5.8, 16.3, 21.9, and 23.9° 2θ, corresponding to peak numbers (No.) 5, 21, 18, 15, 10, 22, 1, 12, 23, and 28, respectively. The three largest peaks by measure of Rel. Int. were at 11.6, 21.3, and 19.30° 2θ.

TABLE 16
X-Ray Powder Diffraction Peak Table for Form XIV

| | Pos. | d-spacing | | Height | Backgr. |
|---|---|---|---|---|---|
| No. | (°2Th.) | (Å) | Rel. Int. (%) | (cts) | (cts) |
| 1 | 5.8373 | 15.14067 | 29.46 | 672.43 | 177.48 |
| 2 | 6.992 | 12.64265 | 10.3 | 235.02 | 192.41 |
| 3 | 9.5914 | 9.22138 | 2.99 | 68.16 | 217.43 |
| 4 | 10.6194 | 8.3309 | 3.67 | 83.85 | 240.69 |
| 5 | 11.5963 | 7.63122 | 100 | 2282.33 | 288.85 |
| 6 | 12.6843 | 6.97897 | 16.86 | 384.74 | 376.56 |
| 7 | 13.0108 | 6.8046 | 6.97 | 159 | 424.81 |
| 8 | 13.2337 | 6.69048 | 9.5 | 216.9 | 457.36 |
| 9 | 13.9725 | 6.33831 | 12.7 | 289.92 | 556.01 |
| 10 | 15.3893 | 5.75784 | 36.8 | 839.96 | 699.51 |
| 11 | 15.9656 | 5.55126 | 3.48 | 79.34 | 739.36 |
| 12 | 16.3219 | 5.43087 | 26.58 | 606.6 | 759.06 |
| 13 | 16.8156 | 5.27254 | 8.83 | 201.52 | 782.95 |
| 14 | 17.0825 | 5.19075 | 13.63 | 311.07 | 792.66 |
| 15 | 17.46 | 5.07935 | 48.49 | 1106.75 | 802.83 |
| 16 | 18.1954 | 4.8757 | 8.25 | 188.2 | 812.9 |
| 17 | 18.6248 | 4.76424 | 7.1 | 162.07 | 815.87 |
| 18 | 19.2712 | 4.60587 | 66.22 | 1511.43 | 814.53 |
| 19 | 19.7668 | 4.49149 | 18.43 | 420.59 | 805.78 |
| 20 | 20.933 | 4.24383 | 12.24 | 279.43 | 781.3 |
| 21 | 21.3177 | 4.16811 | 90.5 | 2065.6 | 779.78 |
| 22 | 21.557 | 4.12238 | 30.48 | 695.73 | 776.46 |
| 23 | 21.9121 | 4.05637 | 23.27 | 531.03 | 767.92 |
| 24 | 22.3433 | 3.97905 | 7.5 | 171.18 | 752.09 |
| 25 | 22.639 | 3.92774 | 8.55 | 195.19 | 737.82 |
| 26 | 23.2577 | 3.82465 | 6.27 | 143.18 | 699.4 |
| 27 | 23.6107 | 3.76826 | 6.66 | 152.07 | 674.5 |
| 28 | 23.9445 | 3.71647 | 19.19 | 437.97 | 647.62 |
| 29 | 24.5069 | 3.63245 | 7.58 | 172.9 | 594.45 |
| 30 | 25.29 | 3.52171 | 9.42 | 215.08 | 539.69 |
| 31 | 25.4983 | 3.49341 | 18.85 | 430.12 | 538.21 |
| 32 | 25.8811 | 3.4426 | 12.08 | 275.6 | 531.59 |
| 33 | 26.9357 | 3.31017 | 12.9 | 294.33 | 494.73 |
| 34 | 27.4391 | 3.25058 | 9.31 | 212.39 | 464.96 |
| 35 | 27.9897 | 3.18787 | 13.45 | 306.97 | 422.92 |
| 36 | 28.5164 | 3.13017 | 4.02 | 91.76 | 384.52 |
| 37 | 28.9251 | 3.08687 | 2.72 | 62.13 | 358.49 |
| 38 | 31.0436 | 2.88088 | 2.94 | 67.11 | 255.4 |
| 39 | 32.2769 | 2.77356 | 2.7 | 61.72 | 239.82 |
| 40 | 32.5251 | 2.75296 | 3.8 | 86.72 | 239.24 |
| 41 | 32.8682 | 2.72501 | 3.64 | 83.02 | 237.6 |
| 42 | 34.5926 | 2.59301 | 3.48 | 79.42 | 223.17 |

### Stability Analysis

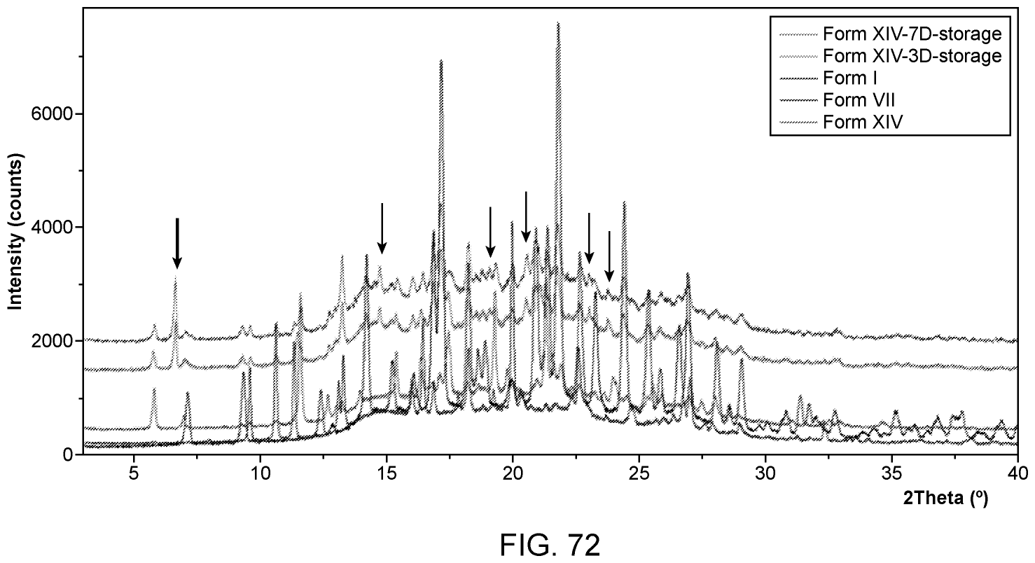

The stability of Form XIV was evaluated over 7 days storage in a sealed vial at room temperature. Portions of the Form XIV sample were taken at 0 (Form XIV), 3 (Form XIV-3D-storage), and 7 days of storage (Form XIV-7D-storage) for XRPD analysis, the results of which are shown in FIG. 72. As seen in the figure, when the XRPD patterns are compared to the reference patterns of Form I and Form VII, Form XIV exhibited partial conversion to Forms I and VII following 3 and 7 days storage, as well as to additional unassigned forms with XRPD signals at 6.7, 14.7, 19.0, 20.5, 23.0, and 23.8020 (highlighted in FIG. 72 with arrows). Some Form XIV remained following 3 and 7 days storage.

### Example 18

### Purification of (Z)-Endoxifen to Yield Amorphous (Z)-Endoxifen

This example describes precipitation of (Z)-endoxifen (Formula (IV)) to yield amorphous (Z)-endoxifen. An endoxifen composition containing ≥95%0 (Z)-endoxifen free base was prepared as described herein in EXAMPLE 3.

Amorphous (Z)-endoxifen was generated by high-temperature evaporation of a solution of (Z)-endoxifen in DMF. The resulting solid appeared as a yellowish solid that converted to an off-white powder when manipulated with a spatula.

### Stability Analysis

The stability of amorphous (Z)-endoxifen was evaluated over 18 days of storage in a sealed vial at room temperature. Portions of the sample of amorphous (Z)-endoxifen were collected at 0 and 18 days of storage for XRPD characterization, the results of which yielded amorphous (Z)-endoxifen and indicate that amorphous (Z)-endoxifen is stable and does not convert into other polymorphic forms during dry, room temperature storage.

### Micro Scale-Up Experiment of Amorphous (Z)-Endoxifen

Three microgram scale-up precipitations were performed for amorphous (Z)-endoxifen. 50 mg of (Z)-endoxifen was dissolved in 5 ml of DMF and stirred for five minutes. The clear solution was then filtered and left to evaporate at 60° C. and the resulting solids were used for XRPD characterization, the results of which yielded amorphous (Z)-endoxifen.

### Example 19

### Solubility of (Z)-Endoxifen

The solubility of (Z)-endoxifen produced according to EXAMPLE 3 was tested for its solubility in a variety of solvents varying in dipole moment, dielectric constant, and boiling point. The solubilities determined from these experiments guided the crystallizations outlined in EXAMPLE 5-EXAMPLE 17 and were used to map the impact of different parameters on polymorphic forms of (Z)-endoxifen. Each solvent allowed for definition of specific crystallization conditions based on its physical-chemical properties. The procedure described below was the procedure employed for all solvent and solvent mixture solubility tests.

### Procedure for Solubility Testing

To test the solubility of (Z)-endoxifen in varying conditions, 10 mg of (Z)-endoxifen was added to a stoppered tube, and 0.01 ml of the selected solvent was added (1000 mg/ml solution). The tube was shaken vigorously for 1 minute and then incubated at a constant temperature of 25.0° C. for 15 minutes. If the solid was not completed dissolved, the tube was shaken again and placed at the constant temperature of 25.0° C. for another 15 minutes. If the solid was completely dissolved at this step, it was considered to be very soluble in the selected solvent system. If the solid was not dissolved by this step, another 0.09 ml was added (yielding a 100 mg/ml solution) and an attempt to further dissolve the solid was made by the same steps of shaking and incubating twice over as described previously. If the solid was completely dissolved at this step, it was considered freely soluble. If the solid was not dissolved by this step another 0.2 ml was added (yielding a 33.3 mg/ml solution) and an attempt to further dissolve the solid was made by the same steps of shaking and incubating twice over as described previously. If the solid was completely dissolved at this step, it was considered soluble. If the solid was not dissolved by this step another, 0.7 ml was added (yielding a 10 mg/ml solution) and an attempt to further dissolve the solid was made by the same steps of shaking and incubating twice over as described previously. If the solid was completely dissolved at this step, it was considered sparingly soluble. If the solid was not dissolved by this step the sample was considered to be slightly soluble or very slightly soluble and the solution was heated until the boiling point of the solvent system (max 100° C.) under stirring with a rate of 0.5° C. per minute temperature ramp rate to verify the solubility at high temperatures. The hot solution was after cooled to room temperature with a cooling rate of 0.5° C. per minute to verify if the sample had precipitated after up to 18 hours under magnetic stirring. The same procedure was also used for the solvent mixture solubility tests. The concentrations investigated for the solvent mixture stability were 40 mg/ml and 100 mg/ml. Some solutions in the following example were precipitated by cooling of the solution from a temperature between 50-100° C. to between 10-25° C. at a rate ranging from −0.2 to −0.5° C./minute resulting in a precipitate.

Solubility of (Z)-endoxifen

(Z)-endoxifen solubility was first tested in single-solvent systems. (Z)-endoxifen was soluble in the range of 250-500 mg/ml in DMF, 125-111 mg/ml in THF, and 20-17 mg/ml in methanol. At room temperature, (Z)-endoxifen was soluble at 10 mg/ml in 2-propanol, acetone, butyl acetate, ethyl acetate, ethanol, 4-met-2-pentanone, and toluene. (Z)-endoxifen exhibited a solubility of <10 mg/ml both at room temperature and high temperature in water, heptane, and t-butyl methyl ether. (Z)-endoxifen also had a solubility of <10 mg/ml in room temperature acetonitrile. At high temperature, (Z)-endoxifen was soluble at less than 50 mg/ml in acetone, 100-200 mg/ml in 4-met-2-pentanone, and 20-100 mg/ml in 2-propanol, acetonitrile, butyl acetate, ethyl acetate, ethanol, and toluene. A summary of the single solvent solubility of (Z)-endoxifen is displayed in TABLE 17.

TABLE 17
Single Solvent Solubility of (Z)-Endoxifen Experiments

| | | Solubility HT (10 mg/mL) | | | | Solubility HT (20 mg/mL) | | | | |
|---|---|---|---|---|---|---|---|---|---|---|
| | Solubility | | Clear | | Cloudy | | Clear | | Cloudy | Re-PPT after |
| | at RT | Sol. | point | Re- | point | Sol. | point | Re- | point | 18 hours |
| ID | (mg/ml) | HT | (° C.) | PPT | (° C.) | HT | (° C.) | PPT | (° C.) | at −20° C. |
| 2-propanol | 10 | YES | 44 | NO | — | YES | 50.6 | YES | — | — |
| acetonitrile | <10 | YES | 56 | YES | — | YES | 75 | YES | — | — |
| acetone | 10 | YES | 30 | NO | — | YES | 38 | — | — | YES |
| butyl acetate | 10 | YES | 50 | NO | — | YES | 56.8 | — | — | NO |
| butyl methyl ether | 10 | NO | — | — | — | | | | | |
| limethylformamide | 250-200 | — | — | — | — | | | | | |
| ethyl acetate | 10 | YES | 38 | NO | — | YES | 49.2 | — | — | NO |
| ethanol | 10 | YES | 34 | NO | — | YES | 34 | YES | — | — |
| water | <10 | NO | — | — | — | | | | | |
| heptane | <10 | NO | — | — | — | | | | | |
| methanol | 20-17 | — | — | — | — | | | | | |
| methyl isobutyl | 10 | YES | 44 | NO | — | YES | 49.3 | — | — | NO |
| ketone (4-methyl-2- | | | | | | | | | | |
| pentanone) | | | | | | | | | | |
| tetrahydrofuran | 125-111 | — | — | — | — | | | | | |
| toluene | 10 | YES | 66 | NO | — | YES | 77.8 | — | — | NO |

| | Solubility HT (100 mg/mL) | | | | | Solubility HT (200 mg/mL) | | | |
|---|---|---|---|---|---|---|---|---|---|
| | | Clear | | Cloudy | Re-PPT after | | Clear | | Cloudy |
| | Sol. | point | Re- | point | 18 hours | Sol. | Point | Re- | point |
| ID | HT | (° C.) | PPT | (° C.) | at −20° C. | HT | (° C.) | PPT | (° C.) |
| 2-propanol | YES | 74.4 | YES | 43.9 | — | | | | |
| acetonitrile | NO | — | — | — | — | | | | |
| acetone | NO | — | — | — | — | | | | |
| butyl acetate | YES | 94.5 | YES | 25 | — | | | | |
| butyl methyl ether | | | | | | | | | |
| limethylformamide | | | | | | | | | |
| ethyl acetate | YES | 74.4 | YES | 54.2 | — | | | | |
| ethanol | YES | 66 | YES | 39.7 | — | | | | |
| water | | | | | | | | | |
| heptane | | | | | | | | | |
| methanol | | | | | | | | | |
| methyl isobutyl | YES | 74.8 | NO | — | YES | YES | 84 | YES | 48.5 |
| ketone (4-methyl-2- | | | | | | | | | |
| pentanone) | | | | | | | | | |
| tetrahydrofuran | | | | | | | | | |
| toluene | YES | 96.6 | YES | 25 | — | | | | |

(Z)-endoxifen solubility was characterized for multi-solvent systems. (Z)-endoxifen was found to be soluble at less than 40 mg/ml in high temperature in acetone/acetonitrile (50/50 v/v), acetone/ethyl acetate (50/50 v/v), water/2-propanol (50/50 v/v), water/acetonitrile (50/50 v/v), water/THF (50/50 v/v), heptane/ethyl acetate (50/50 v/v), and heptane/toluene (50/50 v/v) mixtures. In butyl ethyl ether/methanol (50/50 v/v) at high temperature, (Z)-endoxifen was soluble in the range of 40-100 mg/ml.

At high temperature, (Z)-endoxifen exhibited 100 mg/ml solubility in ethyl acetate/2-propanol (50/50 v/v) and 40 mg/ml solubility in 2-propanol/THF (95/5 v/v), ethyl acetate/THF (95/5 v/v), and heptane/2-propanol (50/50 v/v) solvent mixtures. (Z)-endoxifen precipitated from each of these systems during cooling to between room temperature and −20° C. [0683](Z)-endoxifen was soluble in acetone/2-propanol (63/37 v/v) and in acetone/THF (95/5 v/v) at high temperature but precipitated after 18 hours at −20° C. A summary of the solvent mixture solubility experiments is provided in TABLE 18.

TABLE 18
Solvent Mixture Solubility of (Z)-Endoxifen Experiments

| | | Solubility HT (40 mg/mL) | | | | | Solubility HT (100 mg/mL) | | | | |
|---|---|---|---|---|---|---|---|---|---|---|---|
| | | | Clear | | Cloudy | Re-PPT after | | Clear | | Cloudy | Re-PPT after |
| | Ratio | Sol. | point | Re- | point | 18 hours | Sol. | point | Re- | point | 18 hours |
| Solvent mixture ID | v/v | HT | (° C.) | PPT | (° C.) | at −20° C. | HT | (° C.) | PPT | (° C.) | at −20° C. |
| 2-propanol/tetrahydrofuran | 95/5 | YES | 52.8 | YES | 28.6 | — | NO | — | — | — | — |
| acetone/2-propanol | 67/33 | YES | 36.8 | NO | — | YES | NO | — | — | — | — |
| acetone/acetonitrile | 50/50 | NO | — | — | — | — | NO | — | — | — | — |
| acetone/ethyl acetate | 50/50 | NO | — | — | — | — | NO | — | — | — | — |
| acetone/tetrahydrofuran | 95/5 | YES | 47.5 | NO | — | YES | NO | — | — | — | — |
| butyl methyl | 50/50 | YES | 31.8 | NO | — | NO | NO | — | — | — | — |
| ether/methanol | | | | | | | | | | | |
| ethyl acetate/2-propanol | 50/50 | | | | | | YES | 59.8 | YES | 25 | — |
| ethyl | 95/5 | YES | 56.3 | YES | 51.3 | — | NO | — | — | — | — |
| acetate/tetrahydrofuran | | | | | | | | | | | |
| water/2-propanol | 50/50 | NO | — | — | — | — | NO | — | — | — | — |
| water/acetonitrile | 50/50 | NO | — | — | — | — | NO | — | — | — | — |
| water/tetrahydrofuran | 95/5 | NO | — | — | — | — | NO | — | — | — | — |
| heptane/2-propanol | 50/50 | YES | 64.5 | YES | 33.7 | — | NO | — | — | — | — |
| heptane/ethyl acetate | 50/50 | NO | — | — | — | — | NO | — | — | — | — |
| heptane/toluene | 50/50 | NO | — | — | — | — | NO | — | — | — | — |

### Example 20

### Slurries of (Z)-Endoxifen

This example covers the solubility of (Z)-endoxifen in a variety of solvent mixtures. The solubilities determined in this example were used to calibrate crystallization conditions covered in further examples herein. (Z)-endoxifen slurries were generated with two classes of solvents: a first class in which (Z)-endoxifen has less than 10 mg/ml room temperature solubility, and a second class of solvents in which (Z)-endoxifen has between 10 and 33 mg/ml room temperature solubility.

### Single Solvents Slurries

For the single solvent slurry experiments, 10 mg of (Z)-endoxifen was suspended in 1 ml of 2-propanol, acetone, butyl acetate, butyl methyl ether, dimethyl formamide, or ethyl acetate (solvents in which (Z)-endoxifen has a solubility of less than 10 mg/ml) and stirred at approximately 350 rpm for either: (1) 3 days at room temperature (25° C.), (2) 14 days at room temperature (25° C.), or (3) 1 day at 50° C.

Further solubility analyses were then performed for solvents which formed suspensions at 50° C. (Z)-endoxifen was suspended in 500-600 μl of a single solvent to achieve 40 mg/ml, 50 mg/ml, 100 mg/ml, 170 mg/ml, 200 mg/ml, and 300 mg/ml concentrations. The suspensions were recovered and filtered with a nylon filter (0.22 μm) during centrifugation at 4000 rpm for 3 minutes. The solid (Z)-endoxifen collected through filtration was analyzed by XRPD. (Z)-endoxifen remaining in the filtrate was precipitated by addition of antisolvent and also subjected to XRPD analysis.

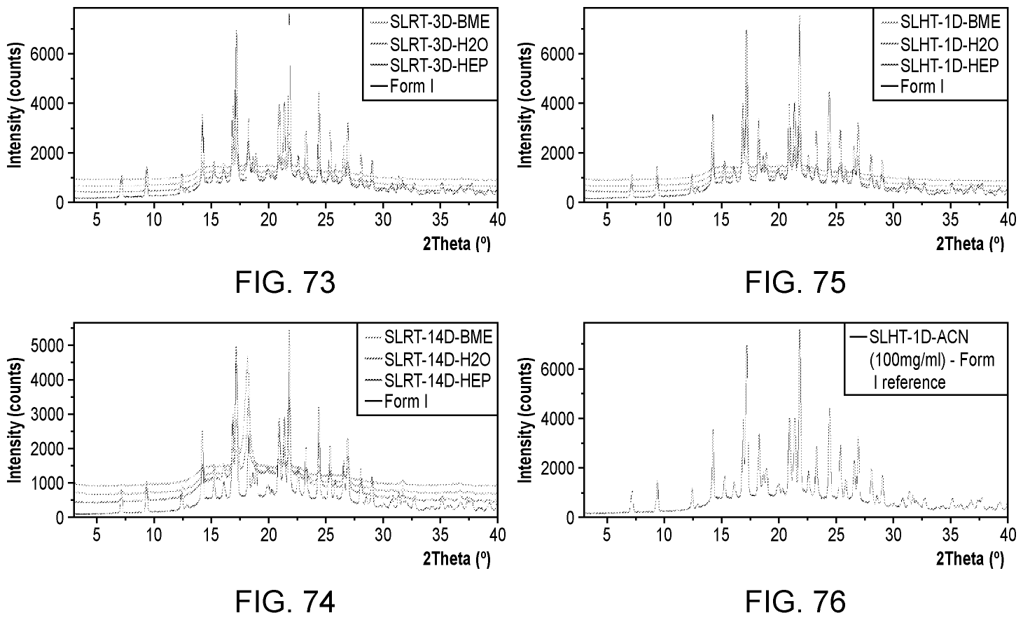

The results of the XRPD analyses are summarized in TABLE 19 and shown in FIG. 73-FIG. 84 and FIG. 92, with the “Experiment name” column in TABLE 19, referring to the name of each of the XRPD spectra displayed in FIG. 73-FIG. 84 and FIG. 92. As displayed in FIG. 73-FIG. 84 and FIG. 92 and in TABLE 19, all single solvent slurries yielded Form I. Three slurry experiments, one utilizing butyl acetate, a second utilizing ethyl acetate, and a third utilizing ethanol yielded Form I with minor amounts of additional polymorphs, as indicated by the presence of additional XRPD peaks.

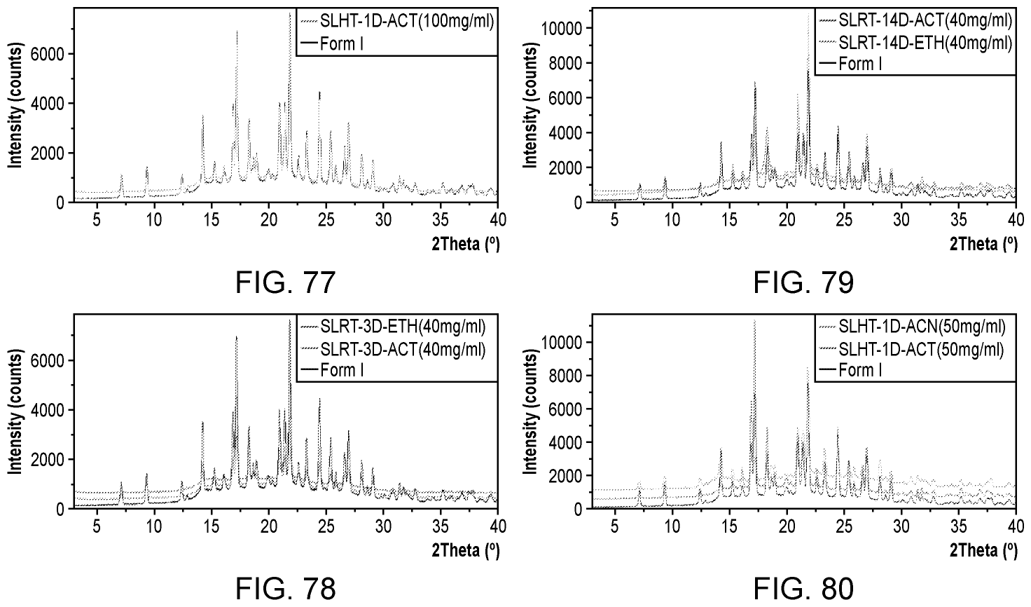

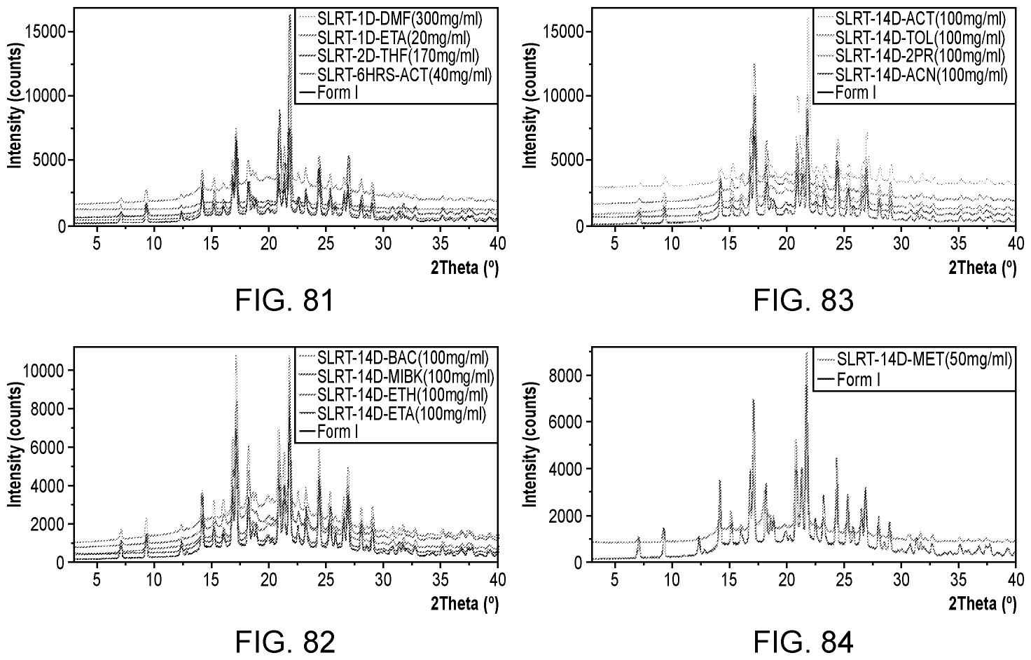

TABLE 19
Single Solvent Slurries of (Z)-Endoxifen Results

| | | | | | Solvent 1 | | | |
|---|---|---|---|---|---|---|---|---|
| | Experim. | Input | Solvent | Solvent 1 | Conc. in | Temp. | Temp | |
| Experiment | Type | Mass | 1 Type | Volume | Solvent 1 | Start | Duration | |
| name | [descript.] | [mg] | [name] | [mL] | [mg/mL] | [° C.] | [days] | |
| SLRT-14D- | SLRT | 50 | 2-propanol | 0.5 | 100 | RT | 14 | d |
| 2PR(100 mg/ml)/ | | | | | | | | |
| FIG. 83 | | | | | | | | |
| SLHT-1D- | SLHT | 25 | acetonitrile | 0.5 | 50 | 50 | 1 | d |
| ACN(50 mg/ml)/ | | | | | | | | |
| FIG. 80 | | | | | | | | |
| SLHT-1D- | SLHT | 25 | acetonitrile | 0.5 | 50 | 50 | 1 | d |
| ACN(50 mg/ml)- | | | | | | | | |
| vial attached/ | | | | | | | | |
| FIG. 92 | | | | | | | | |
| SLRT-14D- | SLRT | 50 | acetonitrile | 0.5 | 100 | RT | 14 | d |
| ACN(100 mg/ml)/ | | | | | | | | |
| FIG. 83 | | | | | | | | |
| SLHT-1D- | SLHT | 50 | acetonitrile | 0.5 | 100 | 50 | 1 | d |
| ACN(100 mg/ml) - | | | | | | | | |
| Form I reference/ | | | | | | | | |
| FIG. 76 | | | | | | | | |
| SLRT-3D- | SLRT | 20 | acetone | 0.5 | 40 | RT | 3 | d |
| ACT(40 mg/ml)/ | | | | | | | | |
| FIG. 78 | | | | | | | | |
| SLRT-14D- | SLRT | 20 | acetone | 0.5 | 40 | RT | 14 | d |
| ACT(40 mg/ml)/ | | | | | | | | |
| FIG. 79 | | | | | | | | |
| SLHT-1D- | SLHT | 25 | acetone | 0.5 | 50 | 40 | 1 | d |
| ACT(50 mg/ml)/ | | | | | | | | |
| FIG. 80 | | | | | | | | |
| SLRT-14D- | SLRT | 50 | acetone | 0.5 | 100 | RT | 14 | d |
| ACT(100 mg/ml)/ | | | | | | | | |
| FIG. 83 | | | | | | | | |
| SLHT-1D- | SLHT | 50 | acetone | 0.5 | 100 | 40 | 1 | d |
| ACT(100 mg/ml)/ | | | | | | | | |
| FIG. 77 | | | | | | | | |
| SLRT-6HRS- | SLRT | 40 | acetone | 1 | 40 | RT | 6 | h |
| ACT(40 mg/ml)/ | | | | | | | | |
| FIG. 81 | | | | | | | | |
| SLRT-14D- | SLRT | 50 | butyl acetate | 0.5 | 100 | RT | 14 | d |
| BAC(100 mg/ml)/ | | | | | | | | |
| FIG. 82 | | | | | | | | |
| SLHT-1D-BME/ | SLHT | 10 | butyl methyl | 1 | 10 | 40 | 1 | d |
| FIG. 75 | | | ether | | | | | |
| SLRT-14D-BME/ | SLRT | 10 | butyl methyl | 1 | 10 | RT | 14 | d |
| FIG. 74 | | | ether | | | | | |
| SLRT-3D-BME/ | SLRT | 10 | butyl methyl | 1 | 10 | RT | 3 | d |
| FIG. 73 | | | ether | | | | | |
| SLRT-1D- | SLRT | 300 | dimethylformamide | 1 | 300 | RT | 1 | d |
| DMF(300 mg/ml)/ | | | | | | | | |
| FIG. 81 | | | | | | | | |
| SLRT-14D- | SLRT | 50 | ethyl acetate | 0.5 | 100 | RT | 14 | d |
| ETA(100 mg/ml)/ | | | | | | | | |
| FIG. 82 | | | | | | | | |
| SLRT-1D- | SLRT | 20 | ethyl acetate | 1 | 20 | RT | 1 | d |
| ETA(20 mg/ml)/ | | | | | | | | |
| FIG. 81 | | | | | | | | |
| SLRT-3D- | SLRT | 20 | ethanol | 0.5 | 40 | RT | 3 | d |
| ETH(40 mg/ml)/ | | | | | | | | |
| FIG. 78 | | | | | | | | |
| SLRT-14D- | SLRT | 20 | ethanol | 0.5 | 40 | RT | 14 | d |
| ETH(40 mg/ml)/ | | | | | | | | |
| FIG. 79 | | | | | | | | |
| SLRT-14D- | SLRT | 50 | ethanol | 0.5 | 100 | RT | 14 | d |
| ETH(100 mg/ml)/ | | | | | | | | |
| FIG. 82 | | | | | | | | |
| SLHT-1D-H2O/ | SLHT | 10 | water | 1 | 10 | 50 | 1 | d |
| FIG. 75 | | | | | | | | |
| SLRT-14D-H2O/ | SLRT | 10 | water | 1 | 10 | RT | 14 | d |
| FIG. 74 | | | | | | | | |
| SLRT-3D-H2O/ | SLRT | 10 | water | 1 | 10 | RT | 3 | d |
| FIG. 73 | | | | | | | | |
| SLHT-1D-HEP/ | SLHT | 10 | heptane | 1 | 10 | 50 | 1 | d |
| FIG. 75 | | | | | | | | |
| SLRT-14D-HEP/ | SLRT | 10 | heptane | 1 | 10 | RT | 14 | d |
| FIG. 74 | | | | | | | | |
| SLRT-3D-HEP/ | SLRT | 10 | heptane | 1 | 10 | RT | 3 | d |
| FIG. 73 | | | | | | | | |
| SLRT-14D- | SLRT | 30 | methanol | 0.6 | 50 | RT | 14 | d |
| MET(50 mg/ml)/ | | | | | | | | |
| FIG. 84 | | | | | | | | |
| SLRT-14D- | SLRT | 50 | methyl isobutyl | 0.5 | 100 | RT | 14 | d |
| MIBK(100 mg/ml)/ | | | ketone | | | | | |
| FIG. 82 | | | | | | | | |
| SLRT-2D- | SLRT | 101.8 | tetrahydrofuran | 0.6 | 169.7 | RT | 2 | d |
| THF(170 mg/ml)/ | | | | | | | | |
| FIG. 81 | | | | | | | | |
| SLRT-14D- | SLRT | 50 | toluene | 0.5 | 100 | RT | 14 | d |
| TOL(100 mg/ml)/ | | | | | | | | |
| FIG. 83 | | | | | | | | |

| | Output | Output | | Output | Output | Output |
|---|---|---|---|---|---|---|
| | Aspect | Aspect | Output | Crystallinity | Main | Unassigned |
| Experiment | start | end | Yield | [high/low/ | phase | Peaks |
| name | [visual] | [visual] | [if appl.] | SC/oriented] | [phase] | [2theta] |
| SLRT-14D- | Suspension | White | | | Form I | |
| 2PR(100 mg/ml)/ | | Powder | | | | |
| FIG. 83 | | | | | | |
| SLHT-1D- | Suspension | White | | | Form I | |
| ACN(50 mg/ml)/ | | Powder | | | | |
| FIG. 80 | | | | | | |
| SLHT-1D- | Suspension | White | | | Form I | |
| ACN(50 mg/ml)- | | Powder | | | | |
| vial attached/ | | | | | | |
| FIG. 92 | | | | | | |
| SLRT-14D- | Suspension | White | | | Form I | |
| ACN(100 mg/ml)/ | | Powder | | | | |
| FIG. 83 | | | | | | |
| SLHT-1D- | Suspension | White | | high | Form I | |
| ACN(100 mg/ml) - | | Powder | | | | |
| Form I reference/ | | | | | | |
| FIG. 76 | | | | | | |
| SLRT-3D- | Suspension | White | | | Form I | |
| ACT(40 mg/ml)/ | | Powder | | | | |
| FIG. 78 | | | | | | |
| SLRT-14D- | Suspension | White | | | Form I | |
| ACT(40 mg/ml)/ | | Powder | | | | |
| FIG. 79 | | | | | | |
| SLHT-1D- | Suspension | White | | | Form I | |
| ACT(50 mg/ml)/ | | Powder | | | | |
| FIG. 80 | | | | | | |
| SLRT-14D- | Suspension | White | | | Form I | |
| ACT(100 mg/ml)/ | | Powder | | | | |
| FIG. 83 | | | | | | |
| SLHT-1D- | Suspension | White | | high | Form I | |
| ACT(100 mg/ml)/ | | Powder | | | | |
| FIG. 77 | | | | | | |
| SLRT-6HRS- | Suspension | White | | | Form I | |
| ACT(40 mg/ml)/ | | Powder | | | | |
| FIG. 81 | | | | | | |
| SLRT-14D- | Suspension | White | | | Form I | [13.9° and |
| BAC(100 mg/ml)/ | | Powder | | | | 23.5°] |
| FIG. 82 | | | | | | |
| SLHT-1D-BME/ | Suspension | White | | | Form I | |
| FIG. 75 | | Powder | | | | |
| SLRT-14D-BME/ | Suspension | White | low | low | Form I | |
| FIG. 74 | | Powder | | | | |
| SLRT-3D-BME/ | Suspension | White | | | Form I | |
| FIG. 73 | | Powder | | | | |
| SLRT-1D- | Suspension | White | | | Form I | |
| DMF(300 mg/ml)/ | | Powder | | | | |
| FIG. 81 | | | | | | |
| SLRT-14D- | Suspension | White | | | Form I | [8.1°] |
| ETA(100 mg/ml)/ | | Powder | | | | |
| FIG. 82 | | | | | | |
| SLRT-1D- | Suspension | White | | | Form I | |
| ETA(20 mg/ml)/ | | Powder | | | | |
| FIG. 81 | | | | | | |
| SLRT-3D- | Suspension | White | | | Form I | |
| ETH(40 mg/ml)/ | | Powder | | | | |
| FIG. 78 | | | | | | |
| SLRT-14D- | Suspension | White | | | Form I | |
| ETH(40 mg/ml)/ | | Powder | | | | |
| FIG. 79 | | | | | | |
| SLRT-14D- | Suspension | White | | | Form I | [14.0°_23.5°] |
| ETH(100 mg/ml)/ | | Powder | | | | |
| FIG. 82 | | | | | | |
| SLHT-1D-H2O/ | Suspension | White | | | Form I | |
| FIG. 75 | | Powder | | | | |
| SLRT-14D-H2O/ | Suspension | White | | | Form I | |
| FIG. 74 | | Powder | | | | |
| SLRT-3D-H2O/ | Suspension | White | | | Form I | |
| FIG. 73 | | Powder | | | | |
| SLHT-1D-HEP/ | Suspension | White | | | Form I | |
| FIG. 75 | | Powder | | | | |
| SLRT-14D-HEP/ | Suspension | White | | | Form I | |
| FIG. 74 | | Powder | | | | |
| SLRT-3D-HEP/ | Suspension | White | | | Form I | |
| FIG. 73 | | Powder | | | | |
| SLRT-14D- | Suspension | White | | | Form I | |
| MET(50 mg/ml)/ | | Powder | | | | |
| FIG. 84 | | | | | | |
| SLRT-14D- | Suspension | White | | | Form I | |
| MIBK(100 mg/ml)/ | | Powder | | | | |
| FIG. 82 | | | | | | |
| SLRT-2D- | Suspension | White | | | Form I | |
| THF(170 mg/ml)/ | | Powder | | | | |
| FIG. 81 | | | | | | |
| SLRT-14D- | Suspension | White | | | Form I | |
| TOL(100 mg/ml)/ | | Powder | | | | |
| FIG. 83 | | | | | | |

### Slurry Procedures in Solvent Mixtures

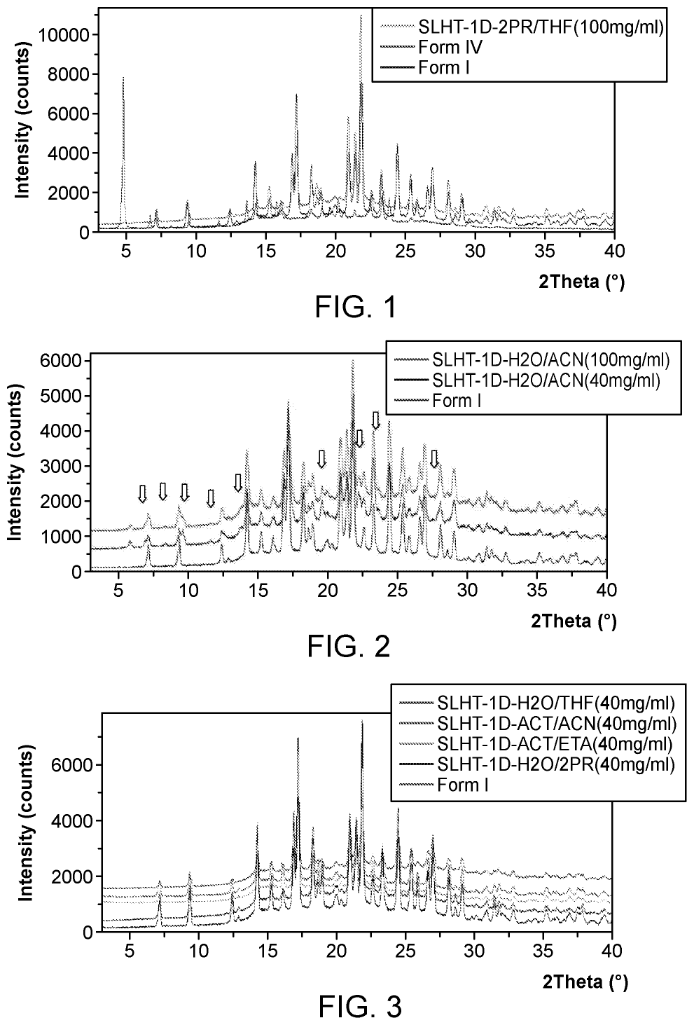

Slurry experiments were performed with solvents with 50° C. (Z)-endoxifen solubilities of less than 40 mg/ml or 100 mg/ml, as listed in TABLE 20. (Z)-endoxifen was suspended in 500 μl of the solvent mixture to achieve the concentrations of 40 mg/ml or 100 mg/ml. The resultant solutions were stored at 50° C. for one day. For solvent mixtures which included acetone, the slurry was instead stored at 40° C. The suspensions were then recovered and filtered with a nylon filter (0.22 μm) during centrifugation at 4000 rpm for 3 minutes, and the collected solid (Z)-endoxifen was analyzed by XRPD as shown in FIG. 85-FIG. 91 and FIG. 1-FIG. 5. The results of the solvent mixture slurry experiments are also displayed in TABLE 20, with the “Experiment name” column in TABLE 20, referring to the name of each of the XRPD spectra displayed in FIG. 85-FIG. 91 and FIG. 1-FIG. 5. As displayed in the table, all solvent mixture slurries generated Form I with varying degrees of purity.

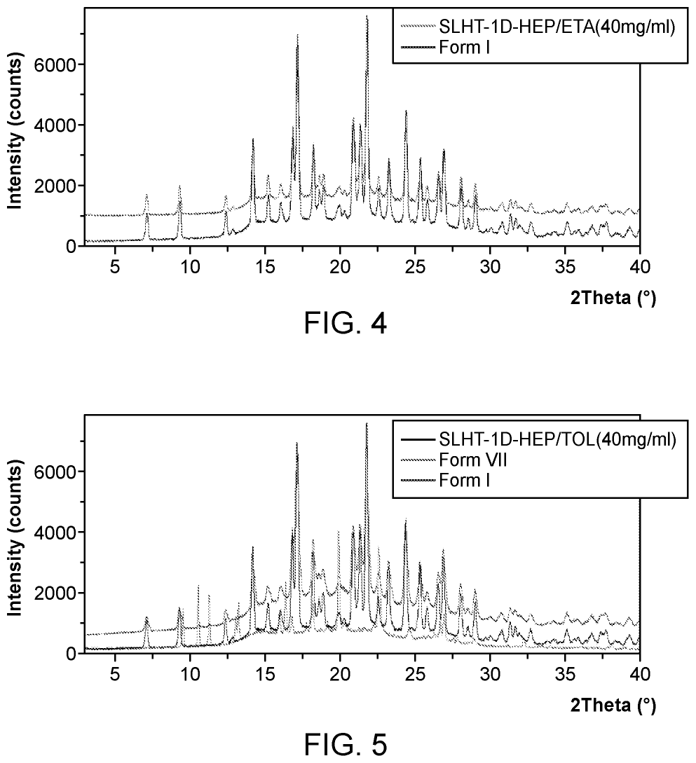

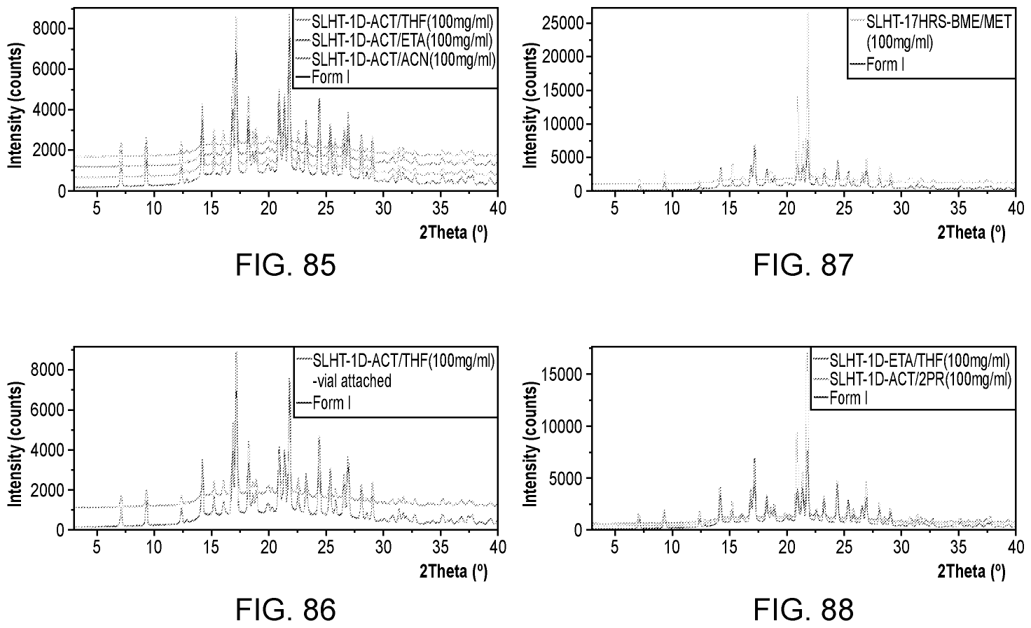

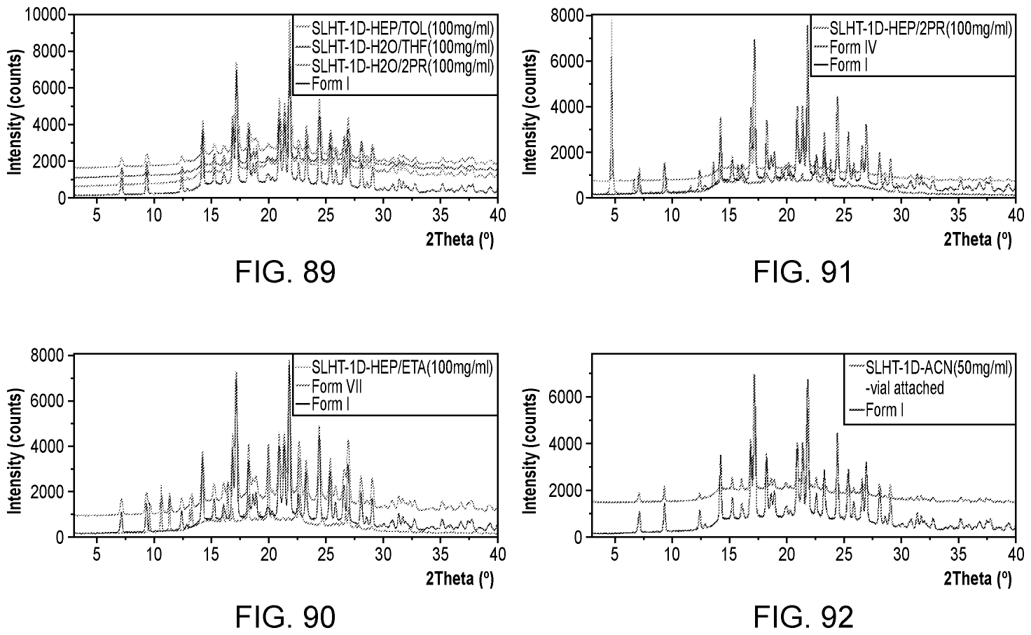

TABLE 20
Solvent Mixture Slurries of (Z)-Endoxifen Results

| | | | | | | | Solvent 2 | | | |
|---|---|---|---|---|---|---|---|---|---|---|
| | | | | | Solvent | Solvent | Conc. in | | | |
| | Experim. | Input | Solvent 1 | Solvent 2 | Mixture 2 | Mixture 2 | Solvent | Temp. | Temp | |
| Experiment name/ | Type | Mass | Type | Type | Volume bin | Compos. bin | mix bin | Start | Duration | |
| FIG. Name | [descript.] | [mg] | [name] | [name] | [mL] | [mL] | [mg/mL] | [° C.] | [days] | |
| SLHT-1D- | SLHT | 50 | 2- | tetrahydrofuran | 0.5 | 95/5 | 100 | 50 | 1 | d |
| 2PR/THF(100 mg/ | | | propanol | | | | | | | |
| ml)/FIG. 1 | | | | | | | | | | |
| SLHT-1D- | SLHT | 20 | acetone | acetonitrile | 0.5 | 50/50 | 40 | 40 | 1 | d |
| ACT/ACN(40 mg/ | | | | | | | | | | |
| ml)/FIG. 3 | | | | | | | | | | |
| SLHT-1D- | SLHT | 20 | acetone | ethyl | 0.5 | 95/5 | 40 | 40 | 1 | d |
| ACT/ETA(40 mg/ | | | | acetate | | | | | | |
| ml)/FIG. 3 | | | | | | | | | | |
| SLHT- | SLHT | 50 | acetone | 2-propanol | 0.5 | 67/33 | 100 | 40 | 1 | d |
| 1D_ACT/2PR(100 mg/ | | | | | | | | | | |
| ml)/FIG. 88 | | | | | | | | | | |
| SLHT-1D- | SLHT | 50 | acetone | acetonitrile | 0.5 | 50/50 | 100 | 40 | 1 | d |
| ACT/ACN(100 mg/ | | | | | | | | | | |
| ml)/FIG. 85 | | | | | | | | | | |
| SLHT-1D- | SLHT | 50 | acetone | ethyl | 0.5 | 50/50 | 100 | 40 | 1 | d |
| ACT/ETA(100 mg/ | | | | acetate | | | | | | |
| ml)/FIG. 85 | | | | | | | | | | |
| SLHT-1D- | SLHT | 50 | acetone | tetrahydrofuran | 0.5 | 50/50 | 100 | 40 | 1 | d |
| ACT/THF(100 mg/ | | | | | | | | | | |
| ml)/FIG. 85 | | | | | | | | | | |
| SLHT- | SLHT | 50 | acetone | tetrahydrofuran | 0.5 | 50/50 | 100 | 40 | 1 | d |
| ACT/THF(100 mg/ | | | | | | | | | | |
| ml)-vial attached/ | | | | | | | | | | |
| FIG. 86 | | | | | | | | | | |
| SLHT-17HRS- | SLHT | 50 | butyl | methanol | 0.5 | 95/5 | 100 | 40 | 17 | h |
| BME/MET(100 mg/ | | | methyl | | | | | | | |
| ml)/FIG. 87 | | | ether | | | | | | | |
| SLHT-1D- | SLHT | 50 | ethyl | tetrahydrofuran | 0.5 | 95/5 | 100 | 50 | 1 | d |
| ETA/THF(100 mg/ | | | acetate | | | | | | | |
| ml)/FIG. 88 | | | | | | | | | | |
| SLHT-1D- | SLHT | 20 | water | 2-propanol | 0.5 | 50/50 | 40 | 50 | 1 | d |
| H2O/2PR(40 mg/ | | | | | | | | | | |
| ml)/FIG. 3 | | | | | | | | | | |
| SLHT-1D- | SLHT | 20 | water | acetonitrile | 0.5 | 50/50 | 40 | 50 | 1 | d |
| H2O/ACN(40 mg/ | | | | | | | | | | |
| ml)/FIG. 2 | | | | | | | | | | |
| SLHT-1D- | SLHT | 20 | water | tetrahydrofuran | 0.5 | 95/5 | 40 | 50 | 1 | d |
| H2O/THF(40 mg/ | | | | | | | | | | |
| ml)/FIG. 3 | | | | | | | | | | |
| SLHT-1D- | SLHT | 50 | water | 2-propanol | 0.5 | 50/50 | 100 | 50 | 1 | d |
| H2O/2PR(100 mg/ | | | | | | | | | | |
| ml)/FIG. 89 | | | | | | | | | | |
| SLHT-1D- | SLHT | 50 | water | acetonitrile | 0.5 | 50/50 | 100 | 50 | 1 | d |
| H2O/ACN(100 mg/ | | | | | | | | | | |
| ml)/FIG. 2 | | | | | | | | | | |
| SLHT-1D- | SLHT | 50 | water | tetrahydrofuran | 0.5 | 95/5 | 100 | 50 | 1 | d |
| H2O/THF(100 mg/ | | | | | | | | | | |
| ml)/FIG. 89 | | | | | | | | | | |
| SLHT-1D- | SLHT | 20 | heptane | ethyl | 0.5 | 50/50 | 40 | 50 | 1 | d |
| HEP/ETA(40 mg/ | | | | acetate | | | | | | |
| ml)/FIG. 4 | | | | | | | | | | |
| SLHT-1D- | SLHT | 20 | heptane | toluene | 0.5 | 50/50 | 40 | 50 | 1 | d |
| HEP/TOL(40 mg/ | | | | | | | | | | |
| ml)/FIG. 5 | | | | | | | | | | |
| SLHT-1D- | SLHT | 50 | heptane | 2-propanol | 0.5 | 50/50 | 100 | 50 | 1 | d |
| HEP/2PR(100 mg/ | | | | | | | | | | |
| ml)/FIG. 91 | | | | | | | | | | |
| SLHT-1D- | SLHT | 50 | heptane | ethyl | 0.5 | 50/50 | 100 | 50 | 1 | d |
| HEP/ETA(100 mg/ | | | | acetate | | | | | | |
| ml)/FIG. 90 | | | | | | | | | | |
| SLHT-1D- | SLHT | 50 | heptane | toluene | 0.5 | 50/50 | 100 | 50 | 1 | d |
| HEP/TOL(100 mg/ | | | | | | | | | | |
| ml)/FIG. 89 | | | | | | | | | | |

| | Output | Output | Output | Output | Output | | Output |
|---|---|---|---|---|---|---|---|
| | Aspect | Aspect | Crystallinity | Main | Other | Output | Unassigned |
| Experiment name/ | start | end | [high/low/ | phase | phases | Traces | Peaks |
| FIG. Name | [visual] | [visual] | SC/oriented] | [phase] | [phase] | [phase] | [2theta] |
| SLHT-1D- | Suspension | White | | Form | | Form | |
| 2PR/THF(100 mg/ | | Powder | | I | | IV | |
| ml)/FIG. 1 | | | | | | | |
| SLHT-1D- | Suspension | White | | Form | | | |
| ACT/ACN(40 mg/ | | Powder | | I | | | |
| ml)/FIG. 3 | | | | | | | |
| SLHT-1D- | Suspension | White | | Form | | | |
| ACT/ETA(40 mg/ | | Powder | | I | | | |
| ml)/FIG. 3 | | | | | | | |
| SLHT- | Suspension | White | | Form | | | [mild |
| 1D_ACT/2PR(100 mg/ | | Powder | | I | | | 13.9°, 18.5°, |
| ml)/FIG. 88 | | | | | | | and 25.2°] |
| SLHT-1D- | Suspension | White | | Form | | | |
| ACT/ACN(100 mg/ | | Powder | | I | | | |
| ml)/FIG. 85 | | | | | | | |
| SLHT-1D- | Suspension | White | | Form | | | |
| ACT/ETA(100 mg/ | | Powder | | I | | | |
| ml)/FIG. 85 | | | | | | | |
| SLHT-1D- | Suspension | White | | Form | | | |
| ACT/THF(100 mg/ | | Powder | | I | | | |
| ml)/FIG. 85 | | | | | | | |
| SLHT- | Suspension | White | | Form | | | |
| ACT/THF(100 mg/ | | Powder | | I | | | |
| ml)-vial attached/ | | | | | | | |
| FIG. 86 | | | | | | | |
| SLHT-17HRS- | Suspension | White | | Form | | | [very mild |
| BME/MET(100 mg/ | | Powder | | I | | | 13.9°_18.5°_{—} |
| ml)/FIG. 87 | | | | | | | 25.2°] |
| SLHT-1D- | Suspension | White | | Form | | | [8.2°] |
| ETA/THF(100 mg/ | | Powder | | I | | | |
| ml)/FIG. 88 | | | | | | | |
| SLHT-1D- | Suspension | White | | Form | | | |
| H2O/2PR(40 mg/ | | Powder | | I | | | |
| ml)/FIG. 3 | | | | | | | |
| SLHT-1D- | Suspension | White | | Form | | | [5.9°_6.9°_{—} |
| H2O/ACN(40 mg/ | | Powder | | I | | | 8.3°_9.6°_{—} |
| ml)/FIG. 2 | | | | | | | 11.7°_13.8°_{—} |
| | | | | | | | 19.6°_22.2°_{—} |
| | | | | | | | 23.7°_27.8°] |
| SLHT-1D- | Suspension | White | low | Form | | | |
| H2O/THF(40 mg/ | | Powder | | I | | | |
| ml)/FIG. 3 | | | | | | | |
| SLHT-1D- | Suspension | White | | Form | | | |
| H2O/2PR(100 mg/ | | Powder | | I | | | |
| ml)/FIG. 89 | | | | | | | |
| SLHT-1D- | Suspension | White | | Form | | | [5.9°_6.9 |
| H2O/ACN(100 mg/ | | Powder | | I | | | (potential)_{—} |
| ml)/FIG. 2 | | | | | | | 8.3°_9.6°_{—} |
| | | | | | | | 11.7°_13.8°_{—} |
| | | | | | | | 19.6°_22.2°_{—} |
| | | | | | | | 23.6°] |
| SLHT-1D- | Suspension | White | | Form | | | |
| H2O/THF(100 mg/ | | Powder | | I | | | |
| ml)/FIG. 89 | | | | | | | |
| SLHT-1D- | Suspension | White | | Form | | | |
| HEP/ETA(40 mg/ | | Powder | | I | | | |
| ml)/FIG. 4 | | | | | | | |
| SLHT-1D- | Suspension | White | | Form | | Form | |
| HEP/TOL(40 mg/ | | Powder | | I | | VII | |
| ml)/FIG. 5 | | | | | | | |
| SLHT-1D- | Suspension | White | | Form | | Form | [6.8°] |
| HEP/2PR(100 mg/ | | Powder | | I | | IV | |
| ml)/FIG. 91 | | | | | | | |
| SLHT-1D- | Suspension | White | | Form | Form | | |
| HEP/ETA(100 mg/ | | Powder | | I | VII | | |
| ml)/FIG. 90 | | | | | | | |
| SLHT-1D- | Suspension | White | low | Form | | | |
| HEP/TOL(100 mg/ | | Powder | | I | | | |
| ml)/FIG. 89 | | | | | | | |

### Slurries With Amorphous (Z)-Endoxifen

Further slurry experiments were performed utilizing amorphous (Z)-endoxifen as an input. 20 mg of amorphous (Z)-endoxifen was suspended in 200 μl of various solvent mixtures. Following 22 days of stirring at room temperature, solid (Z)-endoxifen was recovered from the slurries during centrifugation at 4000 rpm for 3 minutes with a nylon filter (0.22 μm). Collected (Z)-endoxifen was then characterized with XRPD, the results of which are summarized in TABLE 21. As displayed in the table, the ethyl acetate/heptane slurry produced Form IX, the acetone/tetrahydrofuran slurry produced Form I with additional unassigned peaks, and one of the 2-propanol/tetrahydrofuran slurries produced Form IV, while the other yielded insufficient solid for XRPD analysis.

TABLE 21
Slurries of Amorphous (Z)-Endoxifen Results

| | | | | | | Solvent 2 | | |
|---|---|---|---|---|---|---|---|---|
| | | | | Solvent | Solvent | Conc. in | | |
| Experim. | Input | Solvent 1 | Solvent 2 | Mixture 2 | Mixture 2 | Solvent | Temp. | Temp |
| Type | Mass | Type | Type | Volume bin | Compos. bin | mix bin | Start | Duration |
| [descript.] | [mg] | [name] | [name] | [mL] | [mL] | [mg/mL] | [° C.] | [days] |
| SLRT | 20 | ethyl | heptane | 0.2 | 50/50 | 100 | RT | 22 d |
| | | acetate | | | | | | |
| SLRT | 20 | acetone | tetrahydrofuran | 0.2 | 90/10 | 100 | RT | 22 d |
| SLRT | 20 | 2- | tetrahydrofuran | 0.2 | 63/37 | 100 | RT | 22 d |
| | | propanol | | | | | | |
| SLRT- | 20 | 2- | tetrahydrofuran | 0.2 | 63/37 | 100 | RT | |
| EVRT | | propanol | | | | | | |

| | Output | Output | Output | Output | |
|---|---|---|---|---|---|
| Experim. | Aspect | Aspect | Main | Unassigned | |
| Type | start | end | phase | Peaks | |
| [descript.] | [visual] | [visual] | [phase] | [2theta] | Note |
| SLRT | Suspension | White | Form | [10.1°_11.7°_{—} | Immediately after the |
| | | Powder | IX | 16.8°_18.5°_{—} | addition of amorphous to |
| | | | | 19.4°_23.1°] | the solvent mixture |
| | | | | | dissolution occurred. |
| | | | | | After 6 hours under |
| | | | | | stirring. white |
| | | | | | suspension was observed. |
| SLRT | Suspension | White | Form I | | Immediately after the |
| | | Powder | | | addition of amorphous to |
| | | | | | the solvent mixture |
| | | | | | dissolution occurred. |
| | | | | | After 6 hours under |
| | | | | | stirring. off-white |
| | | | | | suspension was observed. |
| SLRT | Clear | Clear | No | | |
| | solution | solution | solid | | |
| SLRT- | Clear | White | Form | | |
| EVRT | solution | Powder | IV | | |

### Example 21

(Z)-Endoxifen Recovery by Solvent Evaporation

This example covers (Z)-endoxifen recovery by evaporation, in which (Z)-endoxifen was dissolved in single and multi-solvent systems, and then recollected through solvent evaporation.

For the single-solvent evaporations, 10 mg (Z)-endoxifen was dissolved in 1 ml of various solvents in which (Z)-endoxifen has >1000 mg/ml solubility (“very soluble”), 100-1000 mg/ml solubility (“freely soluble”), 33-100 mg/ml solubility (“soluble”), and 10-33 mg/ml solubility (“sparingly soluble”). The solutions were stirred for approximately 60 minutes, then left to evaporate. Following (Z)-endoxifen dissolution to a concentration of 10 mg/ml, all solvents generated clear solutions in which the (Z)-endoxifen completely dissolved. The solutions were evaporated at either room temperature (17-25° C.) or elevated temperature (40-60° C.). All evaporations were performed at 1 atmosphere pressure. The solids obtained from the evaporations were analyzed by XRPD.

For the multi-solvent evaporations, 40 mg (Z)-endoxifen was dissolved in either ethanol/water or THF/acetone mixtures. For the water/ethanol evaporations, 20 ml of 70/30 ethanol/water (v/v) mixtures (achieving concentrations of 2 mg/ml (Z)-endoxifen). The white suspensions were incubated for 3 days at 50° C., yielding clear solutions which were then left to evaporate at elevated temperatures. These analyses were repeated over 4 replicates. For the THF/acetone evaporations, 60 mg (Z)-endoxifen were dissolved in 1.5 ml of a multi-solvent system containing tetrahydrofuran (THF) and acetone in a 40/60 (v/v) mixture. The resultant clear solution was filtered and left to evaporate at room temperature.

Results of the single- and multi-solvent system evaporation experiments are summarized in TABLE 22. Butyl acetate, ethyl acetate, and methanol evaporations generated Form I. Acetone evaporation yielded a mixture of Forms I and IV. Acetonitrile evaporation yielded Forms I, V, and VI. 2-propanol evaporation yielded Form IV. Ethanol evaporation yielded Form XVII. Tetrahydrofuran (THF) evaporation yielded Form XVIII. Dimethylformamide (DMF) and methyl isobutyl ketone (MIBK) evaporations yielded amorphous (Z)-endoxifen. Toluene evaporation yielded a combination of Form VII and amorphous (Z)-endoxifen. Evaporation of ethanol/water mixtures generated amorphous (Z)-endoxifen with minor amounts of polymorph. Evaporation of THF:acetone mixture generated a combination of Forms I and IV.

TABLE 22
Evaporation of (Z)-Endoxifen Results

| | | | | Solvent 1 | | | Solvent | Solvent |
|---|---|---|---|---|---|---|---|---|
| Experim. | Input | Solvent 1 | Solvent 1 | Conc. in | Solvent 2 | Solvent 2 | Mixture 2 | Mixture 2 |
| Type | Mass | Type | Volume | Solvent 1 | Type | Volume | Volume bin | Compos. bin |
| [descript.] | [mg] | [name] | [mL] | [mg/mL] | [name] | [mL] | [mL] | [v/v] |
| EVHT | 10 | butyl acetate | 1 | 10 | | | | |
| EVHT | 10 | dimethylformamide | 1 | 10 | | | | |
| EVHT | 40 | ethanol | 14 | | water | 6 | 20 | 70/30 |
| EVHT | 40 | ethanol | 14 | | water | 6 | 20 | 70/30 |
| EVHT | 40 | ethanol | 14 | | water | 6 | 20 | 70/30 |
| EVHT | 40 | ethanol | 14 | | water | 6 | 20 | 70/30 |
| EVHT | 10 | methyl isobutyl | 1 | 10 | | | | |
| | | ketone | | | | | | |
| EVHT | 10 | toluene | 1 | 10 | | | | |
| EVRT | 10 | 2-propanol | 1 | 10 | | | | |
| EVRT | 10 | acetonitrile | 1 | 10 | | | | |
| EVRT | 10 | acetone | 1 | 10 | | | | |
| EVRT | 10 | ethyl acetate | 1 | 10 | | | | |
| EVRT | 10 | ethanol | 1 | 10 | | | | |
| EVRT | 10 | methanol | 1 | 10 | | | | |
| EVRT | 10 | tetrahydrofuran | 1 | 10 | | | | |
| EVRT | 60 | tetrahydrofuran | | | acetone | | 1.5 | 40/60 |

| | Solvent | | | | | | | |
|---|---|---|---|---|---|---|---|---|
| | Mixture 2 | | | | | | | |
| | Conc. in | | Output | Output | Output | Output | | Output |
| Experim. | Solvent | Temp. | Aspect | Aspect | Main | Other | Output | Unassigned |
| Type | mix bin | Start | start | end | phase | phases | Traces | Peaks |
| [descript.] | [mg/mL] | [° C.] | [visual] | [visual] | [phase] | [phase] | [phase] | [2theta] |
| EVHT | | 60 | Clear | White | Form I | | | |
| | | | solution | powder | | | | |
| EVHT | | 60 | Clear | White | amorphous | | | [9.5°] |
| | | | solution | powder | | | | |
| EVHT | 2 | 60 | Clear | Off- | amorphous | | | [8.1°_24.3°]; |
| | | | solution | white | | | | [5.7°_7.3°_{—} |
| | | | | powder | | | | 11.2°_19.2°_{—} |
| | | | | | | | | 20.7°_22.3°_{—} |
| | | | | | | | | 27.6°_32.6°_{—} |
| | | | | | | | | 33.2] |
| EVHT | 2 | 60 | Clear | Off- | amorphous | | | [8.1°_24.3°] |
| | | | solution | white | | | | |
| | | | | powder | | | | |
| EVHT | 2 | 60 | Clear | Off- | amorphous | | | [8.1°_24.3°]; |
| | | | solution | white | | | | [5.7°_7.3°_{—} |
| | | | | powder | | | | 11.2°_19.2°_{—} |
| | | | | | | | | 20.7°_22.3°_{—} |
| | | | | | | | | 27.6°_32.6°_{—} |
| | | | | | | | | 33.2] |
| EVHT | 2 | 60 | Clear | Off- | amorphous | | | [8.1°_24.3°] |
| | | | solution | white | | | | |
| | | | | powder | | | | |
| EVHT | | 60 | Clear | Yellow | amorphous | | | |
| | | | solution | vitreous | | | | |
| | | | | solid | | | | |
| EVHT | | 60 | Clear | White | amorphous | Form | | |
| | | | solution | powder | | VII | | |
| EVRT | | RT | Clear | White | Form IV | | | |
| | | | solution | powder | | | | |
| EVRT | | RT | Clear | White | Form I | Form | Form | |
| | | | solution | powder | | V | VI | |
| EVRT | | RT | Clear | White | Form I | Form | | [19.8°] |
| | | | solution | powder | | IV | | |
| EVRT | | RT | Clear | White | Form I | | | |
| | | | solution | powder | | | | |
| EVRT | | RT | Clear | White | Form | | | |
| | | | solution | powder | XVII | | | |
| EVRT | | RT | Clear | White | Form I | | | |
| | | | solution | powder | | | | |
| EVRT | | RT | Clear | White | Form | | | |
| | | | solution | powder | XVIII | | | |
| EVRT | 40 | RT | Clear | White | Form I | Form | | |
| | | | solution | powder | | IV | | |

### Example 22

### Precipitation of (Z)-Endoxifen

This example covers (Z)-endoxifen precipitation by various methods including cooling and antisolvent addition.

### Precipitation By Cooling

Z)-endoxifen was precipitated by cooling various single and multi- solvent systems. Suspensions were generated by first dissolving (Z)-endoxifen in single and multi-solvent systems by heating these systems to their boiling points, and then cooling these systems to 25° C. to affect (Z)-endoxifen precipitation. The crystallizations utilized cooling rates of 0.5° C./minute or 0.2° C./minute. The samples were then incubated 25° C. with stirring for approximately 15 hours. Some solutions generated precipitate, which was recovered by filtration with a nylon filter (0.22 μm) during centrifugation at 4000 rpm for 3 minutes.

Solutions which did not generate precipitate and which had concentrations greater than 10 mg/ml were stored at low temperature (−20° C.) for 24 hours or 3 days to induce precipitation. The resulting precipitate was recovered by filtration with a nylon filter (0.22 μm) during centrifugation at 4000 rpm for 3 minutes.

Solutions which did not generate precipitate and which had concentrations less than or equal to 10 mg/ml were evaporated to induce precipitation. All precipitates were collected for XRPD analysis. The results of these precipitation experiments are summarized in TABLE 23.

TABLE 23
Gradient Precipitation of (Z)-Endoxifen Results

| | | | | | | | | Solvent | |
|---|---|---|---|---|---|---|---|---|---|
| | | | | | | | | Mixture 2 | |
| | | | | Solvent 1 | | Solvent | Solvent | Conc. in | |
| Experim. | Input | Solvent 1 | Solvent 1 | Conc. in | Solvent 2 | Mixture 2 | Mixture 2 | Solvent | Temp. |
| Type | Mass | Type | Volume | Solvent 1 | Type | Volume bin | Compos bin | mix bin | Start |
| [descript.] | [mg] | [name] | [mL] | [mg/mL] | [name] | [mL] | [v/v] | [mg/mL] | [° C.] |
| GRAD | 10 | 2- | 1 | 10 | | | | | 75 |
| | | propanol | | | | | | | |
| GRAD | 10 | acetonitrile | 1 | 10 | | | | | 75 |
| GRAD | 10 | acetone | 1 | 10 | | | | | 50 |
| GRAD | 10 | butyl | 1 | 10 | | | | | 100 |
| | | acetate | | | | | | | |
| GRAD | 10 | ethyl | 1 | 10 | | | | | 75 |
| | | acetate | | | | | | | |
| GRAD | 10 | ethanol | 1 | 10 | | | | | 75 |
| GRAD | 10 | methyl | 1 | 10 | | | | | 100 |
| | | isobutyl | | | | | | | |
| | | ketone | | | | | | | |
| GRAD | 10 | toluene | 1 | 10 | | | | | 100 |
| GRAD | 10 | 2- | 0.5 | 20 | | | | | 75 |
| | | propanol | | | | | | | |
| GRAD | 50 | 2- | 0.5 | 100 | | | | | 75 |
| | | propanol | | | | | | | |
| GRAD | 10 | acetonitrile | 0.5 | 20 | | | | | 75 |
| GRAD | 10 | acetone | 0.5 | 20 | | | | | 50 |
| GRAD | 10 | acetone | 0.5 | 20 | | | | | 25 |
| GRAD | 10 | acetone | 0.5 | 20 | | | | | 25 |
| GRAD | 10 | butyl | 0.5 | 20 | | | | | 100 |
| | | acetate | | | | | | | |
| GRAD | 10 | butyl | 0.5 | 20 | | | | | 25 |
| | | acetate | | | | | | | |
| GRAD | 50 | butyl | 0.5 | 100 | | | | | 100 |
| | | acetate | | | | | | | |
| GRAD | 10 | ethyl | 0.5 | 20 | | | | | 75 |
| | | acetate | | | | | | | |
| GRAD | 10 | ethyl | 0.5 | 20 | | | | | 25 |
| | | acetate | | | | | | | |
| GRAD | 50 | ethyl | 0.5 | 100 | | | | | 75 |
| | | acetate | | | | | | | |
| GRAD | 10 | ethanol | 0.5 | 20 | | | | | 75 |
| GRAD | 50 | ethanol | 0.5 | 100 | | | | | 75 |
| GRAD | 10 | methyl | 0.5 | 20 | | | | | 100 |
| | | isobutyl | | | | | | | |
| | | ketone | | | | | | | |
| GRAD | 10 | methyl | 0.5 | 20 | | | | | 25 |
| | | isobutyl | | | | | | | |
| | | ketone | | | | | | | |
| GRAD | 50 | methyl | 0.5 | 100 | | | | | 100 |
| | | isobutyl | | | | | | | |
| | | ketone | | | | | | | |
| GRAD | 50 | methyl | 0.5 | 100 | | | | | 25 |
| | | isobutyl | | | | | | | |
| | | ketone | | | | | | | |
| GRAD | 100 | methyl | 0.5 | 200 | | | | | 100 |
| | | isobutyl | | | | | | | |
| | | ketone | | | | | | | |
| GRAD | 10 | toluene | 0.5 | 20 | | | | | 100 |
| GRAD | 10 | toluene | 0.5 | 20 | | | | | 25 |
| GRAD | 50 | toluene | 0.5 | 100 | | | | | 100 |
| GRAD | 20 | 2- | | | tetrahydrofuran | 0.5 | 95/5 | 40 | 60 |
| | | propanol | | | | | | | |
| GRAD | 20 | acetone | | | 2-propanol | 0.5 | 67/33 | 40 | 50 |
| GRAD | 20 | acetone | | | 2-propanol | 0.5 | 67/33 | 40 | 25 |
| GRAD | 20 | acetone | | | tetrahydrofuran | 0.5 | 95/5 | 40 | 50 |
| GRAD | 20 | acetone | | | tetrahydrofuran | 0.5 | 95/5 | 40 | 25 |
| GRAD | 50 | butyl | | | methanol | 0.5 | 50/50 | 100 | 50 |
| | | methyl | | | | | | | |
| | | ether | | | | | | | |
| GRAD | 50 | butyl | | | methanol | 0.5 | 50/50 | 100 | 25 |
| | | methyl | | | | | | | |
| | | ether | | | | | | | |
| GRAD | 20 | ethyl | | | tetrahydrofuran | 0.5 | 95/5 | 40 | 60 |
| | | acetate | | | | | | | |
| GRAD | 50 | ethyl | | | 2-propanol | 0.5 | 50/50 | 100 | 70 |
| | | acetate | | | | | | | |
| GRAD | 20 | heptane | | | 2-propanol | 0.5 | 50/50 | 40 | 75 |

| | | | | Output | Output | | Output | Output | |
|---|---|---|---|---|---|---|---|---|---|
| Experim. | Temp. | Temp. | Temp. | Aspect | Aspect | Output | Crystallinity | Main | Output |
| Type | End | Ramp | Duration | start | end | Yield | [high/low/ | phase | Traces |
| [descript.] | [° C.] | [° C.] | [° C.] | [visual] | [visual] | [if appl.] | SC/oriented] | [phase] | [phase] |
| GRAD | 25 | −0.5 | | Clear | Clear | | | No | |
| | | | | solution | solution | | | solid | |
| GRAD | 25 | −0.5 | | Clear | Clear | | | No | |
| | | | | solution | solution | | | solid | |
| GRAD | 25 | −0.5 | | Clear | Clear | | | No | |
| | | | | solution | solution | | | solid | |
| GRAD | 25 | −0.5 | | Clear | Clear | | | No | |
| | | | | solution | solution | | | solid | |
| GRAD | 25 | −0.5 | | Clear | Clear | | | No | |
| | | | | solution | solution | | | solid | |
| GRAD | 25 | −0.5 | | Clear | Clear | | | No | |
| | | | | solution | solution | | | solid | |
| GRAD | 25 | −0.5 | | Clear | Clear | | | No | |
| | | | | solution | solution | | | solid | |
| GRAD | 25 | −0.5 | | Clear | Clear | | | No | |
| | | | | solution | solution | | | solid | |
| GRAD | 25 | −0.2 | | Clear | White | low | low | Form I | Form |
| | | | | solution | powder | | | | IV |
| GRAD | 25 | −0.2 | | Clear | White | | | Form I | Form |
| | | | | solution | powder | | | | IV |
| GRAD | 25 | −0.2 | | Clear | White | low | low | Form V | |
| | | | | solution | powder | | | | |
| GRAD | 25 | −0.2 | | Clear | Clear | | | No | |
| | | | | solution | solution | | | solid | |
| GRAD | −20 | Not | 1 d | Clear | Clear | | | Few | |
| | | controlled | | solution | solution | | | solid | |
| GRAD | −20 | Not | 3 d | Clear | White | low | low | Form I | |
| | | controlled | | solution | powder | | | | |
| GRAD | 25 | −0.2 | | Clear | Clear | | | No | |
| | | | | solution | solution | | | solid | |
| GRAD | −20 | Not | 3 d | Clear | Clear | | | No | |
| | | controlled | | solution | solution | | | solid | |
| GRAD | 25 | −0.2 | | Clear | White | | low | Form I | |
| | | | | solution | powder | | | | |
| GRAD | 25 | −0.2 | | Clear | Clear | | | No | |
| | | | | solution | solution | | | solid | |
| GRAD | −20 | Not | 3 d | Clear | Clear | | | No | |
| | | controlled | | solution | solution | | | solid | |
| GRAD | 25 | −0.2 | | Clear | White | | | Form I | |
| | | | | solution | powder | | | | |
| GRAD | 25 | −0.2 | | Clear | White | low | low | Form I | |
| | | | | solution | powder | | | | |
| GRAD | 25 | −0.2 | | Clear | White | | | Form I | |
| | | | | solution | powder | | | | |
| GRAD | 25 | −0.2 | | Clear | Clear | | | No | |
| | | | | solution | solution | | | solid | |
| GRAD | −20 | Not | 3 d | Clear | Clear | | | No | |
| | | controlled | | solution | solution | | | solid | |
| GRAD | 25 | −0.2 | | Clear | Clear | | | No | |
| | | | | solution | solution | | | solid | |
| GRAD | −20 | Not | 1 d | Clear | White | | | Form X | |
| | | controlled | | solution | powder | | | | |
| GRAD | 25 | −0.2 | | Clear | White | | | Form | |
| | | | | solution | powder | | | XI | |
| GRAD | 25 | −0.2 | | Clear | Clear | | | No | |
| | | | | solution | solution | | | solid | |
| GRAD | −20 | Not | 3 d | Clear | Clear | | | No | |
| | | controlled | | solution | solution | | | solid | |
| GRAD | 25 | −0.2 | | Clear | White | | | Form | |
| | | | | solution | powder | | | VIII | |
| GRAD | 25 | −0.2 | | Clear | White | | | Form | |
| | | | | solution | powder | | | IV | |
| GRAD | 25 | −0.2 | | Clear | Clear | | | No | |
| | | | | solution | solution | | | solid | |
| GRAD | −20 | Not | | Clear | White | low | | amorphous | Form |
| | | controlled | | solution | powder | | | | IV |
| GRAD | 25 | −0.2 | | Clear | Clear | | | No | |
| | | | | solution | solution | | | solid | |
| GRAD | −20 | Not | | Clear | White | | | Form I | |
| | | controlled | | solution | powder | | | | |
| GRAD | 25 | −0.2 | | Clear | Clear | | | No | |
| | | | | solution | solution | | | solid | |
| GRAD | −20 | Not | 3 d | Clear | Clear | | | No | |
| | | controlled | | solution | solution | | | solid | |
| GRAD | 25 | −0.2 | | Clear | White | | | Form I | |
| | | | | solution | powder | | | | |
| GRAD | 25 | −0.2 | | Clear | White | | | Form | |
| | | | | solution | powder | | | IV | |
| GRAD | 25 | −0.2 | | Clear | White | | | Form | |
| | | | | solution | powder | | | IV | |

### Precipitation by Antisolvent Addition

Further (Z)-endoxifen crystallizations were performed utilizing antisolvents to affect precipitation. (Z)-endoxifen was dissolved in various solvents to obtain suspensions at room temperature that were then stirred overnight and filtered with a nylon filter (0.22 μm) to obtain a clear stock solution. Two different types of precipitation experiments were performed with the resulting clear stock solutions. For precipitation by antisolvent addition to a saturated solution (PAD) experiments, antisolvent was added dropwise to the clear stock solution during stirring at room temperature at a ratio of 1:5 or 1:10 stock solution:antisolvent. For precipitation by saturated solution addition to an antisolvent (PAI) experiments, the clear stock solution was added drop wise to antisolvent under magnetic stirring at room temperature at a ratio of 1:5 or 1:10 stock solution:antisolvent. All obtained precipitates from PAD and PAI experiments were filtered under vacuum and analyzed by XRPD. If no precipitate was formed, the solution was then stored at low temperature (−20° C.) for 24 hours. After storage if there was no precipitate, the solutions were evaporated at room temperature. All obtained precipitates were filtered under vacuum and analyzed by XRPD. PAD and PAI experiments were also performed on the clear solutions with a concentration of 20 mg/ml (Z)-endoxifen recovered from the solubility experiments, described in EXAMPLE 19. The clear solutions recovered were (Z)-endoxifen solutions in ethyl acetate, butyl acetate, 4-met-2-pentanone, and toluene. The procedure for the PAI and PAD was the same as described above. The results of the PAI and PAD experiments are shown in the following tables. TABLE 24 shows the results of precipitation experiments for direct antisolvent addition of ethyl acetate, butyl acetate, 4-methtyl-2-pentanone, and toluene saturated solutions. TABLE 25 shows the results of precipitation experiments for antisolvent addition of ethyl acetate and acetone saturated solution. TABLE 26 shows the results of precipitation experiments for the antisolvent addition of tetrahydrofuran and dimethylformamide saturated solutions.

TABLE 24
Results of Precipitation Experiments for Direct Antisolvent Addition of Ethyl Acetate,
Butyl Acetate, 4-Metthyl-2-Pentanone, and Toluene Saturated Solutions of (Z)-Endoxifen

| | | | | Solvent 1 | | | Solvent | | |
|---|---|---|---|---|---|---|---|---|---|
| Experim. | Input | Solvent 1 | Solvent 1 | Conc. in | Solvent 2 | Solvent 2 | Mixture 2 | Temp. | Temp. |
| Type | Mass | Type | Volume | Solvent 1 | Type | Volume | Volume bin | Start | End |
| [descript.] | [mg] | [name] | [mL] | [mg/mL] | [name] | [mL] | [mL] | [° C.] | [° C.] |
| PAD | 10 | butyl | 0.15 | 20 | butyl | 0.75 | 0.9 | RT | |
| | | acetate | | | methyl | | | | |
| | | | | | ether | | | | |
| PAD | 10 | butyl | 0.15 | 20 | butyl | 1.5 | 1.65 | RT | |
| | | acetate | | | methyl | | | | |
| | | | | | ether | | | | |
| PAD- | | butyl | 0.15 | | butyl | 1.5 | 1.65 | 25 | −20 |
| GRAD | | acetate | | | methyl | | | | |
| | | | | | ether | | | | |
| PAD-- | | butyl | 0.15 | | butyl | 1.5 | 1.65 | RT | |
| GRAD- | | acetate | | | methyl | | | | |
| EVRT | | | | | ether | | | | |
| PAD | 10 | butyl | 0.15 | 20 | heptane | 0.75 | 0.9 | RT | |
| | | acetate | | | | | | | |
| PAD | 10 | butyl | 0.15 | 20 | heptane | 1.5 | 1.65 | RT | |
| | | acetate | | | | | | | |
| PAD | 10 | ethyl | 0.15 | 20 | butyl | 0.75 | 0.9 | RT | |
| | | acetate | | | methyl | | | | |
| | | | | | ether | | | | |
| PAD | 10 | ethyl | 0.15 | 20 | butyl | 1.5 | 1.65 | RT | |
| | | acetate | | | methyl | | | | |
| | | | | | ether | | | | |
| PAD- | | ethyl | 0.15 | | butyl | 1.5 | 1.65 | 25 | −20 |
| GRAD | | acetate | | | methyl | | | | |
| | | | | | ether | | | | |
| PAD- | | ethyl | 0.15 | | butyl | 1.5 | 1.65 | RT | |
| GRAD- | | acetate | | | methyl | | | | |
| EVRT | | | | | ether | | | | |
| PAD | 10 | ethyl | 0.15 | 20 | heptane | 0.75 | 0.9 | RT | |
| | | acetate | | | | | | | |
| PAD | 10 | ethyl | 0.15 | 20 | heptane | 1.5 | 1.65 | RT | |
| | | acetate | | | | | | | |
| PAD | 10 | methyl | 0.15 | 20 | butyl | 0.75 | 0.9 | RT | |
| | | isobutyl | | | methyl | | | | |
| | | ketone | | | ether | | | | |
| PAD | 10 | methyl | 0.15 | 20 | butyl | 1.5 | 1.65 | RT | |
| | | isobutyl | | | methyl | | | | |
| | | ketone | | | ether | | | | |
| PAD- | | methyl | 0.15 | | butyl | 1.5 | 1.65 | 25 | −20 |
| GRAD | | isobutyl | | | methyl | | | | |
| | | ketone | | | ether | | | | |
| PAD- | | methyl | 0.15 | | butyl | 1.5 | 1.65 | RT | |
| GRAD- | | isobutyl | | | methyl | | | | |
| EVRT | | ketone | | | ether | | | | |
| PAD | 10 | methyl | 0.15 | 20 | heptane | 0.75 | 0.9 | RT | |
| | | isobutyl | | | | | | | |
| | | ketone | | | | | | | |
| PAD | 10 | methyl | 0.15 | 20 | heptane | 1.5 | 1.65 | RT | |
| | | isobutyl | | | | | | | |
| | | ketone | | | | | | | |
| PAD- | | methyl | 0.15 | | heptane | 1.5 | 1.65 | 25 | −20 |
| GRAD | | isobutyl | | | | | | | |
| | | ketone | | | | | | | |
| PAD | 10 | toluene | 0.15 | 20 | butyl | 0.75 | 0.9 | RT | |
| | | | | | methyl | | | | |
| | | | | | ether | | | | |
| PAD | 10 | toluene | 0.15 | 20 | butyl | 1.5 | 1.65 | RT | |
| | | | | | methyl | | | | |
| | | | | | ether | | | | |
| PAD- | | toluene | 0.15 | | butyl | 1.5 | 1.65 | 25 | −20 |
| GRAD | | | | | methyl | | | | |
| | | | | | ether | | | | |
| PAD- | | toluene | 0.15 | | butyl | 1.5 | 1.65 | RT | |
| GRAD- | | | | | methyl | | | | |
| EVRT | | | | | ether | | | | |
| PAD | 10 | toluene | 0.15 | 20 | heptane | 0.75 | 0.9 | RT | |
| PAD | 10 | toluene | 0.15 | 20 | heptane | 1.5 | 1.65 | RT | |

| | | | Output | Output | | Output | Output |
|---|---|---|---|---|---|---|---|
| Experim. | Temp. | Temp. | Aspect | Aspect | Output | Crystallinity | Main |
| Type | Ramp | Duration | start | end | Yield | [high/low/ | phase |
| [descript.] | [K/min] | [days] | [visual] | [visual] | [if appl.] | SC/oriented] | [phase] |
| PAD | | | Clear | Clear | | | no solid |
| | | | solution | solution | | | |
| PAD | | | Clear | Clear | | | no solid |
| | | | solution | solution | | | |
| PAD- | not | 1 d | Clear | Clear | | | no solid |
| GRAD | controlled | | solution | solution | | | |
| PAD-- | | | Clear | White | low | low | Form I |
| GRAD- | | | solution | powder | | | |
| EVRT | | | | | | | |
| PAD | | | Clear | White | low | low | no solid |
| | | | solution | powder | | | |
| PAD | | | Clear | White | low | low | Form IX |
| | | | solution | powder | | | |
| PAD | | | Clear | Clear | | | no solid |
| | | | solution | solution | | | |
| PAD | | | Clear | Clear | | | no solid |
| | | | solution | solution | | | |
| PAD- | not | 1 d | Clear | Clear | | | no solid |
| GRAD | controlled | | solution | solution | | | |
| PAD- | | | Clear | White | low | low | Form I |
| GRAD- | | | solution | powder | | | |
| EVRT | | | | | | | |
| PAD | | | Clear | White | low | low | no solid |
| | | | solution | powder | | | |
| PAD | | | Clear | White | low | low | Form IX |
| | | | solution | powder | | | |
| PAD | | | Clear | Clear | | | no solid |
| | | | solution | solution | | | |
| PAD | | | Clear | Clear | | | no solid |
| | | | solution | solution | | | |
| PAD- | not | 1 d | Clear | Clear | | | no solid |
| GRAD | controlled | | solution | solution | | | |
| PAD- | | | Clear | White | | | No |
| GRAD- | | | solution | powder | | | recovery |
| EVRT | | | | | | | |
| PAD | | | Clear | Slightly | | | no solid |
| | | | solution | opalescent | | | |
| PAD | | | Clear | Slightly | | | no solid |
| | | | solution | opalescent | | | |
| PAD- | not | 1 d | Slightly | Clear | | | No |
| GRAD | controlled | | opalescent | solution | | | recovery |
| PAD | | | Clear | Clear | | | no solid |
| | | | solution | solution | | | |
| PAD | | | Clear | Clear | | | no solid |
| | | | solution | solution | | | |
| PAD- | not | 1 d | Clear | Clear | | | no solid |
| GRAD | controlled | | solution | solution | | | |
| PAD- | | | Clear | White | low | low | Form VIII |
| GRAD- | | | solution | powder | | | |
| EVRT | | | | | | | |
| PAD | | | Clear | White | | | no solid |
| | | | solution | powder | | | |
| PAD | | | Clear | White | | low | Amorphous |
| | | | solution | powder | | | (traces of |
| | | | | | | | Form VII) |

TABLE 25
Results of Precipitation Experiments for Antisolvent Addition of
Ethyl Acetate and Acetone Saturated Solutions of (Z)-Endoxifen

| | | | Solvent 1 | | | Solvent | | |
|---|---|---|---|---|---|---|---|---|
| Experim. | Solvent 1 | Solvent 1 | Conc. in | Solvent 2 | Solvent 2 | Mixture 2 | Temp. | Temp. |
| Type | Type | Volume | Solvent 1 | Type | Volume | Volume bin | Start | End |
| [descript.] | [name] | [mL] | [mg/mL] | [name] | [mL] | [mL] | [° C.] | [° C.] |
| PAD | ethyl acetate | 0.8 | sat | butyl | 4 | 4.8 | RT | |
| | | | | methyl | | | | |
| | | | | ether | | | | |
| PAD | ethyl acetate | 0.8 | sat | butyl | 8 | 8.8 | RT | |
| | | | | methyl | | | | |
| | | | | ether | | | | |
| PAD- | ethyl acetate | 0.8 | sat | butyl | 8 | 8.8 | RT | −20 |
| GRAD | | | | methyl | | | | |
| | | | | ether | | | | |
| PAD-- | ethyl acetate | 0.8 | sat | butyl | 8 | 8.8 | RT | |
| GRAD- | | | | methyl | | | | |
| EVRT | | | | ether | | | | |
| PAD | ethyl acetate | 0.8 | sat | heptane | 8 | 8.8 | RT | |
| PAD- | ethyl acetate | 0.8 | sat | heptane | 8 | 8.8 | RT | −20 |
| GRAD | | | | | | | | |
| PAI | ethyl acetate | 0.8 | sat | butyl | 8 | 8.8 | RT | |
| | | | | methyl | | | | |
| | | | | ether | | | | |
| PAI- | ethyl acetate | 0.8 | sat | butyl | 8 | 8.8 | RT | −20 |
| GRAD | | | | methyl | | | | |
| | | | | ether | | | | |
| PAI- | ethyl acetate | 0.8 | sat | butyl | 8 | 8.8 | RT | |
| GRAD- | | | | methyl | | | | |
| EVRT | | | | ether | | | | |
| PAI | ethyl acetate | 0.8 | sat | heptane | 8 | 8.8 | RT | |
| PAI- | ethyl acetate | 0.8 | sat | heptane | 8 | 8.8 | RT | −20 |
| GRAD | | | | | | | | |
| PAD | acetone | 0.9 | sat | butyl | 4.5 | 5.4 | RT | |
| | | | | methyl | | | | |
| | | | | ether | | | | |
| PAD | acetone | 0.9 | sat | butyl | 9 | 9.9 | RT | |
| | | | | methyl | | | | |
| | | | | ether | | | | |
| PAD- | acetone | 0.9 | sat | butyl | 9 | 9.9 | RT | −20 |
| GRAD | | | | methyl | | | | |
| | | | | ether | | | | |
| PAD- | acetone | 0.9 | sat | butyl | 9 | 9.9 | RT | |
| GRAD- | | | | methyl | | | | |
| EVRT | | | | ether | | | | |
| PAD | acetone | 0.9 | sat | water | 4.5 | 5.4 | RT | |
| PAD | acetone | 0.9 | sat | heptane | 4.5 | 5.4 | RT | |
| PAD | acetone | 0.9 | sat | heptane | 9 | 9.9 | RT | |
| PAD- | acetone | 0.9 | sat | heptane | 9 | 9.9 | RT | −20 |
| GRAD | | | | | | | | |
| PAI- | acetone | 0.8 | sat | butyl | 8 | 8.8 | RT | −20 |
| GRAD | | | | methyl | | | | |
| | | | | ether | | | | |
| PAI- | acetone | 0.8 | sat | butyl | 8 | 8.8 | RT | |
| GRAD- | | | | methyl | | | | |
| EVRT | | | | ether | | | | |
| PAI | acetone | 0.8 | sat | water | 4 | 4.8 | RT | |
| PAI | acetone | 0.8 | sat | heptane | 8 | 8.8 | RT | |
| PAI- | acetone | 0.8 | sat | heptane | 8 | 8.8 | RT | −20 |
| GRAD | | | | | | | | |

| | | | Output | Output | Output | | Output |
|---|---|---|---|---|---|---|---|
| Experim. | Temp. | Temp. | Aspect | Aspect | Main | Output | Unassigned |
| Type | Ramp | Duration | start | end | phase | Traces | Peaks |
| [descript.] | [K/min] | [days] | [visual] | [visual] | [phase] | [phase] | [2theta] |
| PAD | | | Clear | Clear | no | | |
| | | | solution | solution | solid | | |
| PAD | | | Clear | Clear | no | | |
| | | | solution | solution | solid | | |
| PAD- | not | 1 d | Clear | Clear | no | | |
| GRAD | controlled | | solution | solution | solid | | |
| PAD-- | | | Clear | White | Form | | |
| GRAD- | | | solution | powder | IX | | |
| EVRT | | | | | | | |
| PAD | | | Clear | Clear | no | | |
| | | | solution | solution | solid | | |
| PAD- | not | 1 d | Clear | White | Form | | |
| GRAD | controlled | | solution | powder | IX | | |
| PAI | | | Clear | Clear | no | | |
| | | | solution | solution | solid | | |
| PAI- | not | 1 d | Clear | Clear | no | | |
| GRAD | controlled | | solution | solution | solid | | |
| PAI- | | | Clear | White | Form | | |
| GRAD- | | | solution | powder | IX | | |
| EVRT | | | | | | | |
| PAI | | | Clear | Clear | no | | |
| | | | solution | solution | solid | | |
| PAI- | not | 1 d | Clear | White | Form | | |
| GRAD | controlled | | solution | powder | VII | | |
| PAD | | | Clear | Clear | no | | |
| | | | solution | solution | solid | | |
| PAD | | | Clear | Clear | no | | |
| | | | solution | solution | solid | | |
| PAD- | | 1 d | Clear | Clear | no | | |
| GRAD | | | solution | solution | solid | | |
| PAD- | | | Clear | White | Form | Form | [3.5°_16.7°] |
| GRAD- | | | solution | powder | IX | VII | |
| EVRT | | | | | | | |
| PAD | | | Suspension | White | Form | | |
| | | | | powder | I | | |
| PAD | | | Clear | Clear | no | | |
| | | | solution | solution | solid | | |
| PAD | | | Clear | Clear | no | | |
| | | | solution | solution | solid | | |
| PAD- | not | 1 d | Clear | White | Form | | |
| GRAD | controlled | | solution | powder | XIX | | |
| PAI- | not | 1 d | Clear | Clear | no | | |
| GRAD | controlled | | solution | solution | solid | | |
| PAI- | | | Clear | White | Form | | [3.5°] |
| GRAD- | | | solution | powder | IX | | |
| EVRT | | | | | | | |
| PAI | | | Suspension | White | Form | | |
| | | | | powder | I | | |
| PAI | | | Clear | Clear | no | | |
| | | | solution | solution | solid | | |
| PAI- | not | 1 d | Clear | White | Form | | |
| GRAD | controlled | | solution | powder | XIX | | |

TABLE 26
Results of Precipitation Experiments for Antisolvent Addition of Tetrahydrofuran
and Dimethylformamide Saturated Solutions of (Z)-Endoxifen

| | | | Solvent 1 | | | Solvent | | | |
|---|---|---|---|---|---|---|---|---|---|
| Experim. | Solvent 1 | Solvent 1 | Conc. in | Solvent 2 | Solvent 2 | Mixture 2 | Temp. | Temp. | Temp. |
| Type | Type | Volume | Solvent 1 | Type | Volume | Volume bin | Start | End | Ramp |
| descript.] | [name] | [mL] | [mg/mL] | [name] | [mL] | [mL] | [° C.] | [° C.] | [K/min] |
| PAD | dimethylformamide | 0.15 | sat | butyl | 0.75 | 0.9 | RT | | |
| | | | | methyl | | | | | |
| | | | | ether | | | | | |
| PAD | dimethylformamide | 0.15 | sat | butyl | 1.5 | 1.65 | RT | | |
| | | | | methyl | | | | | |
| | | | | ether | | | | | |
| PAD- | dimethylformamide | 0.15 | sat | butyl | 1.5 | 1.65 | RT | −20 | not |
| GRAD | | | | methyl | | | | | controlled |
| | | | | ether | | | | | |
| PAD- | dimethylformamide | 0.15 | sat | butyl | 1.5 | 1.65 | RT | | |
| GRAD- | | | | methyl | | | | | |
| EVRT | | | | ether | | | | | |
| PAI | dimethylformamide | 0.15 | sat | butyl | 1.5 | 1.65 | RT | | |
| | | | | methyl | | | | | |
| | | | | ether | | | | | |
| PAI- | dimethylformamide | 0.15 | sat | butyl | 1.5 | 1.65 | RT | −20 | not |
| GRAD | | | | methyl | | | | | controlled |
| | | | | ether | | | | | |
| PAI- | dimethylformamide | 0.15 | sat | butyl | 1.5 | 1.65 | RT | | |
| GRAD- | | | | methyl | | | | | |
| EVRT | | | | ether | | | | | |
| PAD | dimethylformamide | 0.15 | sat | water | 0.75 | 0.9 | RT | | |
| PAI | dimethylformamide | 0.15 | sat | water | 0.75 | 0.9 | RT | | |
| PAD | tetrahydrofuran | 0.03 | sat | acetone | 0.15 | 0.18 | RT | | |
| PAD | tetrahydrofuran | 0.03 | sat | acetone | 0.3 | 0.33 | RT | | |
| PAD- | tetrahydrofuran | 0.03 | sat | acetone | 0.3 | 0.33 | RT | −20 | not |
| GRAD | | | | | | | | | controlled |
| PAD-- | tetrahydrofuran | 0.03 | sat | acetone | 0.3 | 0.33 | RT | | |
| GRAD- | | | | | | | | | |
| EVRT | | | | | | | | | |
| PAI | tetrahydrofuran | 0.03 | sat | acetone | 0.3 | 0.33 | RT | | |
| PAI- | tetrahydrofuran | 0.03 | sat | acetone | 0.3 | 0.33 | RT | −20 | not |
| GRAD | | | | | | | | | controlled |
| PAD | tetrahydrofuran | 0.03 | sat | water | 0.15 | 0.18 | RT | | |
| PAD | tetrahydrofuran | 0.03 | sat | water | 0.3 | 0.33 | RT | | |
| PAI | tetrahydrofuran | 0.03 | sat | water | 0.3 | 0.33 | RT | | |
| PAI- | tetrahydrofuran | 0.03 | sat | acetone | 0.3 | 0.33 | RT | | |
| GRAD- | | | | | | | | | |
| EVRT | | | | | | | | | |
| PAD | tetrahydrofuran | 0.03 | sat | heptane | 0.15 | 0.18 | RT | | |
| PAD | tetrahydrofuran | 0.03 | sat | heptane | 0.15 | 0.18 | RT | | |
| PAI | tetrahydrofuran | 0.03 | sat | heptane | 0.15 | 0.18 | RT | | |

| | | Output | Output | | Output | Output | |
|---|---|---|---|---|---|---|---|
| Experim. | Temp. | Aspect | Aspect | Output | Crystallinity | Main | Output |
| Type | Duration | start | end | Yield | [high/low/ | phase | Traces |
| descript.] | [days] | [visual] | [visual] | [if appl.] | SC/oriented] | [phase] | [phase] |
| PAD | | Clear | Clear | | | no solid | |
| | | solution | solution | | | | |
| PAD | | Clear | Clear | | | no solid | |
| | | solution | solution | | | | |
| PAD- | 1 d | Clear | Clear | | | no solid | |
| GRAD | | solution | solution | | | | |
| PAD- | | Clear | Yellowish | | | Form I | |
| GRAD- | | solution | powder | | | | |
| EVRT | | | | | | | |
| PAI | | Clear | Clear | | | no solid | |
| | | solution | solution | | | | |
| PAI- | 1 d | Clear | Clear | | | no solid | |
| GRAD | | solution | solution | | | | |
| PAI- | | Clear | Yellowish | | | Form I | |
| GRAD- | | solution | powder | | | | |
| EVRT | | | | | | | |
| PAD | | Suspension | Yellowish | low | | amorphous | |
| | | | powder | | | | |
| PAI | | Suspension | White | | | Form I | Form VII (at |
| | | | powder | | | | 10.5°. 13.3°. |
| | | | | | | | 14.7°. 18.0°. |
| | | | | | | | 23.8°2Theta) |
| PAD | | Clear | Clear | | | no solid | |
| | | solution | solution | | | | |
| PAD | | Clear | Clear | | | no solid | |
| | | solution | solution | | | | |
| PAD- | 1 d | Clear | Clear | | | no solid | |
| GRAD | | solution | solution | | | | |
| PAD-- | | Clear | White | | | Form XII | Form I |
| GRAD- | | solution | powder | | | | |
| EVRT | | | | | | | |
| PAI | | Clear | Clear | | | no solid | |
| | | solution | solution | | | | |
| PAI- | 3 d | Clear | Clear | | | no solid | |
| GRAD | | solution | solution | | | | |
| PAD | | Suspension | White | low | low | no solid | |
| | | | powder | | | | |
| PAD | | Suspension | White | low | low | Form IV | |
| | | | powder | | | | |
| PAI | | Suspension | White | low | low | Form IV | |
| | | | powder | | | | |
| PAI- | | Clear | White | | | Form XII | Form XIII |
| GRAD- | | solution | powder | | | | (10.1°2Theta) |
| EVRT | | | | | | | |
| PAD | | Suspension | White | low | low | Form XIX | |
| | | | powder | | | | |
| PAD | | Suspension | White | low | low | Form XIX | |
| | | | powder | | | | |
| PAI | | Suspension | White | low | low | Form XIX | |
| | | | powder | | | | |

### Example 23

### Scale-Up Crystallizations of (Z)-Endoxifen

This example covers scale ups of multiple (Z)-endoxifen crystallization procedures disclosed herein. Specifically, the procedures that generated Form I, described in EXAMPLE 5, Form IV, described in EXAMPLE 6, and Form VII, described in EXAMPLE 10, were performed at dg scale. The polymorphs produced from these crystallizations were characterized by XRPD, DSC, TGA-EGA, as well as stability analyses detailed in EXAMPLE 25.

### Scale-Up of Form I

The acetonitrile slurry method for generating Form I (EXAMPLE 5) was performed on a 800 mg-scale. For this crystallization, 800 mg (Z)-endoxifen was partially dissolved in 8 ml acetonitrile at a concentration of 100 mg/ml and left under magnetic stirring at 50° C. for 2 days. Solid (Z)-endoxifen was collected from the slurry and analyzed by XRPD, confirming that the resultant solid was Form I. The remaining slurry was then filtered with a 0.45p m paper filter under vacuum and analyzed by XRPD, which indicated a yield of 94% for the resultant Form I

The resultant Form I was also characterized with DSC and TGA. DSC was performed with a heating rate of 10 K/minute and identified an endothermic peak at 154° C. (onset: 153° C.) ascribable to Form I melting. The TGA profile of the Form I exhibited no weight loss below degradation onset at 270° C.

### Scale-Up of Form IV

The EXAMPLE 6 procedure for generating Form IV by precipitation from 2-propanol/ethyl acetate (50/50 v/v) systems was scaled to both 500 mg and 1 g. For these crystallizations, (Z)-endoxifen was added to 2-propanol/ethyl acetate 50/50 (v/v) mixtures to achieve concentrations of 100 mg/ml. The solvent mixtures were then heated to 65° C. Once complete dissolution of (Z)-endoxifen occurred, the solvent mixtures were cooled to 25° C. at a rate of 0.2° C./minute. The solutions were maintained at 25° C. with stirring for 18 hours, and then vacuum filtered with a 0.45 μm paper filter. XRPD of the resultant crystals displayed a yield of 77% of Form IV prior to filtration and 84% of Form IV after filtration.

Form IV from the two scale-up experiments were then combined for stability analyses. DSC (performed with a heating rate of 10 K/min) of the Form IV showed an endothermic event at 91° C. (onset: 89° C.) possibly ascribable to solvent release, an exothermic event at 98° C. (onset: 96° C.) possibly ascribable to a re-crystallization process, and an endothermic event at 155° C. (onset: 153° C., Delta H 95.51 J/g) possibly due to Form I melting. While the DSC profile of Form IV generated at milligram scales included a small hump on the low temperature side of the 155° C. peak, potentially ascribable to Form VII, the DSC profile of Form IV generated from the scaled-up crystallizations exhibited a symmetric and sharp 155° C. peak, excluding the presence of Form VII. The TGA profile of Form IV generated from the scaled-up crystallizations demonstrated a weight loss of 15.8% between 25-110° C. ascribable to 2-propanol and ethyl acetate release (as confirmed by the gas evolution in the EGA analysis), and degradation of the Form IV above 270° C. When comparing the TGA-EGA analysis between Form IV generated in EXAMPLE 6 and Form IV generated during the scaled-up crystallization, both observed the loss of the same solvents, 2-propanol and ethyl acetate, supporting the possibility that the Form IV crystal structure includes pores capable of containing solvent.

To confirm the DSC results, Form IV deterioration was subject to thermal treatment by incubation for 1 hour at 90° C. and 50 mbar, storage for 18 hours at room temperature, and a second incubation for 3.5 hours 90° C. and 50 mbar. XRPD analysis was performed on the sample following the 1 hour incubation at 90° C. and 50 mbar, after the 18 hours of storage at room temperature, and after the final 3.5 hour incubation 90° C. and 50 mbar. After 1 hour of Form IV incubation at 90° C. and 50 mbar, XRPD analysis showed Form IV with traces of Form I. The XRPD analysis after the final thermal treatment step of 3.5 hour treatment at 90° C. and 50 mbar also showed Form IV with traces of Form I.

### Scale-Up of Form VII

The ethyl acetate:heptane crystallizations outlined in EXAMPLE 10 were repeated at larger scales. TABLE 27 shows the results of PAD and PAI scale-up experiments in ethyl acetate/heptane 1:5 solvent/antisolvent systems. The powder collected from the micro scale-up experiments of PAD and PAI crystallization conditions in ethyl acetate/heptane solvent mixture with a 1:5 ratio was then resuspended in the minimum solvent amount. The employed solvent was a mixture of ethyl acetate/heptane with a ratio of 1:5. The results are summarized in TABLE 28, with a summary of the experiments that generated Form XV summarized in TABLE 29. Two of the crystallizations yielded new forms, named Form XV and Form XVI. While Form XV was the primary polymorph isolated in some of the ethyl acetate:heptane crystallizations, Form XVI was only observed as a minor component of some Form IX, Form XII, and Form XIV mixture.

TABLE 27
Results of PAD and PAI Experiments of (Z)-Endoxifen in
Ethyl Acetate/Heptane 1:5 Solvent/Antisolvent System

| | | | solv1 | | | | Temp | Temp | | |
|---|---|---|---|---|---|---|---|---|---|---|
| Exper. | Solvl | Vol 1 | conc | solv2 | Vol 2 | Vol bin | start | end | Ramp | |
| type | ID | (mL) | (mg/mL | ID | (mL) | (mL) | ° C. | ° C. | (° C./min) | Duration |
| PAD | ethyl | 0.8 | sat | heptane | 4.000 | 4.8 | RT | | | |
| | acetate | | | | | | | | | |
| PAD- | ethyl | 0.8 | sat | heptane | 4 | 4.8 | RT | −20 | not | 1 d |
| GRAD | acetate | | | | | | | | controlled | |
| PAI | ethyl | 0.8 | sat | heptane | 4.000 | 4.8 | RT | | | |
| | acetate | | | | | | | | | |
| PAI- | ethyl | 0.8 | sat | heptane | 4 | 4.8 | RT | −20 | not | 1 d |
| GRAD | acetate | | | | | | | | controlled | |
| PAD | ethyl | 1.2 | sat | heptane | 6.000 | 7.2 | RT | | | |
| | acetate | | | | | | | | | |
| PAD- | acetate | 1.2 | sat | heptane | 6 | 7.2 | RT | −20 | not | 1 d |
| GRAD | ethyl | | | | | | | | controlled | |
| PAI | ethyl | 1.2 | sat | heptane | 6.000 | 7.2 | RT | | | |
| | acetate | | | | | | | | | |
| PAI- | acetate | 1.2 | sat | heptane | 6 | 7.2 | RT | −20 | not | 1 d |
| GRAD | ethyl | | | | | | | | controlled | |
| PAI | ethyl | 1.2 | sat | heptane | 12.000 | 13.2 | RT | | | |
| | acetate | | | | | | | | | |

| | | | | | | | XRPD |
|---|---|---|---|---|---|---|---|
| | | | | XRPD | XRPD | XRPD | Results |
| | | | | Results | Results | Results | Unassigned |
| Exper. | Appearance | Appearance | | Main | Other | Phase in | peaks |
| type | start | end | Yield | phase | phases | traces | (°2theta) |
| PAD | Clear | Clear | | no | | | |
| | solution | solution | | solid | | | |
| PAD- | Clear | White | Low | Form | | Form XVI | [15.4°; 20.3°; |
| GRAD | solution | powder | | XV | | (9.8°2theta) | 20.7°; 24.3°] |
| PAI | Clear | Clear | | no | | | |
| | solution | solution | | solid | | | |
| PAI- | Clear | White | Low | Form | Form | Form IX | |
| GRAD | solution | powder | | XII | XVI | | |
| PAD | Clear | Clear | | no | | | |
| | solution | solution | | solid | | | |
| PAD- | Clear | White | Low | Form | Form | | [13.5°; 17.1°; |
| GRAD | solution | powder | | XV | IX | | 17.5°; 19.2°; |
| | | | | | | | 20.3°; 20.7°; |
| | | | | | | | 24.3°; 24.9°; |
| | | | | | | | 27.1°] |
| PAI | Clear | Clear | | no | | | |
| | solution | solution | | solid | | | |
| PAI- | Clear | White | Low | Form | Form | Form XVI | |
| GRAD | solution | powder | | IX | VII | (9.8°2theta) + | |
| | | | | | | Form XII | |
| PAI | Clear | Clear | | no | | | |
| | solution | solution | | solid | | | |

TABLE 28
Results of micro-scale-up experiments of Form VII

| | | | | | | | | | | | | | | XRPD | XRPD | XRPD |
|---|---|---|---|---|---|---|---|---|---|---|---|---|---|---|---|---|
| | | | solvl | | | Vol | Temp | Temp | Ramp | | Appear- | Appear- | | Results | Results | Results |
| Exper. | Solvl | Vol 1 | conc | solv2 | Vol 2 | bin | start | end | (° C./ | Dura- | ance | ance | | Main | Other | Phase in |
| type | ID | (mL) | (mg/mL | ID | (mL) | (mL) | ° C. | ° C. | min) | tion | start | end | Yield | phase | phases | traces |
| PAI | ethyl | 1.2 | sat | heptane | 12.000 | 13.2 | RT | | | | Clear | Clear | | no | | |
| | acetate | | | | | | | | | | solution | solution | | solid | | |
| PAI- | ethyl | 1.2 | sat | heptane | 12 | 13.2 | RT | −20 | not | 1 d | Clear | White | Low | Form | | |
| GRAD | acetate | | | | | | | | controlled | | solution | powder | | XV | | |
| PAI | ethyl | 1.2 | sat | heptane | 12.000 | 13.2 | RT | | | | Clear | Clear | | no | | |
| | acetate | | | | | | | | | | solution | solution | | solid | | |
| PAI- | ethyl | 1.2 | sat | heptane | 12 | 13.2 | RT | −20 | not | 1 d | Clear | White | Low | Form | Form | Form |
| GRAD | acetate | | | | | | | | controlled | | solution | powder | | IX | XV | VII |
| PAI | ethyl | 1.2 | sat | heptane | 12.000 | 13.2 | RT | | | | Clear | Clear | | no | | |
| | acetate | | | | | | | | | | solution | solution | | solid | | |
| PAI- | ethyl | 1.2 | sat | heptane | 12 | 13.2 | RT | −20 | not | 1 d | Clear | White | Low | Form | Form | |
| GRAD | acetate | | | | | | | | controlled | | solution | powder | | XV | VII | |
| PAI | ethyl | 1.2 | sat | heptane | 12.000 | 13.2 | RT | | | | Clear | Clear | | no | | |
| | acetate | | | | | | | | | | solution | solution | | solid | | |
| PAI- | ethyl | 1.2 | sat | heptane | 12 | 13.2 | RT | −20 | not | 1 d | Clear | White | Low | Form | Form | Form |
| GRAD | acetate | | | | | | | | controlled | | solution | powder | | IX | XV | VII |
| PAI | ethyl | 1.2 | sat | heptane | 12.000 | 13.2 | RT | | | | Clear | Clear | | no | | |
| | acetate | | | | | | | | | | solution | solution | | solid | | |
| PAI- | ethyl | 1.2 | sat | heptane | 12 | 13.2 | RT | −20 | not | 1 d | Clear | White | Low | Form | Form | Form |
| GRAD | acetate | | | | | | | | controlled | | solution | powder | | IX | XV | VII |
| PAI | ethyl | 1.2 | sat | heptane | 12.000 | 13.2 | RT | | | | Clear | Clear | | no | | |
| | acetate | | | | | | | | | | solution | solution | | solid | | |
| PAI- | ethyl | 1.2 | sat | heptane | 12 | 13.2 | RT | −20 | not | 1 d | Clear | White | Low | Form | Form | Form |
| GRAD | acetate | | | | | | | | controlled | | solution | powder | | XV | VII | IX |
| PAI | ethyl | 1.2 | sat | heptane | 12.000 | 13.2 | RT | | | | Clear | Clear | | no | | |
| | acetate | | | | | | | | | | solution | solution | | solid | | |
| PAI- | ethyl | 1.2 | sat | heptane | 12 | 13.2 | RT | −20 | not | 1 d | Clear | White | Good | Form | | |
| GRAD | acetate | | | | | | | | controlled | | solution | powder | | VII | | |
| PAI | ethyl | 1.2 | sat | heptane | 12.000 | 13.2 | RT | | | | Clear | Clear | | no | | |
| | acetate | | | | | | | | | | solution | solution | | solid | | |
| PAI- | ethyl | 1.2 | sat | heptane | 12 | 13.2 | RT | −20 | not | 1 d | Clear | White | Low | Form | Form | Form |
| GRAD | acetate | | | | | | | | controlled | | solution | powder | | IX | XV | VII |

TABLE 29
Summary of the Experiments in which Form XV was Isolated

| | | | | | | | | Solvent | | |
|---|---|---|---|---|---|---|---|---|---|---|
| | | | | | | | | Mixture 2 | | |
| | | | Solvent 1 | | | Solvent | Solvent | Conc. in | | |
| Experim. | Solvent 1 | Solvent 1 | Conc. in | Solvent 2 | Solvent 2 | Mixture 2 | Mixture 2 | Solvent | Temp. | Temp. |
| Type | Type | Volume | Solvent 1 | Type | Volume | Volume bin | Compos. bin | mix bin | Start | End |
| [descript.] | [name] | [mL] | [mg/m L] | [name] | [mL] | [mL] | [v/v] | [mg/m L] | [° C.] | [° C.] |
| PAD | ethyl | 0.8 | sat | heptane | 4 | 4.8 | | | RT | |
| | acetate | | | | | | | | | |
| PAD- | ethyl | 0.8 | sat | heptane | 4 | 4.8 | | | RT | −20 |
| GRAD | acetate | | | | | | | | | |
| PAD | ethyl | 1.2 | sat | heptane | 6 | 7.2 | | | RT | |
| | acetate | | | | | | | | | |
| PAD- | ethyl | 1.2 | sat | heptane | 6 | 7.2 | | | RT | −20 |
| GRAD | acetate | | | | | | | | | |
| PAI- | ethyl | 1.2 | sat | heptane | 6 | 7.2 | | | RT | −20 |
| GRAD | acetate | | | | | | | | | |
| PAI | ethyl | 1.2 | sat | heptane | 12 | 13.2 | | | RT | |
| | acetate | | | | | | | | | |
| PAI- | ethyl | 1.2 | sat | heptane | 12 | 13.2 | | | RT | −20 |
| GRAD | acetate | | | | | | | | | |
| PAI | ethyl | 1.2 | sat | heptane | 12 | 13.2 | | | RT | |
| | acetate | | | | | | | | | |
| PAI- | ethyl | 1.2 | sat | heptane | 12 | 13.2 | | | RT | −20 |
| GRAD | acetate | | | | | | | | | |
| PAI | ethyl | 1.2 | sat | heptane | 12 | 13.2 | | | RT | |
| | acetate | | | | | | | | | |
| PAI- | ethyl | 1.2 | sat | heptane | 12 | 13.2 | | | RT | −20 |
| GRAD | acetate | | | | | | | | | |
| PAI | ethyl | 1.2 | sat | heptane | 12 | 13.2 | | | RT | |
| | acetate | | | | | | | | | |
| PAI- | ethyl | 1.2 | sat | heptane | 12 | 13.2 | | | RT | −20 |
| GRAD | acetate | | | | | | | | | |
| PAI | ethyl | 1.2 | sat | heptane | 12 | 13.2 | | | RT | |
| | acetate | | | | | | | | | |
| PAI- | ethyl | 1.2 | sat | heptane | 12 | 13.2 | | | RT | −20 |
| GRAD | acetate | | | | | | | | | |
| PAI | ethyl | 1.2 | sat | heptane | 12 | 13.2 | | | RT | |
| | acetate | | | | | | | | | |
| PAI- | ethyl | 1.2 | sat | heptane | 12 | 13.2 | | | RT | −20 |
| GRAD | acetate | | | | | | | | | |
| PAI | ethyl | 1.2 | sat | heptane | 12 | 13.2 | | | RT | |
| | acetate | | | | | | | | | |
| PAI- | ethyl | 1.2 | sat | heptane | 12 | 13.2 | | | RT | −20 |
| GRAD | acetate | | | | | | | | | |

| | | | Output | Output | | Output | Output | | Output |
|---|---|---|---|---|---|---|---|---|---|
| Experim. | Temp. | Temp. | Aspect | Aspect | Output | Main | Other | Output | Unassigned |
| Type | Ramp | Duration | start | end | Yield | phase | phases | Traces | Peaks |
| [descript.] | [K/min] | [days] | [visual] | [visual] | [if appl.] | [phase] | [phase] | [phase] | [2theta] |
| PAD | | | Clear | Clear | | no | | | |
| | | | solution | solution | | solid | | | |
| PAD- | not | 1 d | Clear | White | Low | Form | | Form | [15.4°_20.3°_{—} |
| GRAD | controlled | | solution | powder | | XV | | XVI(9.8°2 | 20.7°_24.3°] |
| | | | | | | | | theta) | |
| PAD | | | Clear | Clear | | no | | | |
| | | | solution | solution | | solid | | | |
| PAD- | not | 1 d | Clear | White | Low | Form | Form | | [13.5°_17.1°_{—} |
| GRAD | controlled | | solution | powder | | XV | IX | | 17.5°_19.2°_{—} |
| | | | | | | | | | 20.3°_20.7°_{—} |
| | | | | | | | | | 24.3°_24.9°_{—} |
| | | | | | | | | | 27.1°] |
| PAI- | not | 1 d | Clear | White | Low | Form | Form | Form | |
| GRAD | controlled | | solution | powder | | IX | VII | XVI (9.8° | |
| | | | | | | | | 2theta) + | |
| | | | | | | | | Form | |
| | | | | | | | | XII | |
| PAI | | | Clear | Clear | | no | | | |
| | | | solution | solution | | solid | | | |
| PAI- | not | 1 d | Clear | White | Low | Form | | | |
| GRAD | controlled | | solution | powder | | XV | | | |
| PAI | | | Clear | Clear | | no | | | |
| | | | solution | solution | | solid | | | |
| PAI- | not | 1 d | Clear | White | Low | Form | Form | Form | |
| GRAD | controlled | | solution | powder | | IX | XV | VII | |
| PAI | | | Clear | Clear | | no | | | |
| | | | solution | solution | | solid | | | |
| PAI- | not | 1 d | Clear | White | Low | Form | Form | | |
| GRAD | controlled | | solution | powder | | XV | VII | | |
| PAI | | | Clear | Clear | | no | | | |
| | | | solution | solution | | solid | | | |
| PAI- | not | 1 d | Clear | White | Low | Form | Form | Form | |
| GRAD | controlled | | solution | powder | | IX | XV | VII | |
| PAI | | | Clear | Clear | | no | | | |
| | | | solution | solution | | solid | | | |
| PAI- | not | 1 d | Clear | White | Low | Form | Form | Form | |
| GRAD | controlled | | solution | powder | | IX | XV | VII | |
| PAI | | | Clear | Clear | | no | | | |
| | | | solution | solution | | solid | | | |
| PAI- | not | 1 d | Clear | White | Low | Form | Form | Form | |
| GRAD | controlled | | solution | powder | | XV | VII | IX | |
| PAI | | | Clear | Clear | | no | | | |
| | | | solution | solution | | solid | | | |
| PAI- | not | 1 d | Clear | White | Low | Form | Form | Form | |
| GRAD | controlled | | solution | powder | | IX | XV | VII | |

The XRPD Spectra of Form XV is shown in FIG. 6 with peaks 1-36 corresponding to the peak numbers (No.) in TABLE 30. Peaks in FIG. 6 and TABLE 30 are provided as relative intensities (Rel. Int.) standardized against the peak at 9.8° 2θ (the peak with the highest intensity). The ten largest peaks by measure of Rel. Int. identified in the XRPD of Form XV, were at 9.8, 4.7, 14.0, 20.2, 7.1, 23.4, 22.4, 21.7, 22.7, and 18.8° 2θ, corresponding to peak numbers (No.) 5, 1, 7, 18, 2, 23, 21, 20, 22, and 15, respectively. The three largest peaks by measure of Rel. Int. were at 9.8, 4.7, and 14.0° 2θ.

TABLE 30
X-Ray Powder Diffraction Peak Table for Form XV

| | Pos. | d-spacing | | | Backgr. |
|---|---|---|---|---|---|
| No. | (° 2Th.) | (Å) | Rel. Int. (%) | Height (cts) | (cts) |
| 1 | 4.6719 | 18.91468 | 55.11 | 1681.84 | 192.51 |
| 2 | 7.0508 | 12.53748 | 28.35 | 865.14 | 206.49 |
| 3 | 7.3551 | 12.01935 | 3.78 | 115.42 | 210.17 |
| 4 | 9.3458 | 9.46318 | 14.58 | 445.04 | 248.19 |
| 5 | 9.8338 | 8.99463 | 100 | 3052.06 | 266.2 |
| 6 | 13.1355 | 6.74026 | 4.69 | 143.16 | 440.33 |
| 7 | 13.9662 | 6.34116 | 40.77 | 1244.17 | 537.17 |
| 8 | 14.7785 | 5.9944 | 5.62 | 171.4 | 615.17 |
| 9 | 15.4099 | 5.75019 | 12.71 | 387.85 | 658.78 |
| 10 | 15.9755 | 5.54785 | 11.87 | 362.38 | 685.17 |
| 11 | 16.603 | 5.33956 | 3.75 | 114.42 | 713.77 |
| 12 | 17.3025 | 5.12523 | 7.08 | 216.06 | 751.21 |
| 13 | 17.6963 | 5.01205 | 5.04 | 153.87 | 767 |
| 14 | 18.2334 | 4.86561 | 4.91 | 149.95 | 790.07 |
| 15 | 18.7967 | 4.72105 | 15.42 | 470.56 | 815.75 |
| 16 | 19.3381 | 4.59009 | 1.91 | 58.38 | 834.94 |
| 17 | 19.8707 | 4.46825 | 3.14 | 95.84 | 844.13 |
| 18 | 20.2498 | 4.38545 | 30.56 | 932.82 | 845.7 |
| 19 | 20.6271 | 4.30607 | 5.78 | 176.47 | 842.69 |
| 20 | 21.7282 | 4.09028 | 21.14 | 645.14 | 808.19 |
| 21 | 22.3563 | 3.97676 | 23.37 | 713.39 | 771.19 |
| 22 | 22.7418 | 3.91022 | 15.79 | 481.81 | 742.82 |
| 23 | 23.4491 | 3.79386 | 24.69 | 753.43 | 675.06 |
| 24 | 24.2316 | 3.67309 | 6.74 | 205.83 | 600.03 |
| 25 | 25.2577 | 3.52614 | 4.15 | 126.61 | 570 |
| 26 | 26.3162 | 3.38666 | 9.8 | 299.04 | 537.96 |
| 27 | 27.2293 | 3.27514 | 6.68 | 203.79 | 479.49 |
| 28 | 28.0366 | 3.18264 | 11.04 | 336.88 | 413.98 |
| 29 | 28.7419 | 3.10612 | 2.19 | 66.77 | 362.15 |
| 30 | 29.6566 | 3.01237 | 2.69 | 82.21 | 309.89 |
| 31 | 29.9606 | 2.9825 | 3.12 | 95.27 | 303.86 |
| 32 | 30.5266 | 2.92848 | 2.13 | 64.96 | 286.05 |
| 33 | 31.9834 | 2.79834 | 1.55 | 47.27 | 232.14 |
| 34 | 34.8402 | 2.57515 | 1.02 | 31.1 | 198.82 |
| 35 | 35.6793 | 2.51649 | 1.37 | 41.85 | 201.57 |
| 36 | 37.1059 | 2.42295 | 0.58 | 17.67 | 190.43 |

DSC was performed on (Z)-endoxifen polymorphs obtained from the scaled-up crystallizations. The DSC profile of Form IX displayed an endothermic peak at 134° C. (onset: 130° C.) ascribable to Form IX melting, an exothermic peak at 137° C. (onset 135° C.) ascribable to recrystallization of Form VII, and an endothermic peak at approximately 131° C. which was observed in the DSC profile of Form XII and Form XIII mixture. These results suggest that Form IX could be the phase obtained after desolvation or dehydration of Form XII or Form XIII. Form XV and Form XVI exhibited limited room temperature stabilities.

### Example 24

### Competitive Slurries of (Z)-Endoxifen

This example covers conversions of (Z)-endoxifen polymorphs in solvent slurries. Slurries of Forms I and VII were generated with Forms I, IV, and VII in various solvents, and incubated for either 3 days at room temperature or for 24 hours at high temperature. Following these incubations, polymorphs were collected from the slurries and characterized by XRPD. The solvents, conditions, and resultant polymorphic Forms are summarized in the following tables. TABLE 31 shows the results of a competitive slurry of (Z)-endoxifen as made as described in EXAMPLE 3. TABLE 32 shows the results of a competitive slurry of (Z)-endoxifen as made as described in EXAMPLE 3. TABLE 33 shows the results of a competitive slurry of (Z)-endoxifen Form I and Form IV. TABLE 34 shows the results of a competitive slurry of (Z)-endoxifen Form I and Form VII. TABLE 35 shows the results of a competitive slurry of (Z)-endoxifen Form I, Form IV, and Form VII. While the majority of slurries yielded high purity Form I, some of the slurries yielded Form I with traces of Form IV or Form VII, and six slurries yielded primarily Form IV.

TABLE 31
Competitive Slurry Experiments of (Z)-Endoxifen

| | | | | | | | | | Solvent |
|---|---|---|---|---|---|---|---|---|---|
| | | | | | | | | | Mixture 2 |
| | Input | | | | Solvent 1 | | Solvent | Solvent | Conc. in |
| Experim. | Crystal | Input | Solvent 1 | Solvent 1 | Conc. in | Solvent 2 | Mixture 2 | Mixture 2 | Solvent |
| Type | Phase | Mass | Type | Volume | Solvent 1 | Type | Volume bin | Compos. bin | mix bin |
| [descript.] | [name] | [mg] | [name] | [mL] | [mg/mL] | [name] | [mL] | [v/v] | [mg/mL] |
| SLRT | Form | 50 | 2- | 0.5 | 100 | | | | |
| | I + | | propanol | | | | | | |
| | Form | | | | | | | | |
| | VII | | | | | | | | |
| SLHT | Form | 75 | 2- | 0.5 | 150 | | | | |
| | I + | | propanol | | | | | | |
| | Form | | | | | | | | |
| | VII | | | | | | | | |
| SLRT | Form | 50 | acetone | 0.5 | 100 | | | | |
| | I + | | | | | | | | |
| | Form | | | | | | | | |
| | VII | | | | | | | | |
| SLHT | Form | 50 | acetone | 0.5 | 100 | | | | |
| | I + | | | | | | | | |
| | Form | | | | | | | | |
| | VII | | | | | | | | |
| SLRT | Form | 50 | ethyl | 0.5 | 100 | | | | |
| | I + | | acetate | | | | | | |
| | Form | | | | | | | | |
| | VII | | | | | | | | |
| SLHT | Form | 75 | ethyl | 0.5 | 150 | | | | |
| | I + | | acetate | | | | | | |
| | Form | | | | | | | | |
| | VII | | | | | | | | |
| SLRT | Form | 50 | ethyl | 0.5 | 100 | water(sat) | | | |
| | I + | | acetate | | | | | | |
| | Form | | | | | | | | |
| | VII | | | | | | | | |
| SLHT | Form | 50 | ethyl | 0.5 | 100 | water(sat) | | | |
| | I + | | acetate | | | | | | |
| | Form | | | | | | | | |
| | VII | | | | | | | | |
| SLRT | Form | 50 | heptane | 0.5 | 100 | | | | |
| | I + | | | | | | | | |
| | Form | | | | | | | | |
| | VII | | | | | | | | |
| SLHT | Form | 50 | heptane | 0.5 | 100 | | | | |
| | I + | | | | | | | | |
| | Form | | | | | | | | |
| | VII | | | | | | | | |
| SLRT | Form | 50 | ethyl | | | heptane | 0.5 | 50/50 | 100 |
| | I + | | acetate | | | | | | |
| | Form | | | | | | | | |
| | VII | | | | | | | | |
| SLHT | Form | 50 | ethyl | | | heptane | 0.5 | 50/50 | 100 |
| | I + | | acetate | | | | | | |
| | Form | | | | | | | | |
| | VII | | | | | | | | |
| SLRT | Form | 50 | ethyl | | | acetone | 0.5 | 50/50 | 100 |
| | I + | | acetate | | | | | | |
| | Form | | | | | | | | |
| | VII | | | | | | | | |
| SLHT | Form | 50 | ethyl | | | acetone | 0.5 | 50/50 | 100 |
| | I + | | acetate | | | | | | |
| | Form | | | | | | | | |
| | VII | | | | | | | | |
| SLRT | Form | 50 | acetone | | | 2-propanol | 0.5 | 67/33 | 100 |
| | I + | | | | | | | | |
| | Form | | | | | | | | |
| | VII | | | | | | | | |
| SLHT | Form | 50 | acetone | | | 2-propanol | 0.5 | 67/33 | 100 |
| | I + | | | | | | | | |
| | Form | | | | | | | | |
| | VII | | | | | | | | |
| SLRT | Form | 75 | 2- | | | tetrahydrofuran | 0.5 | 63/37 | 150 |
| | I + | | propanol | | | | | | |
| | Form | | | | | | | | |
| | VII | | | | | | | | |
| SLHT | Form | 100 | 2- | | | tetrahydrofuran | 0.5 | 63/37 | 200 |
| | I + | | propanol | | | | | | |
| | Form | | | | | | | | |
| | VII | | | | | | | | |

| | | | Output | Output | | Output | Output | |
|---|---|---|---|---|---|---|---|---|
| Experim. | Temp. | Temp. | Aspect | Aspect | Output | Main | Other | Output |
| Type | Start | Duration | start | end | Yield | phase | phases | Traces |
| [descript.] | [° C.] | [days] | [visual] | [visual] | [if appl.] | [phase] | [phase] | [phase] |
| SLRT | 25 | 3 d | Suspension | White | high | Form | | Form IV |
| | | | | powder | | I | | |
| SLHT | 50 | 1 d | Suspension | White | high | Form | | Form |
| | | | | powder | | I | | IV(4.8°2theta) |
| SLRT | 25 | 3 d | Suspension | White | high | Form | | |
| | | | | powder | | I | | |
| SLHT | 40 | 1 d | Suspension | White | high | Form | | |
| | | | | powder | | I | | |
| SLRT | 25 | 3 d | Suspension | White | high | Form | | |
| | | | | powder | | I | | |
| SLHT | 50 | 1 d | Suspension | White | high | Form | | |
| | | | | powder | | I | | |
| SLRT | 25 | 3 d | Suspension | White | high | Form | | |
| | | | | powder | | I | | |
| SLHT | 50 | 1 d | Suspension | White | high | Form | | |
| | | | | powder | | I | | |
| SLRT | 25 | 3 d | Suspension | White | high | Form | | Form VII |
| | | | | powder | | I | | |
| SLHT | 50 | 1 d | Suspension | White | high | Form | Form | |
| | | | | powder | | I | VII | |
| SLRT | 25 | 3 d | Suspension | White | high | Form | | |
| | | | | powder | | I | | |
| SLHT | 50 | 1 d | Suspension | White | high | Form | | Form VII |
| | | | | powder | | I | | |
| SLRT | 25 | 3 d | Suspension | White | high | Form | | |
| | | | | powder | | I | | |
| SLHT | 40 | 1 d | Suspension | White | high | Form | | |
| | | | | powder | | I | | |
| SLRT | 25 | 3 d | Suspension | White | high | Form | | |
| | | | | powder | | I | | |
| SLHT | 40 | 1 d | Suspension | White | high | Form | | Form |
| | | | | powder | | I | | IV(4.8°2theta) |
| SLRT | 25 | 3 d | Suspension | White | high | Form | Form | |
| | | | | powder | | I | IV(split | |
| | | | | | | | at 4.8°. | |
| | | | | | | | 19.7° and | |
| | | | | | | | 23.9°2theta) | |
| SLHT | 50 | 1 d | Suspension | White | high | Form | Form IV | |
| | | | | powder | | I | | |

TABLE 32
Competitive Slurry Experiments of (Z)-Endoxifen

| | | | | | | | | | Solvent |
|---|---|---|---|---|---|---|---|---|---|
| | | | | | | | | | Mixture 2 |
| | Input | | | | Solvent 1 | | Solvent | Solvent | Conc. in |
| Experim. | Crystal | Input | Solvent 1 | Solvent 1 | Conc. in | Solvent 2 | Mixture 2 | Mixture 2 | Solvent |
| Type | Phase | Mass | Type | Volume | Solvent 1 | Type | Volume bin | Compos. bin | mix bin |
| [descript.] | [name] | [mg] | [name] | [mL] | [mg/mL] | [name] | [mL] | [v/v] | [mg/mL] |
| SLRT | Form | 50 | 2- | 0.5 | 100 | | | | |
| | I + | | propanol | | | | | | |
| | Form | | | | | | | | |
| | VII | | | | | | | | |
| SLHT | Form | 75 | 2- | 0.5 | 150 | | | | |
| | I + | | propanol | | | | | | |
| | Form | | | | | | | | |
| | VII | | | | | | | | |
| SLRT | Form | 50 | acetone | 0.5 | 100 | | | | |
| | I + | | | | | | | | |
| | Form | | | | | | | | |
| | VII | | | | | | | | |
| SLHT | Form | 50 | acetone | 0.5 | 100 | | | | |
| | I + | | | | | | | | |
| | Form | | | | | | | | |
| | VII | | | | | | | | |
| SLRT | Form | 50 | ethyl | 0.5 | 100 | | | | |
| | I + | | acetate | | | | | | |
| | Form | | | | | | | | |
| | VII | | | | | | | | |
| SLHT | Form | 75 | ethyl | 0.5 | 150 | | | | |
| | I + | | acetate | | | | | | |
| | Form | | | | | | | | |
| | VII | | | | | | | | |
| SLRT | Form | 50 | ethyl | 0.5 | 100 | water(sat) | | | |
| | I + | | acetate | | | | | | |
| | Form | | | | | | | | |
| | VII | | | | | | | | |
| SLHT | Form | 50 | ethyl | 0.5 | 100 | water(sat) | | | |
| | I + | | acetate | | | | | | |
| | Form | | | | | | | | |
| | VII | | | | | | | | |
| SLRT | Form | 50 | heptane | 0.5 | 100 | | | | |
| | I + | | | | | | | | |
| | Form | | | | | | | | |
| | VII | | | | | | | | |
| SLHT | Form | 50 | heptane | 0.5 | 100 | | | | |
| | I + | | | | | | | | |
| | Form | | | | | | | | |
| | VII | | | | | | | | |
| SLRT | Form | 50 | ethyl | | | heptane | 0.5 | 50/50 | 100 |
| | I + | | acetate | | | | | | |
| | Form | | | | | | | | |
| | VII | | | | | | | | |
| SLHT | Form | 50 | ethyl | | | heptane | 0.5 | 50/50 | 100 |
| | I + | | acetate | | | | | | |
| | Form | | | | | | | | |
| | VII | | | | | | | | |
| SLRT | Form | 50 | ethyl | | | acetone | 0.5 | 50/50 | 100 |
| | I + | | acetate | | | | | | |
| | Form | | | | | | | | |
| | VII | | | | | | | | |
| SLHT | Form | 50 | ethyl | | | acetone | 0.5 | 50/50 | 100 |
| | I + | | acetate | | | | | | |
| | Form | | | | | | | | |
| | VII | | | | | | | | |
| SLRT | Form | 50 | acetone | | | 2-propanol | 0.5 | 67/33 | 100 |
| | I + | | | | | | | | |
| | Form | | | | | | | | |
| | VII | | | | | | | | |
| SLHT | Form | 50 | acetone | | | 2-propanol | 0.5 | 67/33 | 100 |
| | I + | | | | | | | | |
| | Form | | | | | | | | |
| | VII | | | | | | | | |
| SLRT | Form | 75 | 2- | | | tetrahydrofuran | 0.5 | 63/37 | 150 |
| | I + | | propanol | | | | | | |
| | Form | | | | | | | | |
| | VII | | | | | | | | |
| SLHT | Form | 100 | 2- | | | tetrahydrofuran | 0.5 | 63/37 | 200 |
| | I + | | propanol | | | | | | |
| | Form | | | | | | | | |
| | VII | | | | | | | | |

| | | | Output | Output | | Output | Output | |
|---|---|---|---|---|---|---|---|---|
| Experim. | Temp. | Temp. | Aspect | Aspect | Output | Main | Other | Output |
| Type | Start | Duration | start | end | Yield | phase | phases | Traces |
| [descript.] | [° C.] | [days] | [visual] | [visual] | [if appl.] | [phase] | [phase] | [phase] |
| SLRT | 25 | 3 d | Suspension | White | high | Form | | Form |
| | | | | powder | | I | | IV |
| SLHT | 50 | 1 d | Suspension | White | high | Form | | Form |
| | | | | powder | | I | | IV |
| SLRT | 25 | 3 d | Suspension | White | high | Form | | |
| | | | | powder | | I | | |
| SLHT | 40 | 1 d | Suspension | White | high | Form | | |
| | | | | powder | | I | | |
| SLRT | 25 | 3 d | Suspension | White | high | Form | | |
| | | | | powder | | I | | |
| SLHT | 50 | 1 d | Suspension | White | high | Form | | |
| | | | | powder | | I | | |
| SLRT | 25 | 3 d | Suspension | White | high | Form | | |
| | | | | powder | | I | | |
| SLHT | 50 | 1 d | Suspension | White | high | Form | | |
| | | | | powder | | I | | |
| SLRT | 25 | 3 d | Suspension | White | high | Form | Form | |
| | | | | powder | | I | VII | |
| SLHT | 50 | 1 d | Suspension | White | high | Form | Form | |
| | | | | powder | | I | VII | |
| SLRT | 25 | 3 d | Suspension | White | high | Form | | |
| | | | | powder | | I | | |
| SLHT | 50 | 1 d | Suspension | White | high | Form | Form | |
| | | | | powder | | I | VII | |
| SLRT | 25 | 3 d | Suspension | White | high | Form | | |
| | | | | powder | | I | | |
| SLHT | 40 | 1 d | Suspension | White | high | Form | | |
| | | | | powder | | I | | |
| SLRT | 25 | 3 d | Suspension | White | high | Form | | |
| | | | | powder | | I | | |
| SLHT | 40 | 1 d | Suspension | White | high | Form | | |
| | | | | powder | | I | | |
| SLRT | 25 | 3 d | Suspension | White | high | Form | Form | |
| | | | | powder | | I | IV | |
| SLHT | 50 | 1 d | Suspension | White | high | Form | Form | |
| | | | | powder | | I | IV | |

TABLE 33
Competitive Slurry Experiments of (Z)-Endoxifen

| | | | | | | | | | | Solvent |
|---|---|---|---|---|---|---|---|---|---|---|
| | | | | | | | | | | Mixture 2 |
| | | Input | | | | Solvent 1 | | Solvent | Solvent | Conc. in |
| Experiment | Experim. | Crystal | Input | Solvent 1 | Solvent 1 | Conc. in | Solvent 2 | Mixture 2 | Mixture 2 | Solvent |
| Name | Type | Phase | Mass | Type | Volume | Solvent 1 | Type | Volume bin | Compos. bin | mix bin |
| [name] | [descript.] | [name] | [mg] | [name] | [mL] | [mg/mL] | [name] | [mL] | [v/v] | [mg/mL] |
| OL08- | SLRT | Form | 50 | ethyl | 0.5 | 100 | water(sat) | | | |
| P01_P02- | | I + | | acetate | | | | | | |
| SLRT_3D- | | Form | | | | | | | | |
| ethyl acetate | | IV | | | | | | | | |
| sat water | | | | | | | | | | |
| OL08- | SLHT | Form | 50 | ethyl | 0.5 | 100 | water(sat) | | | |
| P01_P02- | | I + | | acetate | | | | | | |
| SLHT_1D- | | Form | | | | | | | | |
| ethyl acetate | | IV | | | | | | | | |
| sat water | | | | | | | | | | |
| OL08- | SLRT | Form | 50 | ethyl | 0.5 | 100 | | | | |
| P01_P02- | | I + | | acetate | | | | | | |
| SLRT_3D- | | Form | | | | | | | | |
| ethyl acetate | | IV | | | | | | | | |
| OL08- | SLHT | Form | 50 | ethyl | 0.5 | 100 | | | | |
| P01_P02- | | I + | | acetate | | | | | | |
| SLHT_1 D- | | Form | | | | | | | | |
| ethyl acetate | | IV | | | | | | | | |
| OL08- | SLRT | Form | 50 | 2- | | | tetrahydrofuran | 0.5 | 90/10 | 100 |
| P01_P02- | | I + | | propanol | | | | | | |
| SLRT_3D-2- | | Form | | | | | | | | |
| propanol_tet- | | IV | | | | | | | | |
| rahydrofuran- | | | | | | | | | | |
| 90_10 | | | | | | | | | | |
| OL08- | SLHT | Form | 50 | 2- | | | tetrahydrofuran | 0.5 | 90/10 | 100 |
| P01_P02- | | I + | | propanol | | | | | | |
| SLHT_1D-2- | | Form | | | | | | | | |
| propanol_tet- | | IV | | | | | | | | |
| rahydrofuran- | | | | | | | | | | |
| 90_10 | | | | | | | | | | |
| OL08- | SLRT | Form | 100 | 2- | | | tetrahydrofuran | 0.5 | 63/37 | 200 |
| P01_P02- | | I + | | propanol | | | | | | |
| SLRT_3D-2- | | Form | | | | | | | | |
| propanol_tet | | IV | | | | | | | | |
| rahydrofuran- | | | | | | | | | | |
| 63_37 | | | | | | | | | | |
| OL08- | SLHT- | Form | 100 | 2- | | | tetrahydrofuran | 0.5 | 63/37 | 200 |
| P01_P02- | GRAD | I + | | propanol | | | | | | |
| SLHT_1D- | | Form | | | | | | | | |
| PPT_LT-2- | | IV | | | | | | | | |
| propanol_tet- | | | | | | | | | | |
| rahydrofuran- | | | | | | | | | | |
| 63_37 | | | | | | | | | | |
| OL08- | SLHT | Form | 100 | 2- | | | tetrahydrofuran | 0.5 | 63/37 | 200 |
| P01_P02- | | I + | | propanol | | | | | | |
| SLHT_1D-2- | | Form | | | | | | | | |
| propanol_tet- | | IV | | | | | | | | |
| rahydrofuran- | | | | | | | | | | |
| 63_37 | | | | | | | | | | |

| | | | | | Output | Output | | Output | Output | |
|---|---|---|---|---|---|---|---|---|---|---|
| Experiment | Temp. | Temp. | Temp. | Temp. | Aspect | Aspect | Output | Main | Other | Output |
| Name | Start | End | Ramp | Duration | start | end | Yield | phase | phases | Traces |
| [name] | [° C.] | [° C.] | [K/min] | [days] | [visual] | [visual] | [if appl.] | [phase] | [phase] | [phase] |
| OL08- | 25 | | | 3 d | Suspension | White | high | Form | | |
| P01_P02- | | | | | | powder | | I | | |
| SLRT_3D- | | | | | | | | | | |
| ethyl acetate | | | | | | | | | | |
| sat water | | | | | | | | | | |
| OL08- | 50 | | | 1 d | Suspension | White | high | Form | | |
| P01_P02- | | | | | | powder | | I | | |
| SLHT_1D- | | | | | | | | | | |
| ethyl acetate | | | | | | | | | | |
| sat water | | | | | | | | | | |
| OL08- | 25 | | | 3 d | Suspension | White | high | Form | | |
| P01_P02- | | | | | | powder | | I | | |
| SLRT_3D- | | | | | | | | | | |
| ethyl acetate | | | | | | | | | | |
| OL08- | 50 | | | 1 d | Suspension | White | high | Form | | |
| P01_P02- | | | | | | powder | | I | | |
| SLHT_1 D- | | | | | | | | | | |
| ethyl acetate | | | | | | | | | | |
| OL08- | 25 | | | 3 d | Suspension | White | high | Form | | Form |
| P01_P02- | | | | | | powder | | I | | IV |
| SLRT_3D-2- | | | | | | | | | | |
| propanol_tet- | | | | | | | | | | |
| rahydrofuran- | | | | | | | | | | |
| 90_10 | | | | | | | | | | |
| OL08- | 50 | | | 1 d | Suspension | White | high | Form | Form | |
| P01_P02- | | | | | | powder | | I | IV | |
| SLHT_1D-2- | | | | | | | | | | |
| propanol_tet- | | | | | | | | | | |
| rahydrofuran- | | | | | | | | | | |
| 90_10 | | | | | | | | | | |
| OL08- | 25 | | | 3 d | Suspension | White | high | Form | Form | |
| P01_P02- | | | | | | powder | | I | IV | |
| SLRT_3D-2- | | | | | | | | | | |
| propanol_tet | | | | | | | | | | |
| rahydrofuran- | | | | | | | | | | |
| 63_37 | | | | | | | | | | |
| OL08- | 50 | 10 | 0.5 | | Clear | White | high | Form | | Form |
| P01_P02- | | | | | solution | powder | | IV | | I |
| SLHT_1D- | | | | | | | | | | |
| PPT_LT-2- | | | | | | | | | | |
| propanol_tet- | | | | | | | | | | |
| rahydrofuran- | | | | | | | | | | |
| 63_37 | | | | | | | | | | |
| OL08- | 50 | | | 1 d | Clear | Clear | | no | | |
| P01_P02- | | | | | solution | solution | | solid | | |
| SLHT_1D-2- | | | | | | | | | | |
| propanol_tet- | | | | | | | | | | |
| rahydrofuran- | | | | | | | | | | |
| 63_37 | | | | | | | | | | |

TABLE 34
Competitive Slurry Experiments of (Z)-Endoxifen

| | | | | | | | Solvent | | |
|---|---|---|---|---|---|---|---|---|---|
| | | | | | | | Mixture 2 | | |
| | Input | | | | Solvent | Solvent | Conc. in | | |
| Experim. | Crystal | Input | Solvent 1 | Solvent 2 | Mixture 2 | Mixture 2 | Solvent | Temp. | Temp. |
| Type | Phase | Mass | Type | Type | Volume bin | Compos. bin | mix bin | Start | End |
| [descript.] | [name] | [mg] | [name] | [name] | [mL] | [v/v] | [mg/mL] | [° C.] | [° C.] |
| SLRT | Form I + | 50 | 2- | tetrahydrofuran | 0.5 | 90/10 | 100 | 25 | |
| | Form VII | | propanol | | | | | | |
| SLHT | Form I + | 50 | 2- | tetrahydrofuran | 0.5 | 90/10 | 100 | 50 | |
| | Form VII | | propanol | | | | | | |
| SLRT | Form I + | 100 | 2- | tetrahydrofuran | 0.5 | 75/25 | 200 | 25 | |
| | Form VII | | propanol | | | | | | |
| SLHT | Form I + | 100 | 2- | tetrahydrofuran | 0.5 | 75/25 | 200 | 50 | |
| | Form VII | | propanol | | | | | | |
| SLRT | Form I + | 100 | 2- | tetrahydrofuran | 0.5 | 50/50 | 200 | 25 | |
| | Form VII | | propanol | | | | | | |
| SLHT | Form I + | 100 | 2- | tetrahydrofuran | 0.5 | 50/50 | 200 | 50 | |
| | Form VII | | propanol | | | | | | |
| SLHT- | Form I + | 100 | 2- | tetrahydrofuran | 0.5 | 50/50 | 200 | 50 | 10 |
| GRAD | Form VII | | propanol | | | | | | |

| | | | Output | Output | | Output | Output | | Output |
|---|---|---|---|---|---|---|---|---|---|
| Experim. | Temp. | Temp. | Aspect | Aspect | Output | Main | Other | Output | Unassigned |
| Type | Ramp | Duration | start | end | Yield | phase | phases | Traces | Peaks |
| [descript.] | [K/min] | [days] | [visual] | [visual] | [if appl.] | [phase] | [phase] | [phase] | [2theta] |
| SLRT | | 3 d | Suspension | White | high | Form I | | | |
| | | | | powder | | | | | |
| SLHT | | 1 d | Suspension | White | high | Form I | Form | | |
| | | | | powder | | | IV | | |
| SLRT | | 3 d | Suspension | White | high | Form I | | Form | |
| | | | | powder | | | | IV | |
| SLHT | | 1 d | Suspension | White | high | Form I | Form | | [6.9°] |
| | | | | powder | | | IV | | |
| SLRT | | 3 d | Suspension | White | high | Form | Form I | | |
| | | | | powder | | IV | | | |
| SLHT | | 1 d | Clear | Clear | | no | | | |
| | | | solution | solution | | solid | | | |
| SLHT- | 0.5 | | Clear | White | high | Form | | Form I | |
| GRAD | | | solution | powder | | IV | | | |

TABLE 35
Competitive Slurry Experiments of (Z)-Endoxifen

| | | | | | | | | | Solvent | |
|---|---|---|---|---|---|---|---|---|---|---|
| | | | | | | | | | Mixture 2 | |
| | Input | | | | Solvent 1 | | Solvent | Solvent | in Conc. | |
| Experim. | Crystal | Input | Solvent 1 | Solvent 1 | Conc. in | Solvent 2 | Mixture 2 | Mixture 2 | Solvent | Temp. |
| Type | Phase | Mass | Type | Volume | Solvent 1 | Type | Volume bin | Compos. bin | mix bin | Start |
| descript.] | [name] | [mg] | [name] | [mL] | [mg/mL] | [name] | [mL] | [v/v] | [mg/mL] | [° C.] |
| SLRT | Form | 50 | ethyl | 0.5 | 100 | water(sat) | | | | 25 |
| | I + | | acetate | | | | | | | |
| | Form | | | | | | | | | |
| | IV + | | | | | | | | | |
| | Form | | | | | | | | | |
| | VII | | | | | | | | | |
| SLHT | Form | 50 | ethyl | 0.5 | 100 | water(sat) | | | | 50 |
| | I + | | acetate | | | | | | | |
| | Form | | | | | | | | | |
| | IV + | | | | | | | | | |
| | Form | | | | | | | | | |
| | VII | | | | | | | | | |
| SLRT | Form | 50 | ethyl | 0.5 | 100 | | | | | 25 |
| | I + | | acetate | | | | | | | |
| | Form | | | | | | | | | |
| | IV + | | | | | | | | | |
| | Form | | | | | | | | | |
| | VII | | | | | | | | | |
| SLHT | Form | 50 | ethyl | 0.5 | 100 | | | | | 50 |
| | I + | | acetate | | | | | | | |
| | Form | | | | | | | | | |
| | IV + | | | | | | | | | |
| | Form | | | | | | | | | |
| | VII | | | | | | | | | |
| SLRT | Form | 50 | 2- | | | tetrahydrofuran | 0.5 | 90/10 | 100 | 25 |
| | I + | | propanol | | | | | | | |
| | Form | | | | | | | | | |
| | IV + | | | | | | | | | |
| | Form | | | | | | | | | |
| | VII | | | | | | | | | |
| SLRT | Form | 100 | 2- | | | tetrahydrofuran | 0.5 | 75/25 | 200 | 25 |
| | I + | | propanol | | | | | | | |
| | Form | | | | | | | | | |
| | IV + | | | | | | | | | |
| | Form | | | | | | | | | |
| | VII | | | | | | | | | |
| SLRT | Form | 100 | 2- | | | tetrahydrofuran | 0.5 | 63/37 | 200 | 25 |
| | I + | | propanol | | | | | | | |
| | Form | | | | | | | | | |
| | IV + | | | | | | | | | |
| | Form | | | | | | | | | |
| | VII | | | | | | | | | |
| SLRT | Form | 100 | 2- | | | tetrahydrofuran | 0.5 | 50/50 | 200 | 25 |
| | I + | | propanol | | | | | | | |
| | Form | | | | | | | | | |
| | IV + | | | | | | | | | |
| | Form | | | | | | | | | |
| | VII | | | | | | | | | |
| SLHT | Form | 50 | 2- | | | tetrahydrofuran | 0.5 | 90/10 | 100 | 50 |
| | I + | | propanol | | | | | | | |
| | Form | | | | | | | | | |
| | IV + | | | | | | | | | |
| | Form | | | | | | | | | |
| | VII | | | | | | | | | |
| SLHT | Form | 100 | 2- | | | tetrahydrofuran | 0.5 | 75/25 | 200 | 50 |
| | I + | | propanol | | | | | | | |
| | Form | | | | | | | | | |
| | IV + | | | | | | | | | |
| | Form | | | | | | | | | |
| | VII | | | | | | | | | |
| SLHT | Form | 100 | 2- | | | tetrahydrofuran | 0.5 | 63/37 | 200 | 50 |
| | I + | | propanol | | | | | | | |
| | Form | | | | | | | | | |
| | IV + | | | | | | | | | |
| | Form | | | | | | | | | |
| | VII | | | | | | | | | |
| SLHT- | Form | 100 | 2- | | | tetrahydrofuran | 0.5 | 63/37 | 200 | 50 |
| GRAD | I + | | propanol | | | | | | | |
| | Form | | | | | | | | | |
| | IV + | | | | | | | | | |
| | Form | | | | | | | | | |
| | VII | | | | | | | | | |
| SLHT | Form | 100 | 2- | | | tetrahydrofuran | 0.5 | 50/50 | 200 | 50 |
| | I + | | propanol | | | | | | | |
| | Form | | | | | | | | | |
| | IV + | | | | | | | | | |
| | Form | | | | | | | | | |
| | VII | | | | | | | | | |
| SLHT- | Form | 100 | 2- | | | tetrahydrofuran | 0.5 | 50/50 | 200 | 50 |
| GRAD | I + | | propanol | | | | | | | |
| | Form | | | | | | | | | |
| | IV + | | | | | | | | | |
| | Form | | | | | | | | | |
| | VII | | | | | | | | | |

| | | | | Output | Output | | Output | Output | |
|---|---|---|---|---|---|---|---|---|---|
| Experim. | Temp. | Temp. | Temp. | Aspect | Aspect | Output | Main | Other | Output |
| Type | End | Ramp | Duration | start | end | Yield | phase | phases | Traces |
| descript.] | [° C.] | [K/min] | [days] | [visual] | [visual] | [if appl.] | [phase] | [phase] | [phase] |
| SLRT | | | 3 d | Suspension | White | High | Form | | |
| | | | | | powder | | I | | |
| SLHT | | | 1 d | Suspension | White | High | Form | | |
| | | | | | powder | | I | | |
| SLRT | | | 3 d | Suspension | White | High | Form | | |
| | | | | | powder | | I | | |
| SLHT | | | 1 d | Suspension | White | High | Form | | |
| | | | | | powder | | I | | |
| SLRT | | | 3 d | Suspension | White | High | Form | | Form |
| | | | | | powder | | I | | IV |
| SLRT | | | 3 d | Suspension | White | High | Form | Form | |
| | | | | | powder | | I | IV | |
| SLRT | | | 3 d | Suspension | White | High | Form | Form | |
| | | | | | powder | | IV | I | |
| SLRT | | | 3 d | Suspension | White | High | Form | | Form |
| | | | | | powder | | I | | IV |
| SLHT | | | 1 d | Suspension | White | High | Form | Form | |
| | | | | | powder | | I | IV | |
| SLHT | | | 1 d | Suspension | White | High | Form | Form | |
| | | | | | powder | | I | IV | |
| SLHT | | | 1 d | Clear | Clear | | no | | |
| | | | | solution | solution | | solid | | |
| SLHT- | 10 | 0.5 | | Clear | White | High | Form | | |
| GRAD | | | | solution | powder | | IV | | |
| SLHT | | | 1 d | Clear | Opalescent | | no | | |
| | | | | solution | solution | | solid | | |
| SLHT- | 10 | 0.5 | | Opalescent | White | High | Form | | Form |
| GRAD | | | | solution | powder | | IV | | I |

### Example 25

### Stability Analysis of (Z)-Endoxifen

This example covers the stability of select endoxifen polymorphs. Forms I, IV, and VI were subjected to mechanical stress (grinding and kneading), thermal stress, and humidity to determine their relative stabilities in multiple conditions.

### Procedures

Stability tests were performed on mixtures of Form I and Form IV; Form I and Form VII; and Form I, Form IV, and Form VII. In a first mechanical stability test, the polymorphs were analyzed by XRPD following 10 minutes of grinding by ball milling at 30 Hz. In a second mechanical stability test, the polymorphs were analyzed by XRPD following 10 minutes of kneading by ball milling at 30 Hz in the presence of a catalytic amount of 2-propanol (2 μl).

The stabilities of the polymorphs were also assessed at various temperatures and relative humidities. These analyses were performed by storing 20 mg of polymorph samples for seven days at: (1) 25° C. and 100% relative humidity, (2) 40° C. and 75% relative humidity, (3) 25° C. and 60% relative humidity, and (4) at 60° C. and 75% relative humidity. XRPD was collected on the samples following these incubations and compared to XRPD of the samples prior to storage.

### Results

As determined by XRPD, mechanical grinding and kneading converted portions of Form I to amorphous endoxifen. Storage of Form I at 25° C. and 100% relative humidity did not induce detectable changes in the Form I XRPD, suggesting that Form I is stable under these conditions. The next storage test of Form I generated during the scale-up at 40° C. and 75% relative humidity (RH) for 7 days resulted in no significant changes to the XRPD pattern suggesting Form I's stability at 40° C. and 75% relative humidity (RH) for 7 days.

The mechanical grinding of Form IV resulted in an XRPD pattern of Form I with a mild signal at 4.8° 20 ascribable to Form IV. The kneading of Form IV resulted in an XRPD pattern of a mixture of Form I and Form IV. The first storage test of Form IV generated during the scale-up sample at 25° C. and 100% relative humidity (RH) for 7 days resulted in an XRPD pattern of Form I with trace of Form IV. The next storage test of Form IV generated during the scale-up sample at 40° C. and 75% relative humidity (RH) for 7 days resulted in an XRPD pattern of Form I with minor traces of Form IV.

Form VII generated from the scale-up experiments was not evaluated under mechanical stability. Form VII generated from the scale-up experiments was also tested for stability for 7 days at both 25° C./100% RH and 40° C./75% RH resulting in no significant change to the XRPD pattern suggesting Form VII's stability during storage at these conditions.

Two mixtures of Form I and Form VII were tested for stability varying in different percentages of Form VII. The mechanical grinding of the first mixture of Form I and Form VII resulted in an XRPD pattern of amorphous (Z)-endoxifen with signals ascribable to Form I and Form VII. The mechanical grinding of the second mixture of Form I and Form VII resulted in an XRPD pattern of Form I and Form VII with slightly lowered crystallinity. The kneading of both mixtures of Form I and Form VII resulted in the XRPD pattern of Form I and Form VII with slightly lowered crystallinity. Both mixtures of Form I and Form VII were also tested for stability for 7 days at both 25° C./60% RH and 60° C./75% RH resulting in XRPD patterns of Form I and Form VII similar to the XRPD patterns taken before storage suggesting this samples stability in these conditions.

A mixture of Form I and Form IV was also tested for stability. The mechanical grinding of the mixture of Form I and Form IV resulted in an XRPD pattern of Form I with traces of Form IV. The kneading of the mixture of Form I and Form IV resulted in an XRPD pattern of Form I with a signal at 4.8° 20 ascribable to Form IV. The mixture of Form I and Form IV was also tested for stability for 7 days at both 25° C./60% RH and 60° C./75% RH. Storage for 7 days at 25° C./60% RH resulted in an XRPD pattern mixture of Form I and Form IV. Storage for 7 days at 60° C./75% RH resulted in an XRPD pattern of Form I.

A mixture of Form I, Form IV and Form VII was also tested for stability. The mechanical grinding of the mixture of Form I, Form IV, and Form VII resulted in an XRPD pattern of Form I with a slightly lowered degree of crystallinity. The kneading of the mixture of Form I, Form IV, and Form VII resulted in an XRPD pattern of Form I with a mild signal at 4.8° 20 ascribable to Form IV. The mixture of Form I, Form IV, and Form VII was also tested for stability for 7 days at both 25° C./60% RH and 60° C./75% RH. Storage for 7 days at 25° C./60% RH resulted in an XRPD pattern mixture of Form I, Form IV, and Form VII suggesting stability of the mixture at 25° C./60% RH for 7 days. Storage for 7 days at 60° C./75% RH resulted in an XRPD pattern mixture of Form I and Form VII.

### Example 26

### Kapton Film

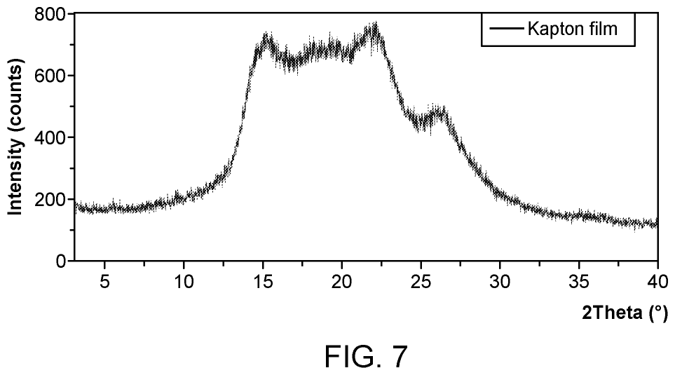

A Kapton film was employed for XRPD analysis of the samples presented herein to mitigate sample loss during preparation and measurement. The Kapton film exhibited an XRPD spectra as seen in FIG. 7.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of purifying a compound of Formula (IV), 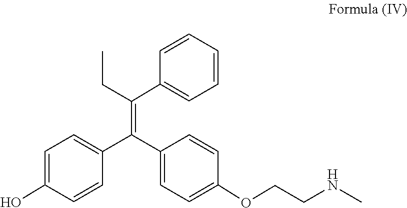 [SMILES: CC/C(=C(\c1ccc(O)cc1)c1ccc(OCCNC)cc1)c1ccccc1]
the method comprising:
providing a compound of Formula (III) that comprises the compound of Formula (IV); 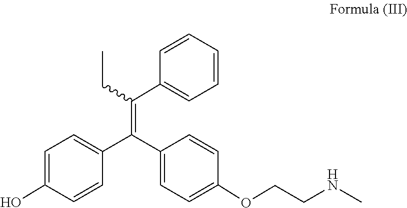 [SMILES: CCC(=C(c1ccc(O)cc1)c1ccc(OCCNC)cc1)c1ccccc1]
performing an ethyl acetate fractional crystallization of the compound of Formula (III) to produce a filtrate comprising the compound of Formula (IV);
performing an acetone recrystallization using the filtrate; and
performing a tetrahydrofuran recrystallization to purify the compound of Formula (IV) with a purity of at least 94% (w/w).

2. The method of claim 1, wherein the providing the compound of Formula (III) comprises:
providing a compound of Formula (II); 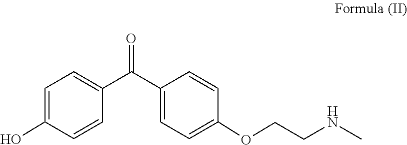 [SMILES: CNCCOc1ccc(C(=O)c2ccc(O)cc2)cc1]
and
performing a McMurry reaction on the compound of Formula (II) and propiophenone to produce the compound of Formula (III).

3. The method of claim 2, wherein the McMurry reaction comprises contacting the compound of Formula (II) and the propiophenone with zinc and titanium (IV) chloride.

4. The method of claim 3, wherein the McMurry reaction further comprises maintaining a temperature of not less than 60° C. for not less than 8 hours.

5. The method of claim 3 or claim 4, wherein the zinc, the titanium (IV) chloride, or both are dissolved or suspended in tetrahydrofuran.

6. The method of any one of claims 3-5, wherein the zinc and the titanium (IV) chloride are mixed at a temperature of not less than 60° C. for not less than 2 hours prior to contacting the compound of Formula (II).

7. The method of any one of claims 3-6, wherein the compound of Formula (II) is in a suspension comprising the propiophenone.

8. The method of claim 7, wherein the suspension is suspended in an organic solvent.

9. The method of claim 8, wherein the organic solvent is selected from the group consisting of 2-methyltetrahydrofuran, dichloromethane, dichloroethane, chloroform, carbon tetrachloride, chlorobenzene, diethyl ether, 1,4-dioxane, tert-butyl methyl ether, tetrahydrofuran, N,N-dimethylformamide, N-methylpyrrolidone, diglyme, nitromethane, 1,2-dimethoxyethane, pyridine, acetone, acetonitrile, benzene, o-xylene, m-xylene, p-xylene, xylenes, hexanes, cyclohexane, heptane, octane, nonane, decane, and combinations thereof.

10. The method of claim 8 or claim 9, wherein the organic solvent is tetrahydrofuran.

11. The method of any one of claims 7-10, wherein the suspension is mixed at a temperature of not less than 60° C. prior to contacting the zinc and the titanium (IV) chloride.

12. The method of any one of claims 3-11, wherein the McMurry reaction further comprises extracting with an organic solvent a product produced upon the contacting the compound of Formula (II) with the zinc and the titanium (IV) chloride.

13. The method of claim 12, wherein the organic solvent is selected from the group consisting of 2-methyltetrahydrofuran, dichloromethane, dichloroethane, chloroform, carbon tetrachloride, chlorobenzene, diethyl ether, 1,4-dioxane, tert-butyl methyl ether, tetrahydrofuran, N,N-dimethylformamide, N-methylpyrrolidone, diglyme, nitromethane, 1,2-dimethoxyethane, pyridine, acetone, acetonitrile, benzene, o-xylene, m-xylene, p-xylene, xylenes, hexanes, cyclohexane, heptane, octane, nonane, decane, and combinations thereof.

14. The method of claim 12 or claim 13, wherein the organic solvent is 2-methyltetrahydrofuran.

15. The method of any one of claims 12-14, wherein the product produced upon the contacting the compound of Formula (II) with the zinc and the titanium (IV) chloride is further extracted with solution comprising a carbonate salt.

16. The method of claim 15, wherein the carbonate salt is selected from the group consisting of potassium carbonate, sodium carbonate, lithium carbonate, magnesium carbonate, and calcium carbonate.

17. The method of claim 15 or claim 16, wherein the carbonate salt is potassium carbonate.

18. The method of any one of claims 12-17, wherein the product produced upon the contacting the compound of Formula (II) with the zinc and the titanium (IV) chloride is further extracted with a solution comprising an Arrhenius base.

19. The method of claim 18, wherein the Arrhenius base is selected from the group consisting of sodium hydroxide, potassium hydroxide, calcium hydroxide, and lithium hydroxide.

20. The method of claim 18 or claim 19, wherein the Arrhenius base is sodium hydroxide.

21. The method of any one of claims 12-20, wherein the product produced upon the contacting the compound of Formula (II) with the zinc and the titanium (IV) chloride is further extracted with a solution comprising a neutral salt.

22. The method of claim 21, wherein the neutral salt is selected from the group consisting of sodium chloride, potassium chloride, potassium sulfate, sodium sulfate, potassium nitrate, sodium nitrate, potassium chlorate, sodium chlorate, potassium perchlorate, and sodium perchlorate.

23. The method of claim 21 or claim 22, wherein the neutral salt is sodium chloride.

24. The method of any one of claims 2-23, wherein the providing the compound of Formula (III) further comprises concentrating a product produced by the McMurry reaction in acetone, acetonitrile, or both.

25. The method of any one of claims 2-24, wherein the providing the compound of Formula (III) further comprises washing a product produced by the McMurry reaction in acetone, acetonitrile, or both.

26. The method of any one of claims 2-25, wherein the providing the compound of Formula (II) comprises:
providing a compound of Formula (I); 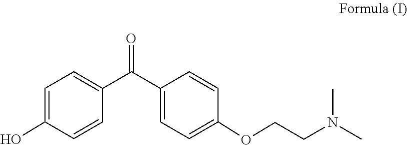 [SMILES: CN(C)CCOc1ccc(C(=O)c2ccc(O)cc2)cc1]
and
performing a demethylation reaction on the compound of Formula (I) to produce the compound of Formula (II).

27. The method of claim 26, wherein the demethylation reaction comprises contacting the compound of Formula (I) with N-ethyldiisopropylamine.

28. The method of claim 27, wherein the compound of Formula (I), the N-ethyldiisopropylamine, or both are dissolved in an organic solvent.

29. The method of claim 28, wherein the organic solvent is selected from the group consisting of 2-methyltetrahydrofuran, dichloromethane, dichloroethane, chloroform, carbon tetrachloride, chlorobenzene, diethyl ether, 1,4-dioxane, tert-butyl methyl ether, tetrahydrofuran, N,N-dimethylformamide, N-methylpyrrolidone, diglyme, nitromethane, 1,2-dimethoxyethane, pyridine, acetone, acetonitrile, benzene, o-xylene, m-xylene, p-xylene, xylenes, hexanes, cyclohexane, heptane, octane, nonane, decane, and combinations thereof.

30. The method of claim 28 or claim 29, wherein the organic solvent is tetrahydrofuran.

31. The method of any one of claims 26-30, wherein the demethylation reaction comprises contacting the compound of Formula (I) with 1-chloroethyl chloroformate.

32. The method of claim 31, wherein the compound of Formula (I) and the 1-chloroethyl chloroformate are heated to reflux.

33. The method of claim 31 or claim 32, wherein the compound of Formula (I) and the 1-chloroethyl chloroformate are mixed at not less than 60° C. for not less than 12 hours.

34. The method of any one of claims 26-33, wherein the demethylation reaction comprises contacting the compound of Formula (I) with a solution comprising an Arrhenius acid.

35. The method of claim 34, wherein the Arrhenius acid is selected from the group consisting of hydrochloric acid, hydrobromic acid, hydroiodic acid, hydrosulfuric acid, acetic acid, nitric acid, nitrous acid, sulfuric acid, sulfurous acid, chloric acid, perchloric acid, chlorous acid, phosphoric acid, phosphorous acid, and carbonic acid.

36. The method of claim 34 or claim 35, wherein the Arrhenius acid is hydrochloric acid.

37. The method of any one of claims 26-36, further comprising increasing a pH of the demethylation reaction to at least about 13 or at least about 13.4.

38. The method of claim 37, wherein increasing the pH comprises adding solution comprising an Arrhenius base to the demethylation reaction.

39. The method of claim 38, wherein the Arrhenius base is selected from the group consisting of sodium hydroxide, potassium hydroxide, calcium hydroxide, and lithium hydroxide.

40. The method of claim 38 or claim 39, wherein the Arrhenius base is sodium hydroxide.

41. The method of any one of claims 26-40, further comprising extracting the compound of Formula (II) with ethyl acetate.

42. The method of any one of claims 1-41, wherein the ethyl acetate fractional recrystallization comprises contacting the compound of Formula (III) with ethyl acetate and an Arrhenius acid to extract the compound of Formula (IV).

43. The method of claim 42, wherein the Arrhenius acid is selected from the group consisting of hydrochloric acid, hydrobromic acid, hydroiodic acid, hydrosulfuric acid, acetic acid, nitric acid, nitrous acid, sulfuric acid, sulfurous acid, chloric acid, perchloric acid, chlorous acid, phosphoric acid, phosphorous acid, and carbonic acid.

44. The method of claim 42 or claim 43, wherein the Arrhenius acid is hydrochloric acid.

45. The method of any one of claims 42-44, wherein the compound of Formula (III), the ethyl acetate, and the Arrhenius acid are mixed at from 50° C. to 70° C., or from 55° C. to 65° C. for not less than 6 hours.

46. The method of any one of claims 42-45, wherein the ethyl acetate fractional recrystallization further comprises increasing the pH of a solution comprising the compound of Formula (III) to not less than 12 by adding an Arrhenius base.

47. The method of claim 46, wherein the Arrhenius base is selected from the group consisting of sodium hydroxide, potassium hydroxide, calcium hydroxide, and lithium hydroxide.

48. The method of claim 46 or claim 47, wherein the Arrhenius base is sodium hydroxide.

49. The method of any one of claims 42-45, wherein the ethyl acetate fractional recrystallization further comprises adding ethyl acetate to extract the compound of Formula (IV).

50. The method of any one of claims 42-45, wherein the ethyl acetate fractional recrystallization further comprises washing the compound of Formula (IV) with a solution comprising a neutral salt.

51. The method of claim 50, wherein the neutral salt is selected from the group consisting of sodium chloride, potassium chloride, potassium sulfate, sodium sulfate, potassium nitrate, sodium nitrate, potassium chlorate, sodium chlorate, potassium perchlorate, and sodium perchlorate.

52. The method of claim 50 or claim 51, wherein the neutral salt is sodium chloride.

53. The method of any one of claims 42-52, wherein the ethyl acetate fractional recrystallization further comprises filtering the compound of Formula (IV).

54. The method of any one of claims 1-53, wherein the acetone crystallization comprises contacting the compound of Formula (IV) with acetone.

55. The method of claim 54, wherein the acetone crystallization further comprises contacting the compound of Formula (IV) and the acetone with 2-propanol.

56. The method of any one of claims 1-55, wherein the tetrahydrofuran crystallization comprises contacting the compound of Formula (IV) with tetrahydrofuran.

57. The method of claim 56, wherein the tetrahydrofuran crystallization further comprises contacting the compound of Formula (IV) and the tetrahydrofuran with 2-propanol.

58. The method of any one of claims 1-57, wherein the ethyl acetate fractional recrystallization, the acetone crystallization, the tetrahydrofuran crystallization, or a combination thereof reduces a level of (E)-endoxifen in the compound of Formula (III) or the compound of Formula (IV).

59. The method of claim 58, wherein the ethyl acetate fractional recrystallization, the acetone crystallization, the tetrahydrofuran crystallization, or the combination thereof reduces the level of (E)-endoxifen in the compound of Formula (III) or the compound of Formula (IV) by at least about 99.9%, 99%, 97%, 75%, 60%, 50%, 45%, 40%, 30%, 20%, 10%, or 5%.

60. The method of claim 58 or claim 59, wherein the level of (E)-endoxifen is measured by HPLC.

61. The method of any one of claims 1-60, wherein the ethyl acetate fractional recrystallization, the acetone crystallization, the tetrahydrofuran crystallization, or a combination thereof reduces a level of mesityl oxide in the compound of Formula (III) or the compound of Formula (IV).

62. The method of claim 61, wherein the ethyl acetate fractional recrystallization, the acetone crystallization, the tetrahydrofuran crystallization, or the combination thereof reduces the level of mesityl oxide in the compound of Formula (III) or the compound of Formula (IV) by at least about 99.9%, 99%, 97%, 75%, 60%, 50%, 40%, 30%, 20%, 15%, 10%, 5%, or 1%.

63. The method of claim 61 or claim 62, wherein the level of mesityl oxide is measured by HPLC.

64. The method of any one of claims 1-63, wherein the ethyl acetate fractional recrystallization, the acetone crystallization, the tetrahydrofuran crystallization, or a combination thereof reduces a level of 4-(1-(4-methoxyphenyl)-2-phenylbut-1-en-1-yl)phenol, a level of 4-(1-(4-isopropoxyphenyl)-2-phenylbut-1-en-1-yl)phenol, or a level of 1-(4-(2-hydroxyethoxy)phenyl)-1,2-bis(4-hydroxyphenyl)-2-(4-(2-(methylamino)ethoxy)phenyl)ethane-1.2-diol in the compound of Formula (III) or the compound of Formula (IV).

65. The method of claim 64, wherein the ethyl acetate fractional recrystallization, the acetone crystallization, the tetrahydrofuran crystallization, or the combination thereof reduces the level 4-(1-(4-methoxyphenyl)-2-phenylbut-1-en-1-yl)phenol by at least about 99.9%, 99%, 97%, 75%, 60%, 50%, 40%, 30%, 20%, 15%, 10%, 5%, or 1%.

66. The method of claim 64 or claim 65, wherein the ethyl acetate fractional recrystallization, the acetone crystallization, the tetrahydrofuran crystallization, or the combination thereof reduces the level 4-(1-(4-isopropoxyphenyl)-2-phenylbut-1-en-1-yl)phenol by at least about 99.9%, 99%, 97%, 75%, 60%, 50%, 40%, 30%, 20%, 15%, 10%, 5%, or 1%.

67. The method of any one of claims 64-66, wherein the ethyl acetate fractional recrystallization, the acetone crystallization, the tetrahydrofuran crystallization, or the combination thereof reduces the level 1-(4-(2-hydroxyethoxy)phenyl)-1,2-bis(4-hydroxyphenyl)-2-(4-(2-(methylamino)ethoxy)phenyl)ethane-1,2-diol by at least about 99.9%, 99%, 97%, 75%, 60%, 50%, 40%, 30%, 20%, 15%, 10%, 5%, or 1%.

68. The method of any one of claims 64-67, wherein the level of 1-(4-(2-hydroxyethoxy)phenyl)-1,2-bis(4-hydroxyphenyl)-2-(4-(2-(methylamino)ethoxy)phenyl)ethane-1,2-diol comprises a level of (1S,2R)-1-(4-(2-hydroxyethoxy)phenyl)-1,2-bis(4-hydroxyphenyl)-2-(4-(2-(methylamino)ethoxy)phenyl)ethane-1,2-diol; a level of (1R,2S)-1-(4-(2-hydroxyethoxy)phenyl)-1,2-bis(4-hydroxyphenyl)-2-(4-(2-(methylamino)ethoxy)phenyl)ethane-1,2-diol; a level of (1S,2S)-1-(4-(2-hydroxyethoxy)phenyl)-1,2-bis(4-hydroxyphenyl)-2-(4-(2-(methylamino)ethoxy)phenyl)ethane-1,2-diol; a level of (1R,2R)-1-(4-(2-hydroxyethoxy)phenyl)-1,2-bis(4-hydroxyphenyl)-2-(4-(2-(methylamino)ethoxy)phenyl)ethane-1,2-diol; or a combination thereof.

69. The method of any one of claims 64-68, wherein the level of 4-(1-(4-methoxyphenyl)-2-phenylbut-1-en-1-yl)phenol, the level of 4-(1-(4-isopropoxyphenyl)-2-phenylbut-1-en-1-yl)phenol, or the level of 1-(4-(2-hydroxyethoxy)phenyl)-1,2-bis(4-hydroxyphenyl)-2-(4-(2-(methylamino)ethoxy)phenyl)ethane-1,2-diol is measured by HPLC.

70. The method of any one of claims 1-69, wherein the compound of Formula (IV) is purified with a purity of at least 96% (w/w).

71. A composition, comprising:
at least 96% w/w of a compound of Formula (IV), 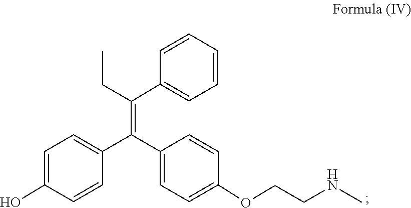 [SMILES: CC/C(=C(\c1ccc(O)cc1)c1ccc(OCCNC)cc1)c1ccccc1]
and
not more than 4% w/w of impurities, wherein the impurities comprise not more than 25 ppm mesityl oxide, with respect to a total amount of the composition.

72. The composition of claim 71, wherein the impurities comprise not more than 3% (w/w) (E)-endoxifen, with respect to the total amount of the composition.

73. A composition comprising a compound of Formula (IV), produced by the method of any one of claims 1-70.

74. The composition of any one of claims 71-73, comprising not more than 3%, 2.5%, 2%, 1.5%, or 1% (E)-endoxifen by weight.

75. The composition of any one of claims 71-74, comprising not less than 97%, 97.5%, 98%, 98.5%, or 99% (Z)-endoxifen by weight.

76. The composition of any one of claims 71-75, comprising not more than 5 ppm, 10 ppm, 15 ppm, 20 ppm, or 25 ppm mesityl oxide.

77. The composition of any one of claims 71-76, comprising not more than 0.1%, 0.15%, 0.2%, 0.25%, 0.3%, 0.4%, or 0.5% 4-(1-(4-methoxyphenyl)-2-phenylbut-1-en-1-yl)phenol by weight.

78. The composition of any one of claims 71-77, comprising not more than 0.1%, 0.15%, 0.2%, 0.25%, 0.3%, 0.4%, or 0.5% 4-(1-(4-isopropoxyphenyl)-2-phenylbut-1-en-1-yl)phenol by weight.

79. The composition of any one of claims 71-78, comprising not more than 0.1%, 0.15%, 0.2%, 0.25%, 0.3%, 0.4%, 0.5%, 0.6%, 0.7%, 0.8%, 0.9%, or 1% 1-(4-(2-hydroxyethoxy)phenyl)-1,2-bis(4-hydroxyphenyl)-2-(4-(2-(methylamino)ethoxy)phenyl)ethane-1,2-diol by weight.

80. The composition of any one of claims 71-79, comprising not more than 0.1%, 0.15%, 0.2%, 0.25%, 0.3%, 0.4%, or 0.5% (1S,2R)-1-(4-(2-hydroxyethoxy)phenyl)-1,2-bis(4-hydroxyphenyl)-2-(4-(2-(methylamino)ethoxy)phenyl)ethane-1,2-diol by weight.

81. The composition of any one of claims 71-80, comprising not more than 0.1%, 0.15%, 0.2%, 0.25%, 0.3%, 0.4%, or 0.5% (1R,2S)-1-(4-(2-hydroxyethoxy)phenyl)-1,2-bis(4-hydroxyphenyl)-2-(4-(2-(methylamino)ethoxy)phenyl)ethane-1,2-diol by weight.

82. The composition of any one of claims 71-81, comprising not more than 0.1%, 0.15%, 0.2%, 0.25%, 0.3%, 0.4%, or 0.5% (1S,2S)-1-(4-(2-hydroxyethoxy)phenyl)-1,2-bis(4-hydroxyphenyl)-2-(4-(2-(methylamino)ethoxy)phenyl)ethane-1,2-diol by weight.

83. The composition of any one of claims 71-82, comprising not more than 0.1%, 0.15%, 0.2%, 0.25%, 0.3%, 0.4%, or 0.5% (1R,2R)-1-(4-(2-hydroxyethoxy)phenyl)-1,2-bis(4-hydroxyphenyl)-2-(4-(2-(methylamino)ethoxy)phenyl)ethane-1,2-diol by weight.

84. The composition of any one of claims 71-83, comprising not more than 5000 ppm ethanol.

85. The composition of any one of claims 71-84, comprising not more than 3000 ppm methanol.

86. The composition of any one of claims 71-85, comprising not more than 5000 ppm acetone.

87. The composition of any one of claims 71-86, comprising not more than 5000 ppm 2-propanol.

88. The composition of any one of claims 71-87, comprising not more than 410 ppm acetonitrile.

89. The composition of any one of claims 71-88, comprising not more than 5000 ppm ethyl acetate.

90. The composition of any one of claims 71-89, comprising not more than 720 ppm tetrahydrofuran.

91. The composition of any one of claims 71-90, comprising not more than 520 ppm 2-methyltetrahydrofuran.

92. The composition of any one of claims 71-91, comprising not more than 5000 ppm n-heptane.

93. The composition of any one of claims 71-92, comprising not more than 130 ppm zinc.

94. The composition of any one of claims 71-93, comprising not more than 2 ppm benzene.

95. A composition comprising a crystalline form of a compound of Formula (III): 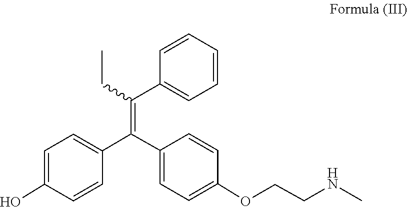 [SMILES: CCC(=C(c1ccc(O)cc1)c1ccc(OCCNC)cc1)c1ccccc1]
wherein the crystalline form of the compound of Formula (III) comprises:
(a) Form IV as characterized by an x-ray powder diffraction pattern comprising major peaks at 4.7±0.3° 2θ, 23.3±0.3° 2θ, and 13.6±0.3° 2θ;
(b) Form V as characterized by x-ray powder diffraction pattern comprising major peaks at 12.5±0.3° 2θ, 19.6±0.3° 2θ, and 8.9±0.3° 2θ;
(c) Form VI as characterized by x-ray powder diffraction pattern comprising major peaks at 9.9±0.3° 2θ, 13.4±0.3° 2θ, and 13.7±0.3° 2θ;
(d) Form VII as characterized by x-ray powder diffraction pattern comprising major peaks at 20.0±0.3° 2θ, 22.6±0.3° 2θ, and 10.6±0.3° 2θ;
(e) Form VIII as characterized by x-ray powder diffraction pattern comprising major peaks at 4.8±0.3° 2θ, 18.9±0.3° 2θ, and 9.5±0.3° 2θ;
(f) Form IX as characterized by x-ray powder diffraction pattern comprising major peaks at 19.0±0.3° 2θ, 12.9±0.3° 2θ, and 15.9±0.3° 2θ;
(g) Form X as characterized by x-ray powder diffraction pattern comprising major peaks at 7.2±0.3° 2θ, 14.3±0.3° 2θ, 18.7±0.3° 2θ, 21.5±0.3° 2θ, and 22.7±0.3° 2θ;
(h) Form XI as characterized by x-ray powder diffraction pattern comprising major peaks at 14.0±0.3° 2θ, 17.7±0.3° 2θ, 11.9±0.3° 2θ, 18.4±0.3° 2θ, 23.9±0.3° 2θ, 17.3±0.3° 2θ, 21.8±0.3° 2θ, 20.8±0.3° 2θ, and 23.0±0.3° 2θ;
(i) Form XII as characterized by x-ray powder diffraction pattern comprising major peaks at 12.5±0.3° 2θ, 15.6±0.3° 2θ, and 19.0±0.3°2θ;
(j) Form XIV as characterized by x-ray powder diffraction pattern comprising major peaks at 11.6±0.3° 2θ, 21.3±0.3° 2θ, and 19.3±0.3° 2θ;
(k) Form XV as characterized by x-ray powder diffraction pattern comprising major peaks at 9.8±0.3° 2θ, 4.7±0.3° 2θ, and 14.0±0.3° 2θ;
(l) Form XIX as characterized by x-ray powder diffraction pattern comprising major peaks at 4.7±0.3° 2θ, 23.6±0.3° 2θ, and 18.9±0.3° 2θ;
or
(m) a combination thereof.

96. The composition of claim 95, wherein at least about 80%, at least about 90%, at least about 95%, at least about 98%, or at least about 99% by weight of the compound of Formula (III) in the composition is the (Z)-isomer.

97. The composition of claim 95 or claim 96, wherein at least about 50%, at least about 60%, at least about 70%, at least about 80%, at least about 90%, at least about 95%, at least about 98%, or at least about 99% of the compound of Formula (III) is Form IV, Form V, Form VI, Form VII, Form VIII, Form IX, Form X, Form XI, Form XII, Form XIII, Form XIV, Form XV, Form XIX, or a combination thereof.

98. The composition of any one of claims 93-95, wherein the crystalline form of the compound of Formula (III) is stable at ambient temperature for at least 1 day, at least 3 days, is stable at ambient temperature for at least 7 days, is stable at ambient temperature for at least 14 days, is stable at ambient temperature for at least 21 days, is stable at ambient temperature for at least 30 days, is stable at ambient temperature for at least 60 days, or is stable at ambient temperature for at least 90 days.

99. The composition of any one of claims 95-98, wherein the crystalline form comprises Form IV, or Form VII.

100. The composition of any one of claims 95-98, wherein the crystalline form comprises Form IV, Form VI, Form VII, Form VIII, Form IX, Form X, or Form XIV.

101. The composition of any one of claims 95-100, wherein the crystalline form comprises Form IV, Form V, Form VI, Form VII, Form VIII, Form IX, Form XII, Form XIV, Form XV, or Form XIX.

102. The composition of any one of claims 95-98 or 101, wherein the crystalline form of the compound of Formula (III) is Form IV.

103. The composition of claim 102, wherein the crystalline form of the compound of Formula (III) is characterized by an x-ray powder diffraction pattern comprising at least one peak, at least two peaks, at least three peaks, or at least four peaks selected from 23.8±0.3° 2θ, 14.2±0.3° 2θ, 22.5±0.3° 2θ, and 15.7±0.3° 2θ.

104. The composition of claim 103, wherein the x-ray powder diffraction pattern further comprises at least one peak or at least two peaks selected from 7.1±0.3° 2θ and 20.2±0.3° 2θ.

105. The composition of claim 103 or claim 104, wherein the x-ray powder diffraction pattern further comprises at least one peak, comprises a peak at 9.5±0.3° 2θ.

106. The composition of any one of claims 103-105, wherein the crystalline form of the compound of Formula (III) is characterized by an x-ray diffraction pattern substantially as set forth in FIG. 13.

107. The composition of any one of claims 102-106, wherein the crystalline form of the compound of Formula (III) comprises between about 1% and about 30% solvent by weight as determined by TGA analysis.

108. The composition of claim 107, wherein the crystalline form loses the solvent upon heating to a temperature between 70° C. and 130° C. as determined by TGA analysis.

109. The composition of claim 107 or claim 108, wherein the solvent comprises 2-propanol, heptane, or a combination thereof.

110. The composition of any one of claims 95-98 or 101, wherein the crystalline form of the compound of Formula (III) is Form V.

111. The composition of claim 110, wherein the crystalline form of the compound of Formula (III) is characterized by an x-ray powder diffraction pattern comprising at least one peak, at least two peaks, at least three peaks, or at least four peaks selected from 21.7±0.3° 2θ, 20.8±0.3° 2θ, 19.8±0.3° 2θ, and 16.0±0.3° 2θ.

112. The composition of claim 111, wherein the x-ray powder diffraction pattern further comprises at least one peak, at least two peaks, or at least three peaks selected from 22.0±0.3° 2θ, 13.5±0.3° 2θ, or 14.4±0.3° 2θ.

113. The composition of any one of claims 110-112, wherein the crystalline form of the compound of Formula (III) is characterized by an x-ray diffraction pattern substantially as set forth in FIG. 27.

114. The composition of any one of claims 95-98 or 100-101, wherein the crystalline form of the compound of Formula (III) is Form VI.

115. The composition of claim 114, wherein the crystalline form of the compound of Formula (III) is characterized by an x-ray powder diffraction pattern comprising at least one peak, at least two peaks, at least three peaks, or at least four peaks selected from 17.6±0.3° 2θ, 18.6±0.3° 2θ, 17.3±0.3° 2θ, and 21.8±0.3° 2θ.

116. The composition of claim 115, wherein the x-ray powder diffraction pattern further comprises at least one peak, at least two peaks, or at least three peaks selected from 10.2±0.3° 2θ, 19.5±0.3° 2θ, and 14.2±0.3° 2θ.

117. The composition of any one of claims 114-116, wherein the crystalline form of the compound of Formula (III) is characterized by an x-ray diffraction pattern substantially as set forth in FIG. 29.

118. The composition of any one of claims 95-101, wherein the crystalline form of the compound of Formula (III) is Form VII.

119. The composition of claim 118, wherein the crystalline form of the compound of Formula (III) is characterized by an x-ray powder diffraction pattern comprising at least one peak, at least two peaks, at least three peaks, or at least four peaks selected from 11.4±0.3° 2θ, 16.4±0.3° 2θ, 9.6±0.3° 2θ, and 13.3±0.3° 2θ.

120. The composition of claim 119, wherein the x-ray powder diffraction pattern further comprises at least one peak, at least two peaks, or at least three peaks selected from 18.2±0.3° 2θ, 13.1±0.3° 2θ, and 27.0±0.3° 2θ.

121. The composition of any one of claims 118-120, wherein the crystalline form of the compound of Formula (III) is characterized by an x-ray diffraction pattern substantially as set forth in FIG. 31.

122. The composition of any one of claims 95-98 or 100-101, wherein the crystalline form of the compound of Formula (III) is Form VIII.

123. The composition of claim 122, wherein the crystalline form of the compound of Formula (III) is characterized by an x-ray powder diffraction pattern comprising at least one peak, at least two peaks, at least three peaks, or at least four peaks selected from 23.7±0.3° 2θ, 21.9±0.3° 2θ, 21.2±0.3° 2θ, and 12.9±0.3° 2θ.

124. The composition of claim 123, wherein the x-ray powder diffraction pattern further comprises at least one peak, at least two peaks, or at least three peaks selected from 25.0±0.3° 2θ, 21.5±0.3° 2θ, and 16.4±0.3° 2θ.

125. The composition of any one of claims 122-124, wherein the crystalline form of the compound of Formula (III) is characterized by an x-ray diffraction pattern substantially as set forth in FIG. 37.

126. The composition of any one of claims 95-98 or 100-101, wherein the crystalline form of the compound of Formula (III) is Form IX.

127. The composition of claim 126, wherein the crystalline form of the compound of Formula (III) is characterized by an x-ray powder diffraction pattern comprising at least one peak, at least two peaks, at least three peaks, or at least four peaks selected from 21.7±0.3° 2θ, 20.8±0.3° 2θ, 21.1±0.3° 2θ, and 8.9±0.3° 2θ.

128. The composition of claim 127, wherein the x-ray powder diffraction pattern further comprises at least one peak, at least two peaks, or at least three peaks selected from 16.4±0.3° 2θ, 4.2±0.3° 2θ, and 12.7±0.3° 2θ.

129. The composition of any one of claims 126-128, wherein the crystalline form of the compound of Formula (III) is characterized by an x-ray diffraction pattern substantially as set forth in FIG. 40.

130. The composition of any one of claims 95-98 or 100, wherein the crystalline form of the compound of Formula (III) is Form X.

131. The composition of claim 130, wherein the crystalline form of the compound of Formula (III) is characterized by an x-ray powder diffraction pattern comprising at least one peak or at least two peaks selected from 17.1±0.3° 2θ, and 22.7±0.3° 2θ.

132. The composition of claim 131, wherein the x-ray powder diffraction pattern further comprises at least one peak, at least two peaks, or at least three peaks selected from 21.8±0.3° 2θ, 27.3±0.3° 2θ, and 29.4±0.3° 2θ.

133. The composition of any one of claims 130-132 wherein the crystalline form of the compound of Formula (III) is characterized by an x-ray diffraction pattern substantially as set forth in FIG. 52.

134. The composition of any one of claims 95-98, wherein the crystalline form of the compound of Formula (III) is Form XI.

135. The composition of claim 134, wherein the crystalline form of the compound of Formula (III) is characterized by an x-ray powder diffraction pattern comprising at least one peak or at least two peaks selected from 22.2±0.3° 20 and 16.6±0.3° 2θ.

136. The composition of claim 134 or claim 135, wherein the crystalline form of the compound of Formula (III) is characterized by an x-ray diffraction pattern substantially as set forth in FIG. 54.

137. The composition of any one of claims 95-98 or 101, wherein the crystalline form of the compound of Formula (III) is Form XII.

138. The composition of claim 137, wherein the crystalline form of the compound of Formula (III) is characterized by an x-ray powder diffraction pattern comprising at least one peak, at least two peaks, at least three peaks, or at least four peaks selected from 21.9±0.3° 2θ, 20.2±0.3° 2θ, 16.0±0.3° 2θ, and 21.6±0.3° 2θ.

139. The composition of claim 138, wherein the x-ray powder diffraction pattern further comprises at least one peak, at least two peaks, or at least three peaks selected from 22.4±0.3° 2θ, 16.8±0.3° 2θ, and 12.8±0.3° 2θ.

140. The composition of any one of claims 137-139, wherein the crystalline form of the compound of Formula (III) is characterized by an x-ray diffraction pattern substantially as set forth in FIG. 56.

141. The composition of any one of claims 95-98 or 100-101, wherein the crystalline form of the compound of Formula (III) is Form XIV.

142. The composition of claim 141, wherein the crystalline form of the compound of Formula (III) is characterized by an x-ray powder diffraction pattern comprising at least one peak, at least two peaks, at least three peaks, or at least four peaks selected from 17.5±0.3° 2θ, 15.4±0.3° 2θ, 21.6±0.3° 2θ, and 5.8±0.3° 2θ.

143. The composition of claim 142, wherein the x-ray powder diffraction pattern further comprises at least one peak, at least two peaks, or at least three peaks selected from 16.3±0.3° 2θ, 21.9±0.3° 2θ, and 23.9±0.3° 2θ.

144. The composition of any one of claims 141-143, wherein the crystalline form of the compound of Formula (III) is characterized by an x-ray diffraction pattern substantially as set forth in FIG. 71.

145. The composition of any one of claims 95-98 or 101, wherein the crystalline form of the compound of Formula (III) is Form XV.

146. The composition of claim 145, wherein the crystalline form of the compound of Formula (III) is characterized by an x-ray powder diffraction pattern comprising at least one peak, at least two peaks, at least three peaks, or at least four peaks selected from 20.2±0.3° 2θ, 7.1±0.3° 2θ, 23.4±0.3° 2θ, and 22.4±0.3° 2θ.

147. The composition of claim 146, wherein the x-ray powder diffraction pattern further comprises at least one peak, at least two peaks, or at least three peaks selected from 21.7±0.3° 2θ, 22.7±0.3° 2θ, and 18.8±0.3° 2θ.

148. The composition of any one of claims 145-147, wherein the crystalline form of the compound of Formula (III) is characterized by an x-ray diffraction pattern substantially as set forth in FIG. 6.

149. The composition of any one of claims 95-98 or 101, wherein the crystalline form of the compound of Formula (III) is Form XIX.

150. The composition of claim 149, wherein the crystalline form of the compound of Formula (III) is characterized by an x-ray powder diffraction pattern comprising at least one peak, at least two peaks, at least three peaks, or at least four peaks selected from 9.4±0.3° 2θ, 23.3±0.3° 2θ, 22.3±0.3° 2θ, and 20.1±0.3° 2θ.

151. The composition of claim 150, wherein the crystalline form of the x-ray powder diffraction pattern further comprises at least one peak, at least two peaks, or at least three peaks selected from 19.6±0.3° 2θ, 7.1±0.3° 2θ, and 15.7±0.3° 2θ.

152. The composition of any one of claims 149-151, wherein the crystalline form of the compound of Formula (III) is characterized by an x-ray diffraction pattern substantially as set forth in FIG. 25.

153. A composition comprising a crystalline form of a compound of Formula (III): 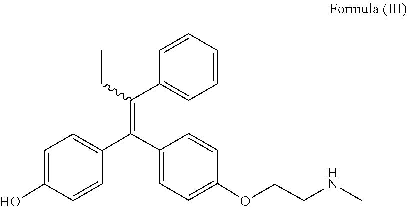 [SMILES: CCC(=C(c1ccc(O)cc1)c1ccc(OCCNC)cc1)c1ccccc1]
wherein the crystalline form of the compound of Formula (III) is Form XVII as characterized by an x-ray diffraction pattern substantially as set forth in FIG. 21.

154. A composition comprising a crystalline form of a compound of Formula (III): 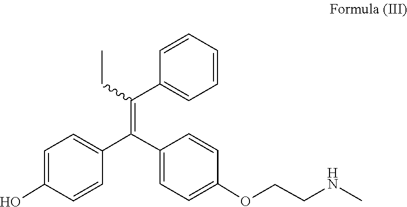 [SMILES: CCC(=C(c1ccc(O)cc1)c1ccc(OCCNC)cc1)c1ccccc1]
wherein the crystalline form of the compound of Formula (III) is Form XVIII as characterized by an x-ray diffraction pattern substantially as set forth in FIG. 23.

155. A method of generating a crystalline form of a compound of Formula (III): 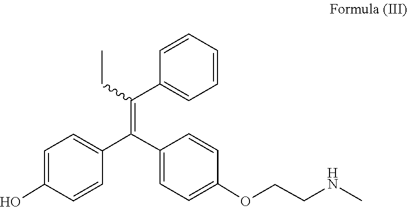 [SMILES: CCC(=C(c1ccc(O)cc1)c1ccc(OCCNC)cc1)c1ccccc1]
the method comprising:
incubating a solid form of the compound of Formula (III) in a solvent system in which at least a portion of the solid form of the compound of Formula (III) remains solid;
converting the portion of the solid form of the compound of Formula (III) to the crystalline form; and
collecting the crystalline form.

156. The method of claim 155, wherein the crystalline form of the compound of Formula (III) is Form I.

157. The method of claim 155 or claim 156, wherein the incubating is for between about 3 and about 120 hours.

158. The method of any one of claims 155-157, wherein the solid form of the compound of Formula (III) comprises at least about 90% isomeric purity prior to the incubating.

159. The method of any one of claims 155-158, wherein the crystalline form of the compound of Formula (III) comprises at least about 90% isomeric purity.

160. The method of any one of claims 155-159, wherein the solid form of the compound of Formula (III) comprises at least one of Forms IV-XIX.

161. The method of any one of claims 155-160, wherein the solid form of the compound of Formula (III) is at least 25%, at least 50%, at least 75%, at least 90%, at least 95%, or at least 99% amorphous.

162. The method of any one of claims 155-161, wherein between about 0.1% and about 75%, between about 0.1% and about 10%, between about 0.1% and about 25%, between about 1% and about 25%, between about 5% and about 50%, between about 20% and about 50%, or between about 25% and about 75% of the solid form of the compound of Formula (III) dissolves in the solvent system.

163. The method of any one of claims 155-162, wherein during the incubating the solvent system comprises a temperature between about 5° C. and about 110° C., between about 5° C. and about 90° C., between about 5° C. and about 25° C., between about 10° C. and about 60° C., between about 25° C. and about 50° C., between about 15° C. and about 50° C., between about 15° C. and about 70° C., between about 25° C. and about 40° C., or between about 35° C. and about 60° C.

164. The method of any one of claims 155-163, wherein the incubating is performed for between about 0.1 and about 700 hours, between about 1 and about 360 hours, between about 3 and about 360 hours, between about 3 and about 36 hours, between about 6 and about 72 hours, between about 12 and about 150 hours, between about 24 and about 150 hours, between about 48 and about 240 hours, or between about 100 and about 700 hours.

165. The method of any one of claims 155-164, wherein the solvent system comprises between about 5 and about 10000 mg/ml, between about 5 and about 1000 mg/ml, between about 5 and about 300 mg/ml, between about 5 and about 50 mg/ml, between about 20 and about 300 mg/ml, between about 50 and about 300 mg/ml, between about 100 and about 1000 mg/ml, or between about 300 and about 10000 mg/ml of the solid form of the compound of Formula (III) during the incubating.

166. The method of any one of claims 155-165, wherein the solvent system comprises 2-propanol, acetonitrile, acetone, butyl acetate, butyl methyl ether, dimethylformamide, ethanol, ethyl acetate, water, heptane, methanol, methyl isobutyl ketone, tetrahydrofuran, toluene, or a combination thereof.

167. The method of any one of claims 155-166, wherein the solvent system comprises a single solvent.

168. The method of any one of claims 155-166, wherein the solvent comprises a plurality of solvents.

169. The method of any one of claims 155-166 or 168, wherein the solvent system comprises two solvents in a ratio of between about 99:1 and about 1:1, between about 19:1 and about 1:1, between about 19:1 and 5:1, between about 8:1 and about 1:1, between about 4:1 and about 1:1, or between about 2:1 and about 1:1.

170. The method of any one of claims 155-169, wherein the solvent system comprises less than about 25%, less than about 10%, less than about 5%, less than about 2%, less than about 1%, or less than about 0.25% water.

171. A method for producing a crystalline form of a compound of Formula (III), the method comprising:
(a) dissolving the compound of Formula (III) in 2-propanol, and evaporating at least a portion of the 2-propanol;
(b) dissolving the compound of Formula (III) in a solvent system comprising 2-propanol and tetrahydrofuran at a first temperature, and cooling the solvent system to a second temperature;
(c) dissolving the compound of Formula (III) in a solvent system comprising heptane and 2-propanol at a first temperature, and cooling the solvent system to a second temperature;
(d) dissolving the compound of Formula (III) in a solvent system comprising 2-propanol and ethyl acetate at a first temperature, and cooling the solvent system to a second temperature;
(e) dissolving the compound of Formula (III) in tetrahydrofuran, and combining the solvent system with water;
(f) dissolving the compound of Formula (III) in a solvent system comprising 2-propanol and tetrahydrofuran, and evaporating at least a portion of the solvent system;
(g) dissolving the compound of Formula (III) in acetone, and evaporating at least a portion of the acetone;
(h) dissolving the compound of Formula (III) in a solvent system comprising acetone and tetrahydrofuran, and evaporating at least a portion of the solvent system; or
(i) a combination thereof;
thereby producing the crystalline form of the compound of Formula (III).

172. The method of claim 171, comprising any one of (a)-(f) or (h).

173. The method of claim 171 or claim 172, wherein the crystalline form of the compound of Formula (III) is Form IV.

174. The method of any one of claims 171-173, wherein the cooling is at a rate of between about 0.02° C./minute and about 10° C./minute.

175. The method of any one of claims 171-174, wherein the first temperature is between about 20° C. and about 100° C., and the second temperature is between about −20° C. and about 25° C.

176. A method for producing a crystalline form of a compound of Formula (III), the method comprising:
dissolving the compound of Formula (III) in acetonitrile at a first temperature, and cooling the acetonitrile to a second temperature, thereby producing the crystalline form of the compound of Formula (III).

177. The method of claim 176, wherein the crystalline form of the compound of Formula (III) is Form V.

178. The method of claim 176 or claim 177, wherein the cooling is at a rate of between about 0.02° C./minute and about 10° C./minute.

179. A method for producing a crystalline form of a compound of Formula (III), the method comprising: storing Form V at a temperature of between about 20° C. and about 50° C., thereby producing the crystalline form of the compound of Formula (III).

180. The method of claim 179, wherein the crystalline form of the compound of Formula (III) is Form VI.

181. A method for producing a crystalline form of a compound of Formula (III), the method comprising:
(a) dissolving the compound of Formula (III) in ethyl acetate, and adding the ethyl acetate to heptane;
(b) dissolving the compound of Formula (III) in toluene, and evaporating at least a portion of the toluene;
(c) dissolving the compound of Formula (III) in toluene, and adding the toluene to heptane;
(d) dissolving the compound of Formula (III) in a solvent system comprising heptane and toluene, and heating the solvent system to between about 20° C. and about 50° C.; or
(e) a combination thereof,
thereby producing the crystalline form of the compound of Formula (III).

182. The method of claim 181, wherein the crystalline form of the compound of Formula (III) is Form VII.

183. A method for producing a crystalline form of a compound of Formula (III), the method comprising:
(a) dissolving the compound of Formula (III) in toluene at a first temperature, and cooling the solvent system to a second temperature;
(b) dissolving the compound of Formula (III) in toluene, adding butyl methyl ether to the toluene to form a solvent system, and evaporating at least a portion of the solvent system; or
(c) a combination thereof;
thereby producing the crystalline form of the compound of Formula (III).

184. The method of claim 183, wherein the crystalline form of the compound of Formula (III) is Form VIII.

185. The method of claim 183 or claim 184, wherein the cooling is at a rate of between about 0.02° C./minute and about 10° C./minute.

186. A method for producing a crystalline form of a compound of Formula (III), the method comprising:
(a) dissolving the compound of Formula (III) in a solvent system comprising ethyl acetate, adding heptane to the solvent system, and incubating the solvent system at a temperature of between about 20° C. and about −20° C.;
(b) dissolving the compound of Formula (III) in a solvent system comprising butyl acetate, adding heptane to the solvent system;
(c) dissolving the compound of Formula (III) in ethyl acetate, adding the ethyl acetate to butyl methyl ether to form a solvent system, and evaporating at least a portion of the solvent system;
(d) dissolving the compound of Formula (III) in ethyl acetate, adding butyl methyl ether to the ethyl acetate to form a solvent system, and evaporating at least a portion of the solvent system;
(e) dissolving the compound of Formula (III) in acetone, combining the acetone with butyl methyl ether to form a solvent system, and evaporating at least a portion of the solvent system; or
(f) a combination thereof;
thereby producing the crystalline form of the compound of Formula (III).

187. The method of claim 186, wherein the crystalline form of the compound of Formula (III) is Form IX.

188. A method for producing a crystalline form of a compound of Formula (III), the method comprising:
dissolving the compound of Formula (III) in 4-methyl-2-pentanone at a first temperature, and cooling the 4-methyl-2-pentanone to a second temperature, thereby producing the crystalline form of the compound of Formula (III).

189. The method of claim 188, wherein the crystalline form of the compound of Formula (III) is Form X.

190. The method of claim 188 or claim 189, wherein the cooling is at a rate of between about 0.02° C./minute and about 10° C./minute.

191. A method for producing a crystalline form of a compound of Formula (III), the method comprising:
dissolving less than about 100 micrograms of the compound of Formula (III) in tetrahydrofuran, adding the tetrahydrofuran to acetone to form a solvent system, and evaporating at least a portion of the solvent system, thereby producing the crystalline form of the compound of Formula (III).

192. The method of claim 191, wherein the crystalline form of the compound of Formula (III) is Form XII.

193. A method for producing a crystalline form of a compound of Formula (III), the method comprising:
dissolving greater than about 100 micrograms of the compound of Formula (III) in tetrahydrofuran, adding the tetrahydrofuran to acetone to form a solvent system, and evaporating at least a portion of the solvent system, thereby producing the crystalline form of the compound of Formula (III).

194. The method of claim 193, wherein the crystalline form of the compound of Formula (III) is Form XIV.

195. A method for producing a crystalline form of a compound of Formula (III), the method comprising:
dissolving the compound of Formula (III) in ethyl acetate, and adding the ethyl acetate to heptane, thereby producing the crystalline form of the compound of Formula (III).

196. The method of claim 195, wherein the crystalline form of the compound of Formula (III) is Form XV.

197. A method for producing a crystalline form of a compound of Formula (III), the method comprising:
(a) dissolving the compound of Formula (III) in acetone at a first temperature, combining the acetone with heptane to form a solvent system, and cooling the solvent system to a second temperature;
(b) dissolving the compound of Formula (III) in tetrahydrofuran at a first temperature, combining the tetrahydrofuran with heptane to form a solvent system, and cooling the solvent system to a second temperature; or
(c) a combination thereof;
thereby producing the crystalline form of the compound of Formula (III).

198. The method of claim 197, wherein the crystalline form of the compound of Formula (III) is Form XIX.

199. The method of claim 197 or claim 198, wherein the cooling is at a rate of between about 0.02° C./minute and about 10° C./minute.

200. A method for producing a crystalline form of a compound of Formula (III), the method comprising:
dissolving the compound of Formula (III) in ethyl acetate at a first temperature, combining the ethyl acetate with a heptane to form a solvent system, and cooling the solvent system to a second temperature, thereby producing the crystalline form of the compound of Formula (III).

201. The method of claim 200, wherein the crystalline form of the compound of Formula (III) is Form XVI.

202. The method of claim 200 or claim 201, wherein the cooling is at a rate of between about 0.02° C./minute and about 10° C./minute.

203. A method for producing a crystalline form of a compound of Formula (III), the method comprising:
dissolving the compound of Formula (III) in ethanol, and evaporating at least a portion of the ethanol, thereby producing the crystalline form of the compound of Formula (III).

204. The method of claim 203, wherein the crystalline form of the compound of Formula (III) is Form XVII.

205. A method for producing a crystalline form of a compound of Formula (III), the method comprising:
dissolving the compound of Formula (III) in tetrahydrofuran, and evaporating at least a portion of the tetrahydrofuran, thereby producing the crystalline form of the compound of Formula (III).

206. The method of claim 205, wherein the crystalline form of the compound of Formula (III) is Form XVIII.
